(19) United States
(12) Patent Application Publication
(10) Pub. No.: US 2017/0368373 A1
(43) Pub. Date: Dec. 28, 2017

(54) Device and Methods for Broadbeam and Microbeam Chemo-Radiosurgery Combined with Its Tumor Exosome Apheresis

(71) Applicant: Velayudhan Sahadevan, Beckley, WV (US)

(72) Inventor: Velayudhan Sahadevan, Beckley, WV (US)

(21) Appl. No.: 15/189,200

(22) Filed: Jun. 22, 2016

(51) Int. Cl. A61N 5/10; A61M 1/34; A61M 1/36; A61G 10/00

(52) U.S. Cl. CPC A61N 5/1084; A61N 5/1067; A61G 10/00; A61M 1/3496; A61M 1/3693; A61N 5/1081; A61M 1/3615; A61N 5/1045; A61N 2005/1088; A61N 2005/1089; A61N 2005/109; A61N 2005/1094

(57) ABSTRACT

Conventional single fraction 20-Gy broadbeam photonbeam or protonbeam chemo-radiosurgery does not sterilize EMT-MET cancer stem cell radiodurans but single fraction 100 to 10,000 Gy microbeam radiosurgery sterilizes them. Device and methods for microbeam chemo-radiosurgery including 250 MeV wakefield electronbeam is disclosed.

Surgery, chemotherapy and broadbeam and microbeam radiosurgery releases billions of abscopal metastasis causing, tumor specific plasma soluble proteins, cell membranes, apoptotic bodies, DNA and RNAs, exosomes like telomere-telomerase, ATM-ATM kinase and others. They and adaptive resistance to chemo-radiosurgery, paraneoplastic and non-paraneoplastic diseases causing immune complexes are removed by pulse flow combined continuous flow ultracentrifugation apheresis and immune affinity chromatography. Chemotherapy and high dose radiation exposed tumor cells and their exosomes are made sensitive to telomerase inhibiting and apoptosis inducing and least toxic epigallocatechin and to heparin bound receptors. They convert triple negative breast tumors into receptor positive tumors which open new avenues for treating most aggressive breast cancers.

### FIELD OF INVENTION

x-ray beam therapy, class 378, 424, 530

### FEDERALLY SPONSORED RESEARCH

None;

### SEQUENCE LISTING

Table of Contents attached

### 1. BACKGROUND OF THE INVENTION

Curative cancer treatment either by surgery, chemotherapy or by conventional radiation therapy is seldom achieved. Such treatments release large quantities of cancer cells and their sub fragments that are not removed after the treatment. They cause abscopal metastasis. Conventional fractionated chemotherapy does not cure most tumors and they are prohibitively expensive. Likewise, conventional radiation therapy and radiosurgery with electron, photon, proton, and carbon ion are also prohibitively expensive. The high cost of advanced radiation therapy is partly due to a few millions to 50 to 100 million costing machines and the need to construct expensive facilities to house such machines. The total cost a present day ordinary radiation therapy center is in the range of about 5 millions. The cost of a center with proton machine is in the range of over 100 to 200 millions. The cost for a carbon ion radiation therapy center is much higher.

There is an acute need for low-cost, affordable total curative cancer treatment that also include removal of cellular fragments and the billions of subcellular fragments released from the cancer cells after surgery, chemotherapy or radiation therapy. The device and methods for interventional pulse flow apheresis combined with immunoadsorption as described in this invention removes circulating tumor cells and its macro and micro molecules. The Interventional continuous flow ultracentrifugation apheresis also as described in this invention removes the billions of subcellular fragments released from the cancer cells after surgery, chemotherapy or radiation therapy. It avoids and or minimizes the abscopal metastasis after cancer treatments.

A single, relatively inexpensive laser wakefield accelerator alone or combined with a Z-pinch or dielectric waveguide or corrugated waveguide system capable of generating five to ten collinear simultaneous beams as described in this invention is an affordable low cost system for single fraction kGy radiosurgery. These beams are transported to a ten to twenty kGy microbeam, nanobeam or minibeam radiosurgical rooms equipped with multiple tissue equivalent collimator systems for all field simultaneous kGy radiosurgery within seconds. The unwanted secondary radiation including neutron and gamma radiation are absorbed by the tissue equivalent collimator systems. Such centralized cancer centers have the capability to treat about 200 patients a day as single fraction kGy radiosurgery. In a year with 250 working days such a radiation therapy center treats 200×250 patients that are 50,000 patients in a year. It brings down the cost for the technical component. Such microbeam generating systems and others like with electron, photon, proton and carbon ion microbeam generating systems are described in this invention.

Single fraction microbeam kGy radiosurgery will lead to adaptive resistance avoiding, cancer stem cell ablating curative rather than the present response based cancer treatments (1). Like several species of radioresistant bacteria, there are cancer stemcells with extreme resistance to radiation and chemotherapy. Survival of Deinococcus radiodurans is not much affected by 16 kGy Cobalt-60 radiations. Hence it is suggested that damage to DNA repair protein in response to radiation is better related to its survival than the classical DNA damage in response to radiation (2). Most tumors contain a small portion of invisible cancer stem cells that are resistant to conventional chemotherapy and or radiation. They will survive from radiation induced stress more than the differentiated cancer cells (3). The histone H2A phosphorylation, the most readily recognizable marker for DNA double strand breaks is markedly reduced in Cancer Stem cell after radiation than in the differentiated cancer cell (4). Cancer stems cells in solid tumors are resistant to conventional cancer treatments (5), say it is radiation therapy or chemotherapy. The glioblastoma and colon carcinoma cancer cell surface marker CD 133+ is more enriched than in differentiated cancer cells. In glioblastoma, there is a three to four fold increase in CD 133+ cells immediately after radiation. This indicates that the surviving cancer cells after radiation injury have relatively higher number of cancer stem cells (6). After radiation, the surviving CD 133+ cells in glioblastoma are capable of proliferation just like the non-radiated glioblastoma cells (7).

It is an evidence for stem cell's capacity for repair after radiation injury. In glioblastomas, the degree of DNA damage caused by radiation in CD 133+ and CD 133− cells are the same but the CD 133+ cells repairs the DNA damage more efficiently than in CD 133− cells indicating its adaptive radiation resistance (8) and rapid recovery from radiation induced injuries. The cancer stem cells are programmed to withstand the stress caused by radiation. The presence of basal level of activation of DNA damage check point, rad 17, in CD 133+ cells also indicates its adaptive radioresistance. The accelerated repopulation of cancer cells, tumor recurrence and metastasis after radiation all are associated with cancer stem cell recovery after radiation which is associated with rapid DNA repair mediated by DNA damage repair protein. Such adaptive response to radiation inducing proteins includes telomerase, poly(ADP-ribose) polymerase-1 (PARP), insulin-like growth factor-1-secretory clusterin, DNA-PK complex and DNA-PK subunit Ku, phosphatases, gamma secretase, Wee-1, small molecule c-Met, tyrosinekinases, RcQ helicase, terminal deoxynucleotidyl transferase (TdT), DNA-Polymerase X-Family, shRNA and SiRNA and so many other protein enzymes. Proteins are more resistant to radiation damage than to DNA. In bacteria like the Deinococcus radiodurans, the damaged DNA is rapidly repaired by these radiation damage repair proteins and the cell survival is rapidly restored even at kGy doses of radiation. The radiation resistance in cancer stem cell has some similarities to those in Deinococcus radiodurans. Since these enzymatic proteins are highly radioresistant, the cancer stem cell DNA damage induced by low dose radiation is rapidly repaired and they survives low dose radiation like the bacteria species including the Deinococcus radiodurans survives.

Compared to daily fractionated radiation therapy at 1.8 to 2.25 Gy dose range, in fewer fractions stereotactic body radiation therapy (SBRT) and stereotactic radiosurgery (SRS) are generally practiced with 20 to 22 Gy dose fractions. Even after three fractions of 20 to 22 Gy SBRT, the median survival rate for early stage, small T1-T2 non small cell lung cancer (NSCLC) is only 42% at 50 months (9). A 3 cm T1 NSCLC tumor weighs about 15 g. A 5 cm T2 NSCLC tumor weighs about 25 g. Sixty-nine Gy completely ablates all the visible malignant cells in a 1 to 10 g tumor (10). If the tumor weight were 10 g, then (69/10), that is 6.9 Gy ablates all the visible malignant cells in a 1 g tumor and 6.9×15, that is a single fraction 103.5 Gy broadbeam ablates all the visible malignant cells in a T1 tumor weighing about 15 g. Likewise, a single fraction 172.5 Gy broadbeam ablates all the visible malignant cells in a T2 tumor weighing about 25 g. Therefore, 60 and 66 Gy broadbeam SBRT do not ablate all the malignant cells in a T1-T2 NSCLC. This accounts for the low 42% overall survival rate at 50 months for patients with T1-T2 NSCLC who were treated with 3 fractions of 20 or 22 Gy broad beam SBRT (9). Under these conditions, invisible EMT/MET cancer stem cells survives.

There are differences between the biological effectiveness of photon broadbeam and microbeam. It is reported that 3.4 to 4.4 Gy broadbeam is equivalent to 112 Gy microbeams (11). Hence, the 69 Gy broadbeam needed to ablate all the visible malignant cells in a 1-10 g tumor (8) is equivalent to 69 divided by the average of 3.4 to 4.4, that is 3.9 times 112, which is 1,981 Gy microbeam, or about 2,000 Gy microbeam. EMT/MET cancer stem cells are five or more times radioresistant than the differentiated cancer cells. Hence about 10,000 Gy microbeam will be required for curative total ablation of malignant cells, including highly radioresistant phenotypic EMT/MET cancer cells and cancer stem cells in T1-T2 NSCLC tumors weighing 15 to 25 g. Most often, an average-sized tumor will weigh 100 g or more. Hence, 30-50 kGy microbeam radiation is required for curative SBRT and SRS of an average-sized tumor.

### 2. MICROBEAM RADIOSURGERY

Microbeam radiosurgery (MRS) at doses ranging from 200 to 4,000 Gy and at dose rate of 16,000 Gy per second is shown to be safe to destroy the caudate nucleus in rat without damaging the normal tissue (12). In several animal experiments, the microbeam radiation therapy has shown its efficacy to treat most radioresistant tumors like the glioblastoma multiforme (13) and aggressive murine SCCVII squamous cell carcinoma (14).

According to American Cancer Society's 2014-2015 report, the estimated combined male and female cancer survival were only 24, 16, 10, 6, 4 and 5 percent at 5, 10, 15, 20, 25 and 30 years (15). The remaining 76, 84, 90, 94, 96 and 95 percent did not survive at 5, 10, 15, 20, 25 and 30 years. During these past years, we have made drastic technological advancements that are suitable for the much needed new approach in cancer treatment that is affordable for patients from everywhere. Surgery cannot eradicate invisible EMT/MET cancer stem cells. Adaptive resistance to radiation and chemotherapy is initiated immediately after the very first fractionated treatments. With fractionated treatments, the tumor becomes increasingly radio- and chemoresistant, responding temporarily as arrested tumor growth and remission. It is followed by tumor recurrence and metastasis. Prolonged chemotherapy becomes ineffective due to adaptive resistance. Single fraction chemotherapy is not feasible due to its severe toxicity. On the other hand, safe, total cancer and cancer stem cell ablative innovative single fraction kGy radiosurgery is feasible. Although the advantage of microbeam radiosurgery for curative cancer treatment was described over 22 years ago (16), it is still not in clinical practice due to difficulties associated with its clinical implementation. In this invention, a much different device and methods than those used in the past for microbeam, nanobeam or minibeam radiosurgery is disclosed. The device and method disclosed in this invention for single fraction, normal tissue-sparing, total EMT/MET cancer stem cell ablative kGy microbeam SBRT and SRS without adaptive resistance and least normal tissue toxicity for more curative cancer treatment is summarized below.

### 3. MAGNETICALLY FOCUSED VERY HIGH ENERGY PENCILBEAM, MICROBEAM AND NANOBEAM HAVING PROTON LIKE PENCILBEAM, MICROBEAM AND NANOBEAM WITH DEEP SKIN PENETRATION AND WITH LEAST SKIN TOXICITY

The magnetically focused electron beam's penetration below the skin is much deeper. Its maximum dose, the dₘₐₓ is much deeper in the skin than those for the unfocused electron beam (17. 18). It removes the unfocused electron beam's higher rate of toxic reaction within the skin surface and in tissue just below it. The severe errythema, edema, pain and ulceration from unfocused conventional high dose electron beam radiation to the skin are avoided by such magnetically focused and high energy electron beam radiation therapy. It is very important for safe administration of single or hypofraction kGy radiosurgery with electron. Furthermore, such magnetically focused and high energy electron beams have much less penumbra. It avoids smearing of the adjacent microbeam's base by each other. It creates much better defined low dose valley region. It helps to heal the normal tissue through which the microbeam, nanobeam or minibeam travels towards an isocentric tumor. The radiation damage in normal tissue is repaired by proliferation of its normal stem cells. The Monte Carlo simulation of 150-250 MeV electron beam has less lateral penumbra and its depth dose at less than 10 cm is similar to photon beam and its practical range Rₚ is greater than 40 cm (37). Combined with magnetic focusing of the very high energy electron pencil beam (17, 18), microbeam, nanobeam and minibeam and the very high energy electron beam's deeper penetration (37) the electron pencil beam, microbeam, nanobeam and minibeam are made like proton pencil beam microbeam, nanobeam and minibeam with less skin toxicity. With sharp well defined microbeam, nanobeam and minibeam without lateral penumbra keeps the peak and valley doses without spreading and smearing with each other. It assures the valley region stem cell's regeneration and migration to peak dose region intact after microbeam, nanobeam or minibeam radiosurgery.

### 4. CHEMOTHERAPY AND RADIOSURGERY INDUCED SURGE OF DNA DSB, TELOMERE—TELOMERASE, EXOSOMES, NUCLEOSOMES AND MICROSOMES AND INCREASED ATM—ATM KINASE ACTIVITY

With kGy radiosurgery, there is complete tumor ablation, including the cancer and the cancer stem cell chemo-radiodurans. It eliminates the primary tumor. However, it releases large quantities tumor associated microsomes, apoptotic bodies, nucleosomes and exosomes locally and into the circulation.

There are already 4,000 known biologically active and purified exosome proteins. It also releases apoptotic bodies and nucleosomes containing telomere—telomerase and ATM—ATM kinase.

The DNA DSB caused by radiation is rapidly repaired by ATM kinase. In the absence of ATM kinase, radiosensitivity is increased as in patients with ataxia telangiectasia. In the presence of ATM kinase the radioresistance and also the chemoresistance are increased due to rapid DNA repair; hence the sublethal damage from radiation is rapidly repaired. In other words, the cancer cells are protected from treatments like radiation and chemotherapy in the presence of spontaneously increased ATM kinase activity. It also causes billions of exosome, nanosomes and microsomes release into the circulation particularly after kGy microbeam, nanobeam or minibeam radiosurgery. After 12 Gy fractions of stereotactic body radiation therapy (SBRT), the ATM kinase in peripheral blood monocytes (PBMC) is significantly increased. It is a good example for the bystander and abscopal effect of higher dose radiation. At low dose radiation as with the conventional radiation therapy and radiosurgery, the DNA damage is repaired by the ATM-telomerase activities. With kGy radiosurgery, both the tumor cell and the cancer stem cell chemo-radiodurans are almost completely ablated. However, it releases also large quantities of tumor exosomes, nanosomes and microbeams into the circulation. It leads to the repair of tumor DNA like the DNA repair after kGy radiation to Deinococcus radiodurans. If these tumor exosomes, nanosomes and microsomes are not removed, it leads to increased local tumor associated bystander effects and the distant abscopal metastasis. To overcome the DNA damage repair in cancer stem cell after kGy microbeam, nanobeam of minibeam radiation, a modified version of previously described pulse flow apheresis (61) is implemented in this invention. Circulating tumor associated nucleosomes are increased after radiation therapy (62). It is also removed by pulse flow apheresis.

Radiation damage causes DNA unwinding and its topological exposure as a different form of histone H2A, the H2AX. It is a substrate for ATM. H2AX is phosphorylated by active ATM at serine 139 which is referred as γ-H2AX. It is a docking station for several DNA damage proteins. It includes the MRN (MRE11, RAD50 and NBS1), MDC1, RPA, RAD51, RAD52, RAD54, BRCA1 and BLM1. These exosomes, nanosomes and microsomes are also bystander effect causing cellular elements released from the tumor in response to radiation/chemotherapy and surgery. They are carried to every tissue in the body. Circulating cell free DNA is associated in nearly all kind of cancers (63) The kGy gamma or electron beam radiation shatters the DNA into small fragments (64) Presence of focal nuclear and peripheral cytoplasmic staining for γ-H2AX after chemotherapy with platinum or topotecan in circulating tumor cell (65,66) is an indication for the release of these nucleosomes. There is a time and dose dependent γ-H2AX increase which is measured by flow cytometry (67).

Radiation causes molecular rearrangements in the exosomes. It modulates the connective tissue growth factor (CTGF) which induces cell migration. These exosomes have increased IGF binding protein. It activates focal adhesion kinase, Paxillin, proto-oncogene tyrosine kinase (Src) and neurotrophic tyrosine kinase receptor type 1 (TrkA). Exosomes are capable of delivering its payloads both locally and to distant sites causing increased CTGF activity leading to tumor specific epithelial mesenchymal (EMT) and mesenchymal epithelial transformation (MET) associated tumor recurrence and metastasis (88).

Circulating exosomes carrying the miRNAs when transferred to tumor cells, tumor phenotype could be modified. Modulation of miR-503 in breast cancer cells alters its proliferative and invasive capabilities. Heat shock proteins, HSP 60, HSP 70 and HSP90 containing exosomes released by the hepatocellular carcinoma in response to chemotherapeutic stress can make the liver cancer chemoresistant.

### 5. SYSTEMIC CANCER IMMUNITY AFTER kGy RADIOSURGERY

The kGy radiosurgery causes severe DNA damage. DNA damage based apoptosis is primarily controlled by CD95 (Fas/APO-1) and its ligand, CD95L. CD 95L in the tumor infiltrating lymphocytes controls the immune response to cancer. Like the immune lymphocytes kill the virus and other pathogens, the immune lymphocyte kills the cancer cells.

### 6. IMMUNOSUPPRESSION BY LOW DOSE RADIATION AND TUMOR IMMUNITY

In conventional fractionated radiation therapy, the daily radiation lasting for about 8 to 10 weeks wipes out the innate local cancer immunity. The daily fractionated radiation therapy eliminates most of the immunity processing cells, the mast cells, phagocytes, natural killer cells, γδ T cells, macrophages, neutrophils, dendritic cells, basophiles and eosinophils. After each day's radiation, these cells attempts to repopulate the locally irradiated tumor but by the next day's radiation, they are destroyed or damaged making them ineffective to help to establish an effective innate and adoptive immunity against the tumor that is treated. In this case, the daily fractionated localized radiation acts as a local immunosuppressive treatment. If on the other hand, the radiation is given as a single fraction, split second duration normal tissue sparing microbeam and nanobeam radiation as in this invention, its tissue inflammatory reaction induced stress defense is called for. It leads to recovery from the injury and acquiring protection against such future injury through activation of innate and adaptive immunity to tumor that is so treated.

### 7. SINGLE FRACTION kGy MICROBEAM RADIOSURGERY INDUCED INFLAMMATION, CYTOKINES SECRETION AND SYSTEMIC TUMOR IMMUNITY

The kGy parallel microbeam radiosurgery to a tumor in split seconds is associated with inflammation at the tumor site. It releases a number of cytokines. Even the lower dose localized radiation in the range of 5 cGy to 2 Gy evokes localized innate immunity. The kGy radiation in split second obviously evokes much stronger innate immunity and secretion of a number of cytokines. Likewise, radiation evokes adoptive immunity through the FAS pathway. In vitro experiments, MC 38 adenocarcinoma cells at 20 Gy dose has increased FAS activity at molecular, phenotypic and functional levels. Higher dose radiation sensitizes the tumor cells to antigen specific cytotoxic-T-lymphocyte's (CTLs) FAS/FAS ligand pathway. In vivo experiments, the same MC 38 adenocarcinoma cells growing subcutaneously also show 8 Gy radiation sensitization and CTL adoptive immunity by up regulation of FAS leading to tumor growth arrest and tumor rejection. Antigen processing dendritic cells are stimulated by radiated highly malignant prostate cancer cells but only at high doses, in the range of 10-60 Gy. Unirradiated cells have no such immunostimulatory effects.

### 8. PULSE FLOW APHERESIS OF CHEMOTHERAPY, RADIOSURGERY AND CHEMO-RADIOSURGERY INDUCED RELEASE OF LARGE QUANTITIES OF TELOMERASE, CIRCULATING TUMOR CELLS, APOPTOTIC BODIES, MICROSOMES, EXOSOMES AND NANOSOMES

Radiation induced sudden burst of tumor associated exosomes increase in circulation can be a cause of metastasis. Radiation causes molecular rearrangements in the exosomes. It modulates the connective tissue growth factor (CTGF) which induces cell migration. These exosomes have increased IGF binding protein. It activates focal adhesion kinase, Paxillin, proto-oncogene tyrosine kinase (Src) and neurotrophic tyrosine kinase receptor type 1 (TrkA). Exosomes are capable of delivering its payloads both locally and to distant sites causing increased CTGF activity leading to tumor specific mesenchymal epithelial transformation, tumor recurrence and metastasis.

Circulating exosomes carrying the miRNAs when transferred to tumor cells, tumor phenotype could be modified. Modulation of miR-503 in breast cancer cells alters its proliferative and invasive capabilities. Heat shock proteins, HSP 60, HSP 70 and HSP90 containing exosomes released by the hepatocellular carcinoma in response to chemotherapeutic stress can make the liver cancer chemoresistant. The sudden release of so much exosomes from the tumor into the circulation in response to radiation and or chemotherapy can cause unintended side effects due to treatment and resulting in tumor recurrence and metastasis.

With kGy radiosurgery, there is complete tumor ablation, including the cancer and cancer stem cell radiodurans. It eliminates the primary tumor. However, it releases large quantities of tumor associated apoptotic bodies, nucleosomes, exosomes and nanosomes locally and into the circulation. There are already about 4,000 known biologically active and purified exosome proteins. It also releases apoptotic bodies and nucleosomes containing telomere—telomerase and ATM—ATM kinase. These exosomes, nanosomes and microsomes are also bystander and abscopal effect causing cellular elements. They are carried to every tissue in the body. One of this nucleosome, the γ-H2AX is an excellent marker to follow up the DNA damage repair. It is also a docking station for several DNA damage repair protein enzymes. It includes MRN (MRE11, RAD50 and NBS1), MDC1, RPA, RAD51, RAD52, RAD54, BRCA1 and BLM1. The MRN complex is an important enzyme complex in DSB repair and telomere telomerase function (60) In this invention, these subcellular components of the tumor cells released after radiosurgery and chemotherapy is removed by pulse flow centrifugation combined with DNA affinity chromatography followed by ultracentrifuge therapeutic apheresis and plasmapheresis.

The pulse flow apheresis combined with affinity chromatography is used to remove the CTCs and most of the apoptotic bodies, microsomes, nucleosomes and exosomes release after chemotherapy, radiosurgery and chemo-radiosurgery but it leaves still present circulating cell debris, cell membranes, and plasma soluble normal tissue derived and tumor tissue derived apoptotic bodies, DNA and RNAs, microsomes, exosomes and nanosomes, telomere and telomerase, ATM and ATM kinase. These tumor derived micro and nanosomes causes bystander effects and abscopal metastasis. They are removed by therapeutic continuous flow ultracentrifugation plasmapheresis.

### 9. OLD CONTINUOUS FLOW BLOOD SEPARATOR

Alternative to pulse flow apheresis, the old continuous flow blood separator could be used for apheresis (95) but it has many disadvantages for the separation and removal of circulating cell debris, cell membranes, and plasma soluble normal tissue derived and tumor tissue derived apoptotic bodies, DNA and RNAs, microsomes, exosomes, nanosomes, telomere and telomerase, ATM and ATM kinase. The old continuous flow blood separator could be used to separate the white blood cells, platelets and the plasma but not white blood cell and the platelets bound exosomes and nanosomes. It does not separate the plasma soluble normal tissue derived and tumor tissue derived apoptotic bodies, DNA and RNAs, microsomes, exosomes, nanosomes, telomere and telomerase, ATM and ATM kinase efficiently. However, it has been used to remove plasma soluble antigen antibody complex and to overcome chemotherapy resistance as in a case of neuroblastoma (95B) in which pre-plasmapheresis chemotherapy showed no increased VMA secretion and post-plasmapheresis highly increased VMA secretion as an indication of chemoresistance elimination. Still, since it does not remove the millions of tumor exosomes and nanosomes, it was proven to be ineffective for plasmapheresis combined chemotherapy. Post pulse flow plasmapheresis and white cells and platelets apheresis and continuous flow ultracentrifuge apheresis of tumor tissue derived apoptotic bodies, DNA and RNAs, microsomes, exosomes, nanosomes, telomere and telomerase, ATM and ATM kinase combined with chemotherapy and radiosurgery overcomes the chemoresistance and the radioresistance.

### 10. THERAPEUTIC ULTRACENTRIFUGATION PLASMAPHERESIS TO REMOVE CIRCULATING TUMOR DERIVED TELOMERE, TELOMERASE, DNA/RNAs, NUCLEOSOMES AND EXOSOMES AND CELL DEBRIS AFTER SURGERY, CHEMOTHERAPY, RADIOSURGERY AND CHEMO-RADIOSURGERY

The plasma purified by pulsed flow plasmapheresis combined with affinity adsorption columns is either returned to the patient or it is diverted to a continuous flow ultracentrifuge for additional sucrose density gradient (SDG) ultracentrifugation for further removal of the circulating plasma soluble normal cell and tumor cell derived cell debris, cell membranes, and tumor associated proteins, apoptotic bodies, DNA and RNAs, microsomes, exosomes and nanosomes, telomere and telomerase, ATM and ATM kinase that could cause bystander effects and abscopal metastasis. The telomerase surge after the therapeutic intervention needs to be managed to control the tumor DNA damage repair after the therapeutic intervention to inhibit adaptive tumor growth. The very high level of plasma soluble tumor derived micro particles, telomere and telomerase and damaged DNA/RNA after high dose radiation/radiosurgery and combined radiation and radiosurgery are removed by therapeutic continuous flow ultracentrifugation plasmapheresis combined with affinity chromatography.

The principle of continuous flow ultracentrifugation was pioneered at the Oak Ridge National Laboratory for the U.S Atomic Energy Commission under the Molecular Anatomy Program (The MAN Program) and was cosponsored by the National Cancer Institute (NCI), the National Institute of General Medical Sciences (NIH), the National Institute of Allergy and Infectious Diseases (NIAID) and the U.S. Atomic Energy Commission and the MAN was conducted under the leadership of N. G. Anderson over 50 years ago (94). It is widely used in vaccine preparation against viruses (89) and in micro and nanoparticle cellular research. Although it is an ideal tool for therapeutic plasmapheresis to remove tumor specific plasma soluble ell debris, cell membranes, tumor derived proteins, apoptotic bodies, DNA and RNAs, microsomes, exosomes and nanosomes, telomere and telomerase, ATM and ATM kinase especially to treat their surge after therapeutic intervention like present and future high dose radiosurgery alone or high dose radiosurgery combined with chemotherapy. Continuous flow cell centrifuge plasmapheresis without ultracentrifugation is used safely to treat a variety of diseases but they are incapable of removing nanometer sized tumor derived particles (95) since its centrifugation speed is only about 1,500 rpm (97) while the nanoparticles like the size of a virus is removed at 40,000 rpm at 100,000 G (98). Although the continuous flow ultracentrifugation technology has richly developed for virus research and vaccine preparation (89, 94), it is not yet in use for routine therapeutic clinical applications especially for nanoparticle plasmapheresis as part of cancer treatment. Such treatments decrease the abscopal metastases. The principles of continuous flow ultracentrifugation of N. G Anderson (94) were originated from the NCI, NIH and U.S. Atomic Energy Commission half a centaury ago; it is high time for its routine clinical use for more curative cancer treatment. Anderson had the foresight even for its use in immunotherapy of cancer, he and his colleagues showed that an adenovirus membrane could effectively immunize against tumor growth (96).

In this invention, the plasma after the pulsed flow combined with affinity chromatography is either returned to the patient or diverted to a continuous flow ultracentrifuge for additional SDG ultracentrifugation plasmapheresis of micro and nano particles followed by immune-affinity chromatography that remaining tumor derived plasma soluble ell debris, cell membranes, tumor specific proteins, apoptotic bodies, DNA and RNAs, microsomes, exosomes and nanosomes, telomere and telomerase, ATM and ATM kinase.

### 11. FDA APPROVED THERAPEUTIC MONOCLONAL ANTIBODIES AND Table 3

| | Tumor Specific | AFM | |
|---|---|---|---|
| No | Antibody | Imaging | Target Cancer |
| 1 | Trastuzumab | x | Breast Cancer |
| | (Herceptin) | | Gastric Cancer |
| | Humanized IgG1 | | |
| 2 | Bevacizumab | x | Colorectal Cancer |
| | (Avastin) | | Non-Small Cell |
| | Humanized IgG1 | | Lung Cancer |
| | | | Glioblastoma, |
| | | | Renal Cell Cancer |
| 3 | Cetuximab | x | Head and Neck |
| | (Erbitux) | | Colorectal |
| | Chimeric/human/murine | | |
| | IgG1 | | |
| 4 | Panitumumab | x | Metastatic |
| | (Vectibix) | | Colorectal cancer |
| | Human IgG2 | | |
| 5 | Ipilimumab | x | Metastatic |
| | (Yervoy) | | Melanoma |
| | IgG1 | | |
| 6 | Rituximab | x | Non-Hodgkin's |
| | (Rituxan and | | Lymphoma |
| | Mabthera) | | CLL |
| | Chimeric/human/murine | | Follicular CD20 |
| | IgG1 | | positive Non- |
| | | | Hodgkin's |
| | | | Lymphoma |
| 7 | Alemtuzumab | x | B-Cell CLL |
| | (Campath) | | |
| | Humanized IgG1 | | |
| 8 | Ofatumumab | x | CLL |
| | (Arzerra) | | |
| | Human IgG1 | | |
| 9 | Gemtuzumab | x | Myeloid leukemia |
| | ozogamicin | | |
| | (Mylotarg) | | |
| | Humanized IgG4 | | |
| 10 | Brentuximab | x | Hodgkin's |
| | vedotin | | lymphoma |
| | (Adcetris) | | Anaplastic NHL |
| | Chimeric IgG1 | | |
| 11 | ⁹⁰Ye-Ibritumob | x | Follicular |
| | Tiuxetan | | lymphoma |
| | (Zevelan) | | |
| | Murine IgG1 | | |
| 12 | ¹³¹I-Tositumomab | x | Low grade, |
| | (Bexxar) | | Follicular |
| | Murine IgG2 | | lymphoma |
| 13 | Vismodegib | x | Skin |
| | (Evrivedge) | | |
| 14 | Everolimus | x | Breast Ca |
| | (Afinitor) | | |
| 15 | Pertuzumab | x | Breast Cancer |
| | (Perjeta) | | |
| 16 | Also-Trastuzumab | x | Breast Cancer |
| | estramustine | | |
| | (Kadcyla) | | |
| 17 | Palbociclib | x | Breast Cancer |
| | (Ibarance) | | |
| 18 | Densosumab | x | Giant Cell Tumor |
| | (Xgeva) | | of the bone |
| 19 | Alitretinoin | x | Kaposi sarcoma |
| | (Paneretin) | | |
| 20 | Sorafinib (Nexavar) | x | Kidney, Thyroid |
| 21 | Sunitinib (Sutent) | x | Kidney, Pancreas |
| 22 | Pazopanib (Votient) | x | Kidney |
| 23 | Temsirolimus | x | Kidney |
| | (Toresel) | | |
| 24 | Everolimus | x | Kidney |
| | (Afinitor) | | |
| 25 | Axitinib (Inlyta) | x | Kidney |
| 26 | Tritionin (Vesanoid) | x | Leukemia |
| 27 | Dasatinib (Sprycel) | x | Leukemia |
| 28 | Nilotinib (Tasigna) | x | Leukemia |
| 29 | Bosutinib (Bosulif) | x | Leukemia |
| 30 | Obinutzumab | x | Leukemia |
| | (Gazyva) | | |
| 31 | Ibrutinib (Ibruvica) | x | Leukemia |
| 32 | Idelalisib (Zydelig) | x | Leukemia |
| 33 | Blinatumomab | x | Leukemia |
| | (Blincyto) | | |
| 34 | Crizotinib (Xalkori) | x | Lung Ca |
| 35 | Gefitinib (Iressa) | x | Lung Ca |
| 36 | Afatinib dimaleate | x | Lung Ca |
| | (Gilotrif) | | |
| 37 | Ceritinib | x | Lung Ca |
| | (LDK378/Zykadia) | | |
| 38 | Ramucirumab | x | Lung Ca |
| | (Cyramza) | | |
| 39 | Denileukin diftitox | x | Lymphoma |
| | (Ontak) | | |
| 40 | vorinostat (Zolinza) | x | Lymphoma |
| 41 | romidepsin | x | Lymphoma |
| | (Istodax) | | |
| 42 | bexarotene | x | Lymphoma |
| | (Targretin) | | |
| 43 | bortezomib | x | Lymphoma |
| | (Velcade) | | |
| 44 | pralatrexate | x | Lymphoma |
| | (Folotyn) | | |
| 45 | lenaliomide | x | Lymphoma |
| | (Revlimid) | | |
| 46 | ibrutinib | x | Lymphoma |
| | (Imbruvica) | | |
| 47 | siltuximab (Sylvant) | x | Lymphoma |
| 48 | belinostat | x | Lymphoma |
| | (Beleodaq) | | |
| 49 | vemurafenib | x | Melanoma |
| | (Zelboraf) | | |
| 50 | trametinib | x | Melanoma |
| | (Mekinist) | | |
| 51 | dabrafenib | x | Melanoma |
| | (Tafinlar) | | |
| 51 | pembrolizumab | x | Melanoma |
| | (Keytruda) | | |
| 52 | nivolumab (Opdivo) | x | Melanoma |
| 53 | Bortezomib | x | Multiple Myeloma |
| | (Velcade) | | |
| 54 | carfilzomib | x | Multiple Myeloma |
| | (Kyprolis) | | |
| 55 | ruxolitinib | x | Myelodysplastic/ |
| | phosphate (Jakafi) | | myeloproliferative |
| | | | disorder |
| 56 | olaparib (Lynparza) | x | Ovarian Cancer |
| 57 | Cabazitaxel | x | Prostate Cancer |
| | (Jevtana) | | |
| 58 | enzalutamide | x | Prostate Cancer |
| | (Xtandi) | | |
| 59 | Pazopanib (Votrien) | x | Soft tissue sarcoma |
| 60 | Cabozantinib | x | Thyroid Cancer |
| | (Cometriq) | | |
| 61 | vandetanib | x | Thyroid Cancer |
| | (Caprelsa) | | |
| 62 | lenvatinib mesylate | x | Thyroid Cancer |
| | (Lenvima) | | |

When the centrifuge comes to a complete stop after the 30 min. centrifugation, the buoyant sediment containing patent specific tumor microparticles, cell membranes, plasma soluble tumor associated proteins, apoptotic bodies, DNA and RNAs, microsomes, exosomes and nanosomes, telomere and telomerase, ATM and ATM kinase is collected from the bottom of the sucrose solution by air injection into the rotor from the top and analyzed for tumor specific elective treatment and unused fractions are saved for future studies. All these steps are done under sterile conditions.

### 12. ALTERNATIVE FRACTIONATED TUMOR SPECIFIC EXOSOME PURIFICATION

Alternatively, the tumor tissue components in the SDG fractions are collected by separate ultracentrifugation.

The cellular debris are centrifuged and removed by preparatory low speed continuous flow ultracentrifugation at 2,000 g for 30 min at 4° C. and analyzed by combined AFM/NTA/DCNA and flow cytometry.

The exosomes, nucleosomes, the cell membrane, DNAs and RNAs, their fragments and the apoptotic vesicles are separated by preparatory low speed continuous flow ultracentrifugation at 12,000 g for 45 min at 4° C. and analyzed by combined AFM/NTA/DCNA and flow cytometry.

The miRNAs, nucleosomes, nucleosomes with ATM and ATM-kinase, exosomes and contaminating proteins are separated by continuous flow ultracentrifugation at 25,000 g for 3 h at 4° C. and analyzed by combined AFM/NTA/DCNA and flow cytometry.

The plasma soluble tumor associated proteins, apoptotic bodies, DNA and RNAs, microsomes, exosomes and nanosomes, telomere and telomerase, ATM and ATM kinase by continuous flow ultracentrifugation at 110,000 g for 1 h at 4° C.

### 13. THERAPEUTIC PULSE FLOW APHERESIS COMBINED WITH ULTRACENTRIFUGATION PLASMAPHERESIS TO INHIBIT EPITHELIAL MESENCHYMAL TRANSFORMATION (EMT) AND TO OVERCOME CHEMOTHERAPY AND RADIOSURGERY RESISTANCE BY REMOVING MICROSOMES, EXOSOMES AND NANOSOMES BOUND TO WHITE BLOOD CELLS AND PLATELETS AND SOLUBLE IN PLASMA

Radiation causes molecular rearrangements in the exosomes. It modulates the connective tissue growth factor (CTGF) which induces cell migration. These exosomes have increased IGF binding protein. It activates focal adhesion kinase, Paxillin, proto-oncogene tyrosine kinase (Src) and neurotrophic tyrosine kinase receptor type 1 (TrkA). Exosomes are capable of delivering its payloads both locally and to distant sites causing increased CTGF activity leading to tumor specific epithelial mesenchymal (EMT) and mesenchymal epithelial transformation (MET) associated tumor recurrence and metastasis (88). Therapeutic pulse flow apheresis combined with ultracentrifugation Plasmapheresis inhibits epithelial mesenchymal transformation (EMT) and overcomes chemotherapy and radiosurgery resistance by removing microsomes, exosomes and nanosomes bound to white blood cells and platelets and soluble in plasma

### 14. THERAPEUTIC PULSE FLOW APHERESIS COMBINED WITH ULTRACENTRIFUGATION PLASMAPHERESIS FOR THE TREATMENT OF HEMATOLOGY/ONCOLOGY DISEASES BY REMOVING MICROSOMES, EXOSOMES AND NANOSOMES BOUND TO WHITE BLOOD CELLS AND PLATELETS AND SOLUBLE IN PLASMA COMBINED WITH MAINTENANCE CHEMOTHERAPY

The frequent and rare hematology/Oncology diseases associated with exosomes when treated by conventional plasmapheresis responds to this treatment only for a short time or not at all. In this invention, they are treated by removing microsomes, exosomes and nanosomes bound to white blood cells and platelets and soluble in plasma combined with maintenance chemotherapy if needed for the maintenance. The brief list of examples of such treatable hematology/Oncology diseases include autoimmune hemolytic anemia, cold hemolytic anemia syndromes, warm antibody hemolytic anemia, in sickle cell anemia, Banti syndrome with high arsenic levels, Chediak-Higashi syndrome by leukapheresis and substitution with normal white cells, Ewing sarcoma by leukapheresis and plasmapheresis especially when associated with fever and removal of MIC2 gene product CD99, Glanzmann thromasthenia by platelet apheresis with normal platelet transfusion and plasmapheresis, glioblastoma multiforme, chronic glaucomatous disease by leukapheresis and plasmapheresis, hereditary hemochromatosis by plasmapheresis, paroxysmal cold hemoglobinuria, paroxysmal nocturnal hemoglobinuria, Lngerhans Cell Histiocytosis, Hyper IgM Syndrome, Large Granular Lymphocyte Leukemia, Lymphangioleiomyomatosis, Hereditary Lymphedema, Hemophagocytic Lymphohistiocytosis, Angioimmunoblastic Lymphadenopathy-Type T-Cell Lymphoma, Mantel Cell Lymphoma, X-linked Lymph proliferative Syndrome, Lynch Syndrome, Mastocytosis, May-Hegglin Anomaly, Medulloblastoma, Melanoma, Mycosis Fungoides, Multiple Myeloma, Nezelof Syndrome, Polycythemia Vera, Acquired Pure Red Cell Aplasia, Henoch-Schonlein Purpura, Idiopathic Thrombocytopenic Purpura, Thrombotic Thrombocytopenic Purpura and Hemolytic Uremic Syndrome of Adults, Thalassemia Major, Thalassemia Minor, Idiopathic Thrombocytosis, and Wegner Granulomatosis.

### 15. THERAPEUTIC PULSE FLOW APHERESIS COMBINED WITH ULTRACENTRIFUGATION PLASMAPHERESIS IN THE TREATMENT OF NON-MALIGNANT DISEASES BY REMOVING MICROSOMES, EXOSOMES AND NANOSOMES BOUND TO WHITE BLOOD CELLS AND PLATELETS AND SOLUBLE IN PLASMA

As an example, a brief summary of the already in routine medical practice plasmapheresis with limited capability devices and which are approved by a major medical insurance company Aetna is shown below (100). In spite of this plasmapheresis do not remove all the patent specific immune complexes and microparticles, cell membranes, plasma soluble disease causing antigen antibodies C-reactive proteins, apoptotic bodies, DNA and RNAs, microsomes, exosomes and nanosomes, they are clinically effective for temporary disease control. With comprehensive removal patent specific immune complexes and microparticles, cell membranes, plasma soluble disease causing antigen antibodies C-reactive proteins, apoptotic bodies, DNA and RNAs, microsomes, exosomes and nanosomes by pulse flow apheresis combined with continuous flow ultracentrifugation as in this invention, these and many more illness could be cured or could be treated with longer lasting remission. It leads to curative renal and liver diseases by plasmapheresis without the need for renal and liver transplants. It leads to curative treatments of myasthenia gravis and similar immune complex disorders.

### 16. LIST OF PRESENTLY INSURANCES APPROVED, MEDICALLY NECESSARY PLASMAPHERESIS (PP), PLASMA EXCHANGE (PE), OR THERAPEUTIC APHERESIS

A. Acute humoral rejection of renal transplants;
B. Acute, severe neurological deficits caused by multiple sclerosis that have a poor response to treatment with high-dose glucocorticoids;
C. Anti-neutrophil cytoplasmic antibody-associated vasculitis (Wegener's granulomatosis, microscopic polyangiitis, Churg-Strauss syndrome) unresponsive to conventional therapy;
D. Babesiosis if member has high-grade parasitemia (greater than or equal to 10%), severe anemia (hemoglobin less than or equal to 10 g/dL), or hepatic, pulmonary, or renal compromise (red blood cell exchange);
E. Catastrophic antiphospholipid syndrome (APS with widespread thromboembolic disease and visceral damage)
F. Chronic relapsing polyneuropathy (chronic inflammatory demyelinating polyneuropathy [CIDP]) with severe or life-threatening symptoms, in persons who have failed to respond to conventional therapy. (Note: Diagnosis of CIDP is documented by symmetric or focal neurological deficits with slowly progressive or relapsing course over 2 or more months with characteristic neurophysiological abnormalities);
G. Essential thrombocythemia (when platelet count is greater than 1,000,000/mm3) (platelet pheresis);
H. Glomerulonephritis associated with antiglomerular basement membrane antibodies and advancing renal failure or pulmonary hemorrhage;
I. Goodpasture's syndrome (glomerulonephritis associated with antiglomerular basement membrane antibodies and advancing renal failure or pulmonary hemorrhage);
J. HELLP (hemolysis, elevated liver enzymes, and low platelets) syndrome of pregnancy, if thrombocytopenia, hemolysis, or renal failure continues to worsen 48-72 hours postpartum;
K. Hemolytic uremic syndrome;
L. Hyperglobulinemias, including (but not limited to) multiple myelomas, cryoglobulinemia, and hyperviscosity syndromes;
M. Last resort treatment of acute disseminated encephalomyelitis, where conventional treatment (including corticosteroids) has failed (i.e., severe neurological deficits have persisted after treatment with corticosteroids);
N. Last resort treatment of life-threatening rheumatoid vasculitis;
O. Last resort treatment of life-threatening systemic lupus erythematosus (SLE) when conventional therapy has failed to prevent clinical deterioration;
P. Leukemia (leukapheresis) (for acute debulking only);
Q. Myasthenia gravis, in persons with any of the following: (i) acute, short-term benefit is critical because of a sudden worsening of symptoms (such as in impending respiratory crisis), (ii) needs rapid improvement of strength before surgery or irradiation, or (iii) requires chronic intermittent treatment because of failure to respond to all other treatments;
R. Paraproteinemic demyelinating neuropathies associated with IgA, IgG or IgM monoclonal gammopathy of undetermined significance (MGUS) (excluding multiple myeloma)
S. Pemphigus vulgaris that is resistant to standard therapy (dapsone, corticosteroids, immunosuppressants such as azathioprine or cyclosporine);
T. Pruritus from cholestatic liver disease (plasma perfusion of charcoal filters), last resort treatment in persons who have failed (unless contraindicated): bile acid resins (cholestyramine or cholestepol), rifampin, ursodeoxycholic acid (in primary biliary cirrhosis), and opioid antagonists (naltrexone, naloxone or nalmefene);
U. Recurrence of focal and segmental glomerulosclerosis in the kidney allograft;
V. Refsum's disease;
W. Renal transplantation from live donor with ABO incompatibility or positive cross-match, where a suitable non-reactive live or cadaveric donor is unavailable;
X. Scleroderma and polymyositis, in persons who are unresponsive to conventional therapy;
Y. Severe (grades 3 to 5) Guillain Barre' syndrome (consistent with guidelines from the American Academy of Neurology, it is generally considered medically necessary to initiate PE within 2 weeks of onset of neuropathic symptoms for ambulant individuals and within 4 weeks of symptom onset for non-ambulant individuals);
Z. Severe hypercholesterolemia in persons refractory to diet and maximum drug therapy who are homozygous for familial hypercholesterolemia (LDL apheresis, also known as heparin-induced extracorporeal LDL precipitation (HELP) or dextra sulfate adsorption) with LDL levels greater than 500 mg/dL, or persons heterozygous for familial hypercholesterolemia with LDL levels greater than 300 mg/dL or greater than 200 mg/dL with documented history of coronary artery disease. (For this policy, maximum drug therapy is defined as a 6-month trial of diet plus maximum tolerated combination drug therapy (defined as a trial of drugs from at least 2 separate classes of hypolipidemic agents such as bile acid sequestrants, HMG-CoA reductase inhibitors, fibric acid derivatives, or niacin/nicotinic acids). Documented history of coronary artery disease is defined as a history of myocardial infarction: coronary artery bypass surgery; percutaneous transluminal coronary angioplasty; alternative revascularization procedure; or angina with coronary artery disease documented by stress test. The frequency of LDL apheresis that is considered medically necessary varies, but typically averages about once every 2 weeks to obtain an intrapheresis level of low density lipoprotein cholesterol (LDL-C) of 120 mg/dL or less. It may be considered medically necessary to treat individuals with homozygous familial hypercholesterolemia more frequently);
AA. Sickle cell disease (therapeutic cytopheresis);
BB. Solid organ transplant from donor with positive cross-match, where a suitable non-reactive donor is unavailable;
CC. Treatment of neuromyelitis optica (Devic's syndrome) that is refractory to glucocorticoids;
DD. Treatment of thrombotic thrombocytopenic purpura (TTP) or microangiopathic hemolytic anemia
EE. Treatment of transverse myelitis when corticosteroid treatment has failed.
FF. Waldenstrom's macroglobulinemia, prophylactic treatment in persons with IgM greater than or equal to 5000 mg/dL while on rituximab or ofatumumab mg/dL, to avoid aggravation of serum viscosity on the basis of IgM flare related to rituximab or ofatumuma

### 17. PARTICLE ANALYSIS WITH ATOMIC FORCE MICROSCOPY (AFM) COMBINED WITH NANOPARTICLE TRACKING ANALYSIS (NTA), DISC CENTRIFUGE NANOPARTICLE ANALYSIS (DCNA) AND FLOW CYTOMETRY

Contact AFM, Dynamic AFM and Antibody Recognition Force Microscopy (IgRM) are used for the image analysis of cancer and cancer stem cell plasma soluble tumor associated proteins, apoptotic bodies, DNA and RNAs, microsomes, exosomes and nanosomes, telomere and telomerase, ATM and ATM kinases. They are incorporated with NTA and DCNA based analysis of these microparticles.

With increasing number of FDA approved monoclonal antibodies based cancer treatment pharmaceuticals are readily available, the antibody recognition force microscopy, the IgRM, has greater application in imaging of cancer specific miRNAs, nucleosome and exosomes.

Disease specific, easily available FDA approved monoclonal antibodies for cancer treatments are used for functionalizing the AFM tip. The antibody is selected from the list shown in Table 3. The AFM tip is also functionalized with cancer stem cell specific monoclonal antibodies listed in Table 1. This helps to map the cancer and cancer stem cell specific antigens on the surface of exosomes and nucleosomes. Exosome's surface is rich in various antigens including the major histocompatibility antigens. Thus the surface of the exosomes and nucleosomes purified by multiple steps SDG-ultracentrifugation is mapped for cancer and cancer stem cell specific antigens.

Along with NTA and DCNA, the AFM refines the cellular and sub cellular imaging, the cellular shape, size, width and height, surface roughness and stiffness, cellular exosomes with DNAs, RNAs and tumor specific enzymes enclosed in them, cell membrane, cell membrane's receptor affinity binding to ligands including hormone receptors and their specific ligands, cytoplasmic microvesicles, the tumor RNAs, the Golgi apparatus and its phenotypic tumor specific Warburg-lactate glycolysis, the nuclear chromosomes, the tumor specific telomere—telomerase, the ALT telomere of the cancer stem cell, the cell divisions, the apoptosis and apoptotic bodies and DNA repair and a long list of other cellular subfractions associated with tumor specific biochemistry.

For AFM/NTA/DCNA analysis of the sucrose density gradient fractionated sediment of micro and nanoparticles, the sediment is reconstituted in PBS for AFM/NTA/DCNA and is followed per the manufacturer's instructions or those adapted to suite a particular study.

Alternatively, the sucrose gradient sediment of the micro and nanoparticle sediments are made as strips of 100 to 200 μm thick and fixed on to a poly-1-lysine-coated 15-mm coverslip and incubated with a primary antibody. After this incubation, these strips are washed and incubated with a secondary antibody conjugated with a fluorescent tag for fluorescent microscopy. After washing such treated sediment strips several times in PBS, it is used immediately or stored at 4 degree centigrade until its AFM Immediately before the AFM, the prepared tissue is fixed on to a 15-mm coverslip that is coated with poly-1-lysine. The tissue strip is examined under an AFM.

### 18. TUMOR CELL'S AND NORMAL CELL'S EXOSOME ANALYSIS BY DCNA AND NTA

DCNA is primarily used for a quick analysis of exosomes with size ranging from 10 nm to 50 nm (86) which are not sufficiently imaged with NTA. The DCNA and the NTA software are used to determine the size, shape and concentration of cellular exosomes. The NTA with its software is used for more detailed analysis of exosomes, its shape, locations, their movements and centre of each and every particle and measures the average distance it moves per frame (87, 88).

### 19. COMPARATIVE AFM/NTM/DCNA PHENOTYPIC ANALYSIS OF CANCER STEM CELL EXOSOMES AND NORMAL TISSUE EXOSOMES IN SDG FRACTIONS

To assess if the exosomes, nanosomes and other microparticles in the fractionated sucrose density gradient ultracentrifugation (FSDGU) are derived from undifferentiated cancer stem cells, differentiated cancer stem cells or normal cells, the following guidelines are followed.

First, the exosomes in an aliquot of FSDGU are tested for undifferentiated cancer stem cell antigens from the list in Table 1 and their specific antibody binding.

Second, the remaining exosomes in the same aliquot of FSDGU are tested for known, differentiated cancer cell markers like those shown in Table 2

If both the first and second group's testing for the antigen antibody binding of exosomes shows their respective antigen-antibody specificity, then those exosomes are marked as derived from undifferentiated cancer stem cells. It is one type of undifferentiated cancer stem cell exosome's phenotype.

If the exosomes have only undifferentiated cancer stem cell antigen-antibody binding, and no or poor binding to generally known cancer antigen markers like those shown in Table 2, then such exosomes are marked as derived from undifferentiated cancer stem cells but of a different phenotype.

If there are no undifferentiated cancer stem cell antigen-antibody binding but has binding only to generally known cancer cell markers like those in Table 2, then such exosomes are marked as derived from more differentiated cancer cells.

The exosomes with poor or no cancer cell antigen-antibody binding are marked as derived from normal tissue. They are the remaining exosome in the same aliquot FSDGU after separation of the exosomes derived from undifferentiated cancer stem cells and those exosomes from differentiated cancer cells.

Thus the characteristics in exosomes derived from the undifferentiated cancer stem cells and differentiated cancer cells and their correlation with presence or absence of generally known cancer cell markers will indicate the predominant phenotype of a tumor from which the tested cancer cell exosome have originated.

TABLE 1
2009 NCI's Prioritized List of Putative Cancer Stem Cell Antigens

| | | | | Expression level | |
|---|---|---|---|---|---|
| | | Cumlative | Stem cell | and % positive | |
| No | Antigen | Score | Expression | cells | Immunogenicity |
| 1 | WT1 | 0.81 | 1.0 (stem cells) | 0.37 (high most) | 1.0 (trials) |
| 2 | MUC1 | 0.79 | 1.0 (stem cells) | 1.0 (high all) | 1.0 (trials) |
| 3 | LMP2 | 0.78 | 1.0 (stem cells) | 0.37 (high most) | 1.0 (trials) |
| 4 | EGFRvIII | 0.76 | 1.0 (stem cells) | 0.37 (high most) | 1.0 (trials) |
| 5 | MAGE A3 | | 1.0 (stem cells) | 0.37 (high most) | 1.0 (trials) |
| 6 | p53, nonmutant | 0.67 | 1.0 (stem cells) | 0.37 (high most) | 1.0 (trials) |
| 7 | NY-ESO-1 | 0.66 | 1.0 (stem cells) | 0.37 (high most) | 1.0 (trials) |
| 8 | bcr-abl | | 1.0 (stem cells) | 0.23 (low all) | 1.0 (trials) |
| 9 | hTERT | | 1.0 (stem cells) | 0.23 (low all) | 1.0 (trials) |
| 10 | Sarcoma | | 1.0 (stem cells) | 1.0 (high all) | 0.39 (trial) |
| | translocation | | | | |
| | breakpoints | | | | |

### 20.

TABLE 2
Partial List of Differentiated Cancer Cell Antigens Identifiable by
AFM

| | | | | | | | Cancer | |
|---|---|---|---|---|---|---|---|---|
| | | | | | | | Cell with | |
| | | | | | | | Apoptosis | |
| | Cancer Cell | | | Cancer Cell | | | Resistant | |
| No | Antigen | AFM | No | Antigen | AFM | No | Antigen | AFM |
| 1 | DNA | x | 18 | PTPRN2 | x | 34 | CD133 | x |
| | Methylation | | | | | | | |
| 2 | Apoptotic | x | 19 | ALDH1(A1) | x | 35 | CD44 | x |
| | changes | | | | | | | |
| 3 | PDGF | x | 20 | CD44 | x | 36 | CD24 | x |
| 4 | IGF | x | 21 | PTEN | x | 37 | CD44v3 | x |
| 5 | CTCF | x | 22 | CD133 | x | 38 | CD44v4 | x |
| | | | | (PROM1) | | | | |
| 6 | Telomere | x | 23 | NKX3-1 | x | 39 | CD44v4/5 | x |
| 7 | Integrin | x | 24 | MYC | x | 40 | CD44v6 | x |
| 8 | ER | x | 25 | ATXN1 | x | 41 | CD44v7 | x |
| | | | | (SCA-1) | | | | |
| 9 | Aldosterone | x | 26 | GATA3 | x | 42 | CD44v7/8 | x |
| | Receptor | | | | | | | |
| 10 | Notch | x | 27 | TNFSF11 | x | 43 | EGFR | x |
| | | | | (RANKL) | | | | |
| 11 | Cruciform | x | 28 | TNFRSF11B | x | 44 | PE | x |
| | DNA | | | | | | | |
| 12 | Telomerase | x | 29 | TACSTD2 | x | 45 | FITC | x |
| 13 | miRNA | x | 30 | DNA | x | 46 | APC | x |
| | | | | methylation and | | | | |
| | | | | apoptosis | | | | |
| | | | | resistance | | | | |
| 14 | JNK | x | 31 | Homologues | x | | | |
| | | | | Recombination | | | | |
| 15 | Androgen | x | 32 | Nucleosome | x | | | |
| | Receptor | | | structures | | | | |
| 16 | ER alpha and | x | 33 | Telomere/Telomerase | x | | | |
| | beta | | | | | | | |
| 17 | Cell adhesion | x | | 5-FUCD-UPRT | | | | |
| | CTCF | | | PARP | | | | |
| | Rad50- | | | Glutamate | | | | |
| | MRN | | | | | | | |
| | Complex | | | | | | | |

### 21.

### 22. EXOSOMES DERIVED FROM CANCER CELLS AND UNDIFFERENTIATED CANCER CELLS AND THEIR PHENOTYPING (EXAMPLES)

a. AFM measurements of shape, height, width, surface roughness and stiffness of exosomes combined with fluorescence microscopy of exosomes bound to undifferentiated cancer stem cell specific antigens selected from the list of cancer stem cell antigens shown in Table 1 and they are have mutated CTCF with different height and length and DNA looping and its comparison with antigen-antibody binding for the list of differentiated cancer cell's antigens listed in Table 2
b. AFM measurements of shape, height, width, surface roughness and stiffness of exosomes combined fluorescence microscopy of exosomes bound to undifferentiated cancer stem cell specific antigens selected from the list of cancer stem cell antigens shown in Table 1 and they are have cancer treatment resistance like to 5-flurouracil (5-FU) when cytosine deaminase-uracil phosphoribosyl transferase (CD-UPRT) fusion gene is present and its comparison with antigen-antibody binding for the list of differentiated cancer cell's antigens listed in Table 2
c. AFM combined fluorescence microscopy of exosomes bound to undifferentiated cancer stem cell specific antigens selected from the list of cancer stem cell antigens shown in Table 1 showing different height and width, surface roughness and stiffness histograms of the exosome DNA and their double strand break and homologues DNA repair deficiency after treatments and its comparison with antigen-antibody binding for the list of differentiated cancer cell antigens listed in Table 2
d. AFM combined fluorescence microscopy of exosomes bound to undifferentiated cancer stem cell specific antigens selected from the list of cancer stem cell antigens shown in Table 1 showing different shape, height and width surface roughness and stiffness exosome in response after treatments and its comparison with antigen-antibody binding for the list of differentiated cancer cell's antigens listed in Table 2
e. AFM combined fluorescence microscopy of exosomes bound to undifferentiated cancer stem cell specific antigens selected from the list of cancer stem cell antigens shown in Table 1 and the cancer stem cell exosome's poly (ADP) ribose polymerase (PARP) cleavage after treatments its associated changes in cancer stem cell's shape, height and width surface roughness and stiffness and its comparison with antigen-antibody binding for the list of differentiated cancer cell's antigens listed in Table 2
f. AFM combined fluorescence microscopy of exosomes bound to undifferentiated cancer stem cell specific antigens selected from the list of cancer stem cell antigens shown in Table 1 and the presence of Rad50/MRE11/NBS1(MRN Complex) in ER, PR and HER2 negative breast cancer patient's exosomes with changes in their shape, height and width surface roughness and stiffness and its comparison with antigen-antibody binding from the list of differentiated cancer cell's antigens in Table 2
g. AFM combined fluorescence microscopy of exosomes bound to undifferentiated cancer stem cell specific antigens selected from the list of cancer stem cell antigens shown in Table 1 and the presence of Warburg glycolytic glutamate in exosomes with associated changes in their shape, height, width, surface roughness and stiffness and its comparison with antigen-antibody binding from the list of differentiated cancer cell's antigens in Table 2
h. AFM combined fluorescence microscopy of exosomes bound to undifferentiated cancer stem cell specific antigens selected from the list of cancer stem cell antigens shown in Table 1 and the cancer stem cell exosomes without or greatly diminished caspase activity and its associated changes in exosomes shape, height and width, surface roughness and stiffness comparison with antigen-antibody binding for the list of differentiated cancer cell's antigens listed in Table 2

### 23. SYSTEMIC CANCER IMMUNITY AFTER kGy RADIOSURGERY

The kGy radiosurgery causes severe DNA damage. DNA damage based apoptosis is primarily controlled by CD95 (Fas/APO-1) and its ligand, CD95L. CD 95L in the tumor infiltrating lymphocytes controls the immune response to cancer. Like the immune lymphocytes kill the virus and other pathogens, the immune lymphocyte kills the cancer cells.

### 24.IMMUNOSUPPRESSION BY SURGERY AND CHEMO-RADIOSURGERY BY BLOCKING ANTIBODIES

In conventional fractionated radiation therapy, the daily radiation lasting for about 8 to 10 weeks wipes out the innate local cancer immunity. The daily fractionated radiation therapy eliminates most of the immunity processing cells, the mast cells, phagocytes, natural killer cells, γδ T cells, macrophages, neutrophils, dendritic cells, basophiles and eosinophils. After each day's radiation, these cells attempts to repopulate the locally irradiated tumor but by the next day's radiation, they are destroyed or damaged. It makes them ineffective to help to establish an effective innate and adoptive immunity against the tumor that is treated. Likewise, surgery, chemotherapy and high dose radiosurgery establish an effective innate and adoptive tumor immunity against the treatments exposed tumor antigens and the innate and adoptive immunity against the treatments induced local inflammatory reaction. This immune cytotoxicity is neutralized by the immune complex against innate and adoptive tumor immunity and against the tumor inflammatory reaction. In this invention the immune complex against innate immunity and adoptive tumor immunity is removed by pulse flow apheresis and continuous flow ultracentrifugation combined with immune affinity absorption. It include aphaeretic removal of the blocking antibodies against immune complex against tumor cell, aphaeretic removal of the blocking antibodies against tumor cell exosome immune complex and non-cancer immune complex diseases appearing in association with or without cancer.

### 25. SINGLE FRACTION kGy MICROBEAM RADIOSURGERY INDUCED INFLAMMATION, CYTOKINES SECRETION AND SYSTEMIC TUMOR IMMUNITY AND ITS BLOCKING ANTIBODIES

The kGy parallel microbeam radiosurgery to a tumor in split seconds is associated with inflammation at the tumor site. It releases a number of cytokines. Even the lower dose localized radiation in the range of 5 cGy to 2 Gy evokes localized innate immunity. The kGy radiation in split second obviously evokes much stronger innate immunity and secretion of a number of cytokines. Likewise, radiation evokes adoptive immunity through the FAS pathway. In vitro experiments, MC 38 adenocarcinoma cells at 20 Gy dose has increased FAS activity at molecular, phenotypic and functional levels. Higher dose radiation sensitizes the tumor cells to antigen specific cytotoxic-T-lymphocyte's (CTLs) FAS/FAS ligand pathway. In vivo experiments, the same MC 38 adenocarcinoma cells growing subcutaneously also show 8 Gy radiation sensitization and CTL adoptive immunity by up regulation of FAS leading to tumor growth arrest and tumor rejection. Antigen processing dendritic cells are stimulated by radiated highly malignant prostate cancer cells but only at high doses, in the range of 10-60 Gy. Unirradiated cells have no such immunostimulatory effects. This immune cytotoxicity is neutralized by the immune complex against innate and adoptive tumor immunity and against the tumor inflammatory reaction. In this invention the immune complex against innate immunity and adoptive tumor immunity is removed by pulse flow apheresis and continuous flow ultracentrifugation combined with immune affinity absorption.

### 26. BRIEF SUMMARY OF THE INVENTION

The innovations disclosed in this invention include:
1. Microbeam, nanobeam and minibeam generation by injecting pencil beam into a defocusing and focusing and beam size controlling magnet that controls the beam size as microbeam, nanobeam or minibeam and their spacing from each other. It drastically differs from the methods of microbeam and minibeam generation with conventional multislit metallic collimator that generates secondary neutron and gamma radiation.
2. High brightness electron microbeam, nanobeam and minibeam generation for radiosurgery with Wakefield accelerator system.
3. High brightness Compton gamma ray microbeam, nanobeam and minibeam generation with Wakefield accelerator system for gamma ray microbeam, nanobeam and minibeam radiosurgery.
4. High brightness proton microbeam, nanobeam and minibeam generation with Wakefield accelerator system for microbeam, nanobeam and minibeam radiosurgery.
5. Magnetically focused very high energy electron pencilbeam, microbeam, nanobeam and minibeam made as proton pencilbeam, microbeam, nanobeam and minibeam with deep skin penetration and with least skin toxicity.
6. Sharp well defined microbeam, nanobeam and minibeam without lateral penumbra and peak and valley doses without spreading and smearing with each other.
7. Keeping the valley region stem cell's regeneration and migration to peak dose region intact after electron microbeam, nanobeam or minibeam radiosurgery.
8. High density tissue equivalent patient specific field shaping collimation with secondary neutron and gamma ray absorption.
9. Group of five tissue equivalent collimator system configured as all the simultaneous beams pointing towards to an isocentric tumor for all field simultaneous microbeam, nanobeam or minibeam radiosurgery.
10. High density tissue equivalent patient specific filed shaping collimation with high density tissue equivalent glass composition without lead that absorbs secondary neutron and gamma rays.
11. Magnetic resonance image (MRI) guided laser wakefield microbeam, nanobeam or minibeam radiosurgery.
12. Photocathode-racetrack microtron laser wakefield accelerator system generating multiple simultaneous collinear very high energy electron beams for high throughput, 10 to 20 radiosurgical rooms setup cancer center.
13. Magnetically focused, below the skin deep penetrating electron microbeam, nanobeam and minibeam for multiple simultaneous beams, kGy radiosurgery.
14. Photocathode-racetrack microtron laser wakefield accelerator system generating very high energy electron for deuterium-tritium reaction based neutron production in a drift tube and their collinear very high energy electron beam separated for microbeam, nanobeam and minibeam radiosurgery and the collinear neutron beam generating radioisotopes for nuclear medicine imaging.
15. Ten to twenty room radiosurgical center equipped with a laser wakefield accelerator system capable of producing ten collinear very high energy electron beams and generating very high energy electron microbeam, nanobeam or minibeam and with neutron and gamma ray absorbing collimator system and it is installed in a multiple story artistic lead free glass building as a high throughput low cost radiosurgical center as part of a modern comprehensive cancer center.

### 27. BRIEF DESCRIPTION OF THE DRAWINGS

FIG. 1A shows a microbeam, nanobeam or minibeam generating collimator system that is used with a modified wakefield accelerator system that generates high energy electron beam or inverse Compton scattering collinear electron and gamma ray or proton beam for kGy dose radiosurgery with least radiation toxicity to normal tissue and least secondary neutron and gamma radiation exposure to the patient and to the areas surrounding the accelerator and treatment rooms.

FIG. 1B is a continuous illustration of FIG. 1A to show the absorption of secondary neutron, ions and gamma radiation generated by the beamline in the treatment room with a tissue equivalent collimator to reduce secondary radiation in the treatment room and its scattered radiation outside the treatment room that are produced by the beamline

FIG. 1C-1, FIG. 1C-2A, FIG. 1C2B and FIG. 1C3 and their associated FIG. 1C-3-1, FIG. 1C-3-2, FIG. 1C3-3 and FIG. 1C3-4 are continuous illustration of the patient specific high density field defining block 55 shown in FIG. 1A.

FIG. 1C-1 shows a patient specific field defining high density tissue equivalent block inserted at the top of the high density tissue equivalent microbeam, nanobeam or minibeam generating collimator.

FIG. 1C-2A and FIG. 1C2-B schematically illustrates the microbeam passing through the central field opening beam aperture in a high density tissue equivalent patient specific field shaping block 55 constructed with mold making Styrofoam cuts as in Cerrobend block making for conventional radiation therapy.

FIG. 1C-3 shows three sections of high density tissue equivalent patient specific field shaping block 55 with their respective Styrofoam cuts and blocks which include an inner section shown in FIG. 1C-3-1, a section with borated Styrofoam cut boarding the inner high density tissue equivalent block that is shown in FIG. 1C3-2, a rectangular Cerrobend block that is shown in FIG. 1C-3-3.

FIG. 1C-3-1 illustrates the rectangular Styrofoam cut 1 with high density tissue equivalent composition in the periphery of the field opening

FIG. 1C-3-2 shows the rectangular borated Styrofoam cut 2 in between the inner high density tissue equivalent composition (HDTEC) and the outer rectangular Cerrobend block.

FIG. 1C-3-3 illustrates Rectangular Cerrobend block sandwiched in between inner borated Styrofoam and outer remaining rectangular Styrofoam block

FIG. 1C-4 shows Semi-Permanent rectangular Cerrobend or lead block attached with borated Styrofoam in the treatment head for attachment of high density patient specific field shaping inner block

FIG. 1C-3-5 illustrates the exchangeable high density patient specific field shaping inner block and its inserting into and removal from the Semi-Permanent rectangular Cerrobend or lead block with attached rectangular borated Styrofoam in the treatment head.

FIG. 1D-1 is a continuous illustration of high density patient specific field shaping block making with high density tissue equivalent glass composition.

FIG. 1D-2 shows high density patient specific field shaping block cut out of high density tissue equivalent glass composition by hotwire cutting similar to hotwire cutting of Styrofoam to make Cerrobend block.

FIG. 1D-3 Illustrates a high density patient specific field shaping block cut out of high density tissue equivalent glass composition together with rectangular cut borated Styrofoam, rectangular Cerrobend block and the holding Styrofoam mounted on to a tray to be inserted above the microbeam or nanobeam or minibeam generating high density tissue equivalent primary collimator 34.

FIG. 1D-4 illustrates high density patient specific field shaping block making with molten high density tissue equivalent glass composition poured into a mold like the molten Cerrobend poured into a mold in Cerrobend block making

FIG. 2 illustrates two sets of interlacing parallel microbeam, or nanobeam or minibeam at the isocentric tumor 52, one set from 0 degree and another set from 90 degrees for 100 to 1,000 Gy single fraction radiosurgery.

FIG. 3 shows five sets of interlacing parallel microbeam, or nanobeam or minibeam at the isocentric tumor 52. The parallel electron beams or Collilinear electron/gamma rays or proton or carbon ion beams, all interlaces at the isocentric tumor.

FIG. 4 illustrates microbeam or nanobeam or minibeam generation out of spread out laser Wakefield Thompson scattering electron or Compton scattering gamma ray with collinear electron beam or proton or carbon ion beam 14 in a cylindrical tissue equivalent primary collimator incorporated with a patient specific collimator.

FIG. 5 shows interlacing microbeam or nanobeams or minibeams from two sets of spread out laser wakefield Thompson scattering electron or Compton scattering gamma ray with collinear electron beam or proton or carbon ion beam 14, one from 0-degree and the other from 90-degree for simultaneous interlaced microbeam or nanobeam radiosurgery of an isocentric tumor.

FIG. 6 is another illustration of the spread out pencil beam's processing as illustrated in FIG. 5 but the spread out beam as first channeled through a semi-patient specific carbon nanotube pre-collimator 80 for beam focusing by carbon nanotube's induced magnetism before it is channeled through the patent specific collimator 55 and the tissue equivalent primary collimator 34 where microbeam, nanobeam or minibeams are generated.

FIG. 7 shows interlacing microbeam or nanobeams from two sets of microbeam, nanobeam, minibeam or proton or carbon ion microbeam, nanobeam or minibeam generating systems with semi-patient specific carbon nanotube pre-collimator, one at 0-degree and the other at 90-degree for simultaneous interlaced microbeam, nanobeam or minibeam radiosurgery of an isocentric tumor.

FIG. 8 illustrates a patient specific radiation therapy field shaping block system 55A made of high density tissue equivalent composition for secondary neutron absorption, borated Styrofoam to moderate the MV gamma rays and Cerrobend to absorb the moderated gamma rays and as it placed above the tissue equivalent primary collimator 34 for spread out Brag peak field shaping and microbeam, nanobeam or minibeam radiosurgery.

FIG. 9 shows a neutron moderating Styrofoam block that is surrounded by a Cerrobend cover 55B and this combined system is placed in the periphery of a field shaping multileaf collimator (MLC) 55-C and this combined blocks as placed above the tissue equivalent primary collimator 34 for spread out Brag peak proton beam field shaping for microbeam, nanobeam or minibeam radiosurgery.

FIG. 10 shows a patient specific filed shaping block system 55-D made of high density tissue equivalent composition for secondary neutron absorption, borated Styrofoam to moderate the MV gamma rays and Cerrobend to absorb the moderated gamma rays for daily fractionated proton radiation therapy or 10 to 20 Gy single fraction proton radiosurgery with lesser secondary neutron and gamma radiation to the patient and for the neutron radiation safety to technical staff treating the patient and in the area surrounding the treatment room without the expense and inconvenience of spot or raster scanning with huge magnets.

FIG. 11 illustrates a patient specific radiation therapy field shaping block system 55-D consisting of multileaf collimator (MLC) 55-C shielded with high density tissue equivalent composition for secondary neutron absorption, borated Styrofoam to moderate the MV gamma rays and Cerrobend to absorb the moderated gamma rays for daily fractionated proton radiation therapy or 10 to 20 Gy single fraction proton radiosurgery with lesser secondary neutron and gamma radiation to the patient and for the neutron radiation safety to technical staff treating the patient and in the area surrounding the treatment room without the expense and inconvenience of spot or raster scanning with huge magnets.

FIG. 12 shows magnetic resonance image (MRI) guided kGy microbeam, nanobeam or minibeam single or hypofractionated rotational radiosurgery system having very high energy (VHE) laser wakefield Thompson scattering electron microbeam or nanobeam or minibeam generating cylindrical tissue equivalent primary collimator and a patient specific conformal filed shaping collimator.

FIG. 13 illustrates kGy microbeam, nanobeam or minibeam single or hypofractionated rotational radiosurgery system without MRI as described in FIG. 12 and having only very high energy (VHE) laser wakefield Thompson scattering electron microbeam or nanobeam or minibeam generating cylindrical tissue equivalent primary collimator and a patient specific conformal field shaping collimator.

FIG. 14A: Shows switching the pencil beam into right and left beams and steering them into the beam lines with steering magnets

FIG. 14B illustrates the 90 degree bending of the Laser wakefield Thompson scattering electron or Compton scattering gamma ray with collinear electron beam or proton or carbon ion pencil beam 14 and its passing through the 90 degree bending beamline 142 towards the microbeam or nanobeam or minibeam generating tissue equivalent collimator system 12.

FIG. 14C: Illustrates the components for steering the beam towards the next microbeam or nanobeam or minibeam generating tissue equivalent collimator systems 12 with two 22.5 degree bending of the beam to make a combined 45 degree bend to guide the beam through the octagonal beam line and the simultaneously switched another segment of the beam by the switching magnet 150 as steered towards the microbeam or nanobeam or minibeam generating tissue equivalent collimator systems 12.

FIG. 15 shows the 90 degree bent laser wakefield Thompson scattering electron or Compton scattering gamma ray with collinear electron beam or proton or carbon ion pencil beam 14 injection into five microbeam or nanobeam or minibeam generating tissue equivalent collimator systems 12 and generating microbeams or nanobeams or minibeams for all fields simultaneous microbeam or nanobeam or minibeam single or hypofractionated kGy radiosurgery.

FIG. 16 illustrates the pencil beam as injected into a mini storage ring 154 from which synchronized multiple simultaneous beams are switched into five microbeam or nanobeam or minibeam generating tissue equivalent collimator systems 12 where the microbeam or nanobeam or minibeams are generated.

FIG. 17A shows a laser wakefield accelerator with dual supersonic gas jet adapted for stable, 50 to 300 MeV range tunable quasi monoenergetic electron microbeam or nanobeam or minibeam and or high brightness collinear inverse Compton scattering gamma ray microbeam or nanobeam or minibeam generation for kGy radiosurgery.

FIG. 17B illustrates the injection and acceleration of a single laser pulse focused on to dual composite gas jets with independently adjustable gas density that generates 50 to 300 MeV stable laser wakefield accelerated quasi monoenergetic electron beam and high brightness collinear inverse Compton scattering gamma rays for radiosurgery.

FIG. 17C illustrates a laser wakefield accelerator with dual supersonic gas jets attached to the beam bending and splitting magnet shown in FIG. 14A for steering of the wakefield accelerated electron beam to two sets of five tissue equivalent collimators shown in FIG. 18 that generates microbeam or nanobeam or minibeam.

FIG. 18 shows a laser wakefield accelerator with dual supersonic gas jet as connected to the bending and splitting magnets and the beam steering to two sets of five tissue equivalent collimator systems installed in their respective radiation protective treatment rooms for electron, Compton gamma, or proton or carbon ion microbeam or nanobeam or minibeam kGy radiosurgery.

FIG. 19 illustrates a laser wakefield accelerator with dual supersonic gas jet as connected to the bending and splitting magnets and the beam steering to two sets of five tissue equivalent collimator systems installed in their respective radiation protective treatment rooms and with floor plan for the office, clinical patient care and research.

FIG. 20 shows a 150 MeV stable very high energy electron beam at dose rate of 2-3 Gy per second generating racetrack laser-photocathode-racetrack microtron system instead of the laser wakefield accelerator shown in FIG. 19 and which is directly connected to the bending and splitting magnets 186 and the split beams as connected to two sets of five tissue equivalent collimator systems installed in their respective radiation protective treatment rooms.

FIG. 21 illustrates 150 MeV electron beam generation with photocathode racetrack microtron and its synchronization with 100 TW 20 fs 10 Hz laser beam generated by laser wakefield acceleration and their Z-pinch acceleration with a Z-pinch gun to stable 150 MeV to a GeV electron beam with dose rate in the range of 109 Gy per second and this system as directly connected to microbeam, nanobeam or minibeam generating high density tissue equivalent collimators in two adjacent treatment rooms for kGy range microbeam, nanobeam or minibeam radiosurgery

FIG. 22-A shows the same 150 MeV electron beam generating photocathode racetrack microtron system and its beam synchronization with 100 TW 20 fs 10 Hz laser beam generated by laser wakefield acceleration and their further Z-pinch acceleration with a Z-pinch gun as illustrated in FIG. 21 but the very high energetic electron beam 334 from the Z-pinch gun and separated from the laser beam is split into 10 beams and the alternate split beams as connected to ten treatment rooms with high density tissue equivalent secondary neutron absorbing collimator systems for kGy microbeam, nanobeam or minibeam radiosurgery.

FIG. 22-B shows a high repetition rate dielectric wave guide for inserting into the racetrack-laser Wakefield accelerator system instead of the Z-pinch gun to improve the quality of the very high electron energy beam and to generate collilinear multiple beams for multiple suits radiosurgery.

FIG. 22-C illustrates a similar collinear multibeam wakefield very high energy electron accelerator system with ten split beams and the five alternate split beams connected to ten radiosurgical rooms with tissue equivalent collimators as in FIG. 21-A but the Z-pinch gun is replaced with a high repetition rate dielectric wave guide 370.

FIG. 22-D shows a similar collinear multibeam wakefield very high energy electron accelerator system with ten split beams and five alternate split beams connected to ten radiosurgical rooms with tissue equivalent collimators as in FIG. 22-C but the high repetition rate dielectric wave guide 370 is replaced with a corrugated pipe waveguide 378.

FIG. 23 illustrates the photocathode racetrack microtron system 338 and the TW laser and laser processing system 440 with Z-pinch gun 342 attached to a drift chamber in which the very high energy electron beam (VHEE) passes through deuterium-tritium gas that generates stable collinear accelerated VHEE beam and 2.45 MeV and 14 MeV neutron beam and their separation into VHEE and neutron beam for VHEE electron microbeam radiosurgery, and the high flux, 1012 to 1015 neutron generating radioisotopes.

FIG. 24 shows removal of circulating tumor cells (CTC), RNA, DNA and DNA fragments, exosomes, microsomes and nanosomes from circulation after kGy radiosurgery and chemotherapy by pulsed flow apheresis to minimize bystander and abscopal effects associated tumor recurrence and metastasis.

FIG. 25A illustrates a continuous flow ultracentrifuge rotor adapted for plasmapheresis where plasma from the pulsed flow apheresis flows through the bottom inlet of the rotor and separation of the remaining plasma soluble larger micro and nano particle cell debris, cell membranes, normal cell and tumor cell associated proteins, apoptotic bodies, DNA and RNAs, microsomes, exosomes and nanosomes, telomere and telomerase, ATM and ATM kinase after pulsed flow apheresis into a sucrose density gradient solution within the rotor and the plasma free of larger soluble cellular components flows through the outlet at the top of the rotor either towards a series of affinity chromatography columns connected with atomic force microscopy (AFM) combined with nanoparticle tracking analysis (NTA), disc centrifuge nanoparticle analysis (DCNA) and flow cytometry for particle tracking or the purified plasma flowing back to the patient.

FIG. 25B shows the same continuous flow ultracentrifuge rotor adapted for plasmapheresis of the pulsed flow apheresis plasma as illustrated in FIG. 25A but the supernatant exiting from the top hollow driveshaft 510 flows through two affinity chromatography columns coated with patient specific tumor nanosomes antibody and connected with AFM, NTA, DCNA and a flow cytometer (FCM) for particle tracking and the effluent supernatant exiting from the chromatographic columns 534 flows back to the high speed rotating cylindrical rotor 508 through its bottom hollow driveshaft 502 and back to the patient or it re-circulates through a set of two affinity chromatography columns.

FIG. 25C illustrates the same continuous flow ultracentrifuge rotor adapted for plasmapheresis of the pulsed flow apheresis plasma as in FIG. 25A and FIG. 25B but the supernatant exiting from the top hollow driveshaft 510 flows through a series of affinity chromatography columns coated with patient specific tumor nanosomes antibody with nanosomes monitoring with AFM, NTA, DCNA and FCM and the effluent purified supernatant from the chromatographic columns 534 flows back to the high speed rotating cylindrical rotor 508 through its bottom hollow driveshaft 502 and back to the patient or it re-circulates through the series of affinity chromatography columns that adsorbs the plasma soluble micro and nano particle cell debris, cell membranes, normal cell and tumor cell associated proteins, apoptotic bodies, DNA and RNAs, microsomes, exosomes and nanosomes, telomere and telomerase, ATM and ATM kinase.

FIG. 26-A shows a photocathode racetrack microtron system 380 with one beamline 346 installed in the basement section of a glass building RT center 382 and its split beamlines attached to a group of five tissue equivalent collimator systems 190 in each of the two adjacent rooms 192 in above the ground glass building RT center 384 for VHEE-beam kGy microbeam, nanobeam or minibeam radiosurgery.

FIG. 26-B illustrates a photocathode racetrack microtron laser wakefield accelerator system 380 as described in FIG. 26-A and installed in the basement of a glass building radiation therapy center but with 10 collinear VHEE beamlines and five of those beamlines connected to 10 treatment rooms with five tissue equivalent collimator systems 190 in each of the treatment rooms 192 for very high energy electron beam kGy microbeam, nanobeam or minibeam radiosurgery.

FIG. 26-C Shows the general view of the glass building radiation therapy cancer treatment center described in FIG. 24-A and FIG. 24-B with the photocathode racetrack microtron laser wakefield accelerator system in the basement and the radiosurgical rooms in the building is visible through front exposed radiation shielding glass panels.

### 28. REFERENCE NUMERALS

12 Microbeam or nanobeam or minibeam generating tissue equivalent collimator systems

14. Laser wakefield Thompson scattering electron or Compton scattering gamma ray with collinear electron beam or proton or carbon ion pencil beam

15A. Emergency beam

15B. Dose monitor

15C. Removable Cerrobend beamline block covered with high density tissue equivalent leather

16. Collimator

18. Quadrupole magnet

20. Negatively charged beam

22. Focusing and beam size controlling magnet

24. Stripper grid

26. Alternating positively and negatively charged beam segments

28. Deflection magnet with DC vertical dipole field

30. Positively charged Wakefield laser electron beam beamlets or collilinear electron/gamma rays beams or proton or carbon ion beams

32. Negatively charged Wakefield laser electron beam beamlets or collilinear electron/gamma rays beams or proton or carbon ion beams

34. High density tissue equivalent primary collimator.

35. Broadbeam neutron absorbing collimator

35-B. Beam guide in broadbeam tissue equivalent collimator

35-C. Opening for the beam exit in broadbeam tissue equivalent collimator

36. Tissue equivalent collimator

37. Gamma and neutron filtered spread out Brag peak proton beam

38. Converging magnetic field in one plane

40. Diverging magnetic field in another plane

41. Pre-patient specific collimation parallel beams

42. parallel beams

43. Post-patient specific collimation parallel beams

44. Microfocus beam guides

45. Microfocus beam guide's openings in the tissue equivalent block

46. Focusing anode

48. Focusing magnet

50. Focused microbeam/nanobeam or minibeam

52. Isocentric tumor

54. Peak dose region

55. High density tissue equivalent patient specific field shaping block

55-A. Patent specific field shaping block

55-B. High density tissue equivalent block-2

55-C. Multileaf collimator (MLC)

55-D. Patient specific collimator system with high density neutron absorbing composition, gamma ray moderating borated Styrofoam and cerrobend

56. Low dose valley region

58. Tissue equivalent universal collimator-1

60. Tissue equivalent universal collimator-2

62. Tissue equivalent universal collimator-3

64. Tissue equivalent universal collimator-4

66. Tissue equivalent universal collimator-5

68. Circular gantry

70. Passive scatterer

72. Nozzle

74. Dose monitor

75. Spread out Brag peak beam

76. Unwanted beam outside of the carbon tube

77. Processed laser wakefield Thompson scattering electron or Compton scattering gamma ray with collinear electron beam or proton or carbon ion beam

78. Filtered parallel microbeam or nanobeam or minibeam

80. Semi-patient specific carbon nanotube pre-collimator

90. Primary electro magnets made of non-ferromagnetic materials

92. Symmetrical partial gradient coils

93. Gradient coil shield

94. Gradient coil

96. High Frequency coils

98. Patient

100. Patient bed

102. Parallel microbeam or nanobeam or minibeam generating tissue equivalent collimator system

104. Microbeam, nanobeam, minibeam focusing magnet

106. Rotating gantry

108. Compact wakefield accelerator

109. Very high energy electron beam (VHE)

110. Dual supersonic plasma jets

111. VHE-beam

112. VHE focusing magnet and vacuum beam transport

114. MRI and the compact wakefield accelerator control unit

115. VHE bending and focusing magnets and vacuum beam transport

116. Display control unit

117. Drive laser control unit

118. Beam deflection and collimator control unit

119. Gantry control unit

120. Rotary joint rotating in horizontal axis

122. Stationary and rotating gantry alignment system

124. Parallel microbeam or nanobeam or minibeam exit out of tissue equivalent collimator system

126. Focusing magnets

128. Deflected laser wakefield Thompson scattering electron or Compton scattering gamma ray with collinear electron beam or proton or carbon ion pencil beam

129. Beam switching magnet

130. To left switched pencil beam-1

132. To right switched pencil beam-2

134. To left focusing magnet

136. To right focusing magnet

138. To right 45 degree bending magnet

140. To left 45 degree bending magnet

142. 90° bending beam line

144. 11.25° bending magnet-1

146. Quadrupole focusing element

148. 11.25° bending magnet-2

150. Beam switching bipolar magnet

152. 45° Bending magnet

154. Mini storage ring

156. Initial 10 ps. 75 MHz pulse generating system

158. Diffraction grating system

160. First Ti-Sapph crystal amplifier system

162. Second Ti-Sapph crystal amplifier system

164. Third Ti-Sapph crystal amplifier system

166. Spatially stretching 50 mm Ti-Sapph crystal

168. Holographic diffraction grating system

170. 30 fs, 100 TW laser pulse

172. Dual stage composite gas target

173. Laser dump mirror and laser beam absorber

174. PW laser pulse

176. He gas jet

178. He/N2 supersonic gas jet

180. 0.5 mm gap

182. Laser focal point

184. Wake field accelerated high energy electron beam

186. Beam bending and splitting magnet system

188. Laser wakefield accelerator system

190. Five simultaneous microbeam, nanobeam or minibeam generating tissue equivalent collimator systems

192. Radiation protective treatment room

194. Treatment room radiation protective entry doors

196. Laser wakefield accelerator system room

197. Room with extracted beam

198. Wakefield accelerator system room radiation protective entry door

200. Corridor in between the two radiation protective treatment rooms

202. Office and Clinical room 1

204. Office and Clinical room 2

206. Office and Clinical room 3

208. Office and Clinical room 4

210. Office and Clinical room 5

212. Office and Clinical room 6

214. Office corridor

216. Entrance to treatment areas

217. Entrance and exit door

218. Entrance to office corridor

220. Additional shielding in laser wakefield accelerator system room

222. High density tissue equivalent block

223. High density tissue equivalent composition

224. Intermediate borated Styrofoam block

226. Borated polyethylene shield

228. Outer Cerrobend block

230. Focused primary beam without neutrons, secondary ions and gammas

232. Primary beam shield

234. Cerrobend block surrounding the borated Styrofoam in patient specific collimator

236. Outer lead block

238. Microbeam or nanobeam or minibeam generating beam line in treatment room

240. Inner block filled with tissue equivalent, high neutron cross section metal incorporated silicon compound

241. Tissue equivalent glass composition with high neutron cross section

242. Intermediate borated Styrofoam block

244. Outer lead or cerrobend lock

246. Treatment room beamline shield

247. Silica optical fiber dosimeters

248. Silica optical fiber dosimeter-1

250. Silica optical fiber dosimeter-2

252. Silica optical fiber dosimeter-3

254. Silica optical fiber dosimeter-4

256. Silica optical fiber dosimeter-5

258. Silica optical fiber dosimeter-6

260. Silica optical fiber dosimeter-7

262. Silica optical fiber dosimeter-8

264. Broad beam or arrays of microbeams before collimation

266. Inner cut

268. Central beam aperture

269. Central field opening beam aperture cut

270. Microbeam passing through the field opening

272. Neutron and gamma ray absorbing high density tissue equivalent composition in the periphery of the field opening

273. Rectangular outer borated Styrofoam

274. Outermost remaining Styrofoam after 3 cuts

275. Entrance of the field opening in inner cut

276. Opposite end of the inner cut opening

278. Densely packed high density tissue equivalent composition

280. High density patient specific block shaped beam

282. First inner cut section

284. Borated Styrofoam cut

286. Rectangular outer Cerrobend block

288. Remaining Styrofoam block

290. High density patient specific field shaping inner block

292. High density tissue equivalent glass composition

294. Inner high density tissue equivalent glass block

296. Holding Styrofoam on a tray

298. Molten glass composition is poured into metal mold

300. Nd-YAG-Laser

302. RF-Gun

304. Solenoid

306. Klystron

308. Accelerating cavity

310. Bending magnet

312. Rev. filed magnet

314. Extraction magnet

316. Focusing magnet

318. Extracted beam

320. 100 TW, 20 fs, 10 Hz laser pulse

322. Electron bunch

324. Laser pulse

326. Synchronized electron bunch-laser pulse

328. Energy modulator

330. Bunch slicer

332. Z-pinch gun

334. Very high energetic electron beam

336. Focusing magnets

338. Photocathode racetrack microtron system

340. TW laser and laser processing system

342. Scanning magnet

346. Scanned beam guiding tube

348. Drift chamber

350. Gas puff pump

352. Gas evacuation pump

354. Deuterium-Tritium gas mixture

356. Collinear very high energy electron and neutron beams

357. Sweeping magnet

358. Deflected very high energy electron beam

360. Deflected electron beam splitting magnet

362. Very high energy split electron beams

364. Multiple MRT rooms

366. Very high energy neutron beam

368. High flux neutron-radioisotope precursor reaction chamber

370. Dielectric waveguide

372. Inner vacuum

374. Dielectric region

376. Metal guide

378. Corrugated pipe waveguide

380. Whole blood reservoir

382. Densitometer-1

384. Pulsed pump

386. CTC, plasma-platelet and exosomes, microsomes and nanosomes reservoir

388. Pulsed pump

390. Densitometer-2

392. DNA/RNA/Telomerase, exosomes, nanosomes affinity column-1 with EGCG

394. Densitometer-3

396. Pulsed pump

398. Purified plasma collecting bag

400. Densitometer-4

402. Reservoir with CTC, platelets, exosomes, microsomes and nanosomes/DNA-Telomerase

404. Pulsed pump

406. DNA/RNA/Telomerase, exosomes, nanosomes affinity column-2 with EGCG

408. Densitometer-5

410. Pulsed pump

412. Purified platelets collecting bag

414. Densitometer-6

416. Pulsed pump

418. Reservoir for RBC plus WBC and CTC, exosomes, microsomes and nanosomes

418B. Reservoir with WBC, CTC, exosomes, microsomes and nanosomes/DNA-Telomerase

420. Densitometer-7

422. Pulsed pump

424. DNA/RNA/Telomerase, exosomes, nanosomes affinity column-3 with EGCG

426. Densitometer-8

428. Pulsed pump

430. CTC, DNA/RNA/Telomerase, exosome, microsomes and nanosomes free WBC collecting bag

432. Densitometer-9

434. Pulsed pump

436. Reservoir with concentrated RBC, CTC, exosomes, microsomes and nanosomes/DNA-Telomerase

438. Densitometer-10

440. Pulsed pump

442. DNA/RNA/Telomerase, exosomes, nanosomes affinity column-4 with EGCG

444. Densitometer-11

446. Pulsed pump

448. Purified RBC collecting bag

450. Pulsed pump

452. Air bubble sensor

454. Densitometer-12

456. Treated return blood in blood flow tubing

458. Reservoir for DNA/RNA/Telomerase, tumor associated exosome, microsomes, nanosomes and CTC free blood after pulse flow purification

460. Blood flow inlet channel with clam and sensor

462. Blood flow return channel with clam and sensor

464. System clamp with sensors

466. Diluting normal saline

468. Anticoagulant reservoir

470. Blood flow tubing

472P. Microfilter for CTC removal from plasma

474P. Microfilter plasma CTC elution collection inlet and outlet

476P. Purified plasma collection inlet and outlet

476W Microfilter for removal of CTC bound to WBC

478R. microfilter for removal of CTC bound to RBC concentrate

478PL. Microfilter for removal of CTC bound to platelet

480PL. Microfilter platelet CTC elution collection inlet and outlet

482PL. Purified platelet collection inlet and outlet

484W. Microfilter WBC bound CTC elution collection inlet and outlet

486W. Purified WBC collection inlet and outlet

488R. Microfilter RBC bound CTC elution collection inlet and outlet

490R. Purified RBC collection inlet and outlet

492. Inlet and outlet tube connection

494. Ultracentrifuge continuous flow rotor

496. Bottom sample inlet

498. Mechanical seal

500. Damper

502. Bottom hollow driveshaft

504. Rotation Chamber

506. Core

508. High speed rotating cylindrical rotor

510. Top hollow driveshaft

512. High frequency motor

514. Top Mechanical seal

516. Supernatant outlet

518. Purified plasma return to patient

520. Control system's LCD

522A. Immunoadsorbent affinity chromatography column-1

522B. Immunoadsorbent affinity chromatography column-2

522C. Immunoadsorbent affinity chromatography column-3

522D. Immunoadsorbent affinity chromatography column-4

522E. Immunoadsorbent affinity chromatography column-5

522F. Immunoadsorbent affinity chromatography column-6

522G. Immunoadsorbent affinity chromatography column-7

522H. Immunoadsorbent affinity chromatography column-8

522I. Immunoadsorbent affinity chromatography column-9

522J. Immunoadsorbent affinity chromatography column-10

524. Plasma injector to rotor

526. Pulsed flow apheresis plasma into plasma injector

528. Pulsed flow apheresis plasma injector

530. Cooling coils

532. Warming coils

534. Effluent from the chromatographic columns

536. AFM

538. NTA

540. DCNA

542. FCM

544. Electronic flow direction control switch

### 29. DETAILED DESCRIPTION OF THE PREFERRED EMBODIMENTS

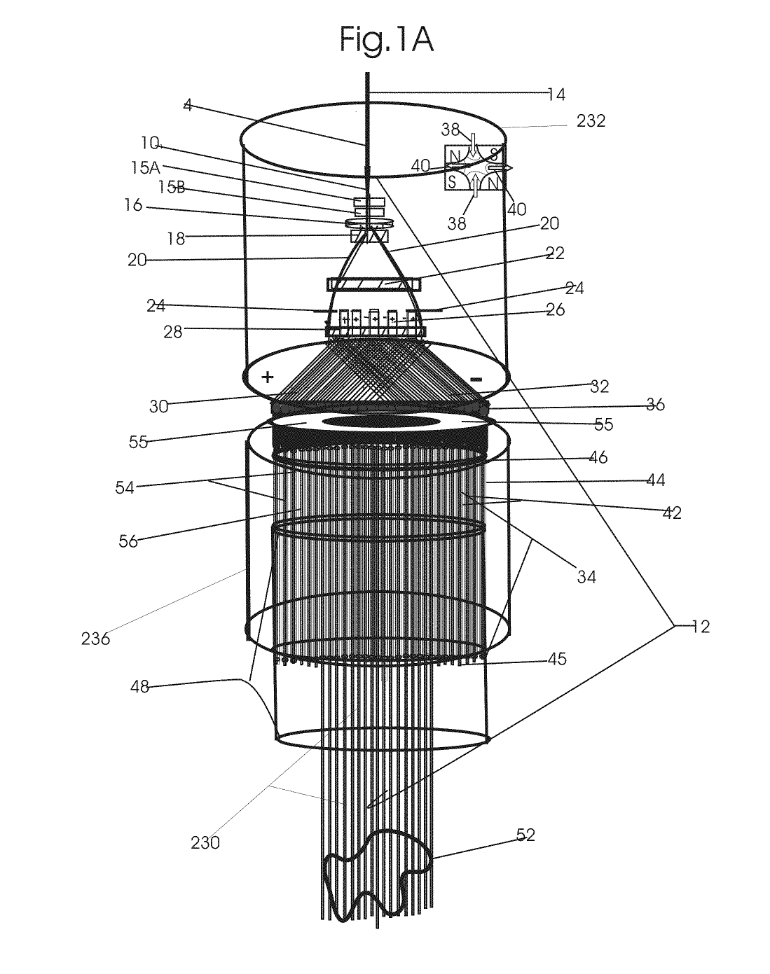

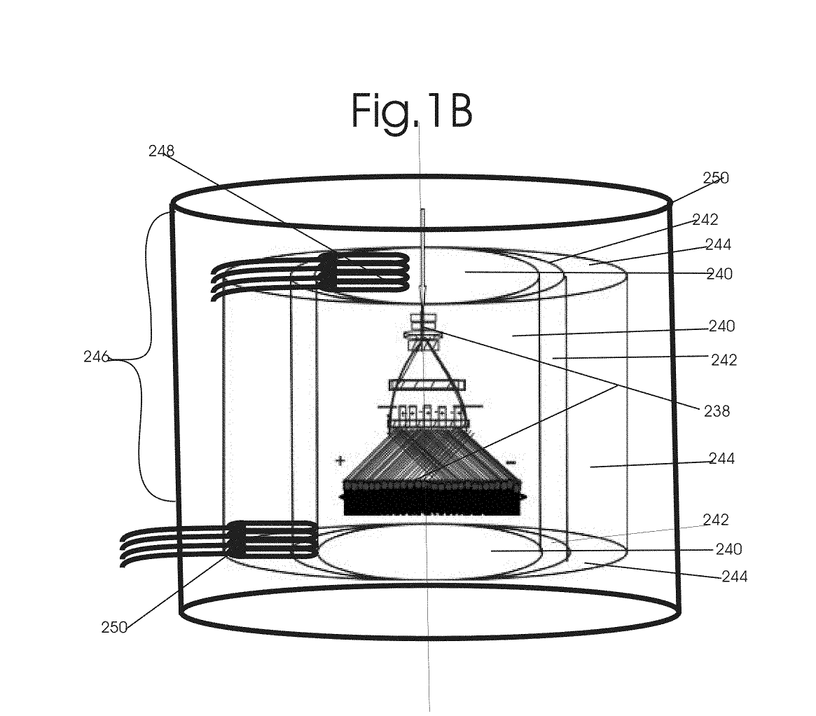

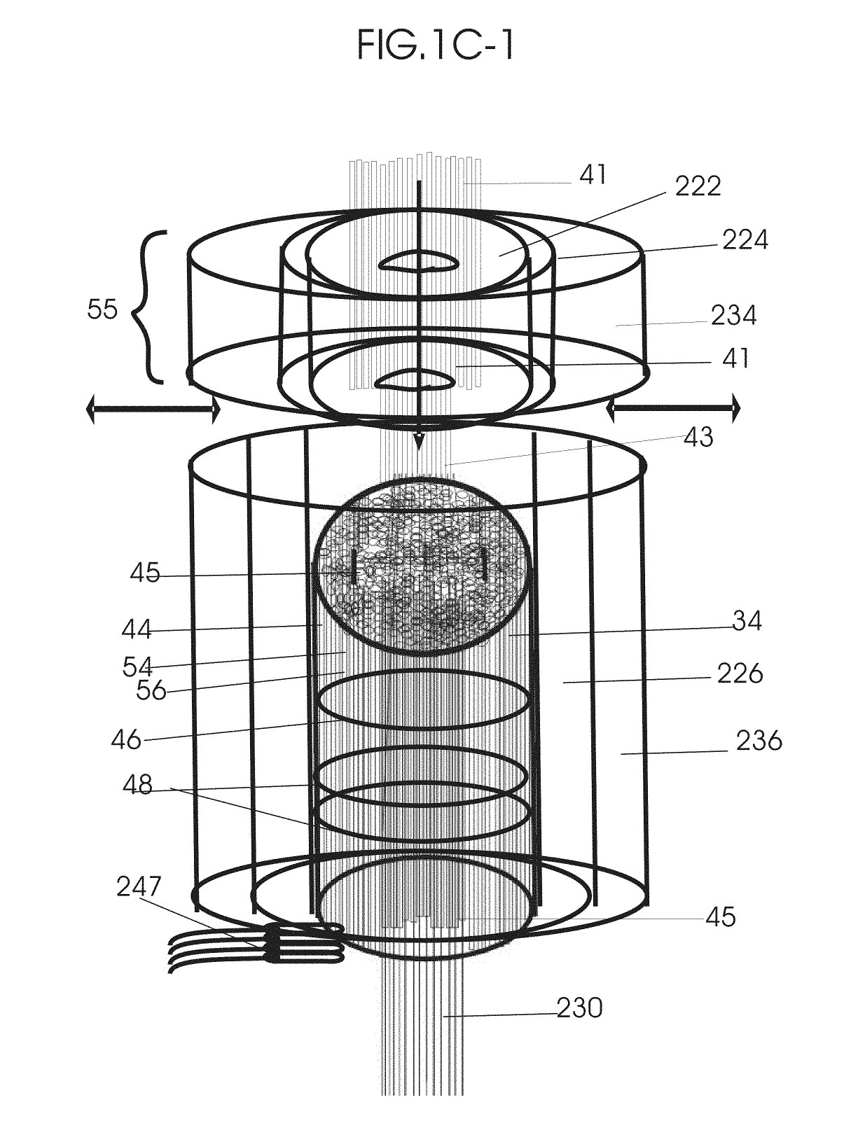

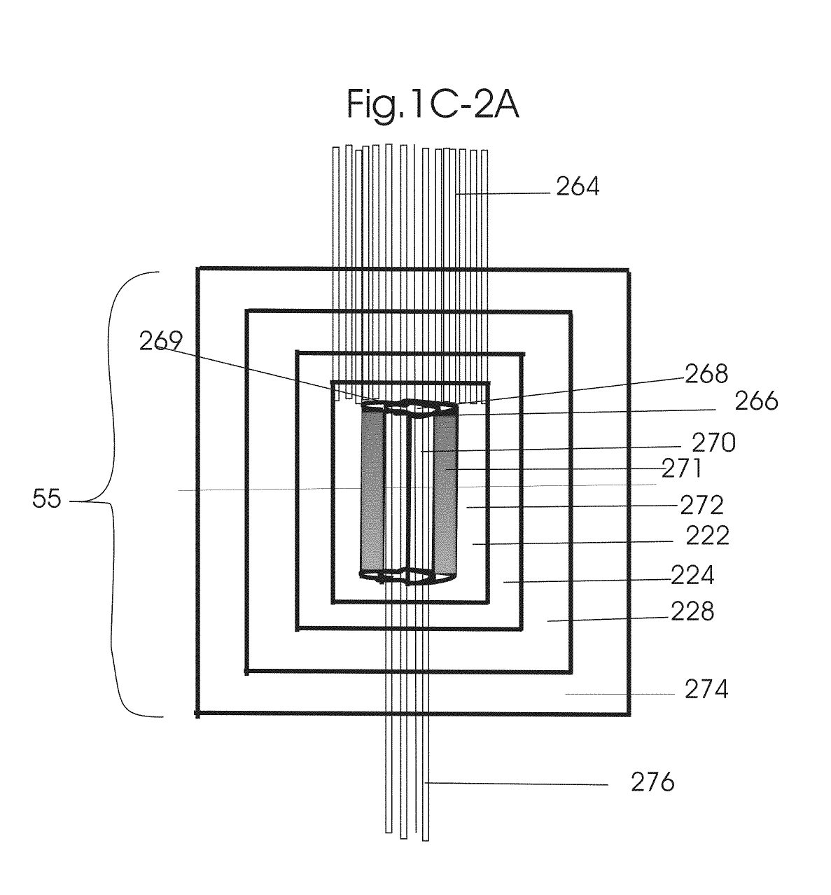

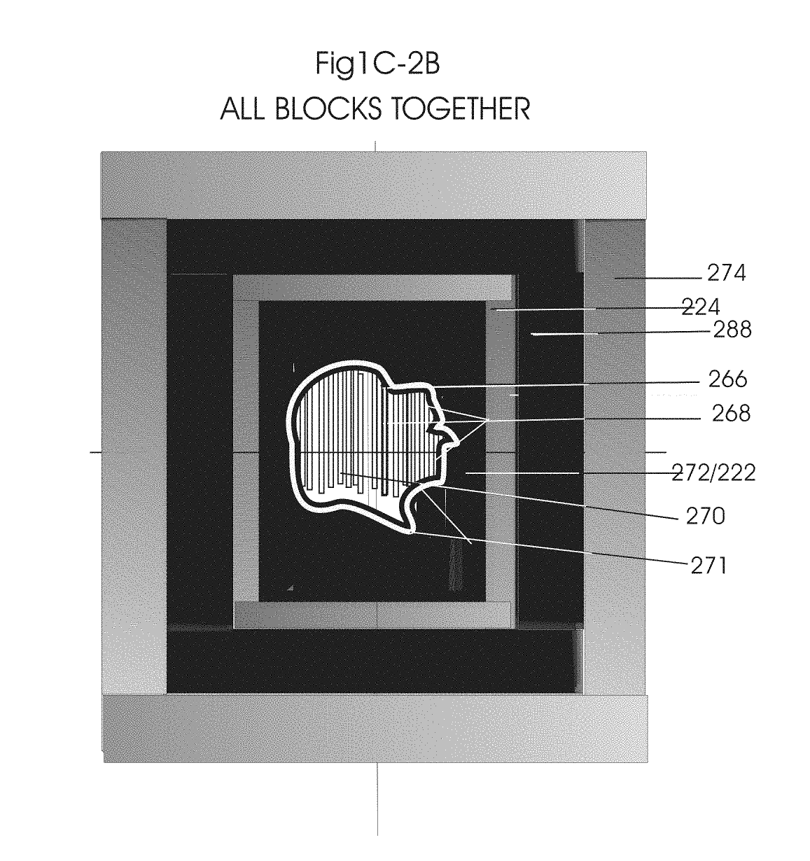

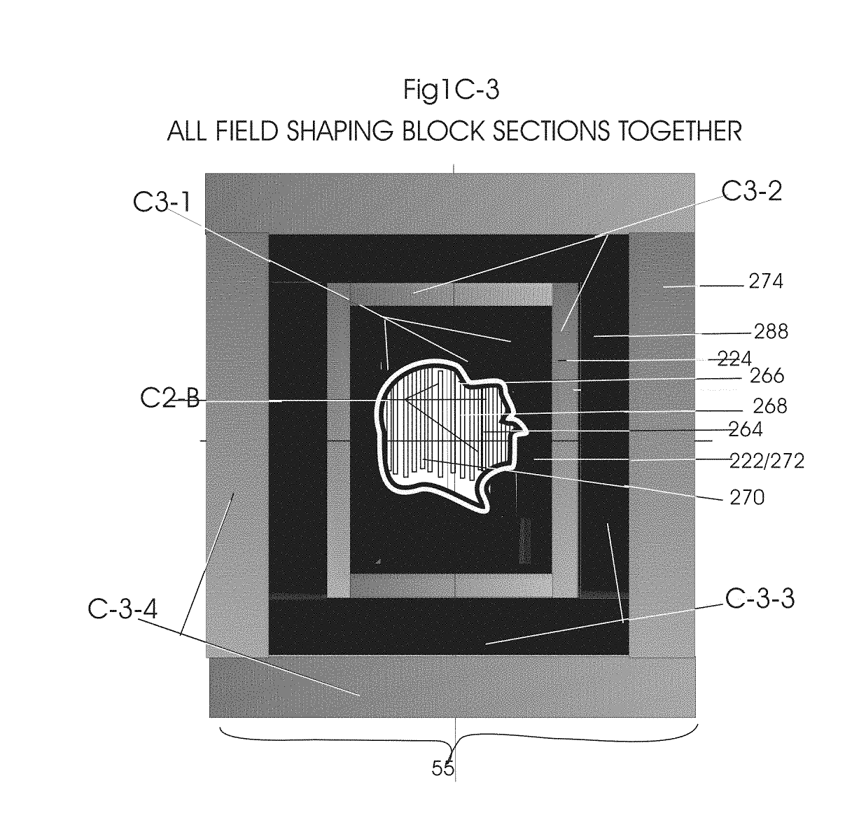

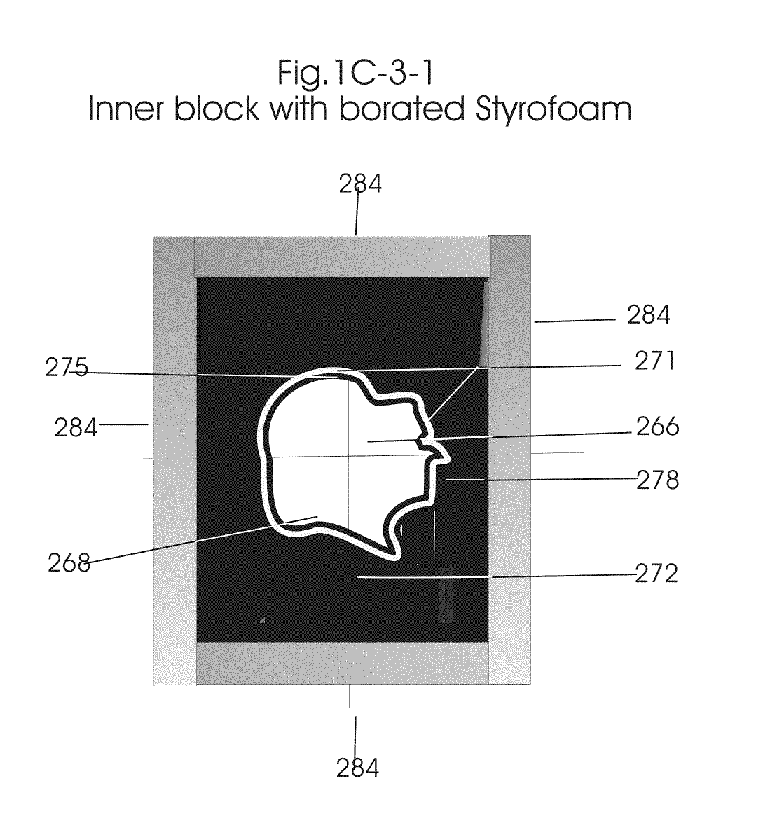

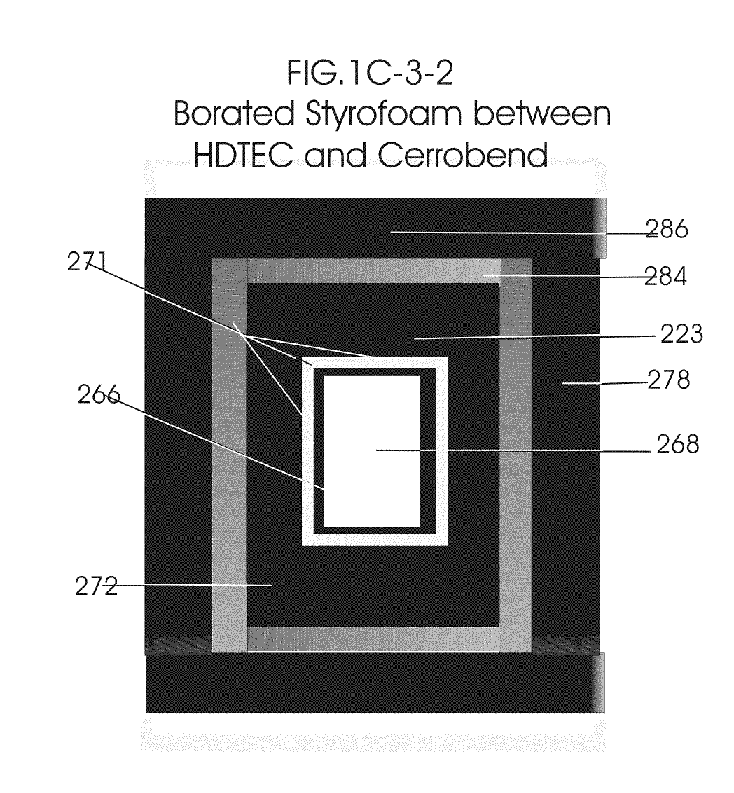

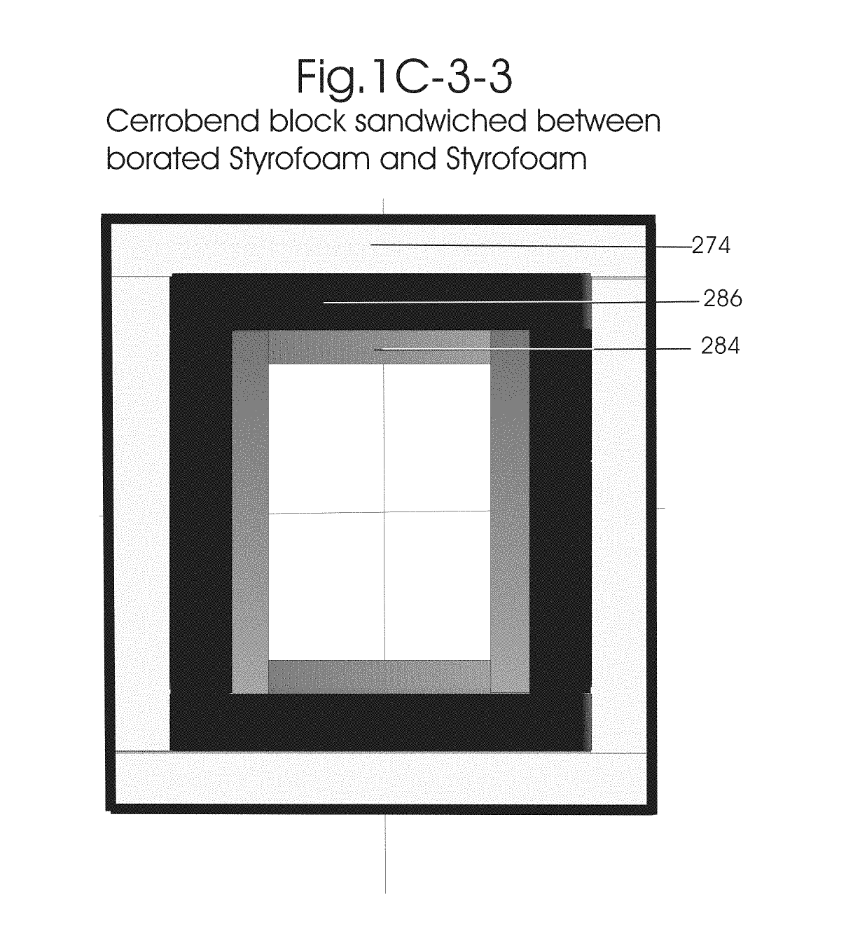

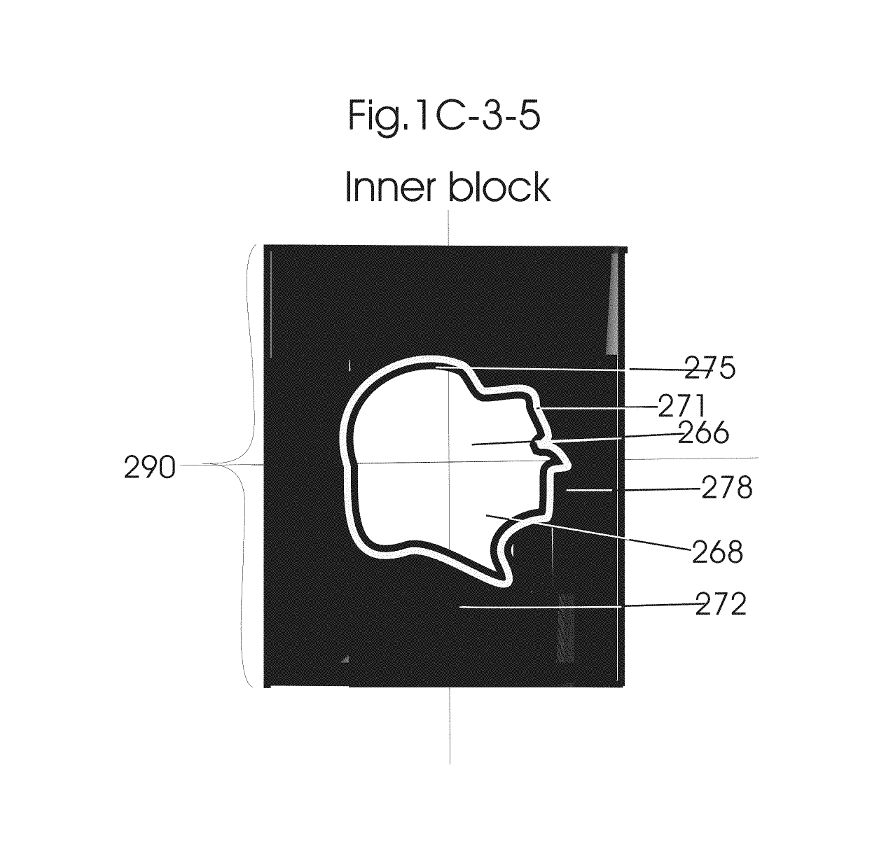

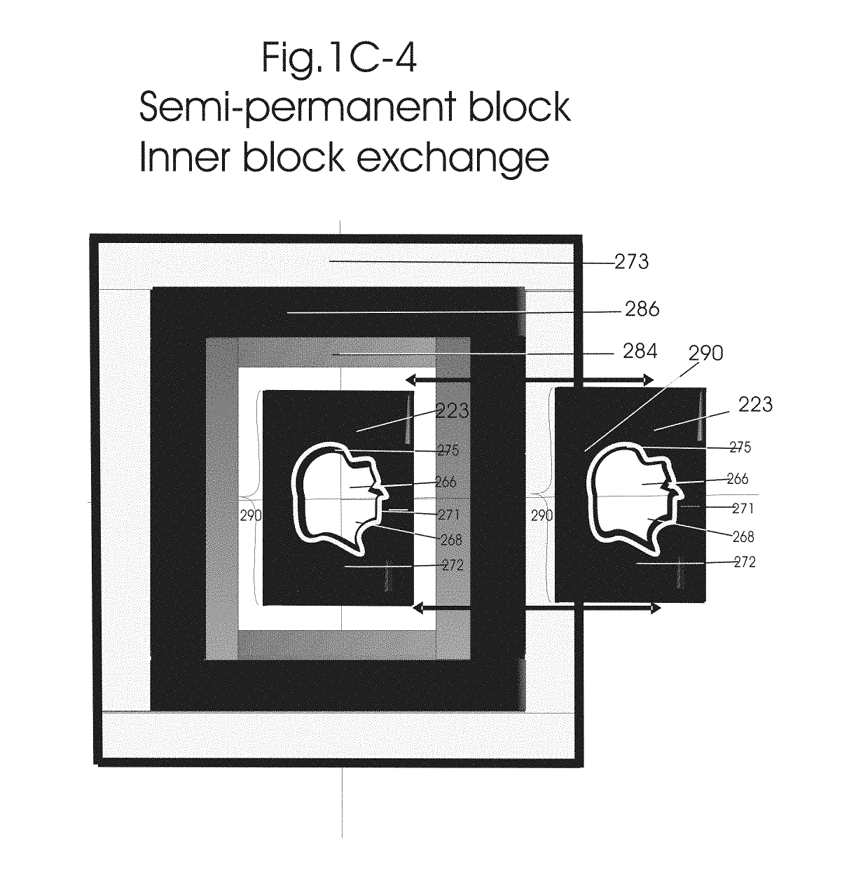

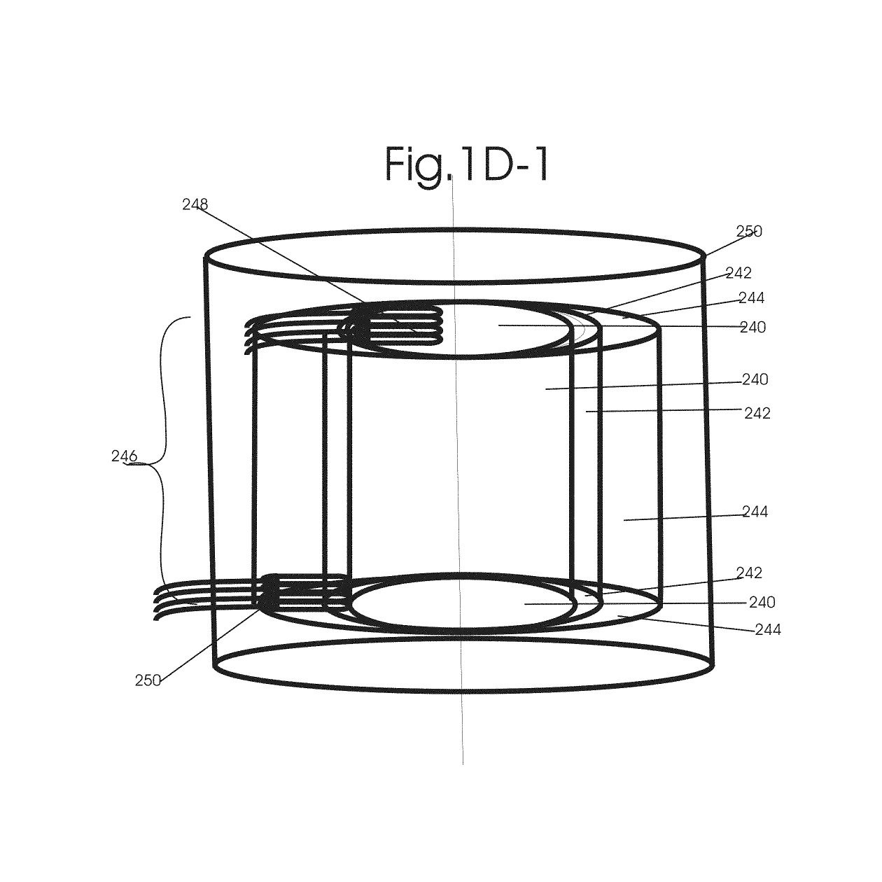

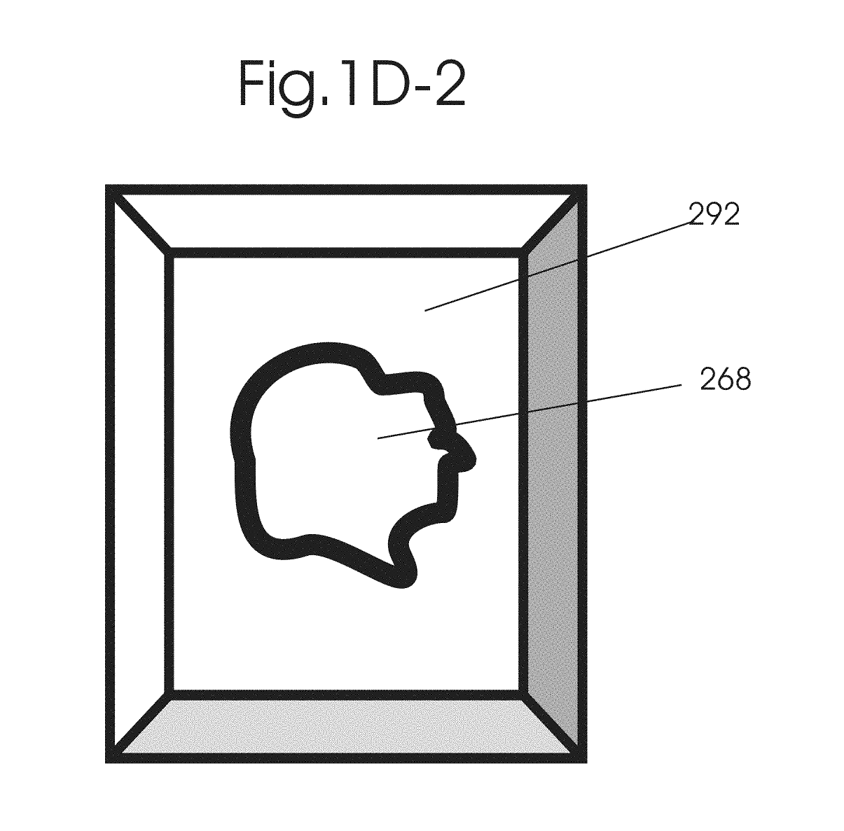

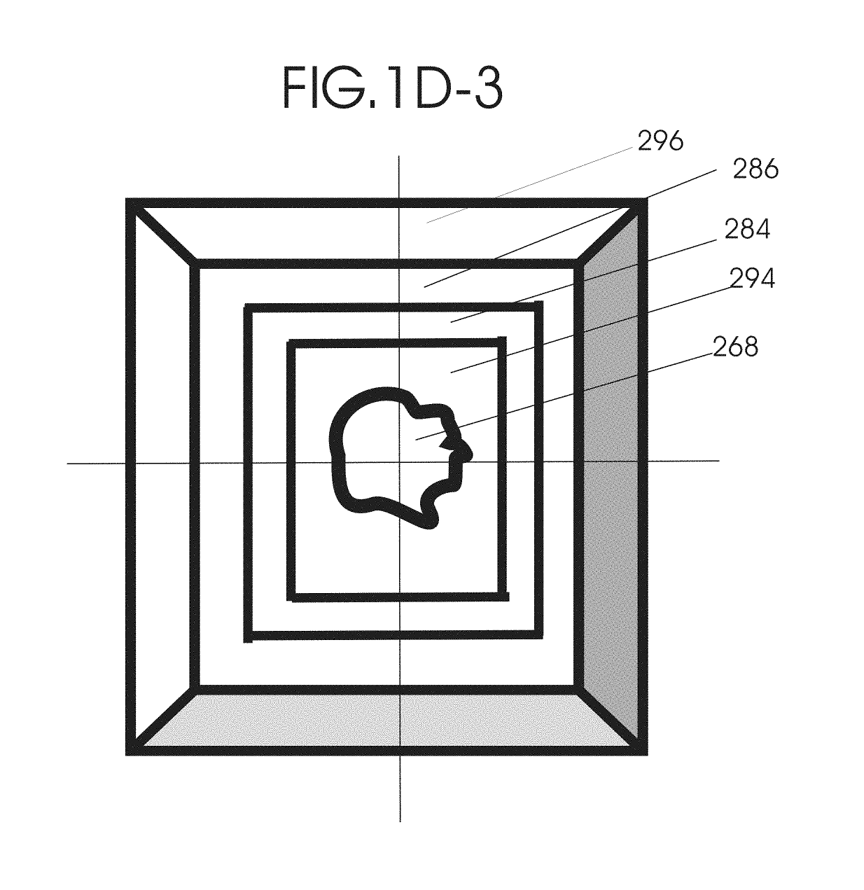

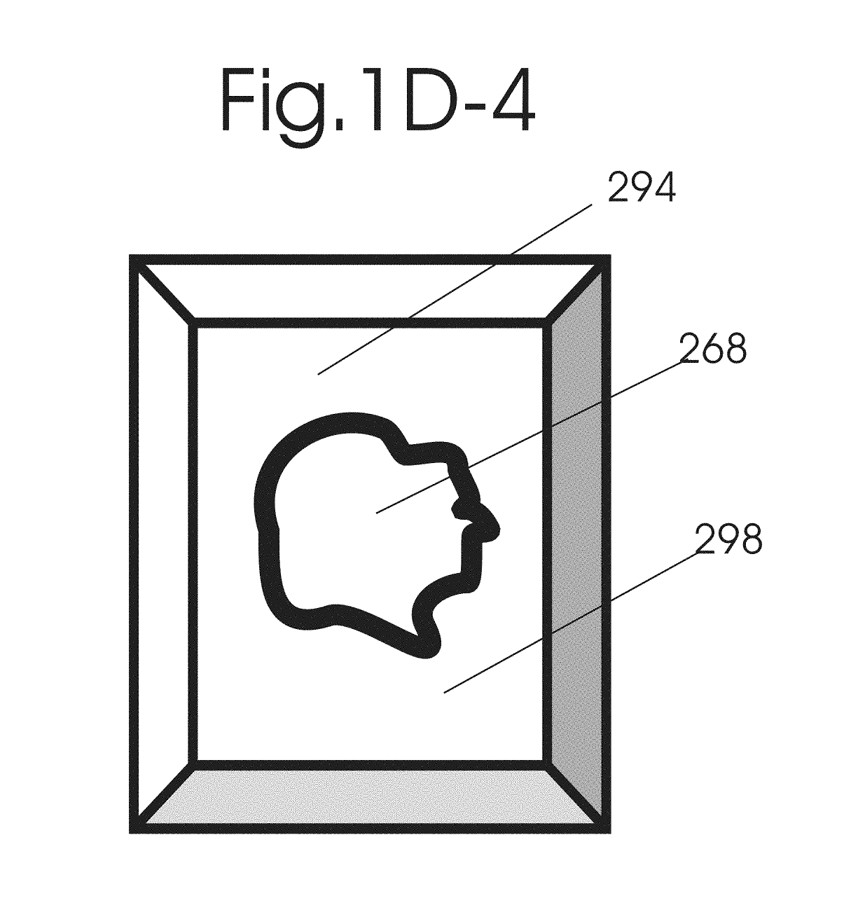

FIG. 1A shows a microbeam, nanobeam or minibeam generating collimator system that is used with a modified wakefield accelerator system that generates high energy electron beam or inverse

Compton scattering collinear electron and gamma ray or proton beam for kGy dose radiosurgery with least radiation toxicity to normal tissue and least secondary neutron and gamma radiation exposure to the patient and to the areas surrounding the accelerator and treatment rooms.

The laser wakefield Thompson scattering electron or Compton scattering gamma ray with collinear electron beam or proton or carbon ion beam 14 is made to pass through an emergency beam stopper 15A and a dose monitor 15B and collimated by a collimator 16. This collimated beam is then defocused in one plane and focused in another plane with a quadrupole magnet 18 which spreads out the beam in one plane and focuses it in another plane. The insert shows the quadrupole magnet with converging magnetic field in one plane 38 and the diverging magnetic field in another plane 40 as arranged symmetrically about the beam axis. The quadrupole magnet 18 with converging magnetic field in one plane 38 which focuses the inverse laser wakefield Thompson scattering electron or Compton scattering gamma ray with collinear electron beam or proton beam 14 and the diverging magnetic field in another plane 40 defocuses it. The one plane defocused and in another plane focused negatively charged beam 20 is injected into a defocusing, focusing and beam size controlling magnet 22. The split beam's size and spacing from each other is controlled with this magnet. This beam deflected in one direction and focused in another passes through a stripper grid 24 that generates alternating positively and negatively charged beam segments 26. They are alternatively charged as positive and negative segments of the beam and they are passed through a deflection magnet with DC vertical dipole field 28. According to the Lawrence law of force, the positively charged collinear beams 30 deflects to the left and the negatively charged collinear beams 32 deflects to the right. The separating distance between each of these beams is dependent on the strength of dipole field. It generates numerous simultaneous parallel collinear microbeams or nanobeams or minibeams. These beams are further processed as microbeams or nanobeams or minibeams within the tissue equivalent primary collimator 34. The size of the beam, microbeam or nanobeam or minibeam also depends on the size of the microfocus beam guides in the tissue equivalent block 44 in the in the tissue equivalent primary collimator 34.

Down stream to the positively charged electron or collinear electron/gamma ray beamlet or proton beams 30 and the negatively charged collilinear electron/gamma ray beams or proton beams 32 a tissue equivalent collimator 36 is placed. The positively and negatively charged wakefield laser electron beam or collilinear electron/gamma ray beams or proton beams 30 and 32 are incident into a tissue equivalent primary collimator 34 which also contains microfocus carbon tubes 44. It is partially filled with tissue equivalent material for absorption of the scattered radiation and secondary neutrons and ions. The clean beam without the contaminating low energy scattered x-rays and secondary protons, neutrons and other ions exits from the microfocus beam guides in the tissue equivalent block 44 at its distal end opening. The focusing anode 46 and the focusing magnet 48 keep the wakefield laser electron beam or collilinear electron/gamma rays or proton beam as focused without any significant penumbra.

The magnetically focused electron beam's penetration below the skin is much deeper. Its maximum dose, the dₘₐₓ is much deeper in the skin than those for the unfocused electron beam (17. 18). It removes the unfocused electron beam's higher rate of toxic reaction within the skin surface and in tissue below it. The severe errythema, edema, pain and ulceration from unfocused conventional high dose electron beam radiation to the skin are avoided by such magnetically focused electron beam radiation therapy. It is very important for safe administration of single or hypofraction kGy radiosurgery with electron. Furthermore, such magnetically focused beams have much less penumbra. It avoids smearing of the adjacent microbeam's base by each other. It creates much better defined low dose valley region 56. It helps to heal the normal tissue through which the microbeam, nanobeam or minibeam travels towards an isocentric tumor. The radiation damage in normal tissue is repaired by proliferation of its normal stem cells. However, the Monte Carlo simulation of 150-250 MeV electron beam was reported to have less lateral penumbra and its depth dose at less than 10 cm is similar to photon beam and as having practical range Rₚ greater than 40 cm (37). Combined with magnetic focusing of the very high energy electron pencil beam (17, 18), microbeam and nanobeam and the very high energy electron beam's deeper penetration as shown by Monte Carlo simulation (37) makes the deep penetrating pencil electron beam, microbeam, nanobeam and minibeam similar to proton pencil beam microbeam, nanobeam and minibeam with less skin toxicity and with well defined microbeam, nanobeam and minibeam peak and valley doses.

To maintain the peak and valley dose differential as in microbeam radiosurgery, the microfocus beam guides in the tissue equivalent block 44 are placed at a distance of one to four ratio of beam width and the distance from each other in the tissue equivalent primary collimator 34. If the beam width is say 75 micrometers then the distance from two adjacent microfocus beam guides in the tissue equivalent block 44 is kept as 300 micrometers. If the beam width were 10 micrometers, then the distance from two adjacent microfocus beam guides in the tissue equivalent block 44 is kept as 40 micrometers apart. Similar ratio of distance from microfocus beam guides in the tissue equivalent block 44 is kept for nanobeams and minibeams. If 500 nanometer width nanobeams were used for nanobeam radiation, then the distance from two adjacent microfocus beam guides in the tissue equivalent block 44 is kept as 2,000 nanometers that is 2 micrometers apart. If it is minibeam with 300 micrometer width, then the distance from two adjacent microfocus beam guides in the tissue equivalent block 44 is kept as 1.2 mm apart. The collinear parallel beams 42 that enter into the microfocus beam guides in the tissue equivalent block 44 are focused by the focusing anode 46 and the focusing magnet 48. Focusing of the wakefield laser electron or collilinear electron/gamma rays or proton beam traveling through the microfocus beam guide 44 eliminates the disadvantages of widening of the beam when it travels through a long tissue equivalent universal collimator 34. The focused microbeam or nanobeam or minibeam leave the microfocus beam guides in the tissue equivalent block 34 as focused primary beam without neutrons, secondary ions and gammas 230 travels towards the isocentric tumor 52. The primary beam shield 232 is made of neutron absorbing chromium ash and chromium powder mixture in borated Styrofoam cut. It is similar to Cerrobend block making for megavoltage radiation therapy. The boron in the borated Styrofoam moderates the gamma ray from 2.2 MeV to 477 keV. It is described in more details under FIG. 1B. An outer, 3 half value layers, 6 cm thick lead block surrounding the borated Styrofoam 234 (in FIG. 1C) absorbs most of the gamma rays. The patient specific collimator 55 is also made of neutron absorbing chromium ash and chromium powder mixture in borated Styrofoam encased in a lead block. Likewise, the tissue equivalent primary collimator 34 is surrounded by an outer lead block 236 for the gamma ray absorption. To shape the microbeam or nanobeam or minibeam in conformity with the tumor volume, varying shape and size patient specific collimators 55 are placed upstream to the tissue equivalent primary collimator 34. The portion of the tissue that is radiated by the narrow parallel beams is the peak dose region 54. The tissue that is separated between the two peak radiation regions in tissue is the low or no dose valley dose 56.

FIG. 1B is a continuous illustration of FIG. 1A to show the absorption of secondary neutrons, ions and gamma radiation generated by the beamline in the treatment room with a tissue equivalent collimator to reduce secondary radiation in the treatment room and outside the treatment room produced by the beamline. The treatment room beamline shield 246 encloses the microbeam or nanobeam or minibeam generating beam line in treatment room 238. It consists of an inner block filled with tissue equivalent, high neutron cross section metal incorporated silicon compound 240 that absorbs neutrons generated in the microbeam or nanobeam or minibeam generating beam line in treatment room 238. It is surrounded by an intermediate borated Styrofoam block 242 that moderates the high energy gamma rays produced by the interaction of hydrogen and proton to 477 keV gamma rays which is easier to block with an outer lead or Cerrobend block 244.

Shielding of neutron with cost-effective silicon containing metallic compounds is known. It is also described in US patent application 2009/0085011 which is incorporated herein in its entirety by reference. Simpler other methods of silicon incorporated metallic compounds synthesis includes melting the metal together with plant wastes like melting chromium with rice husk charcoal as in silicate ornamental glass making (19). Rice husk charcoal contains higher than 80% silicates by mass (19). Similar low cost gadolinium silicate is also prepared (51)

The chromium recovery from liquid and solid tannery waste as chromium ash containing over 25% chromium (21) depends on chromium being incorporated into tannery organic compounds. It is a high density tissue equivalent metal organic compound composition that is useful for making high density tissue equivalent collimators as in this invention. Organic compounds like chromium cobalt hexamine trichloride and triiodide, cobalt hexamine perrhenate, chromium hexamine trichloride and triiodide and chromium hexamine perrhenate are used for shielding of neutron and gamma radiation in nuclear reactors (22).

Among the naturally occurring elements, Gadolinium (Gd) has the highest thermal neutron absorption cross-section (23). ¹⁵⁷Gd and 155Gd have 253,000 and 60,700 barns respectively for neutron absorption cross-sections (23) The glass density of gadolinium glass with 35 mol % gadolinium oxide (Gd2O3) is close to 4.5 (24). The density of gadolinium oxide is 7.407. Gadolinium oxide power or gadolinium glass powder filled Styrofoam cut as in Cerrobend block is an excellent neutron absorber. Gadolinium glass with laser cut micrometer or nanometer sized holes as beam lines is an excellent microbeam, nanobeam or minibeam creating collimator that eliminates the secondary neutron exposure to the patient and to the treatment room and to its surrounding areas.

152Gd 735, 154Gd 85, 155Gd 61.100, 156Gd 1.5, 157Gd 295,000, 158Gd 2,5, 152Gd and 160Gd 0.77. 157Gd's cross section of 295,000 with its highest cross section for thermal neutron capture makes it both ideal for neutron shield and also for neutron dosimetry. The natural Gd contains 15. 65% 157Gd. After excitation, internal conversion and emission of gamma rays, 157Gd converts to 158Gd with 2.5 neutron capture cross section. In contrast to Gd, chromium and chromium isotopes have much lower neutron cross section, just 24 and lower. Still chromium amines were used as effective nuclear reactor neutron and gamma ray shield (22). There are several other high density elements and alloys suitable for making neutron and gamma shields. In this invention, gadolinium silicate organic compound with densities of about 4.5 or chromium silicates organic compounds with density of about 2.5 are used as neutron and gamma ray absorbing microbeam or nanobeam or minibeam generating tissue equivalent collimator.

The scattered and secondary gamma and neutron generated by the high energy electron, Compton gamma, proton and ions both inside and outside the treatment room are monitored with silica optical fiber dosimeters. The commercially available glass fiber scintillateor, its electronics algorithms are used for neutron and gamma radiation detection and dosimetry. Glass optical fibers transports light to its end when exposed to neutron or gamma radiation. This light is enhanced by photomultiplier tube attached to it. The PNNL glass fibers incorporated with silicon are also coated with 6Li and 3Ce. The 6Li with neutron cross section of 940 undergoes 6Li (n,a) reaction that produces a tritium ion and alpha particle. This tritium ion and alpha particle ionizes the glass particles. The ionized glass particles transfer energy to 3Ce ions. As the excited state 3Ce returns to ground stage, it emits flash of light for each neutron absorbed with wavelength of 390 to 600 nm. A part of this light is transmitted through the scintillating glass wire that is connected to a photomultiplier in a detector. With the detector's pulse height discrimination analysis, both these neutron and gamma rays are analyzed.

In this invention, gadolinium silicate is used to absorb neutron and gamma rays generated in the beam blocks. The natural Gd contains over 15% 157Gd. The ¹⁵⁷Gd with neutron cross section of 295,000, is a much more superior neutron and gamma ray detector than the ⁶Li with neutron cross section of 940. Similar to the hot downdraw process neutron sensing silicon glass fiber making from molten silicon glass at the Pacific Northwest National Laboratory PNNL NNL (25), glass fiber is made from molten Gd silicate glass with 3Ce for neutron and gamma ray detection. Alternatively, commercial neutron absorbing glass fiber dosimeters are used.

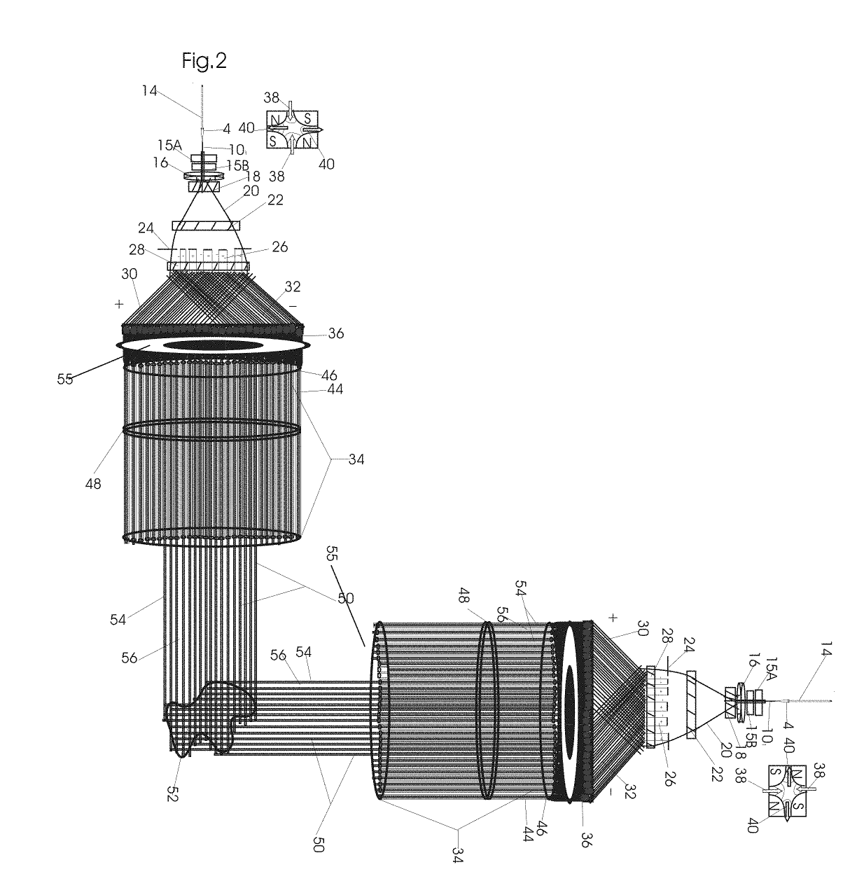

FIG. 1C-1, FIG. 1C-2A, FIG. 1C2B and FIG. 1C3 and their associated FIG. 1C-3-1, FIG. 1C-3-2, FIG. 1C3-3 and FIG. 1C3-4 are continuous illustration of the patient specific high density field defining block 55 shown in FIG. 1A. They show absorption of secondary neutrons, ions and gamma radiation generated in the high density patient specific filed shaping block 55.

FIG. 1C-1 shows a patient specific field defining high density tissue equivalent block inserted at the top of the high density tissue equivalent microbeam, nanobeam or minibeam generating collimator 34.

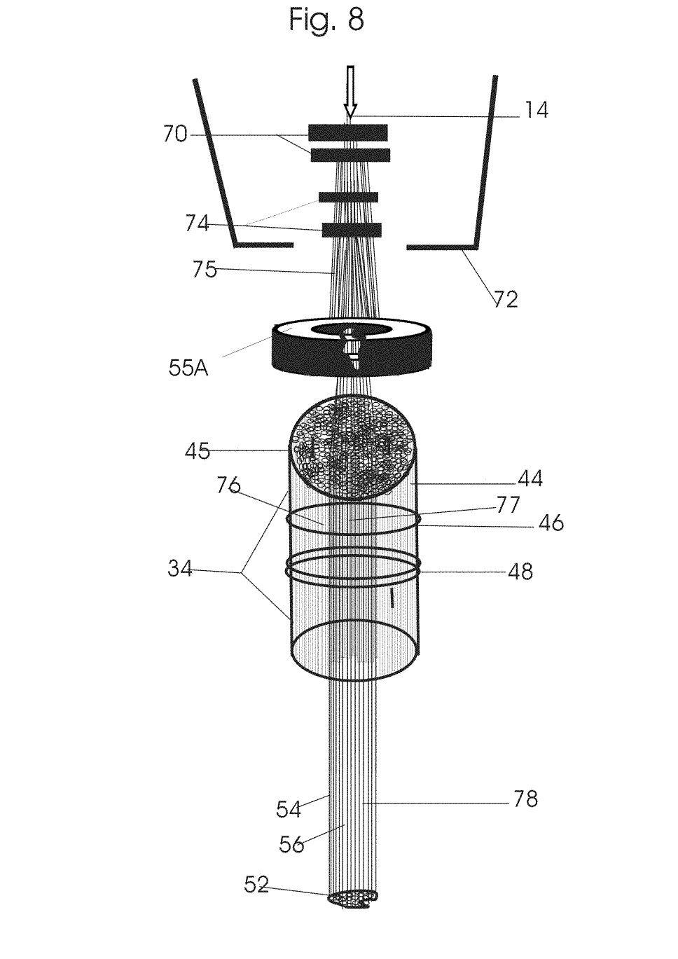

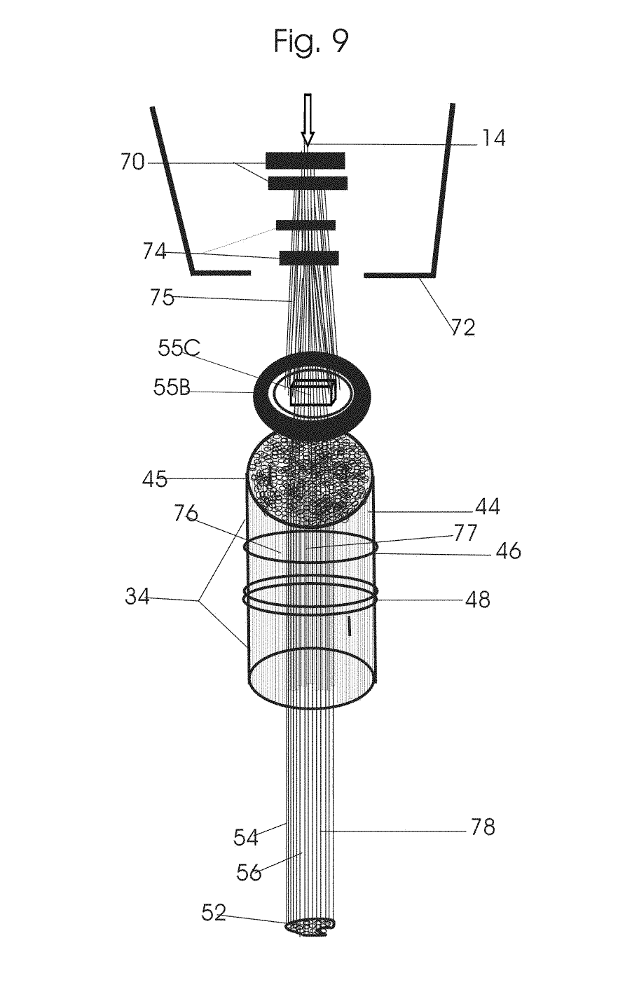

This patient specific field defining high density tissue equivalent block is similar to the treatment room beamline block described under FIG. 1B except it is a smaller interchangeable block that is specifically made for each patient's treatment. It differs from the conventional MLC and Cerrobend method of treatment field defining. Since it is mostly for single fraction, high dose radiosurgery and due to high levels of secondary radiation including neutrons and gamma generated by the MLC, field defining by MLC is not suitable for super high dose and kGy radiosurgery unless they are modified as described under FIG. 8 and FIG. 9. This patient specific field shaping block 55 has three sections, an inner section made of metal incorporated silicon compound, an intermediate section made of polyethylene incorporated Styrofoam and an outer section made of Cerrobend. The inner section of the patient specific block with high density tissue equivalent composition 222 absorbs most of the secondary neutrons and gamma rays but its interaction with hydrogen and neutron generates high energy gamma rays that need to be moderated to lower energy gamma rays so that it can be absorbed in lesser amount of Cerrobend. It absorbs the neutrons generated in the microbeam or nanobeam or minibeam generating beam line in treatment room 238. It is surrounded by an intermediate borated Styrofoam block 224 that moderates the high energy gamma rays produced by the interaction of hydrogen and proton to 477 keV gamma rays which is absorbed by the outer Cerrobend block 228 surrounding the borated polyethylene block. The arrows indicate the directions by which the patent specific field shaping block 55 can be inserted and moved to align it with the microbeam or nanobeam or minibeam generating high density tissue equivalent primary collimator 34 below the patient specific field shaping block 55.

The system components of the microbeam or nanobeam or minibeam generating high density tissue equivalent primary collimator 34 are described under FIG. 1. Briefly, they include microfocus beam guides 44 within the microbeam or nanobeam or minibeam generating high density tissue equivalent primary collimator 34 with microfocus beam guide's openings and exit 45 in the tissue equivalent block, focusing anode 46, focusing magnet 48, peak dose region 54 and low dose valley region 56. It is surrounded by a borated polyethylene shield 226 and an outer lead block 236. The focused primary beam without neutrons, secondary ions and gammas 230 is also shown as exiting from the microbeam or nanobeam or minibeam generating high density tissue equivalent primary collimator 34.

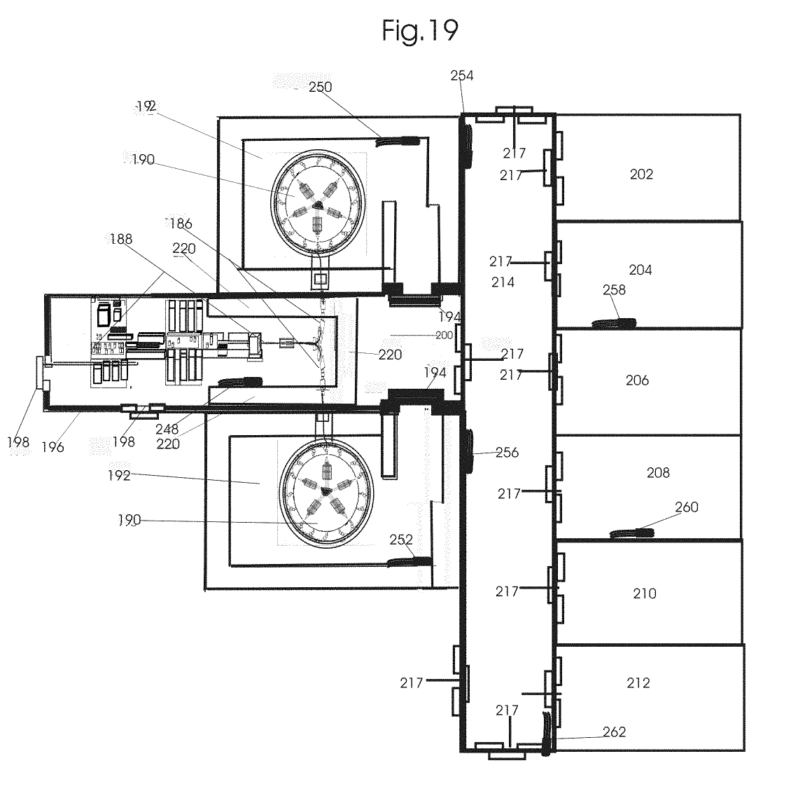

The 100-250 MeV electrons or proton radiation reaching the patient specific field shaping block 55 could generate very low level residual activity in the patient specific field shaping block 55 after radiosurgery. Most of it lasts only for seconds. However, it would be of concern if the patient specific field shaping block 55 were handled repeatedly by the treatment delivering personals. Since the patient specific filed shaping block 55 is a single use, disposable one, it is not a major clinical concern. Still it is tested for any residual activity. If it is found to have higher than safe operational level residual activity as defined in radiation safety guidelines, it is kept safely until the residual activity decays to a safe level before its reuse to make Cerrobend block for another patient. Likewise, other sections of this high density patient specific field shaping block 55 are handled according to the presence or absence of residual activity after exposure to treatment beam. Other sections of beam block and beam handling collimator systems are heavily shielded from radioactivity. Still, they are monitored with several dosimeters as shown in FIG. 1B, FIG. 1C and in FIG. 19 to take corrective actions immediately if needed. These are additional radiation safety measures to the routine radiation safety checks performed in a clinical radiation therapy department.

FIG. 1C-2A and FIG. 1C2-B schematically illustrates the microbeam passing through the central field opening aperture in a high density tissue equivalent patient specific field shaping block 55 constructed with mold making Styrofoam cuts as in Cerrobend block making for conventional radiation therapy. Like in Cerrobend block making, a central cut in conformity with the patient's tumor and safe margin, the PTV and margin plus an additional inner cut representing the border of PTV and margin is made in a Styrofoam block and it is removed. It gives a central beam aperture 268 for passage of the beam through the high density patient specific block. The additional inner cut in the Styrofoam adjacent to the central beam aperture cut 268 forms the inner PTV plus margin limiting Styrofoam wall 271. When the Styrofoam cuts are fixed onto a block holding tray and the central beam aperture cut 269 and the inner cut made for filling with high density tissue equivalent composition 223 is removed, an open space with the inner PTV plus margin limiting Styrofoam wall 271 is formed. It borders the central beam aperture cut 269. High density tissue equivalent composition 223 is packed into this space. Construction of the high density patient specific field shaping block 55 with the aid of Styrofoam mold making cuts is further described below. The inner cuts 266 and the inner PTV plus margin limiting Styrofoam wall 271 outlines the central beam aperture cut 268. A third Styrofoam cut representing the treatment field with its peripheral beam block defines the inner section of the high density tissue equivalent patient specific field shaping block 55, which is the high density tissue equivalent block 222. The beam passing through the field opening 270 radiates the tumor. The neutron and gamma ray absorbing high density tissue equivalent composition in the periphery of the field opening 272 include Gd silicate powder or chromium silicate powder described under FIG. 1B and FIG. 1C-1 or similar ones. As described in FIG. 1C-1, this high density tissue equivalent patient specific field shaping block 55 has three sections, an inner section made of metal incorporated silicon compound, an intermediate section made of polyethylene incorporated Styrofoam and an outer section made of Cerrobend. It is the inner section of the patient specific block with high density tissue equivalent composition 222 that absorbs most of the secondary neutrons and gamma rays. The hydrogen in the high density tissue equivalent inner section interacts with neutron and generates high energy gamma rays. It is moderated with the 2nd section of the block, the borated Styrofoam block 224 to 477 keV gamma rays which is absorbed by the outer cerrobend block 228 surrounding the borated polyethylene block. The outermost Styrofoam block 274 is a holding Styrofoam block that holds all these block together. The beam processed in high density patient specific field defining block 276 exits from the high density patient specific field defining block 55 through the end of the opening 268. It still has some of the remaining neutron and gamma rays which is further processed in the high density tissue equivalent primary collimator 34. In situations where such contaminating neutrons and gamma rays are low and hence it is of lesser concern, especially in low dose radiosurgery, or when treating with broad beam, such high density patient specific field defining block without the high density tissue equivalent primary collimator 34 could be used.

FIG. 1C-3 shows the four sections of high density tissue equivalent patient specific field shaping block 55 with their respective Styrofoam cuts and blocks which include an inner section shown in FIG. 1C-3-1, a section with borated Styrofoam cut boarding the inner high density tissue equivalent block that is shown in FIG. 1C3-2, a rectangular Cerrobend block that is shown in FIG. 1C-3-3. The inner section of the Styrofoam cut is filled with high density tissue equivalent composition 222 that forms the inner section of the high density tissue equivalent patient specific block. It is the first section of the high density tissue equivalent patient specific field shaping block 55 with inner cut field opening 266 is surrounded by neutron and gamma ray absorbing high density tissue equivalent composition in the periphery of the field opening 272 like Gd silicate-rice charcoal or Gd bound to protein or chromium ash. This high density tissue equivalent composition is not limited to those mentioned above. Other such high density tissue equivalent composition can be made and substituted. Like in Cerrobend block making, the inner cut is compactly filled with such compound powder. It absorbs most of the secondary neutrons and gamma rays. Broad beam or arrays of microbeam 264 arriving at the front surface of the high density patient specific field shaping block 55 are blocked except for those passing through the field opening 268. Broad beam or arrays of the beam that passes through the entrance of the inner cut 275 exits through the opposite end of the inner cut opening 276 as the high density patient specific block shaped beam that is in conformity with the treatment field shown in FIG. 1C-2A.

FIG. 1C-3-1 illustrates the rectangular Styrofoam cut 1 with high density tissue equivalent composition in the periphery of the field opening. The inner section of the Styrofoam cut is filled with high density tissue equivalent composition that forms the inner section of the high density tissue equivalent patient specific block 272. It is the first section of the high density tissue equivalent patient specific field shaping block 55. The inner cut field opening 266 is surrounded by an inner PTV plus margin limiting Styrofoam wall 271 which is used to fill the periphery of the central beam aperture 268 with high density tissue equivalent composition 223 as described in FIG. 1C-2A and FIG. 1C2B. Neutron and gamma ray absorbing high density tissue equivalent composition in the periphery of the field opening 272. The high density tissue equivalent composition includes those like Gd silicate-rice charcoal or Gd bound to protein or chromium ash. This high density tissue equivalent composition is not limited to those mentioned above. Other such high density tissue equivalent composition can be made and substituted. The inner cut is compactly filled with such compound powder. It absorbs most of the secondary neutrons and gamma rays. Beam arriving at the front surface of the high density patient specific field shaping block 55 are blocked except for those passing through the field opening 266. Beam that passes through the entrance of the inner cut 275 exits through the opposite end of the inner cut opening as the high density patient specific block shaped beam 276 that is in conformity with the treatment field as shown in FIG. 1C1-2A. Borated Styrofoam cut 284 is placed adjacent to the densely packed high density tissue equivalent composition 278. It moderates the high energy gamma rays produced by the interaction of neutron and hydrogen in the high density tissue equivalent composition 278 to 477 keV gamma rays.

FIG. 1C-3-2 shows the rectangular borated Styrofoam cut 2 in between the inner high density tissue equivalent composition (HDTEC) and the outer rectangular Cerrobend block. The second section of the high density tissue equivalent patient specific field shaping block 55 contains the rectangular borated Styrofoam cut 284 in between the inner densely packed high density tissue equivalent composition 223 and the outer rectangular Cerrobend block 286. The inner cut field opening 266 is surrounded by an inner PTV plus margin limiting Styrofoam wall 271 which is used to fill the periphery of the central beam aperture 268 with high density tissue equivalent composition 223 as described in FIG. 1C-2A and FIG. 1C2B. It forms the neutron and gamma ray absorbing high density tissue equivalent composition in the periphery of the field opening 268. The borated Styrofoam cut 284 moderates the high energy gamma rays produced by the neutron and hydrogen interaction in the high density tissue equivalent composition 223 to 477 keV gamma rays. These lower energy gamma rays are much easier to be absorbed by the adjoining rectangular outer Cerrobend block 286.

FIG. 1C-3-3 Illustrates a rectangular Cerrobend block sandwiched in between inner borated Styrofoam and outer remaining rectangular Styrofoam block. The third section of the high density tissue equivalent patient specific field shaping block 55 contains the rectangular Cerrobend block 286 sandwiched in between the inner borated Styrofoam cut 284 and the outer rectangular remaining Styrofoam block 288 from which other Styrofoam cuts are made. The high energy gamma rays generated by the interaction of neutron with hydrogen, the photoneutrons by the interactions of high energy electron beam and those neutrons and photoneutrons garneted by the beam line reaching the high density patient specific field shaping block 55 is absorbed by the high density tissue equivalent compound 223 and the rectangular cerrobend 286 block. The inner cut field opening 266 is surrounded by an inner PTV plus margin limiting Styrofoam wall 271 which is used to fill the periphery of the central beam aperture 268 with high density tissue equivalent composition 223 as described in FIG. 1C-2A and FIG. 1C2B. The gamma rays reaching the borated Styrofoam cut 284 is moderated to 477 keV and absorbed by the Cerrobend block 286.

FIG. 1C-3-4 shows Semi-Permanent rectangular Cerrobend or lead block attached with borated Styrofoam in a treatment head for attachment of high density patient specific field shaping inner block. The rectangular Cerrobend block sandwiched in between inner borated Styrofoam and outer rectangular Styrofoam are same as illustrated in FIG. 1C-3-3 but they are made as part of a semi permanent patient specific block in the treatment head to which the high density patient specific filed shaping inner block 290 shown in FIG. 1C-5 is inserted. The inner cut field opening 266 is surrounded by an inner PTV plus margin limiting Styrofoam wall 271 which is used to fill the periphery of the central beam aperture 268 with high density tissue equivalent composition 223 as described in FIG. 1C-2A and FIG. 1C2B. The semi-permanent block consists of the rectangular outer cerrobend block 286, rectangular borated Styrofoam 284 and the rectangular outer borated Styrofoam 273. The arrow indicates the exchange of high density, patient specific field shaping inner block 290.

FIG. 1C-3-5 illustrates the exchangeable high density patient specific field shaping inner block and its inserting into and removal from the Semi-Permanent rectangular Cerrobend or lead block with attached rectangular borated Styrofoam in the treatment head. The high energy gamma rays generated by the interaction of neutron with hydrogen, the photoneutrons by the interactions of high energy electron beam and those neutrons and photoneutrons garneted by the beam line reaching the high density patient specific field shaping block 55 is absorbed by the high density tissue equivalent compound 223. The inner cut field opening 266 is surrounded by an inner PTV plus margin limiting Styrofoam wall 271 which is used to fill the periphery of the central beam aperture 268 with high density tissue equivalent composition 223 as described in FIG. 1C-2A and FIG. 1C2B. The arrow shows either insertion or removal of the high density patient specific field shaping block from the semi-permanent rectangular Cerrobend or lead block attached with borated Styrofoam in a treatment head that is shown in FIG. 1-C-3-3. The inner cut field opening 266 is surrounded by neutron and gamma ray absorbing high density tissue equivalent composition 223 in the periphery of the field opening 272. The shaped beam passes through the field opening 268.

FIG. 1D-1 is a continuous illustration of high density patient specific field shaping block making with high density tissue equivalent glass composition. It consists of an inner block made of tissue equivalent glass composition with high neutron cross section 241 that absorbs neutrons generated in this block and those in the beam line scattered into this field shaping block. It is surrounded by an intermediate borated Styrofoam block 242 that moderates the high energy gamma rays produced by the interaction of hydrogen and proton to 477 keV gamma rays which is easier to block with an outer lead or Cerrobend lock 244.

Shielding of neutron with cost-effective silicon containing metallic compounds is described under FIG. 1B. Simple methods of silicon incorporated metallic compounds are used. It includes melting the metal together with plant wastes like melting chromium or gadolinium with rice husk charcoal (19, 20). Rice husk charcoal contains higher than 80% silicates by mass (19). The chromium recovery from liquid and solid tannery waste as chromium ash containing over 25% chromium (21) depends on chromium bound into tannery organic compounds. It is a high density tissue equivalent metal organic compound suitable for making high density tissue equivalent block as in this invention. It could be replaced with other organic compounds like chromium cobalt hexamine trichloride and triiodide, cobalt hexamine perrhenate, chromium hexamine trichloride and triiodide and chromium hexamine perrhenate (22). Other similar compounds with high neutron cross section include gadolinium 157Gd with neutron absorption cross section of 295,000. It is ideal both for neutron shield and neutron dosimetry. After excitation, internal conversion and emission of gamma rays, 157Gd converts to 158Gd with 2.5 neutron capture cross section. In contrast to Gd, chromium and chromium isotopes have much lower neutron cross section, just 24 and lower. Still chromium amines were used as effective nuclear reactor neutron and gamma ray shield (22). In this invention, gadolinium silicate organic compound with densities of about 4.5 and chromium silicates organic compounds with density of about 2.6 is used to make high density patient specific tissue equivalent field shaping glass block for neutron and gamma ray absorption. The scattered and secondary gamma and neutron generated by the high energy electron, Compton gamma, proton and ions reaching the high density tissue equivalent filed shaping block is also monitored with silica optical fiber dosimeters.

FIG. 1D-2 shows high density patient specific field shaping block cut out of high density tissue equivalent glass composition by hotwire cutting similar to hotwire cutting of Styrofoam to make Cerrobend block. Styrofoam cut Cerrobend field shaping block making was the most prominent method of field shaping used in routine daily radiation therapy until the advent of MLC field shaping. This method is adapted for field shaping block cutting out of a block of high density tissue equivalent composition like chromium ask rice husk charcoal glass or Gd silicate-rice husk charcoal. Several other substitutes for rice husk charcoal are also available like wood powder and other plant derivatives. Chromium powder mixed with charcoal ash forms beautiful jade like glass that is cut as ornaments (19). It is an inexpensive, neutron absorbing tissue equivalent substitute. Rice husk-charcoal contains almost 80% silicon as Na2CO3-H3BO3-ZnO—CaO—Al2O3-K2CO3. It is mixed with chromium ash from the leather industry waste in a ceramic a container and heated at 1150° C. for 4 hrs and then poured into metal mold and cooled to 550° C. As it cools, it is cut in shape like cutting a block of Styrofoam to shape the Cerrobend block (26). Alternatively, gadolinium-rice husk charcoal glass block is made. It is processed similar to chromium-rice husk charcoal glass making It has higher density, about 4.5, and forms smaller tissue equivalent neutron block. The method of this block cutting is similar to Styrofoam block cutting for Cerrobend block making which is well known for those familiar with filed shaping with Cerrobend (26=) but with adaptation for microbeam if the beam aperture is for microbeam. In FIG. 1D-2 a high density tissue equivalent glass composition 292 with cut central beam aperture 268 is shown.

FIG. 1D-3 Illustrates a high density patient specific field shaping block cut out of high density tissue equivalent glass composition together with rectangular cut borated Styrofoam, rectangular Cerrobend block and the holding Styrofoam mounted on to a tray to be inserted above the microbeam or nanobeam or minibeam generating high density tissue equivalent primary collimator 34. This block consists of the inner high density tissue equivalent glass block 294 with central beam aperture 268, its adjacent rectangular borated Styrofoam 284, the rectangular outer Cerrobend block 286 and the holding Styrofoam on a tray 296. The inner high density tissue equivalent glass block 294 absorbs the secondary neutron, photoneutrons and ions. Through the central beam aperture 268 the shaped broad beam or microbeam passes through. The borated Styrofoam moderates the MV gamma ray generated by the interaction of proton with hydrogen to 477 keV gamma rays which is absorbed by the rectangular Cerrobend block 286. The holding Styrofoam on the tray 296 holds the sections of the block together. These principles were discussed in details under neutron and gamma ray absorbing blocks made of high density tissue equivalent field shaping block making with high density tissue equivalent powder under FIG. 1C and FIG. 1C3 groups.

FIG. 1D-4 illustrates high density patient specific field shaping block making with molten high density tissue equivalent glass composition poured into a mold like the molten Cerrobend poured into a mold in Cerrobend block making Rice husk charcoal containing more than 80% silica Na2CO3-H3BO3-ZnO—CaO—Al2O3-K2CO3 is mixed with chromium ash or Gd ash in ceramic a container and heated at 1150° C. for 4 hrs like the chromium ash rice-charcoal high density tissue equivalent glass making The molten glass composition is poured into metal mold 298 to form the Inner high density tissue equivalent glass block 294 with central beam aperture 268. When it is cooled, it is mounted on to a tray along with other sections of the high density tissue equivalent filed shaping block as described in FIG. 1D-3.

FIG. 2 illustrates two sets of interlacing parallel microbeam, or nanobeam or minibeam at the isocentric tumor 52, one set from 0 degree and another set from 90 degrees for 100 to 1,000 Gy single fraction radiosurgery. Generation of parallel microbeam or nanobeam or minibeam is described under FIG. 1A. Microbeam, nanobeam or minibeam from two such accelerators, one from 0-degree and another from 90-degree are made to interlace at the isocentric tumor 52. The principles of peak and valley dose differential associated normal tissue sparing from radiation damage is lost at the isocentric tumor 52 where these beams interlace. The whole tumor is radiated with the peak dose 54. There is no valley dose at the isocentric tumor 52 where the beams interlace with each other. Hence there is no tumor tissue sparing from the radiation.

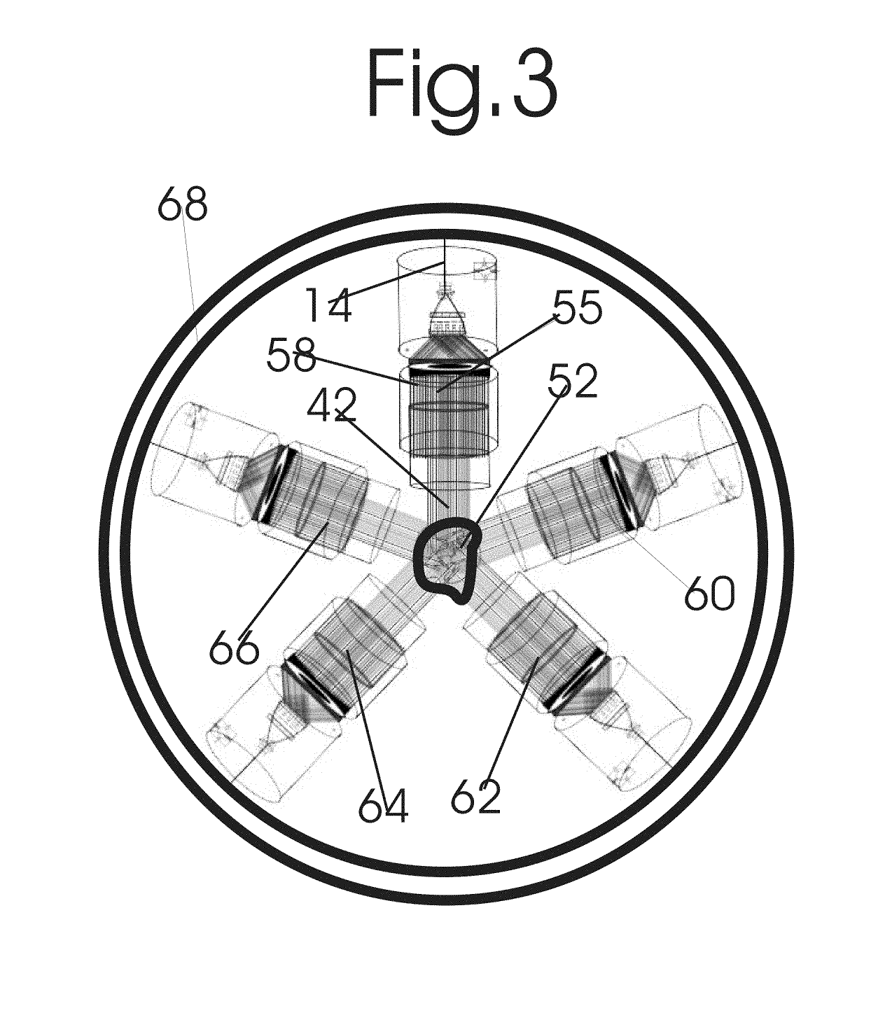

FIG. 3 shows five sets of interlacing parallel microbeam, or nanobeam or minibeam at the isocentric tumor 52. The parallel laser wakefield Thompson scattering electron or Compton scattering gamma ray with collinear electron beam or proton or carbon ion beams all interlaces at the isocentric tumor. This allows simultaneous five field setup radiosurgery at doses ranging from 100 to 1,000 Gy and higher dose in single or fewer fractions. The laser wakefield Thompson scattering electron or Compton scattering gamma ray with collinear electron beam or proton or carbon ion beam 14 is split into parallel beams 42. The parallel beams are generated in tissue equivalent universal collimator-1 58, tissue equivalent universal collimator-2, 60, tissue equivalent universal collimator-3, 62, tissue equivalent universal collimator-4 64, and the tissue equivalent universal collimator-5, 66. They are arranged circularly on to a circular gantry 68. All the elements for electron or Compton scattering gamma rays with collinear electron or proton microbeam or nanobeam or minibeam generation shown in this FIG. 3 are identical to those described under FIG. 1A and FIG. 2. Microbeams or nanobeams or minibeams from five such universal tissue equivalent collimators attached to the circularly configured accelerating units interlace at the isocentric tumor 52. The principles of peak and valley dose differential associated sparing of the normal tissue from radiation damage is lost at the isocentric tumor 52 where all these beams interlace together. The whole tumor is radiated with the peak dose 54 shown in FIG. 1 and FIG. 2. There are no valley dose regions in the tumor where all these five sources of microbeams or nanobeams or minibeams interlace. To shape the microbeam or nanobeam in conformity with the tumor volume, varying shape and size patient specific collimators 55 are placed upstream to the tissue equivalent primary collimator 34 as shown in FIG. 1, Fig.C-1 and FIG. 2.

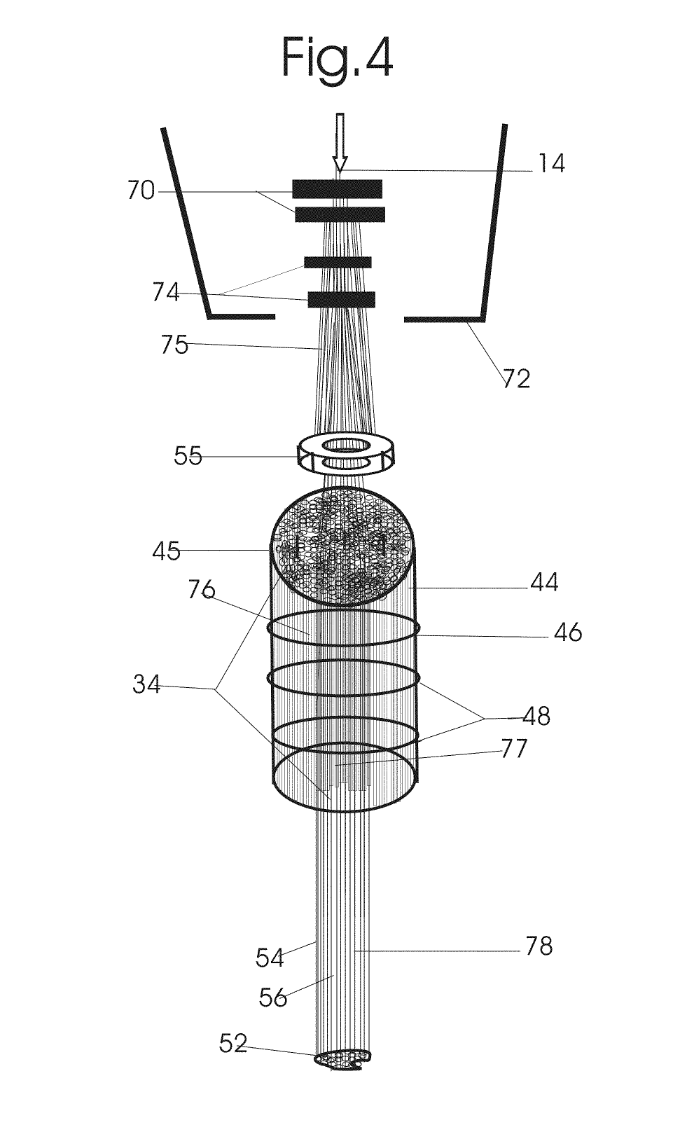

FIG. 4 illustrates microbeam or nanobeam or minibeam generation out of spread out laser wakefield Thompson scattering electron or Compton scattering gamma ray with collinear electron beam or proton beam 14 in a cylindrical tissue equivalent primary collimator incorporated with a patient specific collimator. The size of the beam, microbeam or nanobeam or minibeam depends on the size of the microfocus beam guides 44 in the tissue equivalent primary collimator 34. The pencil beam is spread out by the passive scatterer 70 in a nozzle 72. The dose is monitored by the dose monitors 74. The spread out Brag peak beam 75 is incident onto the patient specific collimator 55. The tissue equivalent primary collimator 34 is equipped with microfocus beam guides 44. To maintain the peak and valley dose differential as in microbeam radiation therapy, the microfocus beam guides 44 are placed at a distance of one to four ratio of beam width and distance from each other in tissue equivalent primary collimator 34. If the beam width is say 75 micrometers then the distance from two adjacent microfocus beam guides 44 is kept as 300 micrometers. If the beam width were 10 micrometers, then the distance from two adjacent microfocus beam guides 44 is kept as 40 micrometers apart. Similar ratio of distance from microfocus beam guides 44 is also kept for nanobeams and minibeams. If 500 nanometer width nanobeams were used for nanobeam radiation, then the distance from two adjacent microfocus beam guides 44 is kept as 2,000 nanometers that is 2 micrometers apart. If it is minibeam with 300 micrometer width, then the distance from two adjacent microfocus beam guides 44 is kept as 1.2 mm apart. The spread out beam 75 that enters into the microfocus beam guides 44 through microfocus carbon tube's openings 45. They are focused by the focusing anode 46 and the focusing magnet 48. Such focusing of the microbeam, nanobeam and minibeam nearly eliminates their already nanoscale sized penumbra as it travels through the tissue equivalent primary collimator 34 towards the isocentric tumor 52.

The magnetically focused electron beam's penetration below the skin and its distance from below the skin to maximum dose, the dmax is much deeper than those for the electron beam not magnetically focused (17, 18). It removes the higher rate of skin's toxic reaction and fibrosis from high dose conventional electron beam radiation therapy. The severe errythema, edema, pain and ulceration from conventional high dose electron beam radiation to the skin are avoided by magnetically focused electron beam radiation therapy. It is also described in more detail in this invention. It is very important for 100 to 1,000 Gy and higher dose microbeam radiosurgery. Furthermore, such magnetically focused beams having less than nm sized penumbra avoids smearing of the adjacent microbeam's base with each other. It has much better defined low dose valley region 56. It makes more efficient normal stem cell proliferation in normal tissue through which the microbeam, nanobeam or minibeam travels. It helps to heal the microbeam radiosurgery's toxic effects in normal tissue more efficiently.

Different patients have different sized tumors. Patient specific collimators 55 of varying size are placed upstream to the tissue equivalent primary collimator 34. The unwanted beam outside of the carbon tube 76 is absorbed by the solid portions of the tissue equivalent primary collimator 34. The low energy secondary ions, neutron and scattered radiation traveling through the microfocus beam guides 44 is absorbed by the tissue equivalent filters at the proximal ends of the microfocus beam guides 44. Such filtered parallel microbeam, nanobeam or minibeam 78 travels towards the isocentric tumor 52. It is modulated in conformity with the shape and configuration of the tumor volume that is treated. The portion of the tissue that is radiated by the filtered parallel microbeam, nanobeam or minibeam 78 is the peak dose region 54. The tissue that is separated between the two peak radiation regions in tissue is the low or no dose region, the valley dose 56 region. The whole tumor is radiated with cross firing peak dose 54 arriving at the isocentric tumor simultaneously from multiple accelerators. They form a network of interlaced microbeam, nanobeam or minibeam 78 within the isocentric tumor 52. Hence there is no tumor tissue sparing by valley dose within the tumor.

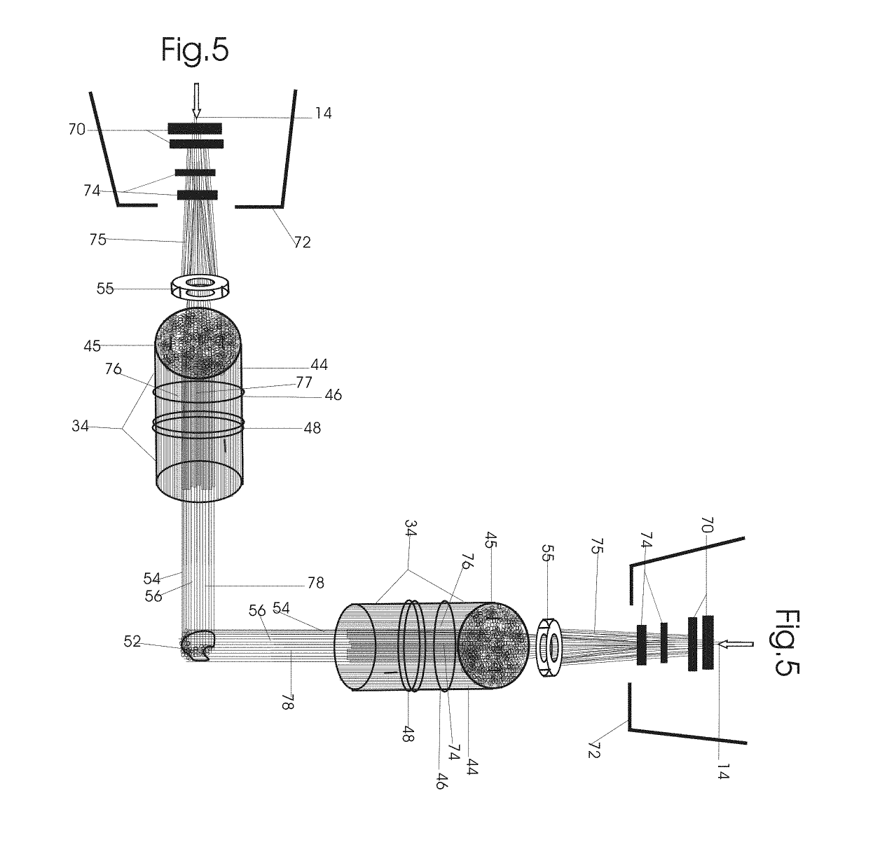

FIG. 5 shows interlacing microbeam or nanobeams or minibeams from two sets of spread out laser wakefield Thompson scattering electron or Compton scattering gamma ray with collinear electron beam or proton or carbon ion beam 14, one from 0-degree and the other from 90-degree for simultaneous interlaced microbeam or nanobeam radiosurgery of an isocentric tumor. The method of generating parallel microbeam or nanobeam or minibeams is shown in FIG. 1 and FIG. 4. The spread out pencil beam is processed and separated as filtered parallel microbeam nanobeam or minibeam 78. It is modulated in conformity with the shape and configuration of the tumor volume that is treated. The parallel beams from the accelerating system at 0-degree are shown as traveling towards the isocentric tumor 52. It is interlaced with identically processed filtered parallel microbeam nanobeam or minibeam 78 arriving from another collimated system placed at 90-dgrees. Sparing of the normal tissue from radiation damage is lost at the isocentric tumor 52 where all the parallel microbeam nanobeam or minibeam 78 from 0-degree and 90-degree interlace. The whole tumor is radiated with the peak dose regions 54. Because of the interlacing beams from 0-degree and 90-degree, there are no valley doses 56 at the isocentric tumor 52. Hence there is no tumor tissue sparing from radiation. In contrast, since there are no interlacing beams in normal tissue, it is protected from radiation damage. Normal clonogenic stem cells from the low or no dose valley region proliferate and migrate to the peak dose regions 54.

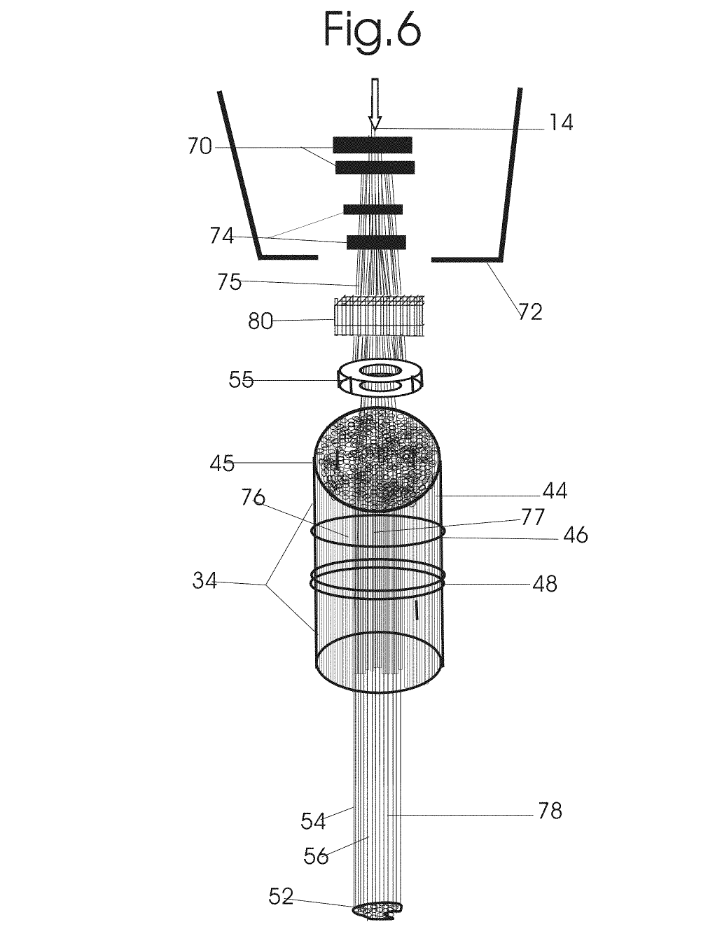

FIG. 6 is another illustration of the spread out pencil beam's processing as illustrated in FIG. 4 but the spread out beam as first channeled through a semi-patient specific carbon nanotube pre-collimator 80 for beam focusing by carbon nanotube's induced magnetism before it is channeled through the patent specific collimator 55 and the tissue equivalent primary collimator 34 where microbeam, nanobeam or minibeams are generated. They are channeled through multi-wall carbon nanotube (MWNT). As the spread out pencil beam pass through the MWCNT in the semi-patient specific carbon nanotube pre-collimator 80, the beam is modulated into microbeams and they are focused by the induced magnetism like the proton beam induced magnetism. These beams then pass through the patient specific collimator 55 and enter into the microfocus carbon tube's openings 45 and travels through the microfocus beam guide 44 in the tissue equivalent primary collimator 34. The beam in the microfocus beam guide 44 is also focused by the focusing anode 46 and focusing magnet 48. The scattered and secondary ions and neutrons are absorbed by the tissue equivalent primary collimator 34 and by the tissue equivalent inserts in the microfocus carbon tubes. The filtered parallel microbeam, nanobeam or minibeam 78 travels towards the isocentric tumor 52. With the tissue equivalent primary collimator 34 placed downstream to patient specific collimator 55, the processed laser wakefield Thompson scattering electron or Compton scattering gamma ray with collinear electron beam or proton or carbon ion beam 77 is modulated in conformity with the shape and configuration of the isocentric tumor 52 that is treated. It renders conformal microbeam, nanobeam or minibeam radiation to the tumor with sparing of the normal tissue from high dose radiation damages.

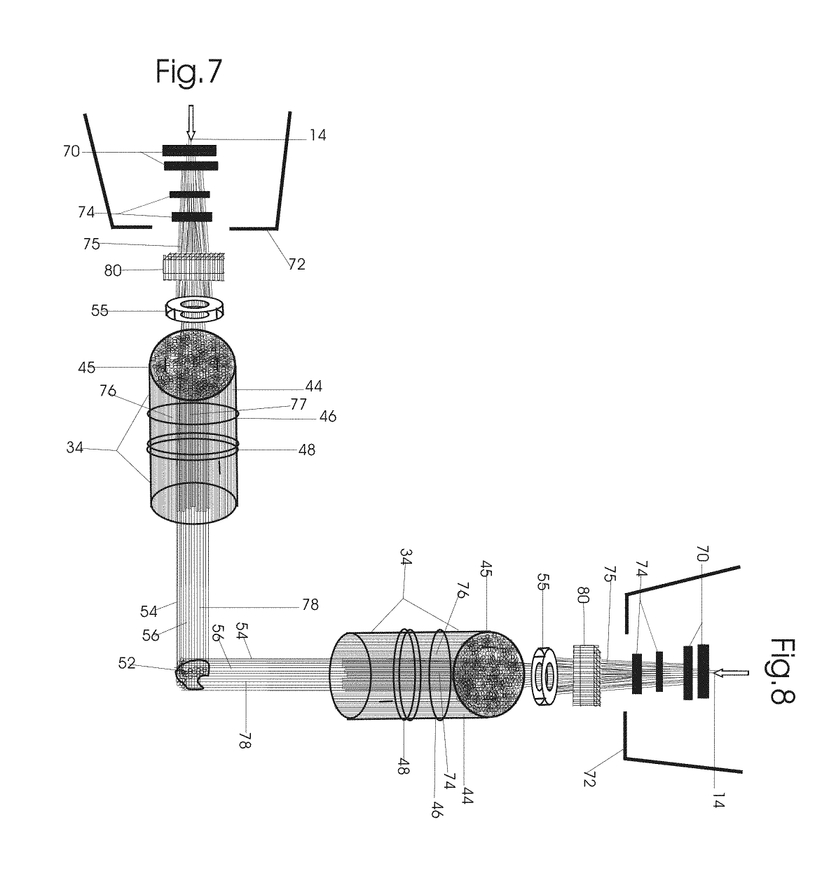

FIG. 7 shows interlacing microbeam or nanobeams from two sets of microbeam, nanobeam, minibeam or proton or carbon ion microbeam, nanobeam or minibeam generating systems with semi-patient specific carbon nanotube pre-collimator, one at 0-degree and the other at 90-degree for simultaneous interlaced microbeam, nanobeam or minibeam radiosurgery of an isocentric tumor. The method of radiosurgery with two accelerating systems without semi-patient specific carbon nanotube pre-collimator 80 is described under FIG. 2 and in FIG. 5. In FIG. 6, the spread out spread out pencil beam is shown as passing through the MWCNT in the semi-patient specific carbon nanotube pre-collimator 80. This beam is focused within the MWCNT by the induced magnetism of the incident beam. As described before under FIG. 2 and in FIG. 5 the spread out beam is processed and separated as filtered parallel microbeam or nanobeam or minibeam 78. It is modulated for conformal radiosurgery. The microbeam or nanobeam or minibeam 78 from the accelerating system with semi-patient specific carbon nanotube pre-collimator 80 at 0-degree is shown as traveling towards the isocentric tumor 52. It is interlaced with identically processed and filtered parallel microbeam or nanobeam or minibeam 78 from the collimator system at 90-degree. Because of the interlacing beams, the whole tumor is radiated with the peak dose region 54. Since there are no interlacing parallel beams in normal tissue, the normal tissue is mostly protected from radiation. The proliferation of normal clonogenic stem cells from the low or no dose valley region 56 regions and their migration to the peak dose regions 54 protects the normal tissue from radiation damages.

FIG. 8 illustrates the patient specific collimator 55A made of high density tissue equivalent composition for neutron absorption, borated Styrofoam to moderate the MV gamma rays and as it is placed above the tissue equivalent primary collimator 34 for spread out Brag peak field shaping and microbeam, nanobeam or minibeam radiosurgery. The patient specific field shaping block 55A contains high density tissue equivalent composition, borated Styrofoam and Cerrobend. They are also described in FIG. 1A through FIG. 1D. The high density tissue equivalent composition absorbs the neutron. The borated Styrofoam moderates the MV gamma rays and the Cerrobend absorbs the moderated gamma rays. To illustrate the Cerrobend block described here better, it is projected forward from the beam. As described in FIG. 4, the pencil beam is spread out by the passive scatterer 70 in a nozzle 72. The dose is monitored by the dose monitors 74. The spread out Brag peak beam 75 is incident onto the patient specific collimator 55A made of high density tissue equivalent composition, borated Styrofoam and Cerrobend. The high density tissue equivalent composition absorbs the neutron. The borated Styrofoam moderates the MV gamma rays and the Cerrobend absorbs the moderated gamma rays. It is placed above the tissue equivalent primary collimator 34 which is equipped with microfocus beam guide 44. The spread out beam 75 enters into the microfocus beam guide 44 through microfocus carbon tube's openings 45. They are focused by the focusing anode 46 and the focusing magnet 48. The unwanted beam outside of the carbon tube 76 is absorbed by the solid portions of the tissue equivalent primary collimator 34. The low energy secondary ions, neutron and scattered radiation traveling through the microfocus beam guide 44 is absorbed by the tissue equivalent filters at the proximal ends of the microfocus beam guide 44. Such filtered parallel microbeam, nanobeam or minibeam 78 travels towards the isocentric tumor 52. It is modulated in conformity with the shape and configuration of the tumor volume that is treated. The portion of the tissue that is radiated by the filtered parallel microbeam, nanobeam or minibeam 78 is the peak dose region 54. The tissue that is separated between the two peak radiation regions in tissue is the low or no dose region, the valley dose 56 region. Compared to other block making material like stainless steel, brass, tungsten, lead, nickel and iron, the Cerrobend has the highest radioactivity after proton radiation within one second but it falls off within one minute to less than its half as compared to tungsten (27). The energy of the secondary neutron produced is about 1-2 MeV. Its average RBE is as high as 25 (28). Field shaping with high density tissue equivalent patient specific collimator 55A and its ability to remove the secondary neutron and gamma rays is a more practical and cheaper method of field shaping for proton radiation therapy than the alternatives spot and raster scanning used to minimize the secondary neutron and gamma rays. It doesn't need the huge magnets used for spot scanning used to reduce the neutron dose. The spot scanning still produce secondary neutron, about 0.002 to 0.004 Sv/Gy (29).

FIG. 9 shows a neutron moderating Styrofoam block that is surrounded by a Cerrobend cover 55B and this combined system is placed in the periphery of a field shaping multileaf collimator (MLC) 55-C and this combined blocks as placed above the tissue equivalent primary collimator 34 for spread out Brag peak proton beam field shaping for microbeam, nanobeam or minibeam radiosurgery. This system consists of the neutron moderating Styrofoam block that is surrounded by Cerrobend cover 55B and this combined system is placed in the periphery of a field shaping multileaf collimator (MLC) 55-C. The convenience of filed shaping with MLC 55-C is maintained but the MLC generates high levels secondary neutrons and gamma radiation which needs to be removed for the sake of safety, technical staff treating the patient and the area surrounding the treatment room.

Even for the daily fractionated radiation therapy and 15 to 20 Gy conventional broadbeam single fractions radiosurgery, MLC based field shaping is not ideal due to its high neutron generation and the proton induced radioactivity in the MLC (27). It is reported in numerous reports and summarized in US patent application 2013/0072744(30). It is incorporated herein in its entirety. While this patent application describes the long term toxic effects of secondary neutron and nuclear activation of the MLC leafs, its proposed MLC leafs with titanium oxide only reduces the secondary neutron production, scatter radiation and nuclear activation only to a little as compared with tungsten leafs. It may be a partial relief for daily fractionated radiation therapy but not for kGy radiosurgery that produce much higher scatter and gamma radiation, nuclear activation and secondary neutron production. It is also more complex and expensive as compared with the modified Cerrobend field shaping block described under FIG. 8. Furthermore, shielding with the modified Cerrobend block helps since it has no radiation leakage compared with the leakage radiation through the micron sized space in between the MLC blades. In this invention, the scattered radiation and gamma radiation and the nuclear reactions associated radiation from induced radioactivity within MLC 55-C are absorbed by the MLC shielding Cerrobend cover 55-B. It contains high density tissue equivalent composition, borated Styrofoam and Cerrobend. The high density tissue equivalent composition absorbs the neutron. The borated Styrofoam moderates the MV gamma rays and the Cerrobend absorbs the moderated gamma rays. As described in FIG. 4 and in FIG. 8 the pencil beam is spread out by the passive scatterer 70 in the nozzle 72. The dose is monitored by the dose monitors 74. The spread out Brag peak beam 75 is incident onto the MLC 55C and to the combined high density tissue equivalent composition, neutron moderating Styrofoam and Cerrobend cover 55B made out of high density tissue equivalent composition, borated Styrofoam and Cerrobend. The high density tissue equivalent composition absorbs the neutron. The borated Styrofoam moderates the MV gamma rays and the Cerrobend absorbs the moderated gamma rays. It is placed above the tissue equivalent primary collimator 34 which is equipped with microfocus beam guides 44. The spread out beam 75 that enters into the microfocus beam guides 44 through microfocus beam guide's openings 45. They are focused by the focusing anode 46 and the focusing magnet 48. The unwanted beam outside of the carbon tube 76 is absorbed by the solid portions of the tissue equivalent primary collimator 34. The low energy secondary ions, neutron and scattered radiation traveling through the microfocus beam guides 44 is absorbed by the tissue equivalent filters at the proximal ends of the microfocus beam guides 44. Such filtered parallel microbeam, nanobeam or minibeam 78 travels towards the isocentric tumor 52. It is modulated in conformity with the shape and configuration of the tumor volume that is treated. The portion of the tissue that is radiated by the filtered parallel microbeam, nanobeam or minibeam 78 is the peak dose region 54. The tissue that is separated between the two peak radiation regions in tissue is the low or no dose region, the valley dose 56 region.

The field shaping with MLC 55C is shielded with a combined high density tissue equivalent composition, neutron moderating Styrofoam and Cerrobend cover 55B. They remove the secondary neutron and gamma rays generated in the MLC 55C. It is a more practical and cheaper method of field shaping for proton radiation therapy than the alternative spot or raster scanning used to minimize the secondary neutron and gamma rays. It doesn't need the huge magnets for spot or raster scanning to reduce the neutron dose. The spot scanning still produce secondary neutron, about 0.002 to 0.004 Sv/Gy (29).

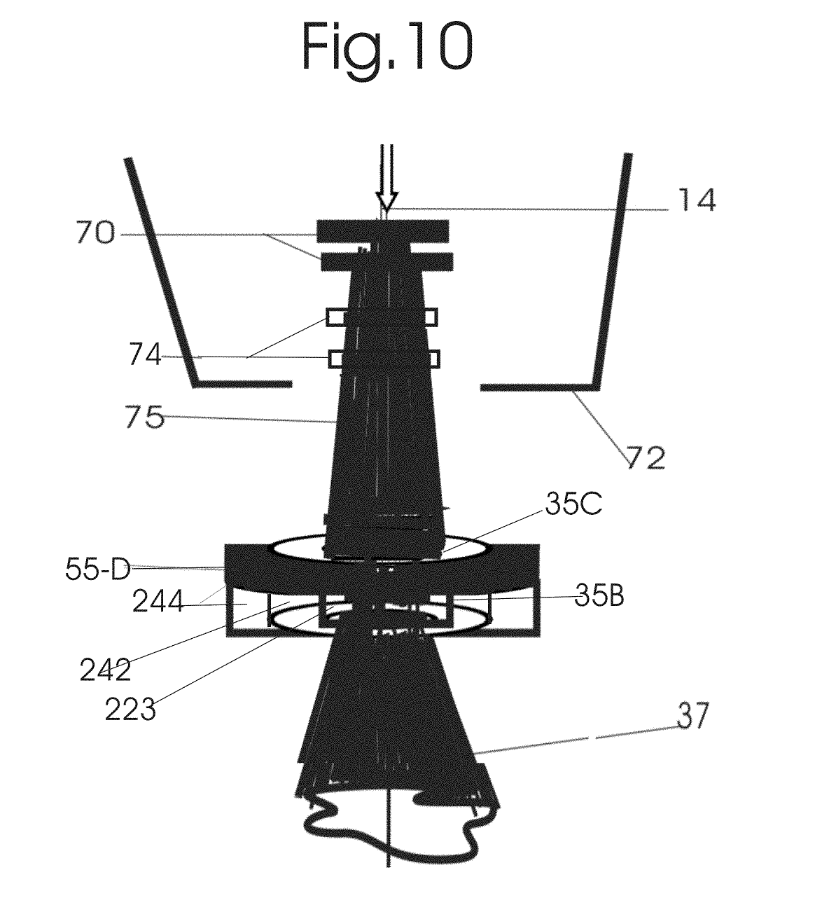

FIG. 10 shows a patient specific filed shaping block system 55-D made of high density tissue equivalent composition for secondary neutron absorption, borated Styrofoam to moderate the MV gamma rays and Cerrobend to absorb the moderated gamma rays for daily fractionated proton radiation therapy or 10 to 20 Gy single fraction proton radiosurgery with lesser secondary neutron and gamma radiation to the patient and for the neutron radiation safety to technical staff treating the patient and in the area surrounding the treatment room without the expense and inconvenience of spot or raster scanning with huge magnets.

To protect the patient from undesirable scattered and gamma radiation and secondary neutron generated by conventional daily fractionated spread out Brag peak or 10-20 Gy lower dose hypofractionated proton or ion radiosurgery a patient specific field shaping block system 55-D containing high density tissue equivalent composition, borated Styrofoam and Cerrobend is constructed. The high density tissue equivalent composition absorbs the neutron. The borated Styrofoam moderates the MV gamma rays and the Cerrobend absorbs the moderated gamma rays. The pencil beam is spread out by the passive scatterer 70 in a nozzle 72. The dose is monitored by the dose monitors 74. The spread out Brag peak beam 75 is incident onto the patient specific collimator 55-D made of high density tissue equivalent composition, borated Styrofoam and Cerrobend. The high density tissue equivalent composition absorbs the neutron. The borated Styrofoam moderates the MV gamma rays and the Cerrobend absorbs the moderated gamma rays. The neutron and gamma ray filtered out beam exits through the beam exit window 35C in patient specific collimator system with high density neutron absorbing composition, gamma ray moderating borated Styrofoam and cerrobend 55D. It travels towards the tumor as gamma ray and neutron filtered spread out Brag peak proton beam 37 through beam guide 35B surrounded by its adjacent high density tissue equivalent composition 223 followed by intermediate borated Styrofoam block 242 and then the outer Cerrobend block 244. Gamma and neutron filtered spread out Brag peak proton beam 37 treats the tumor and its boarders with microscopic tumor.

Compared to other block making material like stainless steel, brass, tungsten, lead, nickel and iron, the Cerrobend has the highest radioactivity after proton radiation within one second but it falls off within one minute to less than its half as compared to tungsten (27). The energy of the secondary neutron produced is about 1-2 MeV. Its average RBE is as high as 25 (28). Field shaping with high density tissue equivalent patient specific collimator 55-D and its ability to remove the secondary neutron and gamma rays is far more practical. It is a cheaper method of field shaping for proton radiation therapy than the alternative spot or raster scanning used to minimize the secondary neutron and gamma rays. It doesn't need the huge magnets used for spot or raster scanning The spot scanning still produce secondary neutron, about 0.002 to 0.004 Sv/Gy (29).

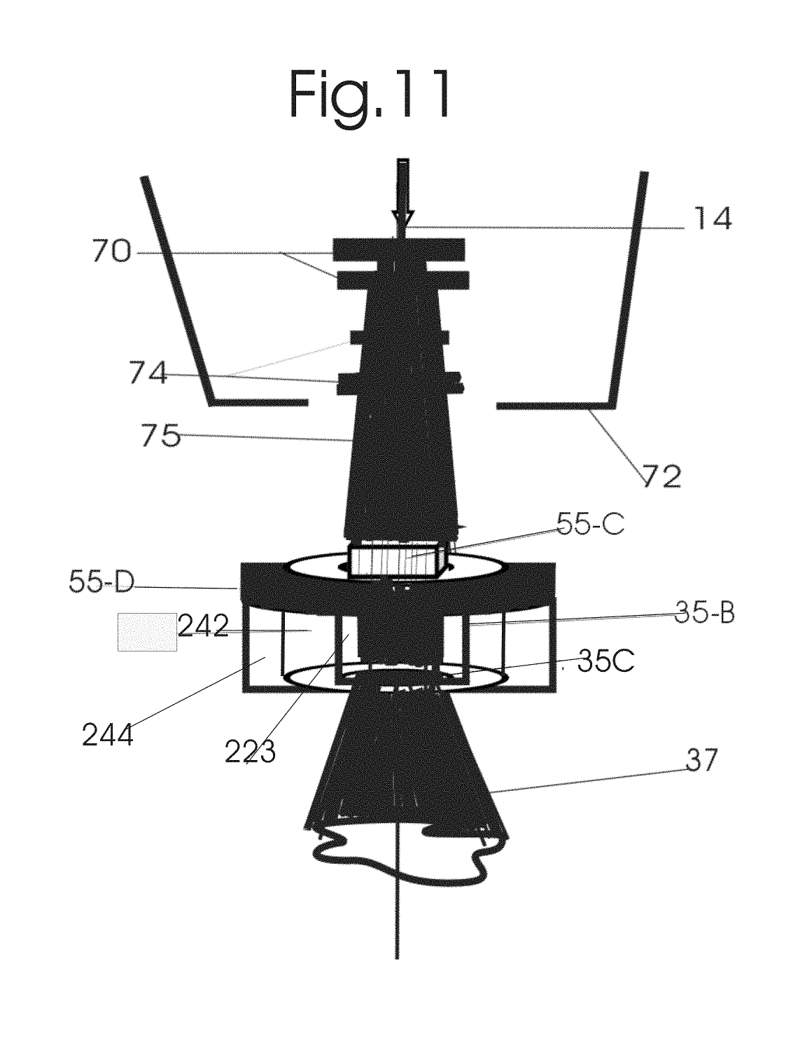

FIG. 11 illustrates a patient specific radiation therapy field shaping block system 55-D consisting of multileaf collimator (MLC) 55-C shielded with high density tissue equivalent composition for secondary neutron absorption, borated Styrofoam to moderate the MV gamma rays and Cerrobend to absorb the moderated gamma rays for daily fractionated proton radiation therapy or 10 to 20 Gy single fraction proton radiosurgery with lesser secondary neutron and gamma radiation to the patient and for the neutron radiation safety to technical staff treating the patient and in the area surrounding the treatment room without the expense and inconvenience of spot or raster scanning with huge magnets. It is similar to the field defining block described under FIG. 10 but with a MLC instead of the modified Cerrobend as in FIG. 10. To protect the patient from undesirable scattered and gamma radiation and secondary neutron generated by conventional daily fractionated spread out Brag peak or 10-20 Gy lower dose hypofractionated proton or ion radiosurgery a patient specific field shaping block system 55-D containing high density tissue equivalent composition, borated Styrofoam and Cerrobend is constructed. It is placed as surrounding the multileaf collimator (MLC) 55-C. The high density tissue equivalent composition in the periphery of the MLC 55-C absorbs the neutron. The borated Styrofoam next to the high density composition moderates the MV gamma rays and the Cerrobend absorbs the moderated gamma rays. The pencil beam is spread out by the passive scatterer 70 in a nozzle 72. The dose is monitored by the dose monitors 74. The spread out Brag peak beam 75 is incident onto the MLC-C which is surrounded by the patient specific collimator system with high density neutron absorbing composition, gamma ray moderating borated Styrofoam and cerrobend 55-D. The MLC shaped beam travels towards the tumor as gamma ray and neutron filtered spread out Brag peak proton beam 37 through beam guide 35B surrounded by its adjacent high density tissue equivalent composition 223 followed by intermediate borated Styrofoam block 242 and then the outer Cerrobend block 244. The gamma ray and neutron filtered spread out Brag peak proton beam 37 treats the tumor and its boarders with microscopic tumor. With such a modified Cerrobend shield to MLC, the advantages of MLC for field shaping are maintained while most of the scattered and secondary gamma radiation of the MLC (30) is eliminated.

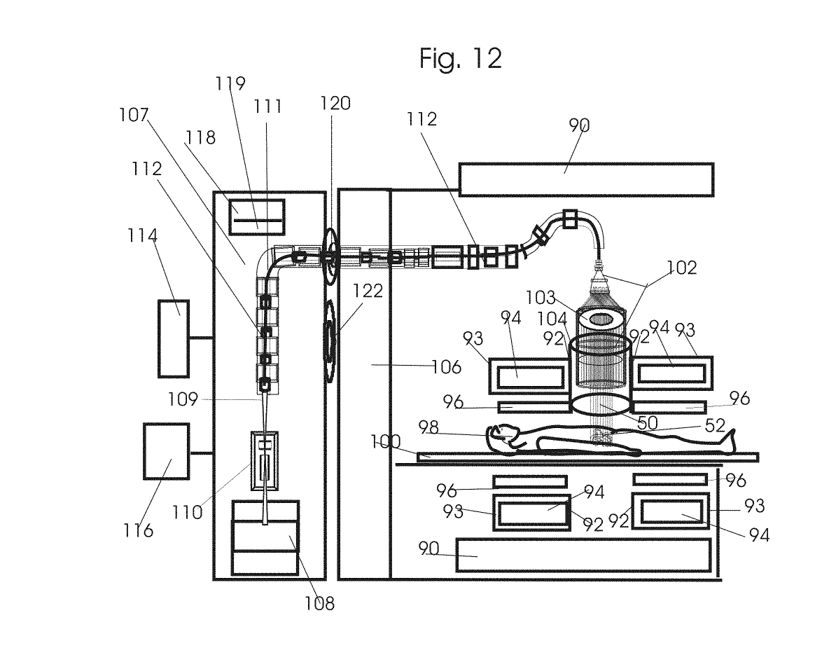

FIG. 12 shows magnetic resonance image (MRI) guided kGy microbeam, nanobeam or minibeam single or hypofractionated rotational radiosurgery system having very high energy (VHE) laser wakefield Thompson scattering electron microbeam or nanobeam or minibeam generating cylindrical tissue equivalent primary collimator and a patient specific conformal filed shaping collimator. A MRI unit with radiation therapy is described in (31) It is incorporated herein in its entirety. Such a system is adapted for MRI image guided microbeam, nanobeam or minibeam radiosurgery. It consists of the primary electro magnets made of non-ferromagnetic materials 90, symmetrical partial gradient coils 92, to gradient coil 94 and to the high frequency coils 96 that is enclosed in gradient coil shield 93 and the radiation therapy accelerator is a laser wakefield accelerator with dual supersonic plasma jet that stabilizes the electron beam. They are connected to MRI and to compact wakefield accelerator control unit 114, to the display control unit and console 116. The radiation therapy unit in this instance consists of very high energy (VHE) laser wakefield Thompson scattering electron microbeam or nanobeam or minibeam generating cylindrical tissue equivalent primary collimator and a patient specific conformal filed shaping collimator. It has no conventional linear electron accelerator or target to generate X-ray beam or beam flattening filter or MLC like beam shaping system as in the case of Siemens's system. The laser wakefield Thompson scattering VHE microbeam, nanobeam or minibeam generating system is an entirely new electron accelerating system. It has no RF system and hence no RF interference with the electron beams. It consists of a compact wakefield accelerator 108, dual supersonic plasma jet 110, VHE focusing magnet and vacuum beam transport 112. With electromagnets for MRI and it being inactive during radiation therapy, there is no Lorentz force interference with the VHE electron beam radiation therapy. Laser driven VHE electron/photon beam radiation therapy system was also proposed before (32) but it is substantially different from this invention with dual stage supersonic plasma jet and microbeam, nanobeam or minibeam generating tissue equivalent collimator for kGy radiosurgery. Furthermore, in this invention, the magnetically focused electron beam's penetration is far below the skin surface. With magnetically focused electron beam, its maximum dose, the dₘₐₓ is much deep below the skin (17, 18). It removes the higher rate of toxic reaction within the skin surface and in tissue below it. In this invention, the severe errythema, edema, pain and ulceration that occur after conventional high dose electron beam radiation to the skin are avoided. Clinically, it is very important for safe administration of single or hypofraction kGy radiosurgery. Such clinical precautions are not described in U.S. Pat. No. 8, 618,521 (32). In this invention, stable electron beam is generated by the dual supersonic plasma jets 110. Furthermore, such magnetically focused beams have much less penumbra. It avoids smearing of the adjacent microbeam, nanobeam or minibeam's base with each other. It creates much better defined low dose valley region 56 illustrated in FIG. 1A to FIG. 10. It helps to heal the normal tissue through which the microbeam, nanobeam or minibeam travels towards an isocentric tumor. The radiation damage in normal tissue is repaired by proliferation of its normal stem cells. A rotary joint rotating in horizontal axis 120 with aligned VHE beam connects the VHE beam 111 between the rotating gantry 106 and the stationary gantry 107. VHE bending and focusing magnets and vacuum beam transport 115 leads the VHE-beam 111 to parallel microbeam or nanobeam or minibeam generating tissue equivalent collimator system 102. The processed microbeam or nanobeam or minibeam is focused with microbeam, nanobeam, or minibeam focusing magnet 104. Such focused microbeam/nanobeam or minibeam 50 has much deeper dmax under the skin. It and the rotational treatment or multiple simultaneous field setup methods of radiation therapy avoids excessive radiation toxicities to the skin. The rotating gantry 106 rotates around the axis of the primary electro magnets made of non-ferromagnetic materials 90 under the control of a gantry control unit 119 which is also connected to beam deflection and collimator control unit 118, display control unit 116, MRI and the drive laser control unit 114 and to the patient bed 100 which is movable in upwards and downward and lateral directions under the control of the gantry control unit 119 for the precise combined stereotactic setup of a patient 98 for radiosurgery.

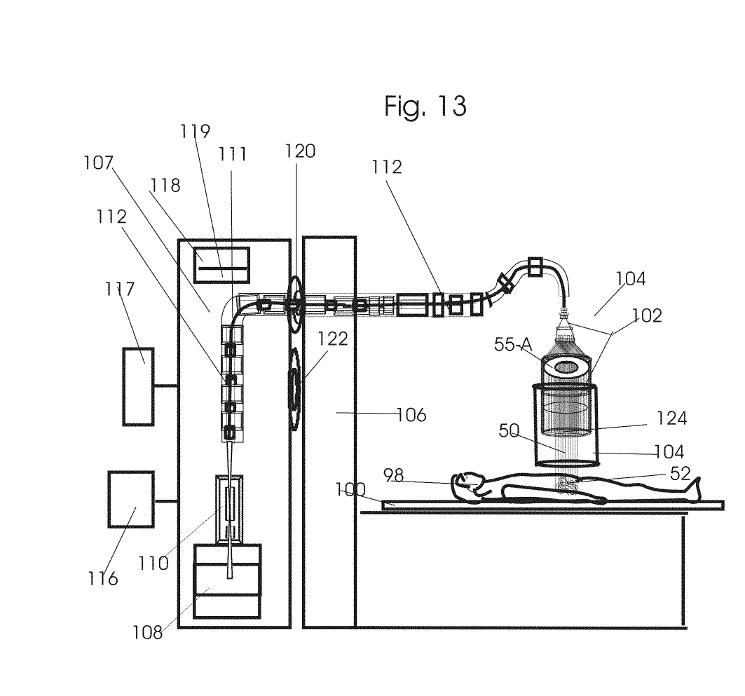

FIG. 13 illustrates kGy microbeam, nanobeam or minibeam single or hypofractionated rotational radiosurgery system without MRI as described in FIG. 12 and having only very high energy (VHE) laser wakefield Thompson scattering electron microbeam or nanobeam or minibeam generating cylindrical tissue equivalent primary collimator and a patient specific conformal field shaping collimator. The MRI unit described in FIG. 12 is removed and the microbeam, nanobeam or minibeam radiosurgery unit consists of only the very high energy (VHE) laser wakefield Thompson scattering electron beam generating system and the microbeam or nanobeam or minibeam generating cylindrical tissue equivalent primary collimator and a patient specific conformal filed shaping collimator. It has no conventional linear electron accelerator or target to generate X-ray beam or beam flattening filter. For single or hypofraction radiosurgery, the patent specific field shaping is performed with the type of patent specific Cerrobend block 55-A described in FIG. 8 or with filed shaping MLC 55-C with filtering out the scattered and gamma radiation by the Cerrobend cover and the secondary neutron it generates by the tissue equivalent collimator 102. The laser wakefield Thompson scattering VHE microbeam, nanobeam or minibeam generating system consists of a Compact wakefield accelerator 108, dual supersonic plasma jets 110, VHE focusing magnet and vacuum beam transport 112. Parallel microbeam or nanobeam or minibeam exiting out of tissue equivalent collimator system 124 is focused with focusing magnets 104. This focused microbeam/nanobeam or minibeam 50 has much better penetration through the skin; it is far below the skin surface. Its maximum dose, the dmax below the skin is much deeper than those for the unfocused beam (17, 18). It removes the higher rate of toxic reaction within the skin surface and in tissue below it. In this invention, the severe errythema, edema, pain and ulceration that occur after conventional high dose electron beam radiation to the skin are avoided. Clinically, it is very important for safe administration of single or hypofraction kGy radiosurgery. As described before, such magnetically focused beams have much less penumbra. It avoids smearing of the adjacent microbeam, nanobeam or minibeam's base with each other. It creates much better defined low dose valley region 56 illustrated in FIG. 1A to FIG. 10. This helps to heal the normal tissue through which the microbeam, nanobeam or minibeam travels towards an isocentric tumor. The radiation damage in normal tissue is repaired by proliferation of its normal stem cells. A rotary joint rotating in horizontal axis 120 with aligned VHE beam connects the VHE beam 111 between the rotating gantry 106 and the stationary gantry 107. VHE bending and focusing magnets and vacuum beam transport 115 leads the VHE-beam 111 to parallel microbeam or nanobeam or minibeam generating tissue equivalent collimator system 102. The rotating gantry 106 rotates around the patient 98 under the control of gantry control unit 119 which is also connected to beam deflection and collimator control unit 118, display control unit 116, the drive laser control unit 117 and to the patient bed 100 which is movable in upwards and downward and lateral directions under the control of the gantry control unit 119 for the precise combined stereotactic setup of a patient 98 for radiosurgery.

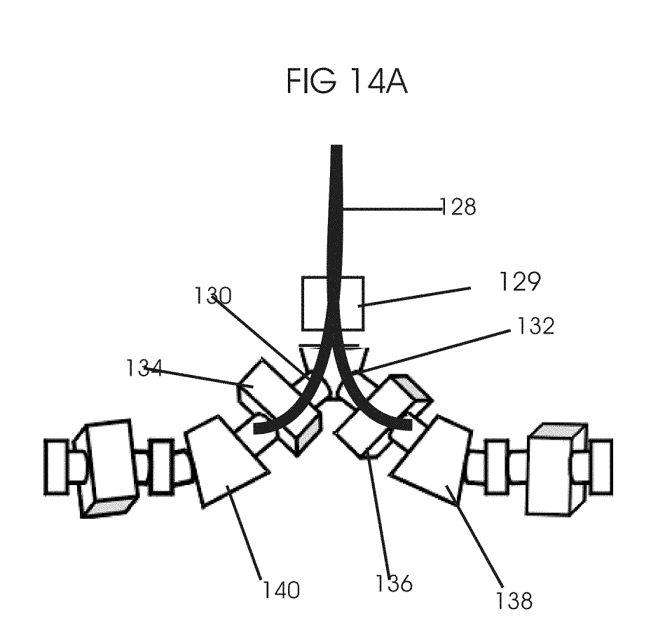

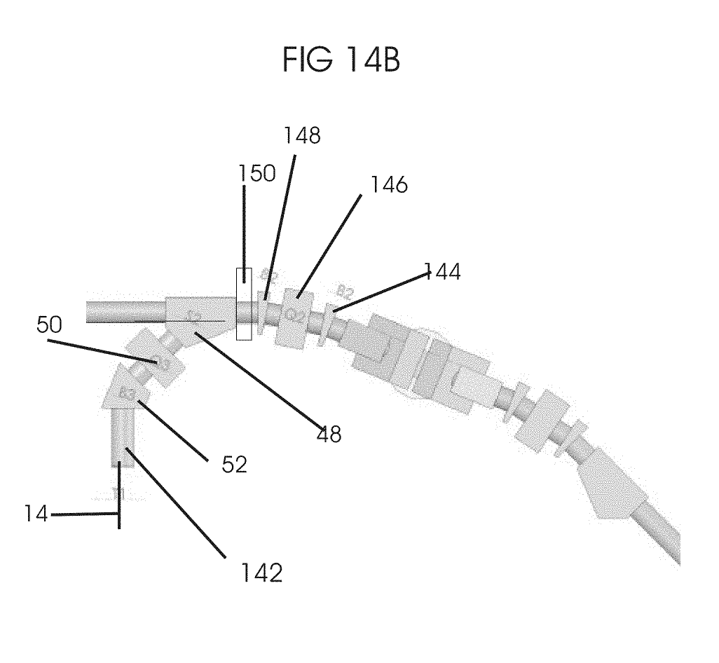

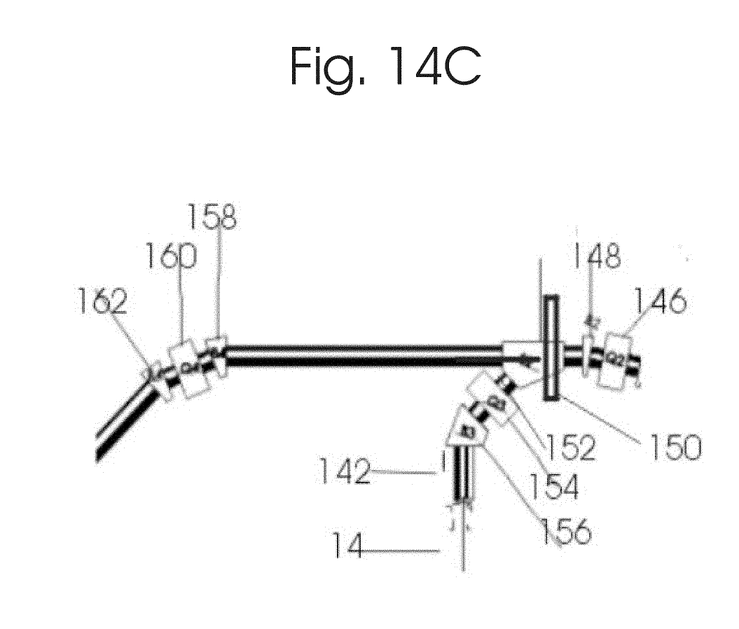

FIG. 14A: Shows switching the pencil beam into right and left beams and steering them into the beam lines with steering magnets. It illustrates the deflected beam 128, the beam switching magnet 129 which switches the beam to left 130 and to right 132 and to left focusing magnets 134 and to right focusing magnet 136 which focuses the switched beams to right and to left. The bending magnets 138 and 140 steers the beam to the right and left with a 45-degree bending.

FIG. 14B illustrates the 90 degree bending of the Laser wakefield Thompson scattering electron or Compton scattering gamma ray with collinear electron beam or proton pencil beam 14 and its passing through the 90 degree bending beamline 142 towards the microbeam or nanobeam or minibeam generating tissue equivalent collimator system 12. The 11.250 bending magnet-1, 144, the quadrupole focusing element 146, another 11.250 bending magnet-2, 148, and the beam switching bipolar magnet 150 steers this beam through the 90 degree bending beamline 142 towards the microbeam or nanobeam or minibeam generating tissue equivalent collimator system 12. The bending and focusing magnets 144, 146 and 148 bends the beam to 22.50. The beam switching bipolar magnet 150 switches the beam at 15-degree angle divergence. One of the switched beams is made to make a 450 bend when the 450 bending magnet 152 is activated. It is then made to travel towards the microbeam or nanobeam or minibeam generating tissue equivalent collimator system 12 by the beam steering system magnets consisting of 152, 154, and 156 where the steering magnet 152 is a 45 degree bending magnet-1, 154 is a quadruple focusing element, and 156 is a 45 degree bending magnet-2. The beam is thus bent to 90 degree and made to pass through the microbeam or nanobeam or minibeam generating tissue equivalent collimator systems 12 to generate the focused microbeam or nanobeam or minibeam 50. Simultaneously, another segment of the split beam is steered towards the next microbeam or nanobeam or minibeam generating tissue equivalent collimator systems 12 to generate similar focused microbeam or nanobeam or minibeam 50 for the multiple fields' simultaneous microbeam or nanobeam or minibeam radiosurgery. Alternatively, the pencil beam is switched sequentially for each of the multiple fields' sequential radiosurgery within split seconds intervals.

FIG. 14C: Illustrates the components for steering the beam towards the next microbeam or nanobeam or minibeam generating tissue equivalent collimator systems 12 with two 22.5 degree bending of the beam to make a combined 45 degree bend to guide the beam through the octagonal beam line and the simultaneously switched another segment of the beam by the switching magnet 150 as steered towards the microbeam or nanobeam or minibeam generating tissue equivalent collimator systems 12. The other switched beam is steered through the octagonal beam line to its next 45 degree bending site. It is accomplished with a 22.5 degree bending magnet-1, 158 and it's focusing with a focusing quadrupole element 160 and another 22.5-degree bent with the bending magnet-2, 162. When this beam reaches the next treatment head, location, it is again switched as two beams as before by the switching magnets. One segment of such beam is steered towards the next microbeam or nanobeam or minibeam generating tissue equivalent collimator systems 12. If a microbeam or nanobeam or minibeam generating tissue equivalent collimator systems 12 is not present or to be bypassed, the beam switching bipolar magnet 150 of that station and that station's beam steering towards its microbeam or nanobeam or minibeam generating tissue equivalent collimator systems 12, the switching magnets 152, 154 and 156 at this location is are switched off. In this instance, the beam is steered to the next treatment head station through the octagonal beam line as described before. Such beam switching at each locations of the microbeam or nanobeam or minibeam generating tissue equivalent collimator systems 12 facilitates all field simultaneous microbeam or nanobeam or minibeam radiosurgery of the tumor in a patient. Alternatively, the pencil beam is switched sequentially for each of the multiple fields' sequential radiosurgery within split seconds intervals.

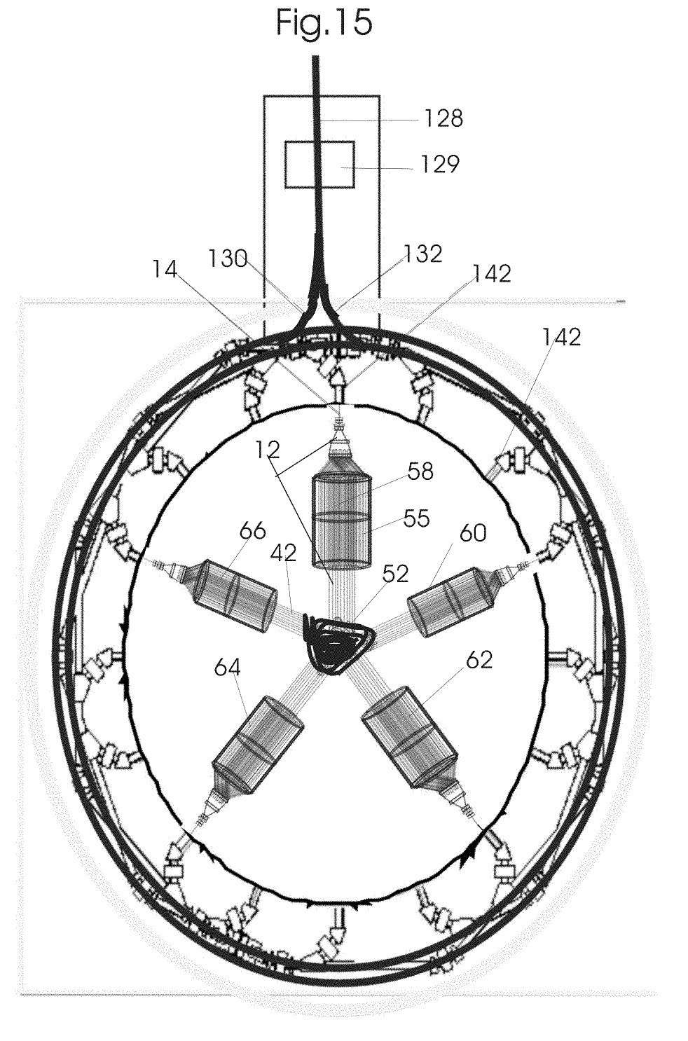

FIG. 15 shows the 90 degree bent laser wakefield Thompson scattering electron or Compton scattering gamma ray with collinear electron beam or proton beam 14 injection into five microbeam or nanobeam or minibeam generating tissue equivalent collimator systems 12 and generating microbeams or nanobeams or minibeams for all fields simultaneous microbeam or nanobeam or minibeam single or hypofractionated kGy radiosurgery. The deflected laser wakefield Thompson scattering electron or Compton scattering gamma ray with collinear electron beam or proton pencil beam 128 is switched by the beam switching magnet 129 to left switched pencil beam-1, 130 and to right switched pencil beam-2, 132 and they are transported to 90 degree bending beam line 142 and delivers this bam as Laser wakefield Thompson scattering electron or Compton scattering gamma ray with collinear electron beam or proton or carbon ion pencil beam 14 to five microbeam or nanobeam or minibeam generating tissue equivalent collimator systems 12 where it is processed as microbeam or nanobeam or minibeam. The microbeam or nanobeam or minibeam from microbeam or nanobeam or minibeam generating tissue equitant universal collimator-1, 58, tissue equitant universal collimator-2, 60, tissue equitant universal collimator-3, 62, tissue equitant universal collimator-4, 64 and tissue equitant universal collimator-5, 66 are focused by the focusing magnet 48 and they converge at the isocentric tumor 52. These parallel beams 42 are shaped in conformity with the tumor by the patient specific collimator 55 which is interchangeable.

To switch and steer the pencil beam first it is switched into multiple simultaneous microbeam or nanobeam or minibeam generating tissue equivalent collimator systems 12, first the pencil beam is split into right and left beams and steered into the beam lines with steering magnets. The right and left beam switching and this switched beam's steering is illustrated in FIG. 14A. The deflected laser wakefield Thompson scattering electron or Compton scattering gamma ray with collinear electron beam or proton or carbon ion pencil beam pencil beam 128 is switched into left and right by the beam switching magnet 129. To left switched pencil beam 130 and to right switched pencil beam 132 are focused by the left focusing magnets 134 and to right focusing magnet 136. To right 45 degree bending magnet 138 and to left 45 degree bending magnet 140 steers the beam to the right and to left with 45-degree bending. Subsequently, this pencil beam is bent to 900 by the 900 bending magnet 142 towards the microbeam or nanobeam or minibeam generating tissue equivalent collimator system 12 as illustrated in FIG. 14B. The 11.250 bending magnet-1, 144, the quadrupole focusing element 146, another 11.250 bending magnet-2, 148, and the beam switching bipolar magnet 150 shown in FIG. 14B steers this beam through the 900 bending beamline 142 towards the microbeam or nanobeam or minibeam generating tissue equivalent collimator system 12. The bending and focusing magnets 144, 146 and 148 bends the beam to 22.50. The beam switching bipolar magnet 150 switches the beam at 15-degree angle divergence. One of the switched beams is made to make a 450 bend when the 450 bending magnet 152 is activated. It is then made to travel towards the microbeam or nanobeam or minibeam generating tissue equivalent collimator system 12 by the beam steering system magnets consisting of 152, 154, and 156 where the steering magnet 152 is a 45 degree bending magnet-1, 154 is a quadruple focusing element, and 156 is a 45 degree bending magnet-2. The beam is thus bent to 900 and made to pass through the microbeam or nanobeam or minibeam generating tissue equivalent collimator systems 12 to generate the focused microbeam or nanobeam or minibeam 50. Simultaneously, another segment of the split beam is steered towards the next microbeam or nanobeam or minibeam generating tissue equivalent collimator systems 12 to generate similar focused microbeam or nanobeam or minibeam 50 for the multiple fields' simultaneous microbeam or nanobeam or minibeam radiosurgery. Alternatively, the pencil beam is switched sequentially for each of the multiple fields' sequential radiosurgery within split seconds intervals.

Similarly, the split pencil beam is steered towards the next microbeam or nanobeam or minibeam generating tissue equivalent collimator systems 12 with two 22.5 degree bending of the beam to make a combined 45 degree bend to guide the beam through the octagonal beam line and for the simultaneous switching of another segment of the beam by the switching magnet 150 towards the microbeam or nanobeam or minibeam generating tissue equivalent collimator systems 12 as shown in FIG. 14C. The other switched beam is steered through the octagonal beam line to its next 45 degree bending site. It is accomplished with a 22.5 degree bending magnet-1, 158 and it's focusing with a focusing quadrupole element 160 and another 22.5-degree bent with the bending magnet-2, 162. When this beam reaches the next treatment head, location, it is again switched as two beams as before by the switching magnets. One segment of such beam is steered towards the next microbeam or nanobeam or minibeam generating tissue equivalent collimator systems 12. If a microbeam or nanobeam or minibeam generating tissue equivalent collimator systems 12 is not present or to be bypassed, the beam switching bipolar magnet 150 of that station and that station's beam steering towards its microbeam or nanobeam or minibeam generating tissue equivalent collimator systems 12, the switching magnets 152, 154 and 156 at this location are switched off. In this instance, the beam is steered to the next microbeam or nanobeam or minibeam generating tissue equivalent collimator systems 12 station through the octagonal beam line as described before. Such beam switching at each locations of the microbeam or nanobeam or minibeam generating tissue equivalent collimator systems 12 facilitates all field simultaneous microbeam or nanobeam or minibeam radiosurgery of the tumor in a patient. Alternatively, the split pencil beam at each locations of microbeam or nanobeam or minibeam generating tissue equivalent collimator systems 12 station is switched sequentially for each of the multiple fields' sequential radiosurgery within split seconds.

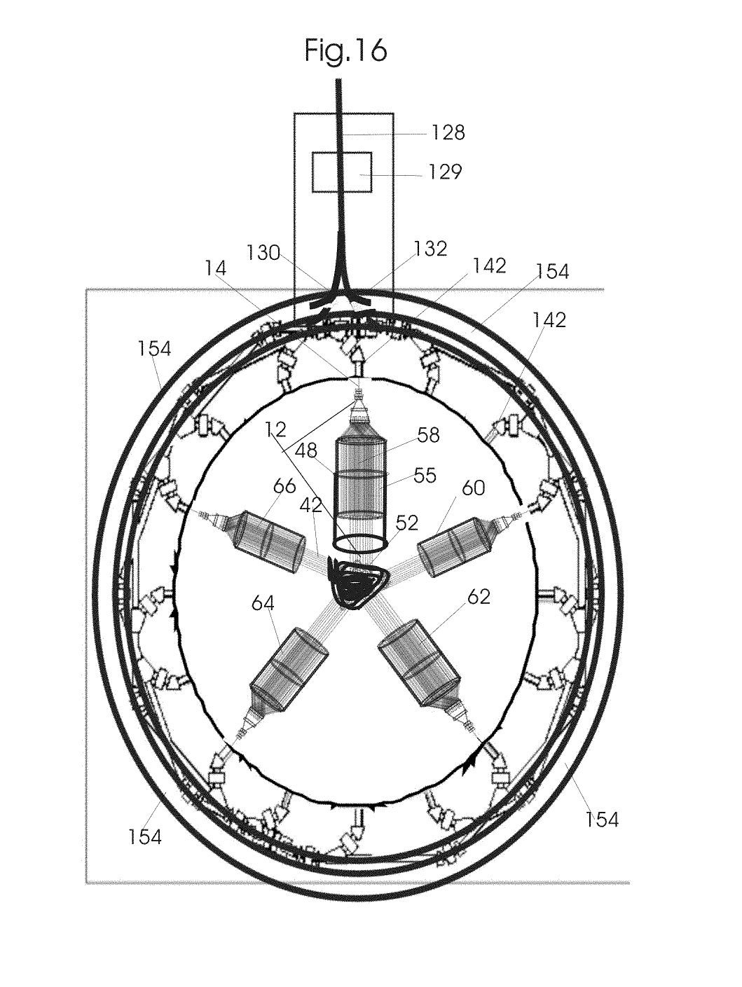

FIG. 16 illustrates the pencil beam as injected into a mini storage ring 154 from which synchronized multiple simultaneous beams are switched to the microbeam or nanobeam or minibeam generating five tissue equivalent collimator systems 12 where the microbeam or nanobeam or minibeams are generated. The deflected laser Wakefield Thompson scattering electron or Compton scattering gamma ray with collinear electron beam or proton or carbon ion pencil beam 128 is switched into the beam storage ring 154. The method of beam switching of multiple simultaneous electron beams from the beam storage ring 154 is described in U.S. Pat. No. 8,173,983 that was issued to this inventor (33). The beam switching from the beam storage ring 154 is similar to those described in FIG. 15. The beam switching from the storage ring 154 to microbeam or nanobeam or minibeam generating tissue equivalent collimator systems 12 are similar to those described in FIG. 15. The beam storage ring 154 contains fast magnetic switches (not shown here) having rising time of less than 0.25 nsec. It divides the beam pulse into a sequence of separate beam pulses by decomposing the beam into separate individual beam segments. The individual beam segments are delayed in a manner so that each beam segments are at their respective beam switching point when they are switched into their respective 90° bending beam line 142. Each beam segment's delayed arrival at 90° bending beam line 142 is achieved by arranging each beam segments to travel in an approximate circular path in the beam storage ring 154 and adjusting the beam segment's transit time from one beam switching point to the next beam switching point 142 in the beam storage ring 154 as equal to the sum of the beam segment length and the magnetic switch's rise time. The beam is focused and steered around the beam storage ring 154 by quadrupole focusing elements 146 and the 11.25 degree bending magnets 144 and 146. When the beam storage ring 154 is filled with such beam segments, all the beam switching magnets, the 11.25 degree bending magnets, the 45° bending magnet 152, quadruple focusing element 146, are activated.

In sequential treatment mode, the segments of the beams arriving at the beam switching points in storage ring 154 are switched on in sequence by sequentially activating the beam switching magnets 148, 152, 154 and 156. The beams are switched from the mini storage ring 154 before it is filled with all segments of the beams as in the case of simultaneous beams switching. Each beamlets arriving at the beam switching points is switched in sequence for the sequential treatment of each field, within a few seconds' intervals.

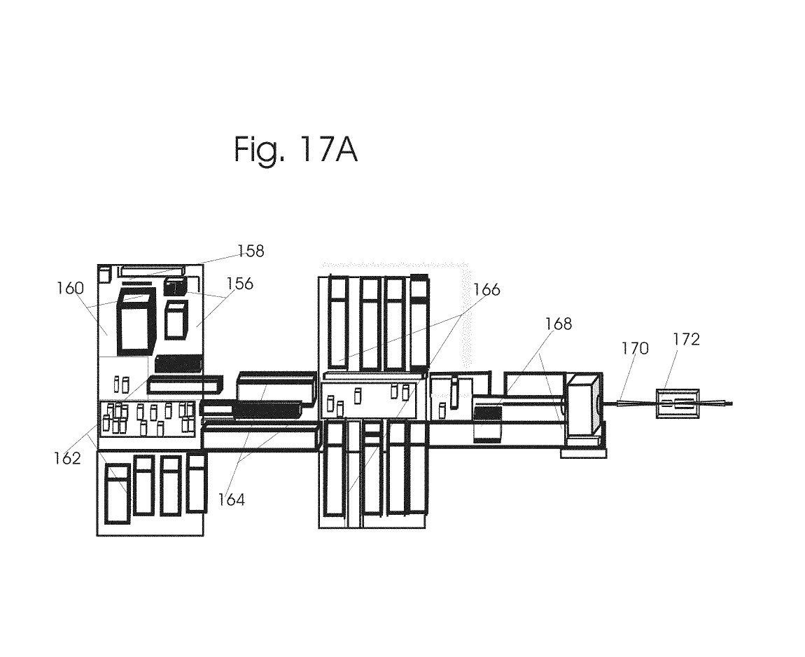

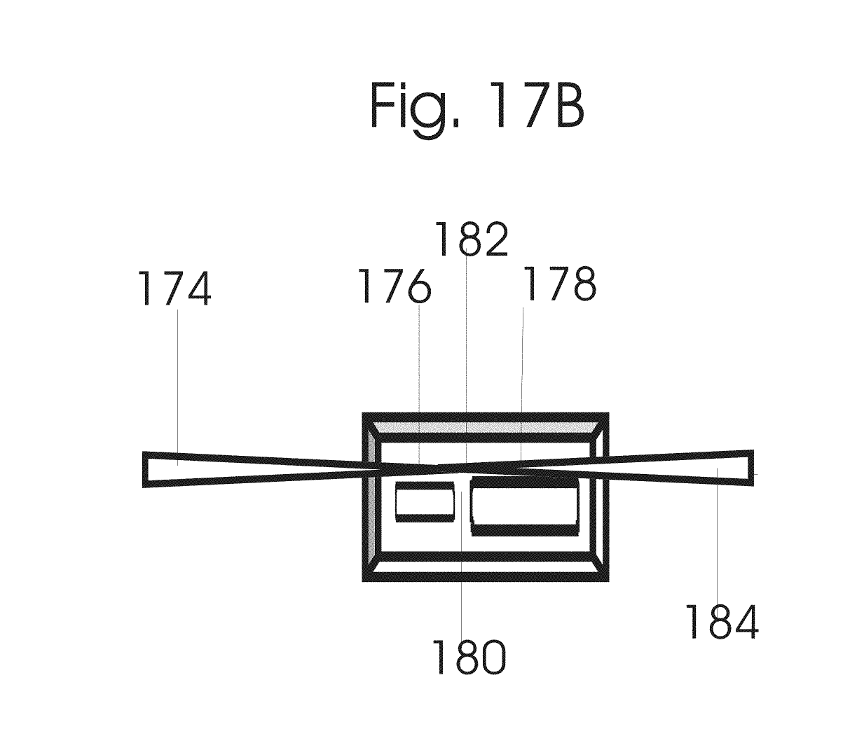

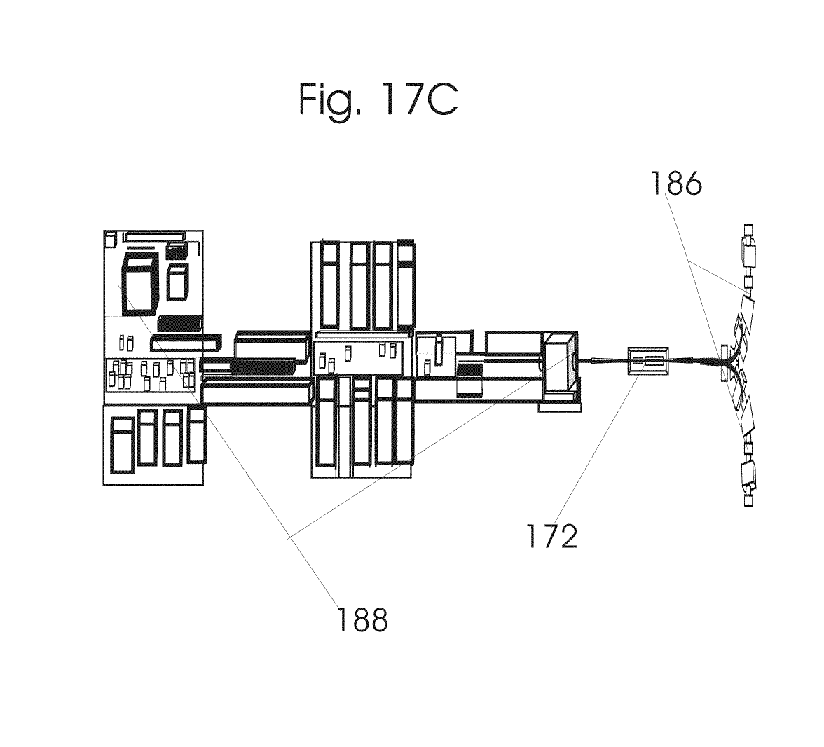

FIG. 17A shows a laser wakefield accelerator with dual supersonic gas jet adapted for stable, 50 to 300 MeV range tunable quasi monoenergetic electron microbeam or nanobeam or minibeam and or high brightness collinear inverse Compton scattering gamma ray microbeam or nanobeam or minibeam generation for kGy radiosurgery. Single gas jet laser wakefield electron acceleration is not satisfactory for radiation therapy since its electron beam and the dose rate are not stable. The recently reported dual gas jet system has more stable electron beam (34, 35, and 36). By reference, they are incorporated herein in their entreaty. Briefly, the details of Diocles laser wakefield accelerator is described in the cited references (34, 37). It consists of an initial 10 ps. 75 MHz pulse generating system 156, a diffraction grating system 158 where the 10 ps pulse is stretched to more than 300 ps, the first Ti-Sapph crystal amplifier system 160 in which the laser is amplified by 9 passes, a second Ti-Sapph crystal amplifier system 162 in which these pulses passes through 5 times and amplifies to 10 Hz frequency and to more than 70 mJ, third Ti-Sapph crystal system 164 in which the pulse is enlarged and amplified to more than 2 J while the pulse frequency is maintained at 10 Hz and finally a spatially stretching 50 mm Ti-Sapph crystal 166 that brings the energy up to 5 J . A holographic diffraction grating system 168 compresses the 300 ps pulse to less than 30 fs and increase the final power to 100 TW. This 30 fs, 100 TW laser pulses 170 interacts with the dual stage composite gas targets 172 for the laser electron injection and acceleration to 50-300 MeV electron which is described below under FIG. 17B. Before the wakefield accelerated high energy electron beam 188 is transported to beam bending and splitting magnet system 186, the laser is dumped away from the wakefield accelerated electron beam by the laser dumping mirror 173.

FIG. 17B illustrates the injection and acceleration of a single laser pulse focused on to dual stage composite gas jets with independently adjustable gas density that generates 50 to 300 MeV stable laser wakefield accelerated quasi monoenergetic electron beam and high brightness collinear inverse Compton scattering gamma rays for radiosurgery. In one such dual gas jet system a 0.5 mm long first gas jet nozzle and 0.5 or 2 mm long second gas jet nozzle separated by a 0.5 mm gap is reported (38) In a second dual gas jet system a 4 mm first and 10 mm long second gas jet is used to boost stable electron energy, which boosts the energy to 3 GeV (36). Such high energy beam is not needed for the medical use, radiation therapy. These prior arts are referred here in their entirety. As shown in this FIG. 17B, such dual stage composite gas jet 172 generates stable electron microbeam, nanobeam or minibeam in tissue equivalent collimators suitable for single or hypofraction kGy radiosurgery. The PW laser pulse 174 is shown first to interact with the 2 mm supersonic He gas jet 176 and then with 4 mm long second He/N2 supersonic gas jet 178 separated by 0.5 mm gap 180 and laser focal point 182 set in between the gas jets at a height of 2 mm above the nozzles and with adjusted gas density. The wakefield accelerated electron beam is shown as forward propagating accelerated beam 184.

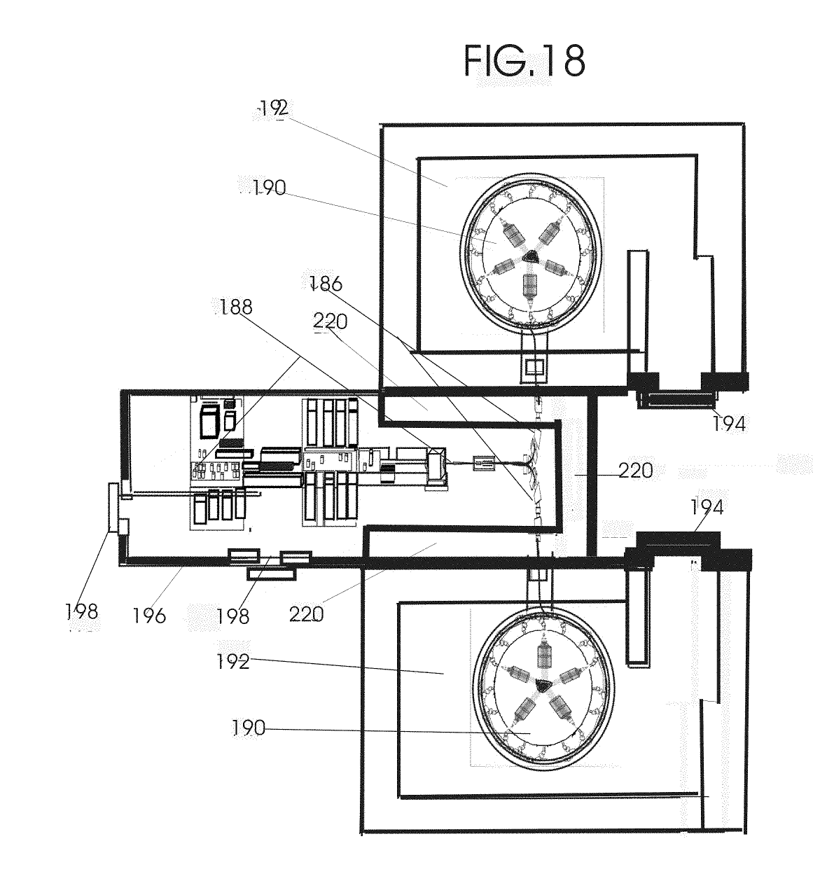

FIG. 17C illustrates a laser wakefield accelerator with dual supersonic gas jets attached to the beam bending and splitting magnet shown in FIG. 14A for steering of the wakefield accelerated electron beam to two sets of five tissue equivalent collimators shown in FIG. 18 that generates microbeam or nanobeam or minibeam. The beam bending and splitting it into two beams is illustrated and described under FIG. 14A. The laser wakefield accelerator system 188 with dual supersonic gas jet adapted for stable, 50 to 300 MeV range tunable quasi monoenergetic electron microbeam or nanobeam or minibeam and or high brightness collinear inverse Compton scattering gamma ray microbeam or nanobeam or minibeam generation for kGy radiosurgery is illustrated and described under FIG. 17A. Injection and acceleration of a single laser pulse focused on to a dual stage composite gas jets 172 with independently adjustable gas density that generates 50 to 300 MeV stable laser wakefield accelerated quasi monoenergetic electron beam and high brightness collinear inverse Compton scattering gamma rays is shown and described under FIG. 17B. The laser is dumped away from the wakefield accelerated electron beam by the laser dumping mirror 173. Switching the laser wakefield pencil electron beam into right and left beams and steering them into the beam lines with steering magnets is illustrated and described in FIG. 14A. In this FIG. 17C, they all together are shown as a laser wakefield accelerator system with dual supersonic gas jets attached to the beam bending and splitting magnet 186.

FIG. 18 shows a laser wakefield accelerator with dual supersonic gas jet generating electron beam as connected to the bending and splitting magnets and the beam steering to two sets of five tissue equivalent collimator systems installed in their respective radiation protective treatment rooms for electron, Compton gamma, or proton microbeam or nanobeam or minibeam kGy radiosurgery. In FIG. 17C a laser wakefield accelerator with dual supersonic gas jets attached to the beam bending, splitting magnets and beam steering to tissue equivalent collimator systems that generates microbeam or nanobeam or minibeam illustrated. Two sets of five tissue equivalent collimator system attached to such beams steered into radiation protective treatment rooms. Sets of microbeam or nanobeam or minibeam generating units each consisting of five simultaneous microbeam, nanobeam or minibeam generating tissue equivalent collimator systems 190 is installed in each radiation protective treatment rooms 192. The laser wakefield accelerator system 188 is sandwiched in between the two radiation protective treatment rooms 192. In dual stage composite gas target 172 stable electron beam is generated. The laser is dumped away from the wakefield accelerated electron beam by the laser dumping mirror 173. The VHEE electron beam is bent and split into two beams by the beam bending and splitting magnet systems 186 and the split beams are steered into each of the five simultaneous microbeam, nanobeam or minibeam generating tissue equivalent collimator systems 190. Entrance and exit to and from the radiation protective treatment rooms 192 are controlled by the treatment room radiation protective entry doors 194. Entrance to and from the laser wakefield accelerator system room 196 is controlled by the two wakefield accelerator system room's radiation protective entry door 198, one on the front and one on the side. The wide corridor in between the two radiation protective treatment rooms 200 separates the two radiation protective treatment rooms 192.

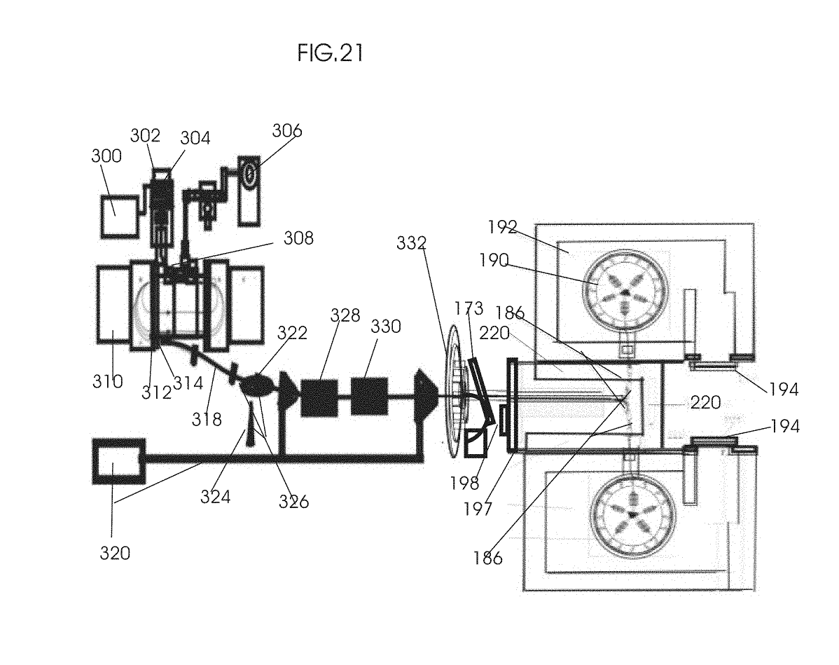

The machine generated secondary radiation to the patient from a laser driven proton system is prohibitively high (39). While a laser driven compact system could be installed in a conventional medical linear accelerator radiation therapy vault and it could be operated very economically, its secondary radiation also far exceeds the maximum allowable limits of radiation to occupationally exposed personals and to the general public outside the treatment areas. Such dangers from the secondary radiation to the patients, occupationally exposed personals and to the public negates the numerous advantages of such compact system's clinical use and its most attractive low cost associated affordability to patients. Such high secondary radian to the patient, to the occupationally exposed persons and to the general public is significantly reduced in this invention by several safety measures. First the source that generate the radiation, the laser wakefield accelerator system 188 and similar one with dielectric waveguide, FIG. 21B and FIG. 21.C or with corrugated pipe waveguide, FIG. 21D are housed in a separate room that is sandwiched between the two radiation therapy vaults. Especially, the source with the dual stage composite gas target 172 and the beam lines, the beam bending and splitting magnet system 186 are sandwiched in between the heavily shielded radiation protective treatment rooms 192. Additional shielding in laser wakefield accelerator system room 220 and additional sliding door shielding reduces the secondary radiation dose to the corridor in between the two radiation protective treatment rooms 200, the area that faces the radiation protective treatment rooms 192 and outside the wakefield accelerator system room 196.

It contrasts the source housing within the treatment room described for laser driven proton radiation therapy (39). In this case, the readings taken for the secondary neutron, gamma radiation, photoneutrons and X-rays are high even for the conventional daily 2 Gy fraction radiation therapy (40). In addition to the separation of the source from the treatment vault 192, the secondary radiation that is generated by the beam handling in the treatment vault is filtered and absorbed by the tissue equivalent collimator system described under FIG. 1A and FIG. 1B. It is further illustrated in the five simultaneous microbeam, nanobeam or minibeam generating tissue equivalent collimator systems 190. It not only generates the microbeam, nanobeam and minibeam but it also absorbs the harmful secondary neutrons, other ions and gamma radiation. It facilitates safe clinical application of laser driven electron and proton machines. Laser photocathode electron accelerated to 50 to 250 MeV as very high energy electron beam for radiation therapy is described in U.S. Pat. No. 8,618,521 (32) but this innovative approach could cause high skin toxicity from the broad electron beam with dₘₐₓ at the skin surface and to protect the patient, the personals caring for the patient and the public from the secondary radiation including the neutron it generates. Electron beam can cause severe radiation toxicity, including severe errythema, painful ulceration and necrosis, and radiation fibrosis. However, the Monte Carlo simulation of 150-250 MeV electron beam was reported to have less lateral penumbra and its depth dose at less than 10 cm is similar to photon beam and as having practical range Rₚ greater than 40 cm (37). Combined with magnetic focusing of the very high energy electron pencil beam (17), microbeam and nanobeam and the very high energy electron beam's deeper penetration as shown by Monte Carlo simulation (37) makes the deep penetrating pencil electron beam, microbeam, nanobeam and minibeam similar to proton pencil beam microbeam, nanobeam and minibeam with less skin toxicity and with well defined microbeam, nanobeam and minibeam peak and valley doses. Still its secondary neutron is of major concern. These systems are not ideal for kGy radiosurgery. Contrary to such electron beam radiation therapy systems, the separation of the radiation source from the treatment room and its housing in the laser wakefield accelerator system room 196 and with tissue equivalent collimators generating microbeam, nanobeam and minibeam and beam focusing with magnets to bring the electron beam's dₘₐₓ much deeper to the skin surface all described in this invention makes the kGy electron beam radiosurgery feasible. Under such treatments, the normal stem cells from the valley regions of the pair of microbeams, nanobeams or minibeams proliferates and migrates to the high dose tracks peak dose regions in normal tissue and heals its radiation damage. Such high dose electron beam radiosurgery, including kGy radiosurgery is safe to the skin and to the deeper normal tissue. It is also safer from secondary, machine producing neutron radiations to the patient, to those treating the patient and also for those outside the treatment room.

FIG. 19 illustrates a laser wakefield accelerator with dual supersonic gas jet as connected to the bending and splitting magnets and the beam steering to two sets of five tissue equivalent collimator systems installed in their respective radiation protective treatment rooms and with floor plan for the office, physics, patient care, imaging and research. As shown in FIG. 18, the laser wakefield accelerator system 188 generating electron beam is connected to the bending and splitting magnets 186 and the beam steering to two sets of five tissue equivalent collimator systems 190 installed in their respective radiation protective treatment rooms 192. The laser wakefield accelerator system 188 is sandwiched in between the two radiation protective treatment rooms 192. Entrance and exit to the radiation protective treatment rooms 192 are controlled by the treatment room radiation protective entry doors 194. Entrance and exit to and from the laser wakefield accelerator system room 196 is controlled by the wakefield accelerator system room's radiation protective entry doors 198, one in its front and another on its side. The wide corridor in between the two radiation protective treatment rooms 200 separates the two radiation protective treatment rooms 192. The scattered and secondary radiation in the radiation protective treatment rooms are monitored with silica optical fiber dosimeter-4 254 and silica optical fiber dosimeter-5 256. The floor plan for supporting services, physics, patient care, imaging and research are at the opposite side of the wide corridor 214. The wakefield accelerator system 188 and the radiation protective treatment room 192 with five simultaneous microbeam, nanobeam or minibeam generating tissue equivalent collimator systems 190 are away from the laser wakefield accelerator system 196 and the two radiation protective treatment rooms 192. It minimizes the outside secondary radiation to less than allowable level. These rooms include clinical room 1-6, numbered as 202, 204, 206, 208, 210 and 212. The corridor in between the two radiation protective treatment rooms 200 is used for access to the radiation protective treatment rooms 192 and to the laser wakefield accelerator system room 196. The wide corridor 214 is used for patient transport to the treatment and to clinical and imaging services. Entry and exit to each of these rooms are through each room's entry and exit doors 217. Under FIG. 18, the radiation protective advantages of such floor planning with wakefield accelerator system room 196 sandwiched in between the two radiation protective treatment rooms 192 is described. The precautionary measures with shielding from secondary neutron, ions and gamma radiation facilitates safe high and kGy radiosurgery with multiple sources of simultaneous microbeam, nanobeam or minibeam. Magnetically focused kGy radiosurgery with electron microbeam, nanobeam or minibeam have the least secondary radiation. The kGy single or hypofraction radiosurgery with highly focused electron microbeam, nanobeam or minibeam is as effective as X-rays, gamma rays, proton and carbon ion. It is more economical and easier to implement. Here kGy radiosurgery with electron and also with Compton gamma, proton and ions are described. With silica optical fiber dosimeter-6 258, silica optical fiber dosimeter-7 260 and silica optical fiber dosimeter-8 262, the dose outside the treatment rooms and are monitored.

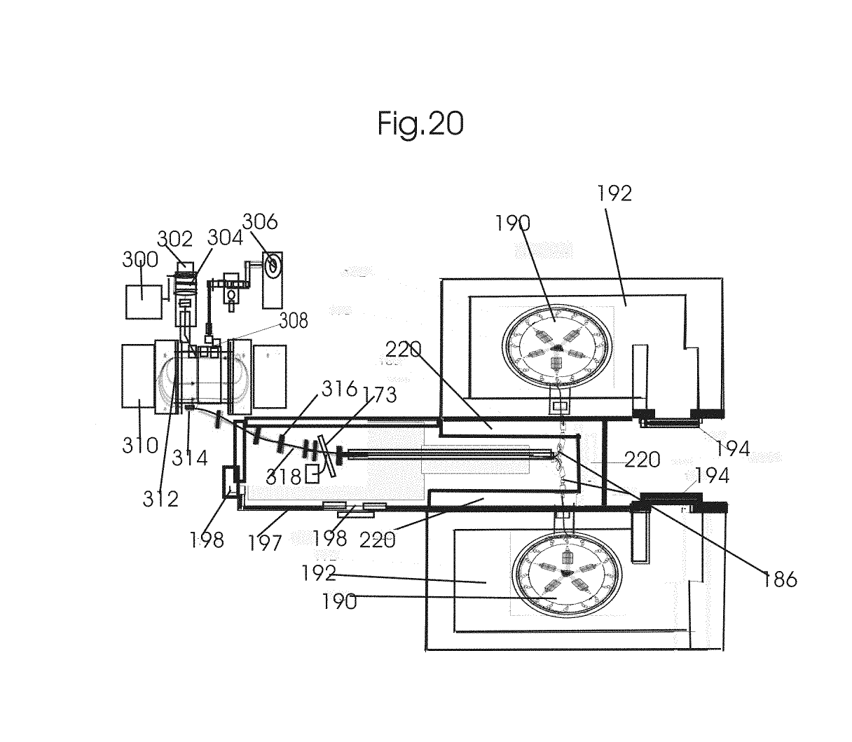

FIG. 20-A shows a 150 MeV stable very high energy electron beam at dose rate of 2-3 Gy per second generating racetrack laser-photocathode-racetrack microtron system instead of the laser wakefield accelerator shown in FIG. 19 and which is directly connected to the bending and splitting magnets 186 and the split beams as connected to two sets of five tissue equivalent collimator systems installed in their respective radiation protective treatment rooms. A laser-photocathode-racetrack microtron Z-pinch system similar to those made and proposed by Sumitomo Heavy Industries Ltd. (41,42,43,44,45) is modified as a laser wakefield accelerator system producing microbeam, nanobeam or minibeam for cancer stem cell ablating kGy radiosurgery in this invention. Referred Sumitomo system (42, 43, 44, and 45) is incorporated here in its entirety as a guide for the development of parallel collinear very high energy beams and microbeam for kGy radiosurgery. Other similar systems are also used to generate parallel very high energy collinear laser Wakefield beam lines and microbeam generation. Such systems consists of an Nd-YAG-laser 300, a RF-gun 302 with solenoid 304 and klystron 306, an accelerating cavity 308, two bending magnets 310, two field magnets 312, beam extraction magnet 314, beam focusing magnets 316. The laser is dumped away from the wakefield accelerated ion beams with a laser dumping mirror 173. The extracted beam 318 passes through the room labeled as room with extracted beam 197 towards the beam bending and splitting magnet system 186. The extracted beam 318 is split into right and left beams and steered to two sets of five tissue equivalent collimator systems 190 installed in their respective radiation protective treatment rooms 192 where radiosurgery is performed.

FIG. 21 illustrates 150 MeV electron beam generation with photocathode racetrack microtron and its synchronization with 100 TW 20 fs 10 Hz laser beam generated by laser wakefield acceleration combined with a Z-pinch gun for additional Z-pinch acceleration like those described in the literature before (45, 46, 47, 48, 49). It generates stable 150 MeV to a GeV electron beams with dose rate in the range of 109 Gy per second. Each of the collinear beams is connected to microbeam, nanobeam or minibeam generating high density tissue equivalent collimators in two adjacent treatment rooms for kGy range microbeam, nanobeam or minibeam radiosurgery. The laser-photocathode-racetrack microtron system is described under FIG. 20. It consists of a an Nd-YAG-laser 300, a RF-gun 302 with solenoid 304 and klystron 306, an accelerating cavity 308, two bending magnets 310, two field magnets 312, beam extraction magnet 314, beam focusing magnets 316. The photocathode-racetrack microtron processed extracted electron bunch 322 and a 100 TW, 20 fs, 10 Hz laser pulse 320 generated in a laser wakefield accelerator system 188 are synchronized to synchronized electron bunch-laser pulse 326 which then passes through the energy modulator 328 and bunch slicer 330 before it is further accelerated a Z-pinch gun 332. The laser is dumped away from the wakefield accelerated electron beam by the laser dumping mirror 173. The beam existing from the Z-pinch gun, Z-pinch gun processed energetic electron beam 334 is split and scanned by the scanning magnet 342. The split beams are kept focused with focusing magnets 336 as it is transported to the beam bending and splitting magnet system 186. This beam is split into right and left beams and steered to two sets of five tissue equivalent collimator systems 190 installed in their respective radiation protective treatment rooms 192 where radiosurgery is performed. Its contaminating neutron, X-ray, are absorbed by the tissue equivalent high density collimator systems described in FIG. 1C-1, FIG. 1C-2A, FIG. 1C2B, FIG. 1C3-1, FIG. 1C3-2, FIG. 1C-3-2, FIG. 1C3-3 and FIG. 1C3-4.

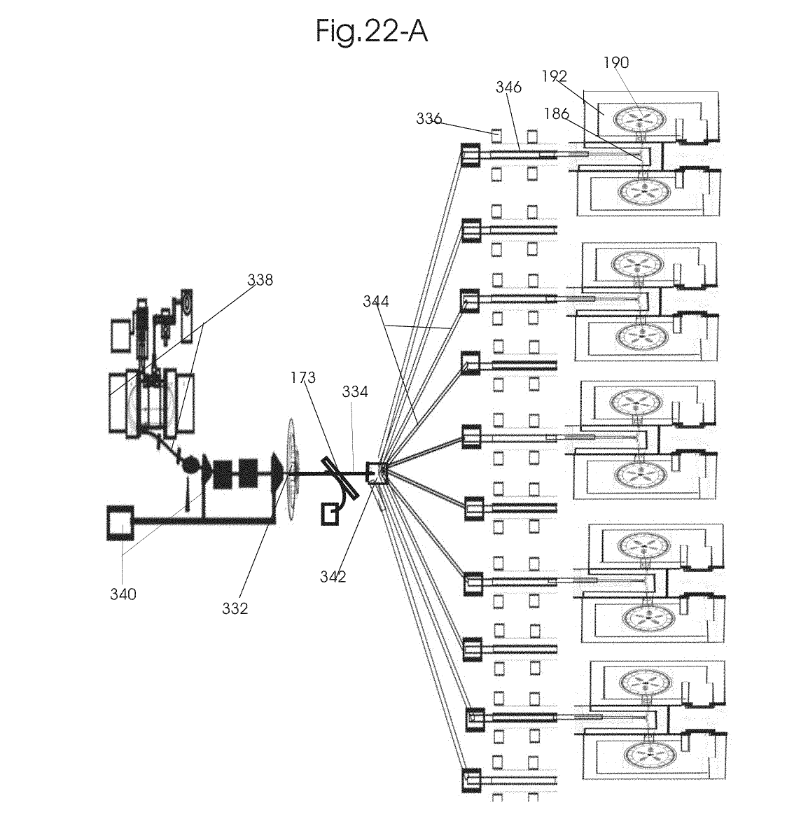

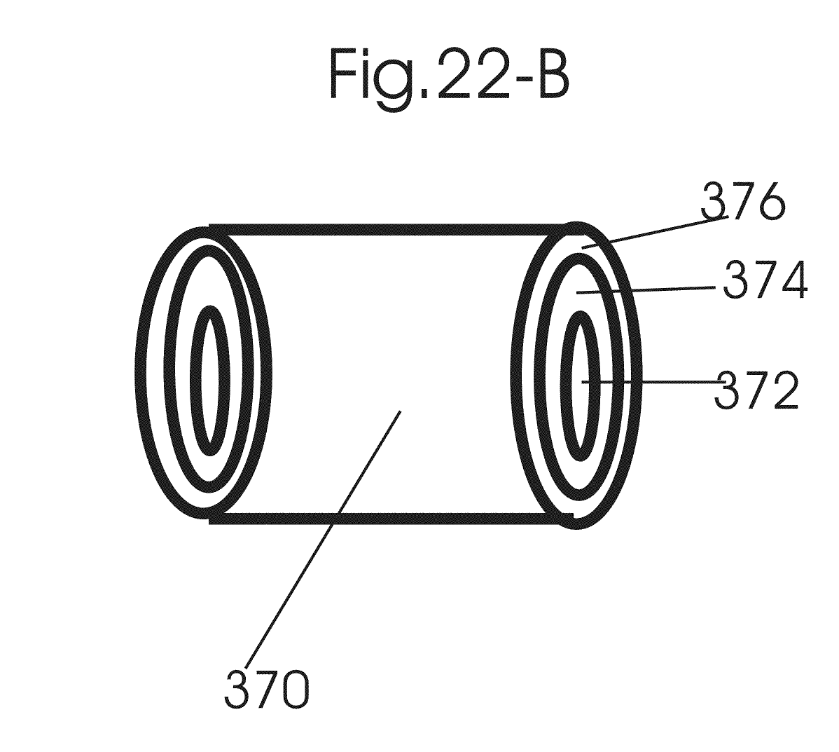

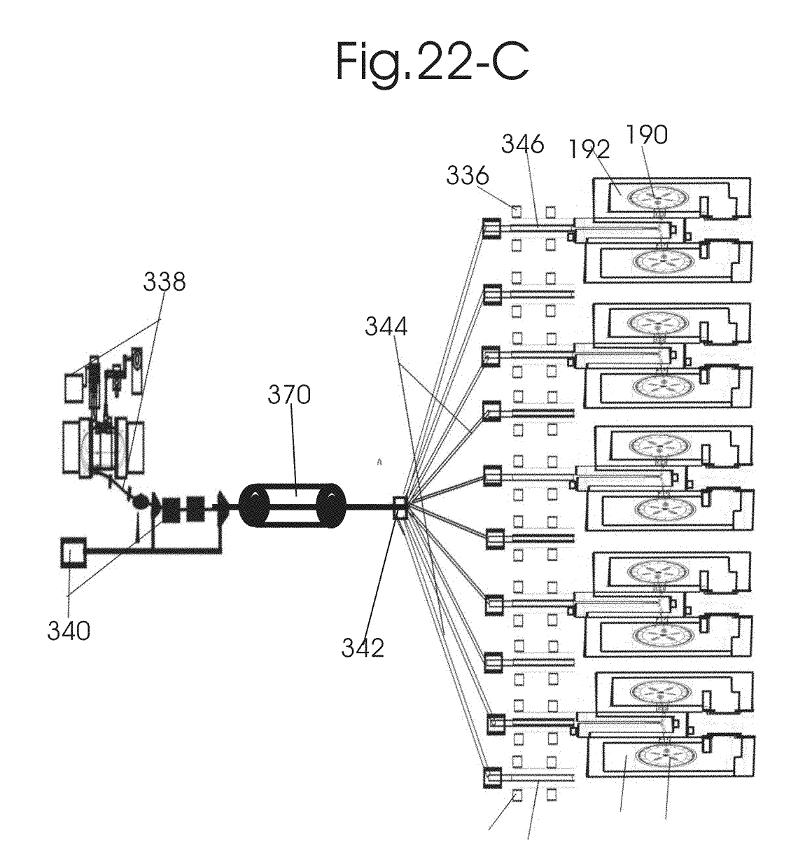

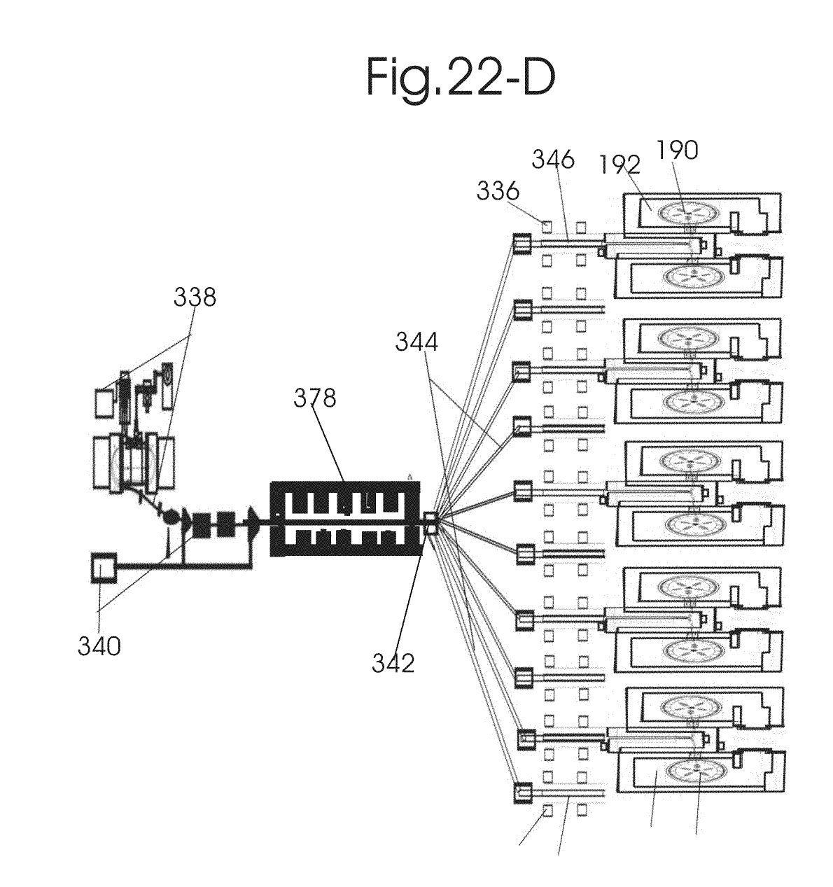

FIG. 22-A shows the same 150 MeV electron beam generating photocathode racetrack microtron system and its beam synchronization with 100 TW 20 fs 10 Hz laser beam generated by laser wakefield acceleration and their further Z-pinch acceleration with a Z-pinch gun as illustrated in FIG. 21 but the very high energetic electron beam 334 from the Z-pinch gun and separated from the laser beam is split into 10 beams and the alternate split beams as connected to ten treatment rooms with high density tissue equivalent secondary neutron absorbing collimator systems for kGy microbeam, nanobeam or minibeam radiosurgery. The photocathode racetrack microtron system 338, the TW laser and laser processing system 340 and the Z-pinch gun 332 are the same as described in FIG. 21. The collinear laser beam and the Z-pinch gun processed energetic electron beam 334 are separated by the laser dump mirror and laser beam absorber 173. The Z-pinch gun processed very high energetic electron beam 334 is scanned by the scanning magnet 342. They form a series of collinear very high energy electron beam. Collinear Wakefield accelerator for a high repetition rate multi beamline soft x-ray FEL has been described before (50). The scanned beams 344 is kept focused by the focusing magnet 336 and guided towards the beam bending and splitting magnet system 186 through scanned beam guiding tube 346. The beam bending and splitting magnet system 186 splits the beam to right and left and sends them to five simultaneous microbeam, nanobeam or minibeam generating tissue equivalent collimator systems 190 in the adjacent right and left radiation protective treatment rooms 192 where the microbeam, nanobeam or minibeam radiosurgery is performed.

FIG. 22-B shows a high repetition rate dielectric loaded circular wave guide inserted into the racetrack-laser wakefield accelerator system instead of the Z-pinch gun to improve the quality of the very high electron energy beam and to generate collilinear multiple beams for multiple suits radiosurgery. Dielectric wakefield accelerators (DWA) (51) has been suggested as a means to generate lower cost high repetition rate single stage collilinear wakefield accelerators (CWA) imbedded into a wiggler with alternating focusing and defocusing quadruples. Both DWA and corrugate pipe wakefield accelerator (CPWA) (52) are able to be CWAs. Combined with a 400 MeV superconducting accelerator and a spreader ten parallel FEL and ten parallel CWAs that supply 2 GeV electrons were proposed (50). Dielectric circular waveguides are described before (53). It is incorporated into the laser wakefield racetrack microtron system to generate collinear multiple beams from which microbeams, nanobeams or minibeams are generated in the tissue equivalent collimators. A modified dielectric circular waveguide 370 system is used in this invention. It consists of an inner vacuum 372, the dielectric 374 in between the inner vacuum 372 and the outer metal guide 376 (50,51). It is incorporated into the photocathode racetrack microtron 338-TW laser and laser processing system 340 as shown in FIG. 22-C.

FIG. 22-C illustrates a similar collinear multibeam wakefield very high energy electron accelerator system with ten split beams and the five alternate split beams connected to ten radiosurgical rooms with tissue equivalent collimators as in FIG. 22-A but the Z-pinch gun is replaced with a high repetition rate dielectric wave guide 370. The 150 MeV electron beam generating photocathode racetrack microtron system and its beam synchronization with 100 TW 20 fs 10 Hz laser beam generated by laser wakefield accelerator is the same as those shown in FIG. 22A. The Z-pinch gun is replaced with a high repetition rate dielectric wave guide. The beam exiting from the high repetition rate dielectric wave guide 370 is split into 10 beams and the alternate split beams are connected to ten treatment rooms with high density tissue equivalent secondary neutron absorbing collimator systems for kGy microbeam, nanobeam or minibeam radiosurgery. The collinear laser beam and the high repetition rate dielectric wave guide 370 processed very high energetic electron beam 334 are separated by the laser dump mirror and laser beam absorber 173. The high repetition rate dielectric wave guide 370 processed very high energetic electron beam 334 is split and scanned by the scanning magnet 342. The scanned beams 344 is kept focused by the focusing magnet 336 and guided towards the beam bending and splitting magnet system 186 through scanned beam guiding tube 346. The beam bending and splitting magnet system 186 splits the beam to right and left and sends them to five simultaneous microbeam, nanobeam or minibeam generating tissue equivalent collimator systems 190 in the adjacent right and left radiation protective treatment rooms 192 where the microbeam, nanobeam or minibeam radiosurgery is performed.

FIG. 22-D shows a similar collinear multibeam wakefield very high energy electron accelerator system with ten split beams and the five alternate split beams connected to ten radiosurgical rooms with tissue equivalent collimators as in FIG. 22-C but the high repetition rate dielectric wave guide 370 is replaced with a corrugated pipe waveguide 378. It removes the residual energy chirp in the beam before it is split to multibeams for radiosurgery. Like in FIG. 22-C, the 150 MeV electron beam generating photocathode racetrack microtron system and its beam synchronization with 100 TW 20 fs 10 Hz laser beam generated by laser wakefield accelerator is the same as those shown in FIG. 22-C. The high repetition rate dielectric wave guide 370 is replaced with a corrugated pipe waveguide 378. The beam exiting from the corrugated pipe waveguide 378 is split into 10 beams and the alternate split beams are connected to ten treatment rooms with high density tissue equivalent secondary neutron absorbing collimator systems for kGy microbeam, nanobeam or minibeam radiosurgery. The collinear laser beam and the corrugated pipe waveguide 378 processed energetic electron beam 334 are separated by the laser dump mirror and laser beam absorber 173. The corrugated pipe waveguide 378 processed very high energetic electron beam 334 is split and scanned by the scanning magnet 342. The scanned beams 344 is kept focused by the focusing magnet 336 and transported to the beam bending and splitting magnet system 186 through scanned beam guiding tube 346. The beam bending and splitting magnet system 186 splits the beam to right and left and sends them to five simultaneous microbeam, nanobeam or minibeam generating tissue equivalent collimator systems 190 in the adjacent right and left radiation protective treatment rooms 192 where the microbeam, nanobeam or minibeam radiosurgery is performed.

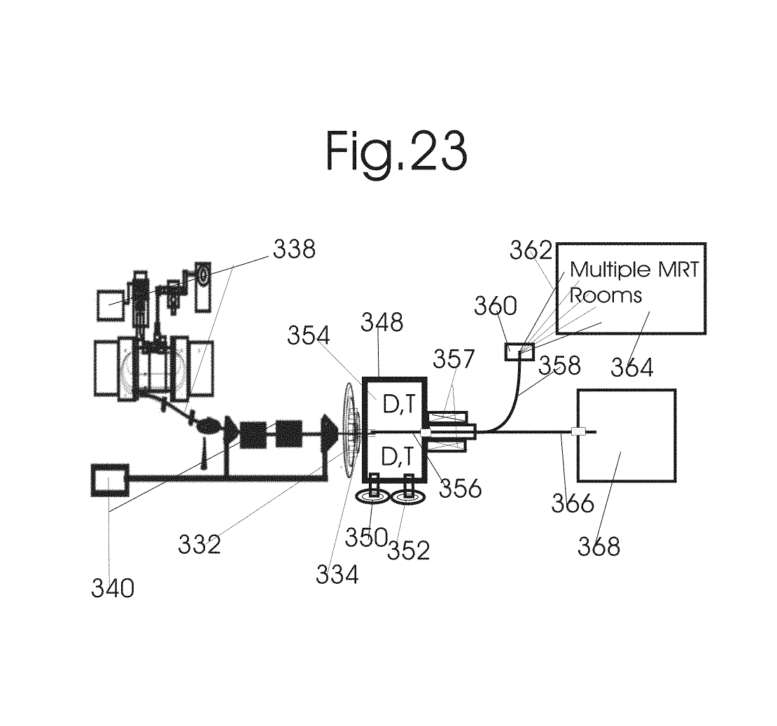

FIG. 23 illustrates the photocathode racetrack microtron system 338 and the TW laser and laser processing system 440 with Z-pinch gun 332 attached to a drift chamber in which the very high energy electron beam (VHEE) passes through deuterium-tritium gas that generates stable collinear accelerated VHEE beam and 2.45 MeV and 14 MeV neutron beam and their separation into VHEE and neutron beam for VHEE electron microbeam radiosurgery, and the high flux, 1012 to 1015 neutron generating radioisotopes.

The principles of collective acceleration of energetic ions by linear electron beams propagating in low pressure neutral gases (54, 55) is adapted to generate collinear very high energy electron beam (VHEE) along with 2.45 MeV or 14 MeV neutron for electron and neutron microbeam radiosurgery. The collective ion acceleration by intense electron beam would provide intense 14 MeV neutron. Electron is considered as a group or collection of charges. Based upon their proportional number of charges, they create accelerating fields capable of generating even up to 10¹³ to 10¹⁵ ions per bunch (54). It generates a reaction similar to Z-pinch. The accelerated collilinear electron and ions could be separated by a magnetic field (54). Energetic electron beam assisted fusion neutron generation is reported in U.S. Pat. No. 3,959,659 (56) and in U.S. Pat. No. 3,946,240 (57). Radioisotope production by high flux neutron generated by staged Z-pinch and deuteron and tritium gas is reported in U.S. Pat. No. 8,837,661 (58). High flux neutron is generated by collective acceleration of electron by linear electron beam propagating through deuteron and tritium gas in this invention. Z-pinches' instability (59) is overcome by staged Z-pinch. Likewise the Z-pinch instability is overcome by collective acceleration of energetic ions by linear electron beams propagating in low pressure neutral gases. Collective ion acceleration by injecting the electron beam into a drift chamber filled with neutral gas is simple. In this invention, stable energetic electron beam is produced by a combination of photocathode racetrack microtron system consisting of TW laser beam system and Z-punch gun. This stable energetic electron is injected into the drift tube along with deuterium and tritium. Accelerated high energy electron beam and the neutron beam generated in the drift chamber are separated by sweeping magnet. The electron beam is split into multiple collinear VHEE beams for radiation therapy. The neutron beam is used for radioisotope production. Because of the energetic racetrack microtron-laser Z-pinch electron interaction in a drift tube that generates high flux stable neutron beam, it is more efficient in radioisotope production than the staged Z-pinch with isotropic high flux neutron generation. Hence the method of low cost high flux neutron generation within a hospital setup by collective acceleration of ion shortens the time needed for desired radioisotope production for clinical use. The cost for clinically useful isotopes are also much reduced. .

The photocathode racetrack microtron system 338, TW laser and laser processing system 340, Z-pinch gun 332, and Z-pinch gun processed energetic electron beam 334 were described before, in FIG. 21 and FIG. 22. They are incorporated in FIG. 23. The Z-pinch gun processed energetic electron beam 334 enters the drift chamber 348 through a small opening. The drift chamber 348 is filled with puffs of deuterium-tritium gas mixture 354 by the gas puff pump 350. The pinch interaction of energetic electron beam with the deuterium tritium gas puffs generates collinear very high energy electron and neutron beams 356. The reacted deuterium and tritium gas mixture is evacuated from the drift chamber by the gas evacuation pump 352. As the collinear very high energy electron and neutron beams 356 exits from the drift chamber 348, the sweeping magnetic filed created by the sweeping magnet 357 deflects the electron beam from the neutron beam. The deflected electron beam splitting magnet 360 splits the deflected very high energy electron beam 358 into very high energy split electron beams 362 which are then guided to multiple MRT rooms 364 where microbeam radiosurgery is performed. The collinear very high energy electron and neutron beams 356 propagates linearly towards the high flux neutron-radioisotope precursor reaction chamber 368 which contains compartmentalized desired radionuclide precursors which are exposed to high flux, 1012 to 1015 neutron pulse for neutron activated radioisotope production. Desired radioisotope precursors include 99Mo, 24Na, 32P, 82Br, 56Mn, 64Cu or 198Au. Presently 99Mo and its daughter 99mTc is the most commonly used radioisotope in nuclear medicine. There is an acute shortage for this radionuclide.

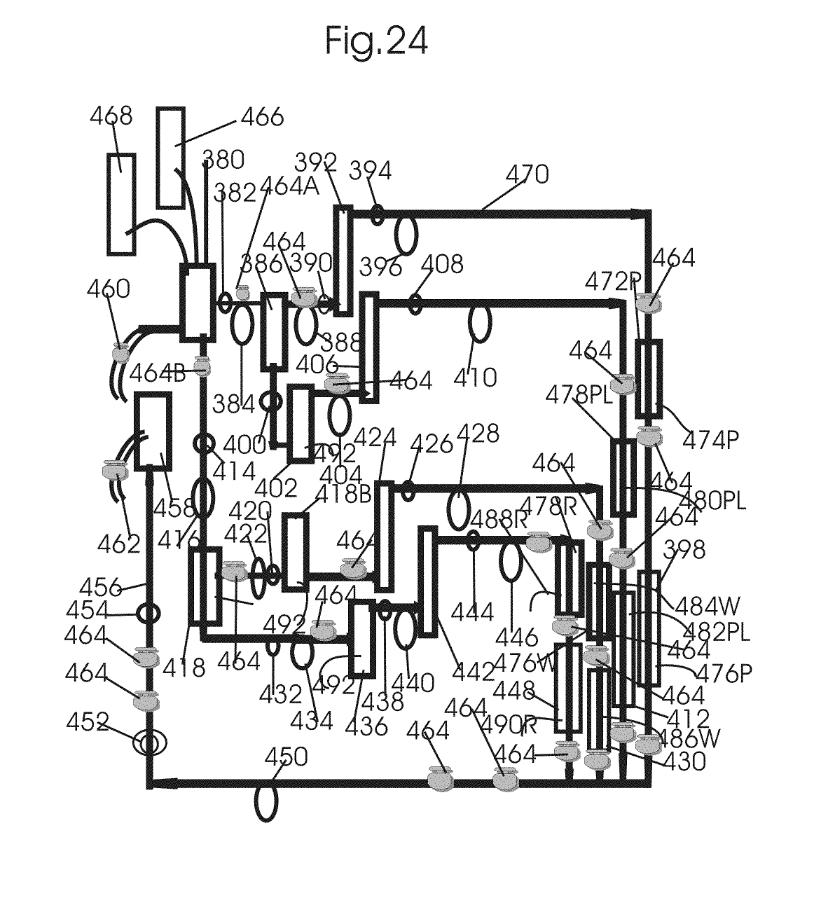

FIG. 24 shows removal of circulating tumor cells (CTC), RNA, DNA and DNA fragments, exosomes, microsomes and nanosomes from circulation after kGy radiosurgery and chemotherapy by pulsed flow apheresis to minimize bystander and abscopal effects associated tumor recurrence and metastasis. In this invention, these subcellular components of the tumor cells released after radiosurgery and chemotherapy is removed by pulse flow system combined with DNA affinity chromatography. Two intermittent pulse flow apheresis systems are run simultaneously to have a continuous flow apheresis of the exosomes, microsomes nanosomes including highly increased release of telomerase after chemotherapy/radiosurgery. One of such intermittent pulse flow system is shown in FIG. 24. It consists of the whole blood reservoir 380 to which the whole blood drawn from the patient at a rate of 15 to 150 ml/min through the blood flow inlet channel with clam and sensor 460 is collected. After drawing about 300 ml for the first intermittent flow apheresis, the blood flow to the whole blood reservoir 380 is stopped by clamping the clamp with sensors 464A and 464B. The whole blood drawn is then mixed with anticoagulant to keep the blood from clotting and to keep the blood at its normal viscosity from the anticoagulant reservoir 468 and normal saline from the normal saline reservoir 466 if needed to adjust the hematocrit reading. By 15 min gravity sedimentation the plasma layer with platelets and at its bottom the heavier white blood cells, the red cells and the very bottom circulating tumor cells (CTCs) if any are separated. A series of system clamps with sensors, 464, a series of densitometers, the densitometer 382, 390, 394, 400, 408, 414, 418, 420, 426, 432, 438, 444 and 454, a series of pulsed pumps, pulsed pump 384, 388, 396, 404, 410, 416, 422, 428, 434, 440, 446 and 450, whole blood reservoir 380 the plasma-platelet and exosomes, microsomes and nanosomes reservoir 386, reservoir for RBC plus WBC, CTC, exosomes, microsomes and nanosomes and 418, to the reservoir with WBC, CTC, exosomes, microsomes and nanosomes/DNA-Telomerase 418B, and to the reservoir for concentrated RBC and CTC exosomes, microsomes, nanosomes and CTC 436, separate reservoir with platelets, exosomes, microsomes and nanosomes/DNA-Telomerase 402, reservoir with WBC, exosomes, microsomes and nanosomes/DNA-Telomerase 418B, reservoir for concentrated RBC and exosomes, microsomes, nanosomes and CTC 436, a series of DNA/RNA/Telomerase affinity columns, DNA/RNA/Telomerase, exosomes, nanosomes affinity column-1 392, DNA/RNA/Telomerase, exosomes, nanosomes affinity column-2, 406, DNA/RNA/Telomerase, exosomes, nanosomes affinity column-3, 424 and DNA/RNA/Telomerase, exosomes, nanosomes affinity column-4, 442, a series of microfilters for separation of CTC, microfilter for CTC removal from plasma 472P with microfilter plasma CTC elution collection inlet and outlet 474P, microfilter for removal of CTC bound to platelet 478PL, with microfilter platelet CTC elution collection inlet and outlet 480PL, microfilter for removal of CTC bound to WBC 476W with microfilter WBC bound CTC elution collection inlet and outlet 484W, microfilter for removal of CTC bound to RBC concentrate 478R with microfilter RBC bound CTC elution collection inlet and outlet 488R, a series of processed blood components collecting bags, purified plasma collecting bag 398 with purified plasma collection inlet and outlet 476P to remove samples of treated plasma for testing and preservation before its transfusion back to the patient, purified platelets collecting bag 412 with purified platelet collection inlet and outlet 482PL to remove samples of treated platelets for testing and preservation before its transfusion back to the patient, CTC, DNA/RNA/Telomerase, exosome, microsomes and nanosomes free WBC 430 with purified WBC collection inlet and outlet 486W to remove samples of purified WBC for testing and preservation before its transfusion back to the patient, purified RBC collecting bag 448 with purified RBC collection inlet and outlet 490R to remove samples of treated RBC for testing and preservation before its transfusion back to the patient, blood flow tubing 470 which interconnects blood and blood component reservoirs and with the reservoir for DNA/RNA/Telomerase, tumor associated exosome, microsomes, nanosomes and CTC free blood after pulse flow purification 458.

CTC separation by microfiltration is fast and simple. After chemotherapy/radiosurgery large volumes of blood apheresis is processed rapidly to remove CTC, CTC-bound to platelets, exosomes, microsomes and nanosomes and to remove the DNA-DNA fragments and telomere-telomerase. Over 90 percent of CTC can be removed by rapid CTC microfiltration (68). Rapid flow cytometry of the cells sampled by pulse apheresis after chemotherapy/radiosurgery is used to monitor gamma H2AX containing cells as an indices for removal of CTC, tumor associated exosomes, nanosomes, DNA, DNA fragments, telomere-telomerase (69). The blood components are passed thorough affinity chromatograms. Heparin mimics as a DNA binding polyanionic structure nucleic acid (70) Partial purification of DNA binding proteins with HiTrap heparin column is commercially available (71) Cellulose activated charcoal coated with heparin is safely used in hemoperfusion for drug overdose treatment (72). Disposable DNA/RNA/telomerase, exosomes, microsomes and nanosomes binding heparin coated cellulose activated charcoal is used to remove the DNA/RNA/telomerase, exosomes, microsomes and nanosomes surge caused by chemotherapy-radiosurgery and surgery by pulsed flow apheresis. It eliminates and or minimizes the bystander and abscopal effects associated tumor recurrence and metastasis.

Air bubble sensor 452 monitors any air bubbles in the final stretch of the blood flow tubing 470 that connects with the reservoir for DNA/RNA/Telomerase, tumor associated exosome, microsomes, nanosomes and CTC free blood after pulse flow purification 458. If there are air bubbles, they are purged out of the blood flow tubing 470 by opening and closing the system clamps with sensors 464 adjacent to the reservoir for DNA/RNA/Telomerase, tumor associated exosome, microsomes, nanosomes and CTC free blood after pulse flow purification 458. The densitometer-12 454 monitors the treated return blood in blood flow tubing 456. The DNA/RNA/Telomerase, tumor associated exosome, microsomes, nanosomes and CTC free blood after pulse flow purification is transfused back to the patients through blood flow return channel with clam and sensor 462.

After the apheresis of about 300 ml with the first apheresis system is completed the pulse flow apheresis of the circulating tumor cells (CTC), RNA, DNA and DNA fragments, exosomes, microsomes and nanosomes from circulation is resumed with the second set of the pulse flow apheresis system connected to the patient at another site, say to the left arm if the first pulse flow apheresis system was connected to the right arm. Intermittent apheresis with two such systems facilitates a continuous flow aphaeresis of the circulating tumor cells (CTC), RNA, DNA and DNA fragments, exosomes, microsomes and nanosomes from circulation.

Alternative Continuous Flow Aphaeresis

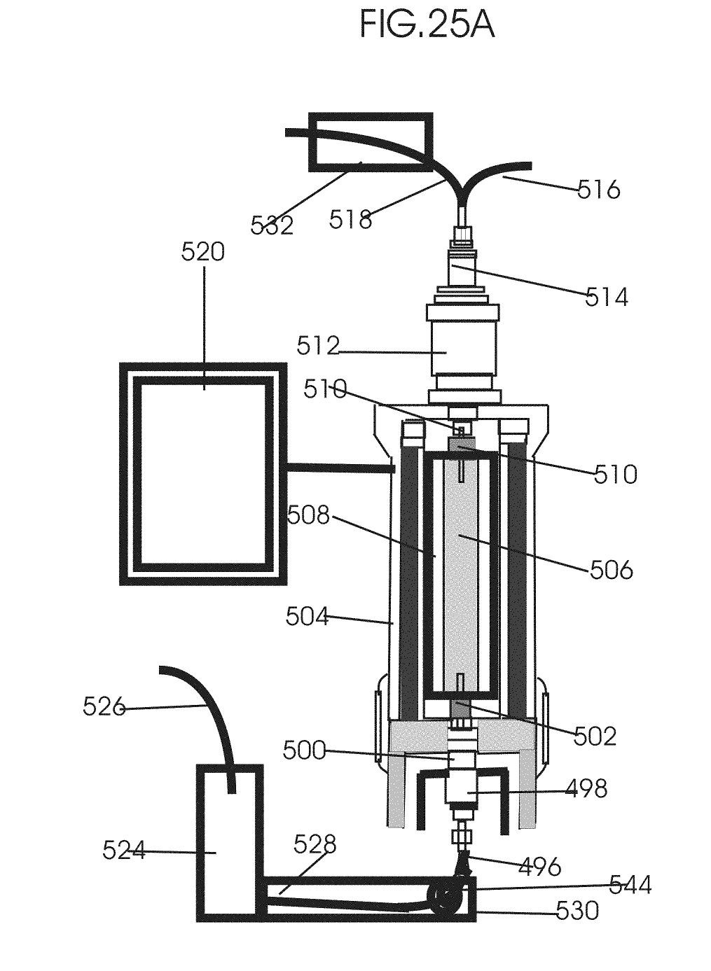

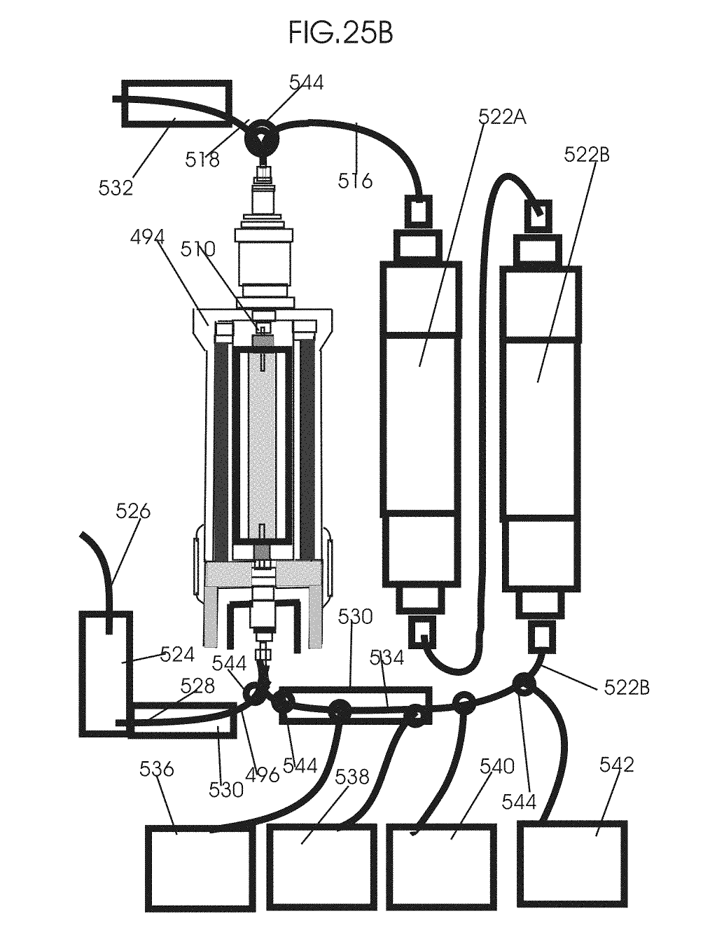

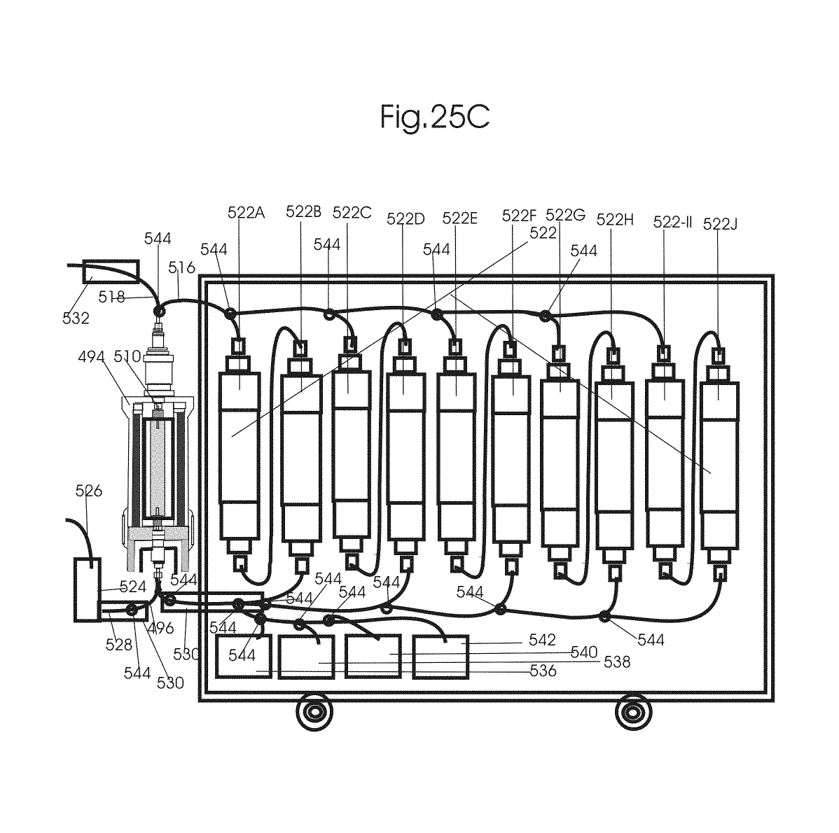

FIG. 25A illustrates a continuous flow ultracentrifuge rotor adapted for plasmapheresis where plasma from the pulsed flow apheresis flows through the bottom inlet of the rotor and separation of the remaining plasma soluble larger micro and nano particle cell debris, cell membranes, normal cell and tumor cell associated proteins, apoptotic bodies, DNA and RNAs, microsomes, exosomes and nanosomes, telomere and telomerase, ATM and ATM kinase after pulsed flow apheresis into a sucrose density gradient solution within the rotor and the plasma free of larger soluble cellular components flows through the outlet at the top of the rotor either towards a series of affinity chromatography columns connected with atomic force microscopy (AFM) combined with nanoparticle tracking analysis (NTA), disc centrifuge nanoparticle analysis (DCNA) and flow cytometry for particle tracking or the purified plasma flowing back to the patient.

Continuous flow ultracentrifuge with continuous flow rotors are generally used to separate micro and nano particles in nanoparticle research and industry. In pharmaceutical industry, they are used to produce vaccines against viral infection. For illustration, such a continuous flow ultracentrifuge rotor described by the Hitachi Koki Co. Ltd is incorporated herein in its entirety (89). Its modified version is described herein to remove remaining nanoparticles after pulse flow apheresis of plasma. Any other modified continuous flow ultracentrifuge and continuous flow rotors could be adapted for additional purification of the plasma from tumor cell derived nanoparticles, exosomes and nanosomes after the pulse flow apheresis. Such continuous flow ultracentrifuges and rotors that could be used in this invention include the Alpha Wassermann continuous flow ultracentrifuge and rotors, Beckman continuous flow ultracentrifuge and rotors the Sorvall continuous flow ultracentrifuge and rotors or any other similar ones.

The pulsed flow apheresis plasma is continuously introduced into the high speed rotating cylindrical rotor 508 through its bottom sample inlet 496. High speed rotating cylindrical rotor 508 is connected to the hollow top driveshaft 510 and to the bottom hollow drive shaft 502 for the sample to pass through and are supported by bearings. The driveshaft at the top is connected to a high frequency motor 512. The mechanical seal at the end of the upper driveshaft 502 and the bottom driveshaft 510 seals the sample from any leaks. The rotating cylindrical rotor 508 rotates at any speeds up to 40,000 rpm/min and up to 100,000 G, that separate the remaining plasma soluble larger micro and nano particle cell debris, cell membranes, normal cell and tumor cell associated proteins, apoptotic bodies, DNA and RNAs, microsomes, exosomes and nanosomes, telomere and telomerase, ATM and ATM kinase. The rotor system is equipped with lift for its insertion into the ultracentrifuge and its removal from it. The operation parameters of the ultracentrifuge with the rotor including electrical, cooling, vacuum and the mechanical seal and status of the motor, are displayed on the control system LCD 520.

For the separation of plasma soluble nanoparticle by sucrose density gradient continuous flow ultracentrifugation, first the sucrose gradient solution consisting of 130 ml phosphate buffered saline, 200 ml 17% (W/W) sucrose (density 1.0675 g/cm²), 130 ml 30% (W/W) sucrose (density 1.1.1268 g/cm²) and 30 ml 45% (W/W) sucrose (density 1.2028 g/cm²) (85) is filled in to the rotor that can hold about 3 L fluid. Any other sucrose density gradient solution that is suitable for the separation of any particular exosomes or nanosomes also could be used. This sucrose gradient solution is filled into the rotor through the bottom hollow driveshaft 502 and the centrifuge is run at 4,000 rpm/min for a few minutes to layer the sucrose gradient solution vertically. It causes the higher concentration sucrose solution to migrate towards the center of the rotor and the lower concentration of sucrose towards the periphery of the rotor forming a density gradient between these two layers. After this density gradient is formed, the pulsed flow apheresis plasma containing soluble micro and nano particle cell debris, cell membranes, normal cell and tumor cell associated proteins, apoptotic bodies, DNA and RNAs, microsomes, exosomes and nanosomes, telomere and telomerase, ATM and ATM kinase is injected into the rotor through the bottom hollow driveshaft 502 at an injection rate of about 5-20 ml/min (when at full speed, about 1 L/h) while the rotor is slowly accelerated to 40,000 rpm/min to facilitate the nanoparticle separation at 100,000 G. Before the injection of the pulse flow apheresis plasma into the rotor, it is chilled to about 0° C. with cooling coils 530 attached to the pulsed flow apheresis plasma injector 528 to avoid plasma coagulation from the heat generated by the rotation of the rotor as an additional precaution to the cooling system attached to the rotor. The slow flow rate of about 1 L/h and high speed rotation of the rotor maintains the sucrose gradient undisturbed (93). The plasma flow rate is reduced if it is clinically warranted. The plasma volume for an adult is about 3 L. (90) It is constantly monitored by bioelectrical impedance analysis (BAL) (91) and maintained at about 3 L total body plasma level with 5% D/0.45 N saline containing supplemental electrolytes like potassium, calcium, magnesium is infused to the patient if needed to maintain electrolytes and fluid balance. A 10 hour continuous plasmapheresis at a rate of about 15-20 ml/min will complete one time complete plasmapheresis of 3 L plasma in about 3 hours. In general when continuous flow centrifuges (not the ultracentrifuge) are used for blood component exchanges, the usual flow rate is 40 ml/min. (92). Since the pulse flow apheresis system is not based on centrifugation, its flow rate is slower. Safe centrifugal apheresis at rate of 50-150 ml/min is in common practice (92B). The average total body plasma volume in an adult patient is about 3 L. Because of the intermingling of the plasma with other body fluid compartments, a one or few times run plasma aphaeresis is not a complete clearance of the plasma soluble nanoparticles in an adult. By continued plasmapheresis for 12 hours, a four time's clearance of the 3 L plasma is achieved. Presence or absence of tumor associated nanoparticle in the plasma is monitored with AFM, NTA and DCNA. According to the size and weight of the nanoparticles in the soluble pulsed flow apheresis plasma, they separate towards the inside of the sucrose gradient solution. At the end of the ultracentrifugation, the speed of the rotor is slowly reduced to 4,000 rpm/min and then slowly brought to stop the rotor rotation and the fractions of the SDG is collected by air injection through the top hollow driveshaft 510.

The continues-flow ultracentrifuge rotor is run at 100,000 g for 12 hrs at 4° C. At the end of this ultracentrifugation, the particles that layers in sucrose density gradient contains most of the larger plasma soluble circulating cell debris, cell membranes, and plasma soluble tumor associated proteins, apoptotic bodies, DNA and RNAs, microsomes, exosomes and nanosomes, telomere and telomerase, ATM and ATM kinase and those derived from normal cells. The supernatant elute without such larger plasma soluble micro and nanoparticles exit from the top hollow driveshaft 510 of the rotor. It is directed to a series of immunoadsorbent columns 522 with magnetic microbeads or Sepharose 2B and coated with selected, patient specific; FDA approved therapeutic monoclonal antibodies shown in Table 3 or with antibodies against putative cancer stem cell antigens shown in Table 1 or with antibodies against differentiated cancer cell antigens shown in Table 2. Selected antigen antibody binding of a patent specific tumor microparticles, cell membranes, plasma soluble tumor associated proteins, apoptotic bodies, DNA and RNAs, microsomes, exosomes and nanosomes, telomere and telomerase, ATM and ATM kinase are monitored with atomic force microscopy (AFM) combined with Nanoparticle Tracking Analysis (NTA), Disc Centrifuge Nanoparticle Analysis (DCNA) and flow cytometry. After several repeated affinity chromatography through a series of interconnected immune affinity chromatography columns, the purified plasma is warmed to 37° C. with a warming coil 532 and such treated plasma is returned back to the patient. The continuous flow ultracentrifuge is kept in sterile conditions and environment and the rotor is sterilized online as per manufacturer's instructions and kept sterile and operated in sterile conditions.

FIG. 25B shows the same continuous flow ultracentrifuge rotor adapted for plasmapheresis of the pulsed flow apheresis plasma as illustrated in FIG. 25A but the supernatant exiting from the top hollow driveshaft 510 flows through two affinity chromatography columns coated with patient specific tumor nanosomes antibody and connected with AFM, NTA, DCNA and a flow cytometer (FCM) for particle tracking and the effluent supernatant exiting from the chromatographic columns 534 flows back to the high speed rotating cylindrical rotor 508 through its bottom hollow driveshaft 502 and back to the patient or it re-circulates through a set of two affinity chromatography columns.

For immune affinity bound separation of patient specific plasma soluble micro and nano particle cell debris, cell membranes, normal cell and tumor cell associated proteins, apoptotic bodies, DNA and RNAs, microsomes, exosomes and nanosomes, telomere and telomerase, ATM and ATM kinase, the supernatant exiting from the top hollow driveshaft 510 is directed towards immunoadsorbent affinity chromatography column 1, 522A and to immunoadsorbent affinity chromatography column 2, 522B. They are coated with patient specific tumor nanosomes antibody and connected with AFM 536, NTA 538, and DCNA 540 and to a flow cytometer (FCM) 542 for particle tracking. The effluent supernatant exiting from the chromatographic columns 534 flows back to the high speed rotating cylindrical rotor 508 through its bottom hollow driveshaft 502 and back to the patient or it re-circulates through the immunoadsorbent affinity chromatography column 1 and the immunoadsorbent affinity chromatography column 2 through the supernatant outlet 516. Before the effluent supernatant exiting from the chromatographic columns 534 is injected back into the high speed rotating cylindrical rotor 508, it is cooled to 0° C. with the cooling coil 530. The supernatant flow into the rotor, out of the rotor, into the immunoadsorbent affinity chromatography columns, into AFM, NTA, DCNA and FCM and back to the patient is controlled by the electronic flow direction control switch 544. Before the effluent supernatant exiting from the high speed rotating cylindrical rotor 508 is returned back to patient, it is warmed to w37° C. with the warming coil 532. Before the pulsed flow apheresis plasma is injected into the high speed rotating cylindrical rotor 508 through its bottom hollow driveshaft 502 for nanoparticles separation, it is cooled to 0° C. with the cooling coil 530. The immunoadsorbent affinity chromatography columns are sterilized and kept in a sterile environment. The continuous flow ultracentrifuge is also kept in sterile condition and environment and the rotor is sterilized online as per manufacturer's instructions and kept sterile and operated in sterile conditions.

FIG. 25C illustrates the same continuous flow ultracentrifuge rotor adapted for plasmapheresis of the pulsed flow apheresis plasma as in FIG. 25A and FIG. 25B but the supernatant exiting from the top hollow driveshaft 510 flows through a series of affinity chromatography columns coated with patient specific tumor nanosomes antibody with nanosomes monitoring with AFM, NTA, DCNA and FCM and the effluent purified supernatant from the chromatographic columns 534 flows back to the high speed rotating cylindrical rotor 508 through its bottom hollow driveshaft 502 and back to the patient or it re-circulates through the series of affinity chromatography columns that adsorbs the plasma soluble micro and nano particle cell debris, cell membranes, normal cell and tumor cell associated proteins, apoptotic bodies, DNA and RNAs, microsomes, exosomes and nanosomes, telomere and telomerase, ATM and ATM kinase.

For simultaneous separation of several tumor derived patient specific plasma soluble micro and nano particle cell debris, cell membranes, normal cell and tumor cell associated proteins, apoptotic bodies, DNA and RNAs, and nanosomes, telomere and telomerase, ATM and ATM kinase the supernatant exiting from the top hollow driveshaft 510 is directed towards five pairs of immunoadsorbent affinity chromatography columns, immunoadsorbent affinity chromatography column-1, 522A, immunoadsorbent affinity chromatography column-2 522B and immunoadsorbent affinity chromatography column-3 522C; immunoadsorbent affinity chromatography column-4 522D and immunoadsorbent affinity chromatography column-5 522E; immunoadsorbent affinity chromatography column-6 522F and immunoadsorbent affinity chromatography column-7 522G; immunoadsorbent affinity chromatography column-8 522H and immunoadsorbent affinity chromatography column-9 522-I and immunoadsorbent affinity chromatography column-10 522J. Each pairs of the immunoadsorbent affinity chromatography columns are coated with a patient specific tumor nanosomes antibody. The five pairs of immunoadsorbent affinity chromatography columns shown here is only an example. There are over 60 FDA approved, tumor specific therapeutic antibodies. All of them are arranged as interconnected columns as in this example. Alternatively, a series of patent specific tumor antibody coated sets of immunoadsorbent affinity chromatography columns are interconnected as shown here. They are also connected with AFM 536, NTA 538, and DCNA 540 and to FCM 542 for particle tracking. The effluent supernatant exiting from the chromatographic column 522B, 522D, 522F, 522H and 522J flows back to the high speed rotating cylindrical rotor 508 through its bottom hollow driveshaft 502 and back to the patient or it re-circulates through the immunoadsorbent affinity chromatography columns through the supernatant outlet 516. Before the effluent supernatant exiting from the chromatographic columns is injected back into the high speed rotating cylindrical rotor 508, it is cooled to 0° C. with the cooling coil 530. The supernatant flow into the rotor, out of the rotor, into the immunoadsorbent affinity chromatography columns, into AFM, NTA, DCNA and FCM and back to the patient is controlled by the electronic flow direction control switch 544. Before the effluent supernatant exiting from the high speed rotating cylindrical rotor 508 is returned back to patient, it is warmed to w37° C. with the warming coil 532. Before the pulsed flow apheresis plasma is injected into the high speed rotating cylindrical rotor 508 through its bottom hollow driveshaft 502 for nanoparticles separation, it is cooled to 0° C. with the cooling coil 530. The series of immunoadsorbent affinity chromatography columns are placed in a portable trailer that is sterilized and kept in a sterile environment. The continuous flow ultracentrifuge is also kept in sterile condition and environment and the rotor is sterilized online as per manufacturer's instructions and kept sterile and operated in sterile conditions.

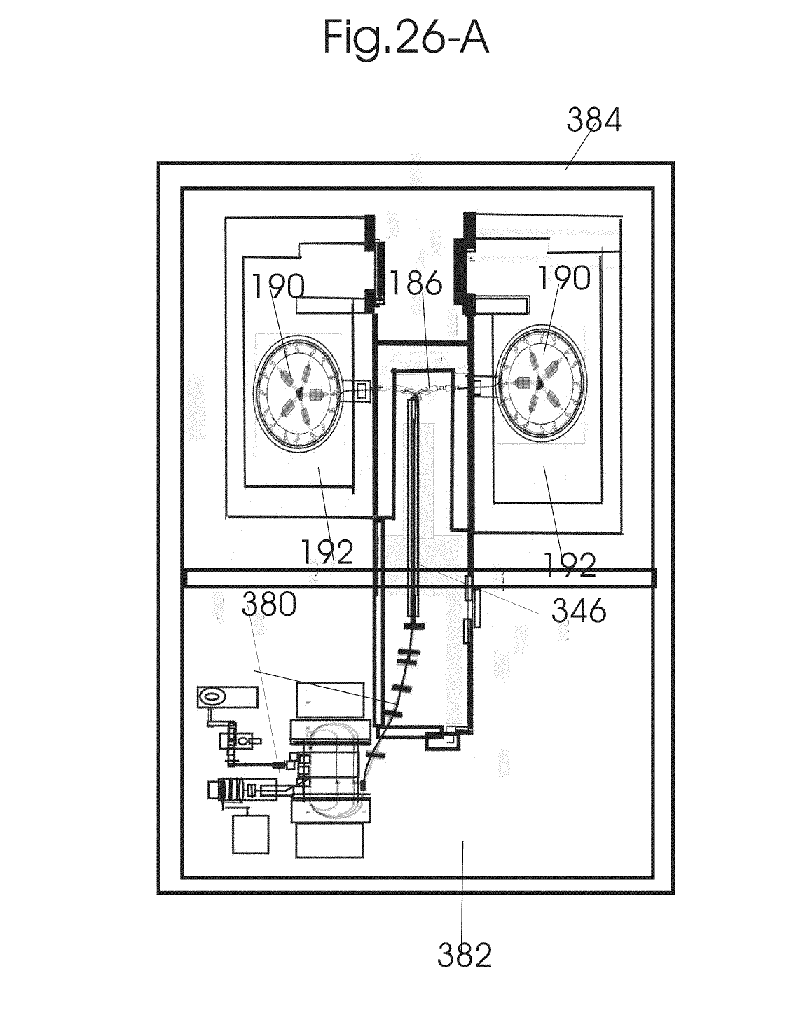

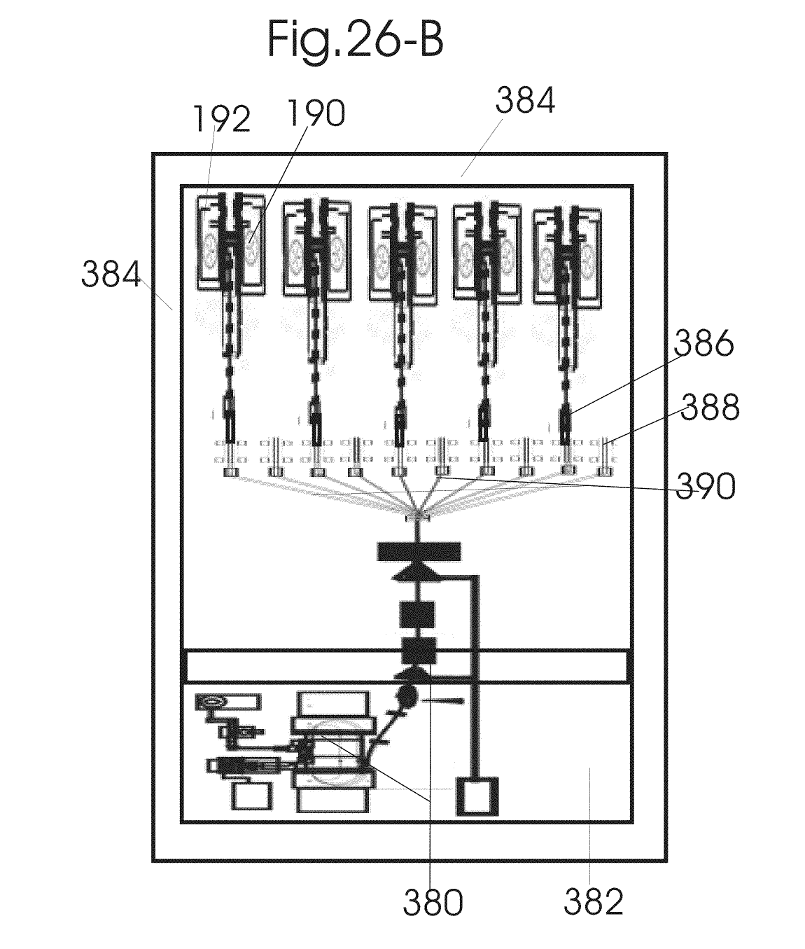

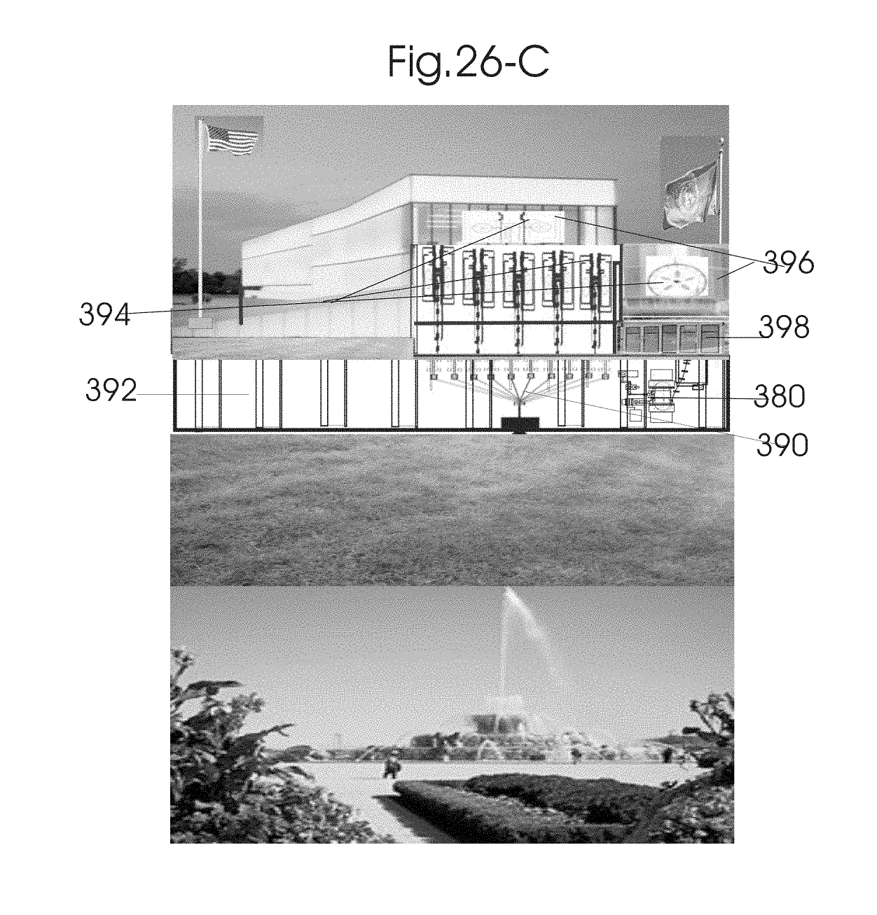

FIG. 26-A shows a photocathode racetrack microtron system 380 with one beamline 346 installed in the basement section of a glass building RT center 382 and its split beamlines attached to a group of five tissue equivalent collimator systems 190 in each of the two adjacent rooms 192 in above the ground glass building RT center 384 for VHEE-beam kGy microbeam, nanobeam or minibeam radiosurgery. FIG. 26-A and in its following figures, FIGS. 26-B and 26-C illustrates the cost-effective housing of a VHEE-beam kGy radiosurgical center in a glass building. To illustrate the tissue equivalent collimator beamline systems 190 from a high energy microtron as enlarged and connected to two treatment rooms is shown in FIG. 26-A. In FIG. 26-B and FIG. 26-C a microtron-laser wakefield accelerator with 10 collinear beam lines and its 5 beamlines connected to the 10 treatment rooms are shown. To minimize the cost of buildings with radiation protection, most radiation therapy centers are built in the basements. Those radiation therapy centers located above the ground, the megavoltage (MV) radiation producing machines containing treatment rooms are built with several foot thick concrete walls. Such constructions are very expensive. Construction alone of an ordinary radiation therapy room with MV accelerator will cost over a million dollars. The cost for the rest of an ordinary megavolt radiation therapy building can reach additional several million dollars. The cost for a proton radiation therapy center ranges from 150 to 200 millions. About 50 to 75 million is spent for the proton accelerator and the rest for the building. Usually, a single megavoltage accelerator can treat only about 20 patients a day. It takes more time to setup and to treat a patient with proton than with the photon. A proton treatment facility can have three beam-3 rooms and might be able to treat about 40 patients a day. Since about 25 to 30 fractionated treatments are given to each patient with photon and or proton, the maximum number of patients that can be treated with a megavoltage machine in year with five days per week treatment regime is limited to about 200 to 250. For a proton treatment facility with 3 rooms and treating 40 patients a day, the number of patients that can be treated in a year is limited to about 350 to 400. Because of the adaptive resistance due to interrupted fractionated overall treatment, most often, they are not EMT-MET cancer stem cell ablative treatment. The radioresistant bacteria, Deinococcus radiodurans, can withstand 12-20 Gy single fraction gamma ray radiation (2). Like the Deinococcus radiodurans, the EMT/MET cancer stemcells are radiodurans. Hence the cancer stem cell radiodurans are not completely ablated by present daily fractionated radiation doses of 1.8 to 2.25 Gy or even with single or fewer fraction 12 to 18 Gy radiosurgery. Hence even the 12 to 20 Gy fewer fractionated radiosurgery is not a curative cancer treatment.

On the other hand, the EMT-MET cancer stem cell ablative, single fraction kGy microbeam radiosurgery is more curative. With super high dose and dose rate, the kGy microbeam radiosurgery lasts only seconds. With VHEE electron multibeam, multi-room simultaneous treatment capabilities as illustrated in this FIG. 22-A, and in figures it follows, in FIG. 22-C, FIG. 22-D, FIG. 26-B and in FIG. 26-C, the daily patient throughput is substantially very high. Using only five of the ten VHEE electron multibeams attached to 10 treatment rooms and allowing an hour for each patient setup and treatments, ten patients could be treated in each room on each day. The actual beam exposure treatment time only seconds. With 10 room simultaneous treatment capability, it is about treating 100 patients a day. It is not like the prolonged, daily fractionated radiation therapy; it is a one time kGy treatment. Taking 250 normal working days in a year, a cancer center with such 10 treatment room setup could treat 25,000 patients a year. It could be doubled if all the 10 beamlines are attached to 20 treatment rooms and all the rooms are utilized. It will reduce the cost of each patient's more curative kGy radiosurgery to a fraction of today's radiation therapy. The present cost of treating a patient with photon IMRT is about 18,000 dollars. The cost of proton radiation therapy to a patient is over 30,000 dollars. Most often they are not curative. The low cost EMT/MET cancer stem cell ablative single fraction kGy radiosurgery on the other hand is more curative. The VHEE electron beam single fraction kGy radiosurgery costs only a very small fraction of the cost of present photon IMRT and the present proton radiation therapy.

With magnetic focusing of VHEE-electron beam, its dₘₐₓ is brought much deeper to the skin (17, 18). The magnetically focused, 100 to 300 MeV electron beam have almost similar depth dose as the 100 to 250 MeV proton beam. The cost for a 100 to 300 MeV modified wakefield electron accelerator described in this invention is far below the cost of a proton machine. Because of the much reduced radiation protective shielding requirements for a VHE electron beam treatment facility, its construction is much cheaper. It can be housed in a high density glass building. The high density glass manufacturing like those described in this invention is ideally suited for the construction of beautiful glass buildings to house a modern radiation therapy, cancer treatment center. The density of chromium-rice husk ash is about 2.6 and the density of gadolinium-rice husk ash glass is about 5. Areas requiring more radiation protection from VHEE beam and the neutron that it could produce are protected with such high density glass. The areas the do not require shielding from radiation is built either with glass that are ordinarily used for building or with artistic chromium-rice husk ash glass with its deep emerald green ornamental color. It gives an overall beautiful appearance to the building and its environments. It kindles hope and a psychological stimulus to the patients than when they are surrounded in a basement or a concrete encased room. Generally, cancer patients with psychologically adjusted hopes and forward looking do much better than those with a depressive outlook; there is bio-endocrine stimulus that leads to such outcomes. It is further illustrated in FIG. 24-C.

FIG. 26-B illustrates a photocathode racetrack microtron laser wakefield accelerator system 380 as described in FIG. 26-A and installed in the basement of a glass building radiation therapy center but with 10 collinear VHEE beamlines and five of those beamlines connected to 10 treatment rooms with five tissue equivalent collimator systems 190 in each of the treatment rooms 192 for very high energy electron beam kGy microbeam, nanobeam or minibeam radiosurgery.

Features of the glass building radiation therapy center and the advantages of multibeam-multi-room radiosurgery is described in FIG. 24-A. In FIG. 24A, a single beam is shown as split into two and attached to five tissue equivalent collimator system 190 and such collimator system installed in each of the two radiation therapy rooms 192. In this FIG. 24-B, all the collinear 10 VHEE-beamlines 390 from the photocathode racetrack microtron laser wakefield accelerator system 380 is shown as installed in the basement section of the glass building RT center. Alternate collinear VHEE beam lines are connected to beam transport lines 386 with connection to two adjacent radiation therapy rooms in above the ground glass building RT center 384. The alternate unconnected VHEE-beam lines 388 are left for future expansion with additional rooms. As described in FIG. 24-A, this 10 room radiation therapy center connected with VHEE beamlines from the photocathode racetrack microtron laser wakefield accelerator system 380 and attached to five tissue equivalent collimator system 190 is capable of rendering more curative EMT/MET cancer stem cell ablative kGy microbeam or nanobeam or minibeam radiosurgery to 100 patients a day or about 2,500 patients a year at very low cost. Furthermore, this treatment is given in a beautiful environment created by the artistic glass building to kindle hope and psychological will to overcome the trauma of being unfortunate to fight with cancer. It is more emphasized in next FIG. 24-C with full illustration of the glass building RT center.

FIG. 26-C Shows the general view of the glass building radiation therapy cancer treatment center described in FIG. 24-A and FIG. 24-B with the photocathode racetrack microtron laser wakefield accelerator system in the basement and the radiosurgical rooms in the building is visible through front exposed radiation shielding glass panels.

The interior details of the glass building radiation therapy cancer treatment center are the same as those described in FIG. 26-A and FIG. 26-B. The building is in a pleasant well exposed environment surrounded by wide green parks and waterfront fountain. The basement of the building is equipped with the photocathode racetrack microtron laser wakefield accelerator system 380 with collinear 10 VHEE-beamlines 390. It is visible through a see through glass walkway 392. The radiosurgical rooms with tissue equivalent collimators 394 are visible through exposed glass panels in the front of the building. Special radiosurgical rooms 396 are visible through the glass panels in the second floor and in the building section above the entrance doors 398 of the building.

It is quite a contrast to the present basement cancer centers and cancer centers with rooms encased in concrete blocks. It is an artistic building with functional high density radiation shielding, deep emerald green chromium rice husk glass, leather industry waste chromium ash-rice husk glass and gadolinium-rice husk glass. Its technological advancements for cancer treatment without adaptive resistance are unparallel. It is more affordable than the present photon and proton radiation therapy. Its capabilities for more curative cancer stem cell ablative kGy microbeam, nanobeam or minibeam radiosurgery is combined with its artistic and esthetic appearances. It kindles hope and leads to will to fight against cancer and its psychological traumas.

### 30. METHODS OF CHEMO-RADIODURANS CANCER STEMCELLS TOTAL ABLATION WITH kGy MICROBEAM, NANOBEAM OR MINIBEAM IN THIS INVENTION

All the segments of the radiosurgical laser Wakefield accelerator is constantly monitored electronically with safety measures for the whole system shutdown if any of the system components malfunctions. Patient is setup for radiosurgery in any of the multiple radiosurgical room for all field simultaneous kGy radiosurgery lasting only a few seconds with all the precautions for patient immobilization and precise delivery of radiation to the tumor. The isocenter for the interlaced parallel very high energy electron or gamma ray or proton beam microbeam or nanobeam or minibeam that correlates with the isocenter in a patient's tumor site is predetermined before the day of radiosurgery by image guided treatment simulation. This isocentric correlation with the tumor site in the patient lying on treatment table is verified with additional imaging as the patient is immobilized and placed on the treatment table. Laser wakefield accelerator system produces collinear multiple simultaneous beams. These beam's isocentric path is verified by both non-radiative light field setup checks and by treatment setup checks with a few cGy verification films as in routine radiation therapy procedures. Room background radiation both for photon and neutron radiation are determined with a series of silica optical fiber dosimeters installed in treatment rooms, its corridors and in adjacent office areas as described under FIG. 1B, Fig.B-1, FIG. 1C-1 and FIG. 19. Similar readings are taken immediately after the treatment and 15 min later to check the presence of any secondary radiation generated by the kGy radiosurgery in these areas. The split laser wakefield accelerated beams are processed in the tissue equivalent primary collimator to generate microbeam, nanobeam or minibeam and to absorb the secondary radiations including the gamma radiation and the neutron radiation. The magnetically focused microbeam, nanobeam and minibeam's dmax is determined each day before the treatment starts for the day. The combined very high energy electron beam's ability for deep tissue penetration below the skin and the magnetic focusing of the electron beam for its deeper tissue penetration than the lower dose electron beam renders the very high energy electron beam nearly like the pencil proton beam. Such proton beam like depth dose in phantom is determined by spot checking for magnetically focused very high energy electron beam for dosimetry daily for the dosimetric calculations. The macrobeam, nanobeam and minibeams surface dose, spacing and beam penumbra in the tissue equivalent collimator is determined weekly and monthly by film dosimetry. All these readings are recorded as part of radiation safety measures for each day.

Based upon the elected width of microbeam or nanobeam such as 10 μm, 75 μm, or 500 nm, the width between the two microbeams or nanobeams transport tubes in the tissue equivalent collimator is set as 40 μm, 300 μm or 2,000 nm. A beam width to valley distance is kept 1:4 ratios. If the treatment mode is minibeam setup and minibeam width is 300 μm, then the distance between the two minibeams is set as 1 mm to keep the valley distance close to 1:4 ratios but not to exceed more than 1 mm. The treatment room for a patient is selected based upon such microbeam, nanobeam or minibeam widths ratios and the room that is equipped with such ratios tissue equivalent collimators.

Patient specific field defining high density tissue equivalent block is made with the aid of image guided treatment simulation for each patient. The method of Cerrobend block making but with tissue equivalent material is made for each patient. As described under FIG. 1-C-1, the patient specific field shaping block 55 has three sections, an inner section made of metal incorporated silicon compound, an intermediate section made of polyethylene incorporated Styrofoam and an outer section made of Cerrobend. The inner section absorbs most of the secondary neutrons and gamma rays.

It is cut out of high density tissue equivalent glass composition by hotwire cutting similar to hotwire cutting of Styrofoam to make Cerrobend block. Field shaping block is cut out of a block of high density tissue equivalent glass composition like chromium ash-rice husk charcoal glass or Gd silicate-rice husk charcoal glass described under Fig.D-2. First the melted glass composition is cooled to 550° C. As it cools, it is cut in shape like cutting a block of Styrofoam to shape the Cerrobend block. Alternatively, gadolinium-rice husk charcoal glass block is made. It has higher density, about 4.5, and hence forms smaller tissue equivalent neutron absorbing block. In FIG. 1D-2 a high density tissue equivalent glass composition 292 with cut central beam aperture 268 is shown. It absorbs the secondary neutrons and the gamma radiation. Alternative method of high density tissue equivalent incorporated patient specific block making is illustrated in Fig.D1-3 and FIG. 1-D-4. It is part of the methods of routine patient treatment procedures.

The interaction secondary neutrons with hydrogen also generate high energy gamma rays. It is moderated to lower energy gamma rays with the intermediate section of the block, the borated Styrofoam. The moderated gamma ray is absorbed by the outer section Cerrobend in the patient specific block. In preparation for radiosurgery, this patent specific field shaping block 55 is inserted above the primary tissue equivalent collimator 34 and aligned with it and checked for beam alignment with film dosimetry. After the treatment, the patient specific collimator 55 for the just treated patient is removed and the next patient's filed defining, patient specific collimator is inserted in preparation of treating the next patient.

The 100-250 MeV electrons or proton radiation reaching the patient specific field shaping block 55 would generate very low level residual activity in the patient specific field shaping block 55 after radiosurgery. Most of it lasts only for seconds. Since the patient specific filed shaping block 55 is of a single use, disposable block, it is not a major clinical concern. Still it is tested for residual activity and if found to have higher than safe operational level residual activity as defined in radiation safety guidelines, it is kept safely until the residual activity decays before its reuse to make Cerrobend block for another patient. Likewise, other sections of this high density patient specific field shaping block 55 are handled according to the presence or absence of residual activity after exposure to kGy radiation while treating a patient with the treatment beam. Other sections of beam block and beam handling collimator systems are heavily shielded from radioactivity. Still, they are monitored with several dosimeters as shown in FIG. 1B, FIG. 1C and in FIG. 19 to take corrective actions immediately if needed. These are additional radiation safety measures in delivering kGy radiosurgery to a patient and to the routine radiation safety checks performed in a clinical radiation therapy department. The methods of isocentric beam setup for the daily treatment are illustrated in FIG. 2, FIG. 3, FIG. 5, and FIG. 7.

The present customary methods of field shaping with MLC are not ideal for kGy microbeam radiosurgery since it generates high levels secondary neutrons and gamma radiation. Still it is adapted with modified methods of using MLC as shown in FIG. 9. In this instance, the MLC is surrounded by a high density tissue equivalent block-2 55-B. The scattered radiation and the gamma radiation and the nuclear reactions associated, MLC generated neutron are absorbed by the MLC shielding Cerrobend cover 55-B containing high density tissue equivalent glass composition, borated Styrofoam and Cerrobend. Cerrobend block incorporated with tissue equivalent high density glass composition and borated Styrofoam (FIG. 10) or MLC cover with tissue equivalent high density glass, borated Styrofoam and Cerrobend layer (FIG. 11) are used when the methods of treatment is lower dose, 1.8 to 2.25 Gy daily fractionated proton beam radiation or 15 to 20 Gy proton beam radiosurgery with adequate protection from secondary neutron and gamma radiation.

In the methods of rotational radiosurgery of a patient's tumor aimed at total ablation of cancer stem cell radiodurans with kGy dose of very high energy laser wakefield electron microbeam, nanobeam or minibeam beam, the patient is setup on a treatment table with the beam centered at the isocentric tumor as illustrated in FIG. 13. The treatment field is defined with the patient specific collimator 55-A. Microbeam, nanobeam, minibeam focusing magnet 104 and high energy of the beam keep the dₘₐₓ dose much below the skin. It avoids excessive skin toxicity from the electron beam. The laser wakefield accelerator is rotated to predetermined degrees as per the treatment plan while the patient is kept as immobilized. The dose rate of the accelerator is adjusted with the rotational speed of the accelerator system. The peak and valley dose differential based normal stem cell regeneration in normal tissue protects the normal tissue from radiation toxicity. It allows safe rotational kGy radiosurgery aimed at total ablation of cancer stem cell radiodurans.

The methods of MRI image guided treatment simulation and kGy radiosurgery is illustrated in FIG. 12. The detail of the MRI system is described under FIG. 12. The patient is setup on the treatment table and treated as described above, under FIG. 13. The tumor size and its depth from the skin surface are determined like in conventional treatment planning simulation.

Other methods of microbeam or nanobeam or minibeam generation for kGy cancer stem cell radiodurans include the pencil beam 14 injection into microbeam or nanobeam or minibeam generating tissue equivalent collimator systems 12 and generating microbeams or nanobeams or minibeams as described under FIG. 15. In this case, the deflected laser wakefield electron or Compton scattering gamma ray with collinear electron beam or proton pencil beam 128 is switched by the beam switching magnet 129 to left switched pencil beam-1, 130 and to right switched pencil beam-2, 132 and they are transported to 90 degree bending beam line 142 and delivers into tissue equivalent collimator systems 12 where microbeam or nanobeam or minibeam are generated. They are focused by the focusing magnet 48. These magnetically focused very high energy microbeam or nanobeam or minibeam converges at the isocentric tumor 52. Such processed electron beam has high penetrating power with dₘₐₓ much below the skin. Hence electron beam kGy radiosurgery has lesser skin toxicity. For each patient's treatment fields are shaped with single use, interchangeable, patient specific collimator 55 as shown in FIG. 3. A similar method of microbeam, nanobeam or minibeam is generated but with the pencil laser Wakefield accelerated stored in a mini storage ring 154 as shown in FIG. 16.

In the methods of laser wakefield microbeam, nanobeam or minibeam radiosurgery with two radiosurgical room setup as illustrated in FIG. 18, the laser wakefield accelerator with dual supersonic gas jet generating electron beam is connected to the bending and splitting magnets 186 and the split beams are steered to two sets of five tissue equivalent collimator systems installed in their respective radiation protective treatment rooms. The patients are setup as immobilized on a treatment table as shown in FIGS. 12 and 13. The methods of removing the secondary neutron and gamma radiation are illustrated in FIG. 19. The machine generated secondary neutron and gamma radiation are absorbed by the tissue equivalent collimators. The laser wakefield accelerator is housed in a separate room 196 that is located in between the adjacent two radiosurgical rooms 192. The secondary neutron and gamma radiation from the wakefield accelerator system's photoneutrons reactions is thus separated from the radiosurgical rooms. It avoids unsafe secondary neutron and gamma radiation from kGy radiosurgery in the treatment rooms. As also shown in FIG. 19, the secondary neutron and gamma radiation in the radiosurgical rooms and its vicinity office spaces are monitored constantly, before, during and after the treatments with silica optical fiber dosimeters 250, 252, 254, 256,258, 260 and 262.

The methods of cancer stem cell radiodurans total ablation by 150 MeV electron beam generated by laser-photocathode-racetrack microtron system instead of the laser wakefield accelerator is shown in FIG. 19. The beam is directly connected to the bending and splitting magnets 186 and the split beams are connected to two sets of five tissue equivalent collimator systems installed in their respective radiation protective treatment rooms 192. The methods of kGy single fraction microbeam, nanobeam or minibeam radiosurgery is as described earlier in FIG. 18 and FIG. 19. Radiosurgery with a similar two radiosurgical room 192 system with a laser-photocathode-racetrack microtron but with a Z-pinch gun to accelerate the beam to 2-3 Gy per second as shown in FIG. 21 is another choice for cancer stem cell radiodurans total ablation by single fraction all fields simultaneous kGy microbeam, nanobeam or minibeam radiosurgery.

The methods of simultaneous radiosurgery in 10 to 20 radiosurgical rooms with five to ten collinear laser wakefield accelerated beams from a single photocathode racetrack microtron laser system combine with a Z-pinch gun or with a dielectric waveguide or a corrugated pipe wave guide as shown in FIG. 22A, FIG. 22C and FIG. 22D is the choice for high quality, less costly cancer stem cell radiodurans total ablation for more curative cancer treatment.

The very high energetic electron beam 334 from the Z-pinch gun or the dielectric waveguide or corrugated pipe waveguide is split into 10 beams and all the ten split beams or the alternate split beams are connected to twenty or ten treatment rooms equipped with high density tissue equivalent secondary neutron and gamma radiation absorbing collimator systems. The collinear energetic electron beam 334 is separated by the laser dump mirror and laser beam absorber 173. The very high energetic electron beam 334 is scanned and split by the scanning magnet 342 to collinear 10 very high energy laser wakefield electron beams with high repetition rate. The scanned beams 344 is kept focused by the focusing magnet 336 and guided towards the beam bending and splitting magnet system 186 through scanned beam guiding tube 346. The beam bending and splitting magnet system 186 splits the beam to right and left and sends them to five simultaneous microbeam, nanobeam or minibeam generating tissue equivalent collimator systems 190 in the adjacent right and left radiation protective treatment rooms 192 where the microbeam, nanobeam or minibeam radiosurgery is performed. If the cancer stem cell radiodurans total ablative kGy radiosurgery is performed in all the 10 or 20 radiosurgical rooms in a centralized cancer center, then the total patient throughput in a day is 100 to 200. Since it not fractionated radiation therapy, in a day 100 to 200 patient's radiosurgery is completed in a single day. With 250 working days in a year, such a centralized cancer treatment facility treats 25,000 to 50,000 patients in a year. With such high number of patient throughput, the equipment cost for the treatment of each patient is reduced to a fraction of today's radiation therapy-radiosurgery.

The methods of byproduct radioactive isotope collection for combined nuclear imaging and kGy microbeam, nanobeam and minibeam radiosurgery is illustrated in FIG. 23. It illustrates the photocathode racetrack microtron system 338 and the TW laser and laser processing system 440 with Z-pinch gun 332 attached to a drift chamber in which the very high energy electron beam (VHEE) passes through deuterium-tritium gas that generates stable collinear accelerated VHEE beam and 2.45 MeV and 14 MeV neutron beam and their separation into VHEE and neutron beam for VHEE electron microbeam radiosurgery, and the high flux, 10¹³ to 10¹⁵ neutron per bunch that generates radioisotopes. It uses the principles of collective acceleration of energetic ions by linear electron beams propagating in low pressure neutral gases (54, 54, and 55). The electron beam is split into multiple collinear VHEE beams for microbeam, nanobeam or minibeam for radiosurgery. The neutron beam is used for radioisotope production. Desired radioisotope precursors are radiated with high flux neutron to generate radioisotopes as a byproduct of the very high energy electron beam produced for radiosurgery. Such precursors for radioisotope production include ⁹⁹Mo, ²⁴Na, ³²P, 82Br, ⁵⁶Mn, ⁶⁴Cu or ¹⁹⁸Au. Presently ⁹⁹Mo and its daughter ⁹⁹mTc are the most commonly used radioisotope in nuclear medicine. There is an acute shortage for these radionuclides. It is an ideal combination of radiation therapy nuclear medicine in a hospital or in a centralized cancer treatment center as described above.

### 31. METHODS OF PRE AND POST BROADBEAM AND MICROBEAM RADIOSURGERY AND CHEMO-RADIOSURGERY AND PULSE FLOW APHAERESIS AND CONTINUOUS FLOW ULTRACENTRIFUGATION COMBINED WITH IMMUNE AFFINITY CHROMATOGRAPHY DETERMINATION OF CIRCULATING CTC, MICRO AND MACRO FRAGMENTS DNA/RNA, TELOMERASE, MICROSOMES AND EXOSOMES

Blood is withdrawn from the patient before the treatment and after the treatment to determine rate of DNA repair, the rate of DNA repair enzymes increase after the treatment and its return to normal level and to determine abscopal and bystander effects in circulating blood cells, including the circulating cancer stemcells, granulocytes, macrophages and platelets. Such measurements are repeated day after the treatment and afterwards as it is needed. In cases of combined radiosurgery and chemotherapy, the chemotherapy before the radiation is administered according to the pre-established protocols. Patient's vital signs are electronically monitored during and immediately after the kGy microbeam, nanobeam or minibeam radiosurgery. Based on each patient's needs, they are pre-medicated against nausea and or any other anticipated clinical needs.

### 32. METHODS OF INHIBITION OF CIRCULATING TELOMERASE INHIBITION AND INCREASING TUMOR CELL APOPTOSIS AFTER BROADBEAM AND MICROBEAM RADIOSURGERY AND CHEMO-RADIOSURGERY WITH EPIGALLOCATECHIN (ECG) COMBINED WITH PULSE FLOW APHAERESIS AND CONTINUOUS FLOW ULTRACENTRIFUGATION AND IMMUNE AFFINITY CHROMATOGRAPHY

Green tea contains a polyphenol epigallocatechin (EGCG) which is known to regulate the telomerase activity in breast cancer cells and cause cellular apoptosis (81). EGCG also inhibits ER and PR positive breast cancer proliferation by its binding to ER-alpha (80).

EGCG in combination with histone deacetylase inhibitor reactivates the ER-alpha in estrogen receptor negative tumors (78,). There is cross talk between ER-alpha and HER-2 (75B) which leads to resistance to anti-estrogen treatments and to anti-tyrosine kinase HER-2. ER directly and indirectly activates EGFR, HER2 and IGFR 1 (75B). Patients with high EGFR receptor do not respond well to treatments and they have a shorter life (in 75B) HER2 monoclonal antibody trastuzumab (Herceptin) is a standard treatment for breast but in course of time it becomes ineffective due to antibody complex buildup against this monoclonal antibody itself. The pulse flow aphaeresis and continuous flow ultracentrifugation combined with immune affinity chromatography described in this invention removes this antibody complex against Herceptin and similar drugs and the EGCG enhances the tumor cell apoptosis.

### 33. METHODS OF CONVERTING ER NEGATIVE BREAST CANCER TO ER POSITIVE BREAST CANCER BY DELIVERY OF HEPARIN BOUND RECEPTOR COMPLEX TO ER NEGATIVE BREAST CANCER IN COMBINATION WITH BROADBEAM AND MICROBEAM RADIOSURGERY AND CHEMO-RADIOSURGERY AND PULSE FLOW APHAERESIS AND CONTINUOUS FLOW ULTRACENTRIFUGATION AND IMMUNE AFFINITY CHROMATOGRAPHY

Heparin binds to EGF like growth factor receptor (HBEGF) that binds to HER receptor (75) and to IGF-I receptor (76). Heparin bound estrogen receptor, progesterone receptor, HBEGF and IGF-I deliver these receptors to receptor positive and negative breast cancer CTC (74) and by fluorescent immunocytohistochemistry (76, 77) Trastuzumab-resistant HER2-dependent breast cancer is sensitive to EGCG. The cross talk between ER-alpha and HER-2 (75B) and the resistance to anti-estrogen treatments and to anti-tyrosine kinase HER-2, EGFR, HER2 and IGFR 1 (75B), the breast cancer treatment HER2 monoclonal antibody trastuzumab are all revised with pulse flow exosome aphaeresis and continuous flow ultracentrifugation exosome apheresis combined with immune affinity chromatography. Conversion of estrogen receptor negative tumors to estrogen receptor positive tumors changes therapeutic options for the worst kind of breast cancer substantially.

### 34. METHODS OF CIRCULATING CTC, MONONUCLEAR WHITE BLOOD CELLS AND PLATELETS CARRYING TUMOR SPECIFIC EXOSOMES, MICRO AND MACRO FRAGMENTS DNA/RNA, TELOMERASE, MICROSOMES AND EXOSOMES REMOVAL AFTER BROADBEAM AND MICROBEAM RADIOSURGERY AND CHEMO-RADIOSURGERY BY PULSE FLOW APHAERESIS COMBINED WITH IMMUNE AFFINITY CHROMATOGRAPHY

After broad beam or kGy radiosurgery and chemo-radiosurgery, the CTC, mononuclear white blood cells and platelets carrying tumor specific exosomes, circulating RNA, DNA and DNA fragments, exosomes, microsomes and nanosomes are removed from circulation first by pulsed flow apheresis combined with immune affinity chromatography followed by continuous flow ultracentrifugation apheresis. Two intermittent pulse flow apheresis systems are run simultaneously to have a continuous flow apheresis of the exosomes, microsomes nanosomes including highly increased release of telomerase after chemotherapy/radiosurgery. By 15 min gravity sedimentation the RBC, WBC, platelets and plasma are separated. The heavier white blood cells, the red cells and the very bottom circulating tumor cells forms in layers. The plasma with platelets at its bottom collects at the top of the heavier cells. They are separated by gravity differential sedimentation as described in FIG. 24. A series of telomerase affinity columns and a series of microfilters separate and removes the telomerase, CTC, mononuclear white cells and platelets, DNA/RNA/Telomerase, exosome, microsomes and nanosomes from the plasma. Rapid flow cytometry of the cells sampled by pulse apheresis after chemotherapy/radiosurgery is used to monitor gamma H2AX containing cells as an indices for removal of CTC, tumor associated exosomes, nanosomes, DNA, DNA fragments, telomere-telomerase (69). The blood components are also passed thorough affinity chromatograms. Heparin mimics as a DNA binding polyanionic structure nucleic acid (70) Disposable DNA binding proteins with HiTrap heparin column or cellulose activated charcoal coated with heparin is used for these element's separation. Heparin bound receptor complexes are delivered to receptor negative cells like the ER negative breast cancer cells. CTC chromatography with heparin-ER complex affinity columns converts ER negative breast cancer cells to ER positive cells.

### 35. THE METHODS AND STEPS FOR CTC, DNA/DNA FRAGMENTS, TELOMERASE, EXOSOMES AND NANOSOMES REMOVING PULSE FLOW APHERESIS COMBINED WITH CONTINUOUS FLOW ULTRACENTRIFUGATION APHERESIS BEFORE AND AFTER SURGERY, RADIOSURGERY AND CHEMO/RADIOSURGERY

1. Pretreatment CTC assessment by small volume CTC collection with commercially available systems for CTC detection
2. Test for pretreatment classical CTC markers, as positive epithelial adhesion molecule (EpCAM), cytokeratin (CK+) and negative CD-45
3. Test for pretreatment CTC cell surface heparin by cytometry and immunofluorescence (74)
4. Test for pretreatment patient specific cell membrane receptor binding for known tumor specific ligands.
5. Test for patient specific pretreatment CTC bound and circulating tumor antigen exosomes including HER-2, CEA, PSA, Melan A, Mesothelin, Silv and other relevant antigens for immunotherapy
6. Estimate the pretreatment level of stress protein hsp70 and hsp90 as for their interaction with dendritic cells and immunotherapy
7. Estimate the pretreatment CTC's hTERT expression with and without epigallocatechin gallate (EGCG) (81) and calculate its percent inhibition by EGCG.
8. Estimate the pretreatment serum hTERT expression with and without epigallocatechin gallate (EGCG) (81) and calculate its percent inhibition by EGCG.
9. Using siRNA estimate the down-regulated hTERT expression and associated cellular apoptosis in presence and absence of EGCG by PCR (82)
10. In the case of breast cancer CTC, test for its pretreatment estrogen receptor binding to estrogen, progesterone receptor binding to progesterone, heparin binding EGF like growth factor receptor (HBEGF) binding to HER receptor (75) and IGF-I binding to IGF-I receptor (76).
11. In the case of breast cancer, using siRNA estimate the down-regulated hTERT expression and associated cellular apoptosis in ER-positive and ER-negative breast cancer cells by PCR (82).
12. In the case of breast cancer CTC, test for its pretreatment estrogen receptor binding to estrogen and progesterone receptor binding to progesterone and heparin binding EGF like growth factor receptor (HBEGF) binding to HER receptor (75) and IGF-I binding to IGF-I receptor (76).
13. In case of breast cancer CTC determine the pretreatment conversion of ER negative CTCs to ER positive CTCs by incubating ER negative CTCs with heparin bound estrogen receptor, progesterone receptor HBEGF and IGF-I and assess the heparin delivery of these receptors to receptor positive and negative breast cancer CTC by cytometry, immunofluorescence (74) and by fluorescent immunocytohistochemistry (76, 77).
14. In case of breast cancer CTC determine the pretreatment conversion of ER negative CTCs to ER positive CTCs incubating ER negative CTCs with DNA methyltransferase (DNMT) inhibitor 5-aza-25-deoxycytidine (5-aza-dC) and histone deacetylase (HDAC) inhibitor trichostatin A (TSA) that could increase 300-400-fold ER transcript in human ER negative breast cancer cell lines (83) and compare it with the ER negative breast cancer CTC conversion to ER positive breast cancer CTC by EGCG and trichostatin A (TSA) for selective ER alpha conversion with EGCG or TSA in combination with HDAC.
15. In case of breast cancer CTC, pretreatment CTC culture and autoradiography with ³H-Estradiol, ³H-Progesterone, and ³H-EGFR for comparison with pre and post treatment such autoradiograms for induced receptor activities
16. In case of breast cancer, incubate its CTC with heparin bound estrogen receptor, progesterone receptor HBEGF and IGF-I and assess the heparin delivery of these receptors to receptor positive and negative breast cancer CTC by cytometry, immunofluorescence (74) and by fluorescent immunocytohistochemistry (76, 77).
17. In case of breast cancer, incubate its CTCs with epigallocatechin gallate (EGCG) and histone deacetylase inhibitor together for synergetic epigenetic reactivation of estrogen receptor-α, and progesterone receptor negative breast cancer CTC to estrogen receptor-α, and progesterone receptor positive breast cancer CTCs (78, 79, 80) and assess their receptor activities by cytometry, immunofluorescence (74) and by fluorescent immunocytohistochemistry (76, 77).
18. Prepare the intermittent apheresis system one and two by connecting the disposable reservoirs and DNA/RNA/Telomerase, exosomes, nanosomes affinity columns with connecting blood flow tubes 470 and with the diluting NS from 466 and anticoagulants from 468
19. Give instructions to the on the procedure and premedicate the patients as may be needed
20. With flow directed towards the whole blood reservoir 380, slowly draw about 300 ml blood from the patient using the blood flow inlet channel with clam and sensor 460 to the whole blood reservoir 380 stop this blood flow by clamping the clamp with sensors 464A and 464B and let the blood stand for the first and second 15 min gravity sedimentation of the blood elements
21. While waiting to sediment the blood elements in the whole blood reservoir 380 of the first intermittent apheresis system, draw blood from the patient with the second intermittent apheresis system as in step 3 to have a continuous apheresis after the chemo/radiosurgery
22. After blood has sedimented in the whole blood reservoir 380 of the first intermittent apheresis system, connect the upper layer of the whole blood containing CTC, platelets, exosomes, microsomes and nanosomes to the CTC, plasma-platelet and exosomes, microsomes and nanosomes reservoir 386 by running the pulse pump 384 in between the whole blood reservoir 380 and the plasma platelet reservoir 386. Check the hematocrit and viscosity of this fraction with the densitometer-1, 382. Adjust the hematocrit and viscosity with anticoagulant and NS if needed.
23. Let the CTC, platelet, exosomes, microsomes and nanosomes in the plasma platelet reservoir 386 to sediment at its bottom by gravity sedimentation for additional 15 min
24. After the platelets and the CTC in the plasma platelet reservoir 386 has sedimented, direct the upper layer plasma in the plasma platelet reservoir 386 to the DNA/RNA/Telomerase, exosomes, nanosomes affinity column-1 392 with EGCG by running the pulse pump 388 in between the plasma platelet reservoir 386 and the DNA/RNA/Telomerase, exosomes, nanosomes affinity column-1 392 with EGCG and remove the DNA/RNA/Telomerase, exosomes, nanosomes in the plasma fraction by adsorption and affinity binding to DNA/RNA/Telomerase, exosomes, nanosomes affinity column-1 392 with EGCG.
25. Check the HCT and viscosity of the plasma fraction with the densitometer-2, 390 and adjust the HCT and viscosity if needed as described earlier.
26. At the end of the entire apheresis of the patient, remove the DNA/RNA/Telomerase, exosomes, nanosomes binding affinity column-1 392 with EGCG, wash with buffered NS, label it as plasma fraction DNA/DNA fragments, telomerase, exosomes, microsomes and nanosomes and save it for its microscopic analysis and to study its tumor characteristics.
27. Determine the chemo-radiosurgery released CTC in the plasma fraction filtrate for its receptor bindings after heparin-ligand treatment by cytometry and immunofluorescence (74) and by fluorescent immunocytohistochemistry staining (76, 77) to direct post chemo-radiosurgery treatments if needed
28. In the case of breast cancer, determine the chemo-radiosurgery released CTC in the plasma fraction filtrate for conversion of estrogen, progesterone and EGFR negative CTCs into estrogen, progesterone and EGFR positive CTCs after heparin ligand estrogen receptor, heparin ligand progesterone receptor and the heparin ligand EGF like growth factor receptor (HBEGF) binding to HER receptor by immunohistochemistry and immuno fluorescence, by cytometry and immunofluorescence (74,77).
29. Plasma fraction CTC culture and autoradiography with ³H-Estradiol, ³H-Progesterone, and ³H-EGFR and compare with pre and post treatment such autoradiograms for induced receptor activities
30. After the plasma fraction DNA/DNA fragments, telomerase, exosomes, microsomes and nanosomes are removed by their binding to DNA/RNA/Telomerase, exosomes, nanosomes affinity column-1 392 with EGCG, the pulse pump 396 in between the DNA/RNA/Telomerase, exosomes, nanosomes affinity column-1 392 with EGCG and the system clamp with sensors 464 above the microfilter for CTC removal from plasma 472P is activated with flow direction towards the microfilter for CTC removal from plasma 472P which filters out CTC and other floating cellular elements from the plasma.
31. Check the HCT and viscosity of the plasma fraction with the densitometer-3, 394 and adjust the HCT and viscosity if needed as described earlier.
32. Collect samples of plasma fraction with CTC from the microfilter plasma CTC elution collection inlet and outlet 474P for analysis.
33. At the end of the entire apheresis of the patient, remove the accumulated CTC from the microfilter for CTC removal from plasma 472P, wash with buffered NS, and label as plasma fraction CTC and save it for its microscopic analysis and to study its tumor characteristics.
34. After the plasma is free of plasma fraction CTC, the plasma is collected into the purified plasma collecting bag 398 by opening the system clamp with sensors 464 below the microfilter for CTC removal from plasma 472P. The purified plasma fills the purified plasma collecting bag 398.
35. Collect samples of purified plasma through the purified plasma collection inlet and outlet 476P for testing and future use or it is let to flow towards the reservoir for DNA/RNA/Telomerase, tumor associated exosome, microsomes, nanosomes and CTC free blood after pulse flow purification 458 and re-infuse back to the patient.
36. After the upper layer of the whole blood containing CTC, platelets, exosomes, microsomes and nanosomes is pumped to processing, plasma-platelet and exosomes, microsomes and nanosomes reservoir 386 by running the pulse pump 384 in between the whole blood reservoir 380 and the plasma platelet reservoir 386. Check the hematocrit and viscosity of this fraction with the densitometer-1, 382. Adjust the hematocrit and viscosity with anticoagulant and NS if needed.
37. Let the CTC, platelet, exosomes, microsomes and nanosomes in the plasma platelet reservoir 386 to sediment at its bottom by gravity sedimentation for additional 15 min
38. After the upper layer plasma with floating CTC in the plasma platelet reservoir 386 has pumped to the DNA/RNA/Telomerase, exosomes nanosomes affinity column-1 392, start processing the sedimented bottom layer of the plasma platelet reservoir 386 simultaneously while the plasma fraction mingled with floating CTC is processed in an another line by closing the system clamps with sensor 464 in between the plasma platelet reservoir 386 and letting its bottom layer with platelet, CTC and other plasma components to flow towards the reservoir with CTC, platelets, exosomes, microsomes and nanosomes/DNA-Telomerase 402 by gravity flow.
39. Check the HCT and viscosity of the plasma fraction with the densitometer-4, 400 and adjust the HCT and viscosity if needed as described earlier.
40. Add NS and or anticoagulant to the reservoir with CTC, platelets, exosomes, microsomes and nanosomes/DNA-Telomerase 402 through the inlet and outlet tube connection 492.
41. Direct the Platelet/CTC/cell elements flow from the reservoir with CTC, platelets, exosomes, microsomes and nanosomes/DNA-Telomerase 402 to DNA/RNA/Telomerase, exosomes, nanosomes affinity column-2 with EGCG 406 by running the pulse pump 404 in between the reservoir with CTC, platelets, exosomes, microsomes and nanosomes/DNA-Telomerase 402 and the DNA/RNA/Telomerase, exosomes, nanosomes affinity column-2 EGCG 406 and remove the DNA/RNA/Telomerase, exosomes, nanosomes in the platelet fraction by adsorption and affinity binding to DNA/RNA/Telomerase, exosomes, nanosomes affinity column-2 with EGCG 406.
42. After the platelet fraction DNA/DNA fragments, telomerase, exosomes, microsomes and nanosomes are removed by their binding to DNA/RNA/Telomerase, exosomes, nanosomes affinity column-2 with EGCG 406, the pulse pump 410 in between the DNA/RNA/Telomerase, exosomes, nanosomes affinity column-2 with EGCG 406 and the system clamp with sensors 464 above the microfilter for CTC removal from platelet 478PL is activated with flow direction towards the microfilter for CTC removal from plasma 478PL which filters out CTC and other floating cellular elements from the platelet fraction.
43. At the end of the entire apheresis of the patient, remove the DNA/RNA/Telomerase, exosomes, nanosomes affinity column-2 with EGCG 406, wash it with buffered NS, label it as platelet fraction DNA/DNA fragments, telomerase, exosomes, microsomes and nanosomes and save it for its microscopic analysis and to study its tumor characteristics.
44. Determine the chemo-radiosurgery released CTC in the platelet fraction filtrate for its receptor bindings after heparin-ligand treatment by cytometry and immunofluorescence (74) and by fluorescent immunocytohistochemistry staining (76, 77) to direct post chemo-radiosurgery treatments if needed.
45. In the case of breast cancer, determine the chemo-radiosurgery released CTC in the platelet fraction filtrate for conversion of estrogen, progesterone and EGFR negative CTCs into estrogen, progesterone and EGFR positive CTCs after heparin ligand estrogen receptor, heparin ligand progesterone receptor and the heparin ligand EGF like growth factor receptor (HBEGF) binding to HER receptor by immunohistochemistry and immuno fluorescence, by cytometry and immunofluorescence (74, 77).
46. Platelet fraction CTC culture and autoradiography with ³H-Estradiol, ³H-Progesterone, and ³H-EGFR and compare with pre and post treatment such autoradiograms for induced receptor activities
47. Check the HCT and viscosity of the plasma fraction with the densitometer-5, 408 and adjust the HCT and viscosity if needed as described earlier.
48. Collect samples of platelet fraction with CTC from the microfilter plasma CTC elution collection inlet and outlet 480PL for analysis.
49. At the end of the entire apheresis of the patient, remove the accumulated CTC from the microfilter for CTC removal from plasma 480PL, wash with buffered NS, and label as plasma fraction CTC and save it for its microscopic analysis and to study its tumor characteristics.
50. After the platelet is free of platelet fraction CTC, the plasma is collected into the purified platelet collecting bag 482PL by opening the system clamp with sensors 464 below the microfilter for CTC removal from platelet 478PL. The purified platelet fills the purified platelet collecting bag 482PL.
51. Collect samples of purified platelet through the purified platelet collection inlet and outlet 482PL for testing and future use or it is let to flow towards the reservoir for DNA/RNA/Telomerase, tumor associated exosome, microsomes, nanosomes and CTC free blood after pulse flow purification 458 and re-infuse back to the patient.
52. After blood has sedimented in the whole blood reservoir 380 of the first intermittent apheresis system and the upper layer plasma is removed to CTC, plasma-platelet and exosomes, microsomes and nanosomes reservoir 386, connect the bottom layer of the whole blood containing RBC, WBC, CTC, exosomes, microsomes and nanosomes to the reservoir for RBC plus WBC and CTC, exosomes, microsomes and nanosomes 418 by running the pulse pump 416 in between the whole blood reservoir 380 and the reservoir for RBC plus WBC and CTC, exosomes, microsomes and nanosomes 418.
53. Check the hematocrit and viscosity of this fraction with the densitometer-6, 414. Adjust the hematocrit and viscosity with anticoagulant and NS if needed.
54. Let the RBC, WBC, CTC, exosomes, microsomes and nanosomes in the reservoir for RBC plus WBC and CTC, exosomes, microsomes and nanosomes 418 to sediment at its bottom by gravity sedimentation for additional 15 min
55. After the RBC, WBC and the CTC in the reservoir for RBC plus WBC and CTC, exosomes, microsomes and nanosomes 418 has sedimented, direct its upper layer containing WBC, CTC, exosomes, microsomes and nanosomes to reservoir with WBC, CTC, exosomes, microsomes and nanosomes/DNA-Telomerase 418B by closing the system clamps with sensors 464 below the whole blood reservoir 380 and after the reservoir for RBC plus WBC and CTC, exosomes, microsomes and nanosomes 418 and by running the pulse pump 422 at the tube line in between the upper region of the reservoir for RBC plus WBC and CTC, exosomes, microsomes and nanosomes 418 and the reservoir with WBC, CTC, exosomes, microsomes and nanosomes/DNA-Telomerase 418B.
56. Add NS and or anticoagulant to the reservoir with WBC, CTC, exosomes, microsomes and nanosomes/DNA-Telomerase 418B through its inlet and outlet tube connection 492.
57. Check the hematocrit and viscosity of this fraction with the densitometer-7, 420. Adjust the hematocrit and viscosity with anticoagulant and NS if needed.
58. Direct the flow of the contents of the reservoir with WBC, CTC, exosomes, microsomes and nanosomes/DNA-Telomerase 418B through its tube connecting to the DNA/RNA/Telomerase, exosomes, nanosomes affinity column-3 with EGCG 424 by running the pulse pump 422 in between the reservoir with WBC, CTC, exosomes, microsomes and nanosomes/DNA-Telomerase 418B and opening the system clamp with sensors 464 adjacent to it to the DNA/RNA/Telomerase, exosomes, nanosomes affinity column-3 with EGCG 424 and remove the DNA/RNA/Telomerase, exosomes, nanosomes in the WBC, CTC, exosomes, microsomes and nanosomes fraction by adsorption and affinity binding to DNA/RNA/Telomerase, exosomes, nanosomes affinity column-3 with EGCG 424.
59. Check the HCT and viscosity of the WBC fraction with the densitometer-8, 426 and adjust the HCT and viscosity if needed as described earlier.
60. After the WBC, CTC, exosome, microsomes and nanosomes fraction's DNA/DNA fragments, telomerase, exosomes, microsomes and nanosomes are removed by their binding to DNA/RNA/Telomerase, exosomes, nanosomes affinity column-3 with EGCG 424, the pulse pump 428 in between the DNA/RNA/Telomerase, exosomes, nanosomes affinity column-3 with EGCG 424 and the system clamp with sensors 464 above the microfilter for CTC removal from WBC 476W is activated with flow direction towards the microfilter for CTC removal from WBC 476W which filters out CTC and other cellular elements from the WBC fraction.
61. At the end of the entire apheresis of the patient, remove the DNA/RNA/Telomerase, exosomes, nanosomes affinity column-3 with EGCG 424, wash with buffered NS, label it as WBC fraction DNA/DNA fragments, telomerase, exosomes, microsomes and nanosomes and save it for its microscopic analysis and to study its tumor characteristics.
62. Determine the chemo-radiosurgery released CTC in this WBC fraction filtrate for its receptor bindings after heparin-ligand treatment by cytometry and immunofluorescence (Ref 74) and by fluorescent immunocytohistochemistry staining (76, 77) to direct post chemo-radiosurgery treatments if needed.
63. In the case of breast cancer, determine the chemo-radiosurgery released CTC in the WBC fraction filtrate for conversion of estrogen, progesterone and EGFR negative CTCs into estrogen, progesterone and EGFR positive CTCs after heparin ligand estrogen receptor, heparin ligand progesterone receptor and the heparin ligand EGF like growth factor receptor (HBEGF) binding to HER receptor by immunohistochemistry and immuno fluorescence, by cytometry and immunofluorescence (74, 77).
64. WBC fraction CTC culture and autoradiography with ³H-Estradiol, ³H-Progesterone, and ³H-EGFR and compare with pre and post treatment such autoradiograms for induced receptor activities
65. Collect samples of WBC fraction with CTC from the microfilter WBC CTC elution collection inlet and outlet 476W for analysis.
66. At the end of the entire apheresis of the patient, remove the accumulated CTC from the microfilter for CTC removal from WBC 476W, wash it with buffered NS, label it as WBC fraction CTC and save it for its microscopic analysis and to study its tumor characteristics.
67. After the WBC is made free of CTC, exosomes, microsomes and nanosomes, the purified WBC is collected into the CTC, DNA/RNA/Telomerase, exosome, microsomes and nanosomes free WBC collecting bag 430 by opening the system clamp with sensors 464 below the microfilter for CTC removal from WBC 476W and it is filled with WBC free of CTC.
68. Collect samples of purified WBC through the purified WBC collection inlet and outlet 486W for testing and future use or it is let to flow towards the reservoir for DNA/RNA/Telomerase, tumor associated exosome, microsomes, nanosomes and CTC free blood after pulse flow purification 458 and re-infuse back to the patient.
69. After the flow of the upper layer containing WBC, CTC, exosomes, microsomes and nanosomes from the reservoir for RBC plus WBC and CTC, exosomes, microsomes and nanosomes 418 to reservoir with WBC, CTC, exosomes, microsomes and nanosomes/DNA-Telomerase 418B has completed, direct its bottom layer containing RBC, CTC, exosomes, microsomes and nanosomes to the reservoir with concentrated RBC, CTC, exosomes, microsomes and nanosomes/DNA-Telomerase 436 by closing the system clamps with sensors 464 below the whole blood reservoir 380 and the system clamps with sensors 464 that controls the blood flow to reservoir with WBC, CTC, exosomes, microsomes and nanosomes/DNA-Telomerase 418B and by running the pulse pump 416 at the tube line in between the reservoir for RBC plus WBC and CTC, exosomes, microsomes and nanosomes 418 and the pulse pump 434 close to reservoir with concentrated RBC, CTC, exosomes, microsomes and nanosomes/DNA-Telomerase 436.
70. Check the hematocrit and viscosity of this fraction with the densitometer-9 432. Adjust the hematocrit and viscosity with anticoagulant and NS if needed.
71. Add NS and or anticoagulant to the reservoir with concentrated RBC, CTC, exosomes, microsomes and nanosomes/DNA-Telomerase 436 through its inlet and outlet tube connection 492.
72. Check the hematocrit and viscosity of this fraction with the densitometer-10, 438. Adjust the hematocrit and viscosity with anticoagulant and NS if needed.
73. Direct the flow of the contents of the reservoir with concentrated RBC, CTC, exosomes, microsomes and nanosomes/DNA-Telomerase 436 through its tube connecting to the DNA/RNA/Telomerase, exosomes, nanosomes affinity column-4 442 by running the pulse pump 440 in between the reservoir with concentrated RBC, CTC, exosomes, microsomes and nanosomes/DNA-Telomerase 436 and closing the system clamp with sensors 464 in between the reservoir for RBC plus WBC and CTC, exosomes, microsomes and nanosomes 418 and the reservoir with concentrated RBC, CTC, exosomes, microsomes and nanosomes/DNA-Telomerase 436 and remove the DNA/RNA/Telomerase, exosomes, nanosomes in the concentrated RBC, CTC, exosomes, microsomes and nanosomes fraction by adsorption and affinity binding to DNA/RNA/Telomerase, exosomes, nanosomes affinity column-4 442.
74. Check the HCT and viscosity of the WBC fraction with the densitometer-11, 444 and adjust the HCT and viscosity if needed as described earlier.
75. After the concentrated RBC, CTC, exosome, microsomes and nanosomes fraction's DNA/DNA fragments, telomerase, exosomes, microsomes and nanosomes are removed by their binding to DNA/RNA/Telomerase, exosomes, nanosomes affinity column-4 with EGCG 442, the pulse pump 446 in between the DNA/RNA/Telomerase, exosomes, nanosomes affinity column-4 with EGCG 442 and the microfilter for removal of CTC bound to RBC concentrate 478R is activated and fill the microfilter for removal of CTC bound to RBC concentrate 478R and filter out the CTC fraction contaminating the RBC by microfiltration.
76. At the end of the entire apheresis of the patient, remove the DNA/RNA/Telomerase, exosomes, nanosomes affinity column-4 with EGCG 442, wash it with buffered NS, label it as RBC fraction CTC, DNA/DNA fragments, telomerase, exosomes, microsomes and nanosomes and save it for its microscopic analysis and to study its tumor characteristics.
77. Collect samples of RBC fraction with CTC from the microfilter RBC CTC elution collection inlet and outlet 488R for analysis.
78. At the end of the entire apheresis of the patient, remove the accumulated CTC from the microfilter for removal of CTC bound to RBC concentrate 478R, wash it with buffered NS, label it as RBC fraction CTC and save it for its microscopic analysis and to study its tumor characteristics.
79. Determine the chemo-radiosurgery released CTC in this RBC fraction filtrate for its receptor bindings after heparin-ligand treatment by cytometry and immunofluorescence (74) and by fluorescent immunocytohistochemistry staining (76, 77) to direct post chemo-radiosurgery treatments if needed.
80. In the case of breast cancer, determine the chemo-radiosurgery released CTC in the RBC fraction filtrate for conversion of estrogen, progesterone and EGFR negative CTCs into estrogen, progesterone and EGFR positive CTCs after heparin ligand estrogen receptor, heparin ligand progesterone receptor and the heparin ligand EGF like growth factor receptor (HBEGF) binding to HER receptor by immunohistochemistry and immuno fluorescence, by cytometry and immunofluorescence (74, 77).
81. RBC-fraction CTC culture and autoradiography with ³H-Estradiol, ³H-Progesterone, and ³H-EGFR and compare with pre and post treatment such autoradiograms for induced receptor activities
82. After the RBC is made free of CTC, exosomes, microsomes and nanosomes, the purified RBC is collected into the purified RBC collecting bag 448.
83. Collect samples of purified RBC through the purified RBC collection inlet and outlet 490R for testing and its future use or it is let to flow towards the reservoir for DNA/RNA/Telomerase, tumor associated exosome, microsomes, nanosomes and CTC free blood after pulse flow purification 458 and re-infuse back to the patient.
84. Control the purified plasma flow with system clam and sensors attached to blood flow tubing 470
85. Check for air bubbles with the air bubble sensor 452 and purge the air bubbles if present with the aid of system clamps with sensors 464 that are attached close to air bubble sensor 452.
86. Connect the reservoir for DNA/RNA/Telomerase, tumor associated exosome, microsomes, nanosomes and CTC free blood after pulse flow purification 458 to the patient through the blood flow return channel with clam and sensor 462
87. Proceed with steps 1-58 with the second intermittent apheresis system to create a continuous flow apheresis together with first intermittent apheresis system and a second intermittent apheresis system
88. Posttreatment CTC assessment 72 hours after apheresis by small volume CTC collection with commercially available systems for CTC detection
89. Test for posttreatment classical CTC markers 72 hours after apheresis, as positive epithelial adhesion molecule (EpCAM), cytokeratin (CK+) and negative CD-45
90. 72 hours after apheresis, test for posttreatment CTC cell surface heparin by cytometry and immunofluorescence (Ref. 74)
91. 72 hours after apheresis, test for posttreatment patient specific cell membrane receptor binding for known tumor specific ligands.
92. 72 hours after apheresis test for patient specific posttreatment CTC bound and circulating tumor antigen exosomes including HER-2, CEA, PSA, Melan A, Mesothelin, Silv and other relevant antigens for immunotherapy
93. 72 hours after apheresis Estimate the posttreatment level of stress protein hsp70 and hsp90 as for their interaction with dendritic cells and immunotherapy
94. 72 hours after apheresis Estimate the posttreatment CTC's hTERT expression with and without epigallocatechin gallate (EGCG) (81) and calculate its percent inhibition by EGCG.
95. 72 hours after apheresis Estimate the posttreatment serum hTERT expression with and without epigallocatechin gallate (EGCG) (81) and calculate its percent inhibition by EGCG.
96. 72 hours after the apheresis, using siRNA estimate the posttreatment down-regulated hTERT expression and associated cellular apoptosis in presence and absence of EGCG by PCR (82)
97. In the case of breast cancer CTC, 72 hours after the apheresis, test for posttreatment estrogen receptor binding to estrogen, progesterone receptor binding to progesterone, heparin binding EGF like growth factor receptor (HBEGF) binding to HER receptor (75) and IGF-I binding to IGF-I receptor (76).
98. In the case of breast cancer, 72 hours after the apheresis, using siRNA estimate the posttreatment down-regulated hTERT expression and associated cellular apoptosis in ER-positive and ER-negative breast cancer cells by PCR (82).
99. In the case of breast cancer CTC, 72 hours after the apheresis test for posttreatment estrogen receptor binding to estrogen and progesterone receptor binding to progesterone and heparin binding EGF like growth factor receptor (HBEGF) binding to HER receptor (75) and IGF-I binding to IGF-I receptor (76).
100. In case of breast cancer CTC, 72 hours after apheresis culture posttreatment CTC and repeat comparative autoradiography with ³H-Estradiol, ³H-Progesterone, and ³H-EGFR for comparison with pre and post treatment such autoradiograms for induced receptor activities
101. In case of breast cancer, 72 hours after the apheresis incubate the CTCs with heparin bound estrogen receptor, progesterone receptor HBEGF and IGF-I and assess the heparin delivery of these receptors to receptor positive and negative breast cancer CTC by cytometry, immunofluorescence (74) and by fluorescent immunocytohistochemistry (76, 77).
102. In case of breast cancer, 72 hours after apheresis, incubate the CTCs with epigallocatechin gallate (EGCG) and histone deacetylase inhibitor together for synergetic epigenetic reactivation of estrogen receptor-α, and progesterone receptor negative breast cancer CTC to estrogen receptor-α, and progesterone receptor positive breast cancer CTCs (78, 79, 80) and assess the comparative pre and post apheresis receptor activities by cytometry, immunofluorescence (74) and by fluorescent immunocytohistochemistry (76, 77).
103. In case of breast cancer, 72 hours after apheresis, incubate the CTCs with DNA methyltransferase (DNMT) inhibitor 5-aza-25-deoxycytidine (5-aza-dC) and histone deacetylase (HDAC) inhibitor trichostatin A (TSA) that could increase 300-400-fold ER transcript in human ER negative breast cancer cell lines (Ref.83) and compare it with the ER negative breast cancer CTC conversion to ER positive breast cancer CTC by EGCG and trichostatin A (TSA) for selective ER alpha conversion with EGCG or TSA in combination with HDAC.
104. A week after apheresis treatment, start maintenance treatment with heparin bound EGCG nanoparticles (84) with weekly follow up testing for serum telomerase activity until it drops to in measurable levels
105. A week after apheresis and heparin bound EGCG nanoparticle and MEM mediated EGCG treatment, if it is found that there are still circulating high levels of tumor associated exosomes, microsomes and nanosomes, proceed with the exosomes, microsomes and nanosomes removing continuous flow ultracentrifugation and remove the remaining tumor associated exosomes, microsomes and nanosomes as described in section ultracentrifugation of tumor associated exosomes, microsomes and nanosomes

### 36. METHODS OF CIRCULATING NANOPARTICLES, DNA/RNA, TELOMERASE, EXOSOMES AND NANOSOMES REMOVAL AFTER SURGERY, CONVENTIONAL AND SINGLE FRACTION kGy MICROBEAM RADIOSURGERY AND CHEMOTHERAPY BY CONTINUOUS FLOW ULTRACENTRIFUGATION APHAERESIS COMBINED WITH IMMUNE AFFINITY CHROMATOGRAPHY

Removal of plasma soluble larger micro and nano particle cell debris, cell membranes, normal cell and tumor cell associated proteins, apoptotic bodies, DNA and RNAs, microsomes, exosomes and nanosomes, telomere and telomerase, ATM and ATM kinase after pulsed flow apheresis by continuous flow ultracentrifugation and are described in FIG. 25A, FIG. 25B and in FIG. 25C.

After the pulsed flow apheresis, its plasma containing soluble micro and nanoparticles derived both from tumor cells and the normal cells is injected into a sucrose density gradient solution in the ultracentrifuge rotor as described under FIG. 25A. The pulsed flow apheresis plasma is continuously introduced into the high speed rotating cylindrical rotor 508 through its bottom sample inlet 496. High speed rotating cylindrical rotor 508 is connected to the hollow top driveshaft 510 and to the bottom hollow drive shaft 502 for the sample to pass through. The rotating cylindrical rotor 508 rotates at any speeds up to 40,000 rpm/min and up to about 100,000 G, that separate the remaining plasma soluble larger micro and nano particle cell debris, cell membranes, normal cell and tumor cell associated proteins, apoptotic bodies, DNA and RNAs, microsomes, exosomes and nanosomes, telomere and telomerase, ATM and ATM kinase. The operation parameters of the ultracentrifuge with the rotor including electrical, cooling, vacuum and the mechanical seal and status of the motor, are displayed on the control system LCD 520. A sucrose gradient solution consisting the mixture of 130 ml phosphate buffered saline, 200 ml 17% (W/W) sucrose (density 1.0675 g/cm²), 130 ml 30% (W/W) sucrose (density 1.1.1268 g/cm²) and 30 ml 45% (W/W) sucrose (density 1.2028 g/cm²) is filled in to the rotor through the bottom hollow driveshaft 502 and the centrifuge is run at 4,000 rpm/min for a few minutes to layer the sucrose gradient solution vertically. Any other suitable sucrose gradient solution may be used. It causes the higher concentration sucrose solution to migrate towards the center of the rotor and the lower concentration of sucrose towards the periphery of the rotor forming a density gradient between these two layers. After this density gradient is formed, the pulsed flow apheresis plasma is injected into the rotor through the bottom hollow driveshaft 502 at an injection rate of about 5-20 ml/min (when at full speed, about 1 L/h) while the rotor is slowly accelerated to 40,000 rpm/min to facilitate the nanoparticle separation at 100,000 G. Before the injection of the pulse flow apheresis plasma into the rotor, it is chilled to about 0° C. with cooling coils 530 attached to the pulsed flow apheresis plasma injector 528 to avoid plasma coagulation from the heat generated by the rotation of the rotor as an additional precaution to the cooling system attached to the rotor. The slow flow rate of about 1 L/h and high speed rotation of the rotor maintains the sucrose gradient undisturbed (93). The plasma flow rate is reduced if it is clinically warranted. The plasma volume for an adult is about 3 L. (90) It is constantly monitored by bioelectrical impedance analysis (BAL) (91) and maintained at about 3 L total body plasma level with 5% D/0.45 N saline containing supplemental electrolytes like potassium, calcium, magnesium is infused to the patient if needed to maintain electrolytes and fluid balance. A 10 hour continuous plasmapheresis at a rate of about 15-20 ml/min will complete one time complete plasmapheresis of 3 L plasma in about 3 hours. In general when continuous flow centrifuges (not the ultracentrifuge) are used for blood component exchanges, the usual flow rate is 40 ml/min. (92). The average total body plasma volume in an adult patient is about 3 L. Because of the intermingling of the plasma with other body fluid compartments, it is not a complete clearance of the plasma soluble nanoparticles in an adult. By continuing this plasmapheresis for 12 hours, a four time's clearance of the 3 L plasma is achieved. Presence or absence of tumor associated nanoparticle in the plasma is monitored with AFM, NTA and DCNA. According to the size and weight of the nanoparticles in the soluble pulsed flow apheresis plasma, they separate towards the inside of the sucrose gradient solution. At the end of the ultracentrifugation, the speed of the rotor is slowly reduced to 4,000 rpm/min and then slowly brought to stop the rotor rotation and the fractions of the SDG is collected by air injection through the top hollow driveshaft 510.

The ultracentrifuge continues flow rotor is run at 100,000 g for 12 hrs at 4° C. At the end of this ultracentrifugation, the particles that layers in sucrose density gradient contains most of the larger plasma soluble circulating cell debris, cell membranes, and plasma soluble tumor associated proteins, apoptotic bodies, DNA and RNAs, microsomes, exosomes and nanosomes, telomere and telomerase, ATM and ATM kinase and those derived from normal cells. The supernatant elute without such larger plasma soluble micro and nanoparticles exit from the top hollow driveshaft 510 of the rotor. It is directed to a series of immunoadsorbent columns 522 with magnetic microbeads or Sepharose 2B and coated with selected, patient specific; FDA approved therapeutic monoclonal antibodies shown in Table 3 or with antibodies against putative cancer stem cell antigens shown in Table 1 or with antibodies against differentiated cancer cell antigens shown in Table 2. Selected antigen antibody binding of a patent specific tumor microparticles, cell membranes, plasma soluble tumor associated proteins, apoptotic bodies, DNA and RNAs, microsomes, exosomes and nanosomes, telomere and telomerase, ATM and ATM kinase are monitored with atomic force microscopy (AFM) Combined with Nanoparticle Tracking Analysis (NTA), Disc Centrifuge Nanoparticle Analysis (DCNA) and flow cytometry. After several repeated affinity chromatography through a series of interconnected immune affinity chromatography columns, the purified plasma is warmed to 37° C. with a warming coil 532 and such treated plasma is returned back to the patient. The continuous flow ultracentrifuge is kept in sterile conditions and environment and the rotor is sterilized online as per manufacturer's instructions and kept sterile and operated in sterile conditions.

### 37. SUMMARY

In summary, cancer and cancer stem cell radiodurans are shattered by adaptive resistance inhibiting single fraction kGy radiosurgery. It is combined with systemic single fraction chemotherapy aimed at neutralizing the tumor exosomes and nanosomes released in response to kGy radiosurgery. Alternatively, lower dose, 10-30 Gy single fraction conventional radiosurgery is performed. The billions of larger microsomes, nucleosomes and nanosomes released form the tumor into the circulation in response to kGy radiosurgery or in response to 10-30 Gy radiosurgery is removed by pulse flow sedimentation apheresis. The remaining billions of smaller nucleosomes and nanosomes released form the tumor in response to kGy radiosurgery or conventional 10-30 Gy single fraction radiosurgery is removed by continuous flow ultracentrifugation. It avoids and or minimizes the tumor recurrence and metastasis. The kGy radiosurgery's local inflammatory response initiates the tumor specific systemic tumor immunity which also guards the patient from tumor recurrence and metastasis.

The disclosures of all references cited herein are hereby incorporated herein by reference. Listing of references herein is not intended to be a representation that a complete search of all relevant art has been made, or that no more pertinent art than that listed exists, or that the listed art is material to patentability. Nor should any such representation be inferred.

Thus while this inventor has described what are presently the prescribed embodiments of the present invention, other and further changes and modifications could be made without departing from the scope of the invention and it is intended by this inventor to claim all such changes and modifications. Accordingly, it should be also understood that the present disclosure has been presented for purposes of example rather than limitation, and does not preclude inclusion of such modifications, variations and/or additions to the present subject matter as would be readily apparent to one of ordinary skill in the art.

What is claimed:

1. Apparatus for microbeam and nanobeam radiosurgery and removal of surgery, radiosurgery and chemotherapy released circulating tumor cell, RNA, DNA, exosomes, microsomes and nanosomes comprising:
a. laser wakefield Thompson scattering 50 to 300 MeV electron accelerator;
b. laser wakefield Thompson scattering gamma ray generating accelerator;
c. collinear electron and proton generating laser wakefield accelerator;
d. magnetically focused 50 to 300 MeV laser wakefield electron beam with least penumbra;
e. Microbeam generating collimators;
f. Microbeam generating tissue equivalent, secondary neutron, and gamma radiation absorbing collimators;
g. beamline shield consisting of an inner high neutron cross section metal incorporated silicon compound block, high energy gamma ray moderating borated Styrofoam intermediate block and gamma ray absorbing outer Cerrobend block;
h. treatment room beamline shield consisting of an inner high neutron cross section metal incorporated silicon compound block, high energy gamma ray moderating borated Styrofoam intermediate block and gamma ray absorbing outer Cerrobend block;
i. 90 degree bent laser wakefield Thompson scattering electron beam, collinear Compton scattering gamma ray and proton beam injection into five microbeam, nanobeam and minibeam generating tissue equivalent collimators and generating microbeams, nanobeams and minibeams;
j. neutron absorbing silicon containing metallic blocks;
k. neutron absorbing silicon containing metallic blocks made of silicon containing rice husk melted with chromium and made as glass;
l. high density tissue equivalent neutron absorbing metallic blocks made of chromium bound tannery waste;
m. high density tissue equivalent neutron absorbing metallic blocks made of chromium cobalt hexamine trichloride and triiodide, cobalt hexamine perrhenate, chromium hexamine trichloride and chromium hexamine perrhenate;
n. high density tissue equivalent neutron and gamma ray absorbing metallic blocks made of gadolinium glass;
o. optical fiber dosimeters as secondary neutron and gamma radiation monitors inside and outside the radiosurgery rooms;
p. patient specific high density tissue equivalent disposable bock;
q. patient specific high density tissue equivalent disposable bock made of Styrofoam cuts as in Cerrobend block making;
r. patient specific high density tissue equivalent disposable block with safe operational level residual radioactivity;
s. high density tissue equivalent patient specific field shaping block with a beam guiding inner section, a gamma ray moderating borated Styrofoam block bordering the inner section and a rectangular block that absorbs the gamma ray;
t. semi-permanent patient specific rectangular Cerrobend or lead block attached with borated Styrofoam as high density patient specific field shaping inner block;
u. high density patient specific field shaping block cut out of high density tissue equivalent glass composition by hotwire cutting;
v. high density patient specific field shaping block cut out of high density tissue equivalent glass composition by hotwire cutting surrounded by rectangular borated Styrofoam block for high energy gamma ray moderation and rectangular Cerrobend block for gamma ray absorption;
w. high density patient specific filed shaping block made by pouring molten high density tissue equivalent glass composition into a mold;
x. microbeam, nanobeam and minibeam generating high density tissue equivalent primary collimator with microfocus beam guide, focusing anode, and focusing magnet;
y. multiple simultaneous microbeam generating proton accelerators with a common isocentric point;
z. interlacing parallel microbeam, or nanobeam or minibeam at the isocentric tumor one set from 0 degree and another set from 90 degrees;
aa. parallel laser wakefield Thompson scattering electron or Compton scattering gamma ray with collinear electron beam or proton beams all interlacing at the isocentric tumor;
bb. microbeam or nanobeam or minibeam generation out of spread out laser wakefield Thompson scattering electron or Compton scattering gamma ray with collinear electron beam or proton beam in a cylindrical tissue equivalent primary collimator incorporated with a patient specific collimator;
cc. carbon nanotube's induced magnetism focused spread out beam channeled through a semi-patient specific carbon nanotube pre-collimator;
dd. spread out beam focused in carbon nanotube and channeled through patent specific collimator and tissue equivalent primary collimator and generating microbeam, nanobeam or minibeams;
ee. high density patient specific tissue equivalent collimator with borated Styrofoam moderating MV gamma rays and shaping spread out Brag peak beam before microbeam, nanobeam or minibeam generation in primary tissue equivalent collimator;
ff. multileaf collimator surrounded by neutron moderating Styrofoam block with Cerrobend cover and shaping spread out Brag peak beam before microbeam, nanobeam or minibeam generation in primary tissue equivalent collimator;
gg. daily fractionated proton radiation therapy collimator with patient specific filed shaping block containing high density tissue equivalent secondary neutron absorbing borated Styrofoam for moderating MV gamma rays and surrounded by Cerrobend for absorbing moderated gamma rays;
hh. collimator for 10 to 20 Gy single fraction proton radiosurgery with patient specific filed shaping block containing high density tissue equivalent secondary neutron absorbing borated Styrofoam for moderating MV gamma rays and surrounded by Cerrobend for absorbing moderated gamma rays;
ii. multileaf collimator shield with an inner high density tissue equivalent secondary neutron absorbing block, an intermediate borated Styrofoam block for MV gamma ray moderation and an outer Cerrobend block to absorb moderated gamma rays for daily fractionated proton radiation therapy and 10 to 20 Gy single fraction proton radiosurgery;
jj. a rotating very high energy laser wakefield Thompson scattering electron accelerator combined with a MRI;
kk. a rotating very high energy laser wakefield Thompson scattering electron accelerator combined with a MRI and a microbeam, nanobeam or minibeam generating patient specific and tissue equivalent primary collimator;
ll. a rotating very high energy laser wakefield Thompson scattering electron accelerator combined with a MRI and a microbeam, nanobeam or minibeam generating patient specific collimator and tissue equivalent primary collimator and magnetically focused electron beam with deeper maximum depth dose;
mm. a rotating very high energy laser wakefield Thompson scattering electron accelerator combined with microbeam, nanobeam or minibeam generating patient specific and tissue equivalent primary collimator;
nn. right and left beam switching and steering magnets;
oo. 90 degree beam bending and beam steering magnets for beam steering towards microbeam, nanobeam and minibeam generating tissue equivalent collimator;
pp. 90 degree bent laser wakefield Thompson scattering electron beam, collinear Compton scattering gamma ray and proton beam injection into multiple microbeam, nanobeam and minibeam generating tissue equivalent collimators and generating microbeams, nanobeams and minibeams;
qq. synchronized simultaneous switching and injecting the laser wakefield Thompson scattering electron, Compton scattering gamma ray with collinear electron beam and proton pencil beam from a storage ring into multiple microbeam, nanobeam and minibeam generating tissue equivalent collimators;
rr. very high energy electron laser wakefield microbeam generating accelerator;
ss. 50 to 300 MeV electron microbeam generating with laser wakefield laser accelerator;
tt. 50 to 300 MeV electron microbeam generating with dual composite gas jet laser wakefield accelerator;
uu. right and left bent 50 to 300 MeV laser wakefield electron beam with bending magnets and steering the bent beams to two sets of five tissue equivalent collimators;
vv. steering of the laser wakefield accelerator and dual supersonic gas jet generated electron beam to bending and splitting magnets and steering them to right and left treatment rooms equipped with beam storage rings and synchronized simultaneous switching and injecting the stored beam into multiple microbeam, nanobeam and minibeam generating tissue equivalent collimators;
ww. laser wakefield accelerator and dual supersonic gas jet generated beam steered to two sets of storage rings installed in adjacent right and left treatment rooms and beam injected into two sets of five tissue equivalent collimators and their respective radiation protective treatment rooms and radiation protective floor plan for office, physics, patient care, imaging and research;
xx. a very high energy laser-photocathode-racetrack microtron system with stable electron beam and dose rate of 2-3 Gy per second directly connected to the bending and splitting magnets and the split beams connected to two sets of five tissue equivalent collimators installed in a right and in left radiation protective treatment rooms;
yy. a very high energy laser-photocathode-racetrack microtron system with stable electron beam and dose rate of up to 109 Gy per second connected to a laser wakefield accelerator combined with a Z-pinch gun and connected to two sets of five tissue equivalent collimators installed in a right and in left radiation protective treatment rooms;
zz. a very high energy laser-photocathode-racetrack microtron system with stable electron beam and dose rate of up to 109 Gy per second connected to a laser wakefield accelerator combined with a Z-pinch gun and after separation of its laser beam its very high energy electron beam split to ten beams;
aaa. microtron, laser wakefield accelerator and Z-pinch gun accelerated very high energy electron beam split into ten beams and five alternate split beams steered to ten tissue equivalent collimators installed in ten radiation protective treatment rooms;
bbb. microtron, laser wakefield accelerator and high repetition rate dielectric wave guide accelerated very high energy electron beam split into ten beams and five alternate split beams steered to ten tissue equivalent collimators installed in ten radiation protective treatment rooms;
ccc. microtron, laser wakefield accelerator and corrugated pipe waveguide accelerated very high energy electron beam split into ten beams and five alternate split beams steered to ten tissue equivalent collimators installed in ten radiation protective treatment rooms;
ddd. a very high energy electron beam generating laser-photocathode-racetrack microtron Z-pinch accelerator and its stable electron beam steered into a drift tube chamber filled with puffs of deuterium-tritium gas mixture from a gas pump and generating collinear high energy electron and 2.45 and 14 MeV neutron beams and their separation into very high energy electron beam for very high energy electron microbeam radiosurgery, and high flux neutron for generating radioisotopes;
eee. apparatus for pulse flow aphaeresis for removing radio surgery and radiosurgery combined with chemotherapy released circulating tumor cells, micro RNA, DNA and DNA fragments, exosomes, microsomes and nanosomes;
fff. pulse flow aphaeretic removal of radiosurgery and chemotherapy released circulating tumor cells;
ggg. pulse flow aphaeretic removal of surgery, radiosurgery and chemotherapy released circulating tumor cells combined with microfiltration;
hhh. pulse flow apheresis combined with rapid flow cytometry for monitoring gamma H2AX containing circulating tumor cells, cancer cell DNA, DNA fragments, exosomes, nanosomes and telomere-telomerase after chemotherapy/radio surgery;
iii. pulse flow apheresis combined with immune affinity columns for removal of surgery, radiosurgery and chemotherapy released circulating DNA/RNA/telomerase, exosomes, microsomes and nanosomes;
jjj. pulse flow apheresis combined with affinity chromatography with heparin coated cellulose activated charcoal for removal of surgery, radiosurgery and chemotherapy released circulating DNA/RNA/telomerase, exosomes, microsomes and nanosomes;
kkk intermittent dual pulse flow apheresis as continuous flow apheresis of circulating tumor cells, RNA, DNA and DNA fragments, exosomes, microsomes;
lll. a continuous flow ultracentrifuge for plasmapheresis for separation of plasma soluble nano particle cell debris, cell membranes, normal cell and tumor cell proteins, apoptotic bodies, DNA and RNAs, exosomes and nanosomes, telomere and telomerase, ATM and ATM kinase after pulsed flow apheresis;
mmm. a continuous flow ultracentrifuge rotor for plasmapheresis for separation of plasma soluble nano particle cell debris, cell membranes, normal cell and tumor cell proteins, apoptotic bodies, DNA and RNAs, exosomes and nanosomes, telomere and telomerase, ATM and ATM kinase after pulsed flow apheresis;
nnn. pulse flow apheresis plasma chilled to 0° C. with cooling coils attached to pulsed flow apheresis plasma injector;
ooo. a continuous flow ultracentrifuge rotor into which plasma from pulsed flow apheresis is injected at flow rate of 15 ml/min through its bottom inlet;
ppp. several cycles of continuous flow ultracentrifugation plasmapheresis of pulse flow plasma from pulsed flow apheresis plasma injector within 12 hour continuous flow ultracentrifugation plasmapheresis with one continuous flow ultracentrifuge;
qqq. several cycles of continuous flow ultracentrifugation plasmapheresis of pulse flow plasma from pulsed flow apheresis plasma injector within less than 12 hours continuous flow ultracentrifugation plasmapheresis with multiple continuous flow ultracentrifuges;
rrr. multiple combined continuous flow ultracentrifugation plasmapheresis of pulse flow plasma from pulsed flow apheresis plasma injector within an hour;
sss. a continuous flow ultracentrifuge rotor in which plasma soluble nano particle cell debris, cell membranes, normal cell and tumor cell exosomes, proteins, apoptotic bodies, DNA and RNAs, microsomes, exosomes and nanosomes, telomere and telomerase, ATM and ATM kinase are separated in a gradient solution;
ttt. a continuous flow ultracentrifuge rotor in which plasma soluble larger micro and nano particle cell debris, cell membranes, normal cell and tumor cell exosomes, proteins, apoptotic bodies, DNA and RNAs, microsomes, exosomes and nanosomes, telomere and telomerase, ATM and ATM kinase separates in center higher concentration gradient solution;
uuu. a continuous flow ultracentrifuge rotor in which plasma soluble small and very small nano particle cell debris, cell membranes, normal cell and tumor cell exosomes, proteins, apoptotic bodies, DNA and RNAs, microsomes, exosomes and nanosomes, telomere and telomerase, ATM and ATM kinase free plasma separates in peripheral lower concentration gradient solution;
vvv. a continuous flow ultracentrifuge rotor in which plasma free of larger soluble cellular components flows through its top outlet towards a series of affinity chromatography columns;
www. pulse flow apheresis combined with continuous flow ultracentrifugation aphaeresis and affinity immune chromatography for removal of blocking antibodies against immune complex against tumor cell;
xxx. pulse flow apheresis combined with continuous flow ultracentrifugation aphaeresis and affinity immune chromatography for removal of blocking antibodies against tumor cell exosome immune complex;
yyy. pulse flow apheresis combined with continuous flow ultracentrifugation aphaeresis and affinity immune chromatography for removal of exosome immune complex causing paraneoplastic syndrome;
zzz. exosome apheresis by pulse flow apheresis combined with continuous flow ultracentrifugation apheresis and affinity immune chromatography for removal of exosome immune complex diseases;
aaaa. exosome aphaeresis combined with continuous flow ultracentrifugation apheresis and affinity immune chromatography for removal of immune complex causing non-cancer diseases;
bbbb. monitoring patient specific tumor microparticles, cell membranes, plasma soluble tumor associated proteins, apoptotic bodies, DNA and RNAs, microsomes, exosomes and nanosomes, telomere and telomerase, ATM and ATM kinase by atomic force microscopy combined with nanoparticle tracking Analysis and disc centrifuge nanoparticle analysis and flow cytometry:
cccc. monitoring of antigen-antibody binding of a patent specific tumor microparticles, cell membranes, plasma soluble tumor associated proteins, apoptotic bodies, DNA and RNAs, microsomes, exosomes and nanosomes, telomere and telomerase, ATM and ATM kinase by atomic force microscopy combined with nanoparticle tracking Analysis and disc centrifuge nanoparticle analysis and flow cytometry;
dddd. monitoring of antigen-antibody binding of a patent specific tumor microparticles, cell membranes, plasma soluble tumor associated proteins, apoptotic bodies, DNA and RNAs, microsomes, exosomes and nanosomes, telomere and telomerase, ATM and ATM kinase by atomic force microscopy combined with nanoparticle tracking Analysis and disc centrifuge nanoparticle analysis and flow cytometry for purified plasma reinfusion back to patient;
eeee. Therapeutic pulse flow apheresis combined with continuous flow ultracentrifugation plasmapheresis to inhibit epithelial mesenchymal transformation by removing microsomes, exosomes and nanosomes bound to white blood cells, platelets and soluble in plasma;
ffff. therapeutic pulse flow apheresis combined with continuous flow ultracentrifugation plasmapheresis to overcome chemotherapy and radiosurgery resistance by removing microsomes, exosomes and nanosomes bound to white blood cells and platelets and soluble in plasma;
gggg. Therapeutic pulse flow apheresis combined with continuous flow ultracentrifugation plasmapheresis treatments for hematology/oncology diseases by removing microsomes, exosomes and nanosomes bound to white blood cells and platelets and soluble in plasma;
hhhh. Therapeutic pulse flow apheresis combined with continuous flow ultracentrifugation plasmapheresis treatments for non-malignant diseases by removing microsomes, exosomes and nanosomes bound to white blood cells and platelets and soluble in plasma;
iiii. A multi-beam kGy radiation therapy center in a cost-effective above ground glass building with a series of radiosurgical rooms;
jjjj. a photocathode racetrack microtron with one beamline installed in basement section of a glass building with its split beamlines attached to a group of five tissue equivalent collimators in each of two adjacent above ground level glass radiosurgical rooms for very high energy electron beam kiloGy microbeam, nanobeam or minibeam radiosurgery;
kkkk above ground level several radiosurgical rooms in a glass building for EMT-MET cancer stem cell ablative, single fraction chemo-kGy microbeam radiosurgery;
llll. a photocathode racetrack microtron combined with a laser wakefield accelerator installed in basement section of a glass building with its split ten simultaneous beams magnetically guided to multiple above ground level glass radiosurgical rooms for daily very high volume kGy radiosurgery;
mmmm. a photocathode racetrack microtron combined with a laser wakefield accelerator installed in basement section of a glass building with its split ten simultaneous beams magnetically guided to multiple above ground level glass radiosurgical rooms for daily very high volume chemo-kGy radiosurgery;

2. Methods of microbeam and nanobeam radiosurgery and removal of surgery, radiosurgery and chemotherapy released circulating tumor cell, RNA, DNA, exosomes, microsomes and nanosomes comprising:
a. methods of radiosurgery with magnetically focused 50 to 300 MeV laser wakefield electron beam with least penumbra;
b. methods of radiosurgery with the aid of microbeam generating tissue equivalent, secondary neutron, and gamma radiation absorbing collimators;
c. methods of radiosurgery with the aid of secondary neutron and gamma ray absorbing beamline shield consisting of an inner high neutron cross section metal incorporated silicon compound block, high energy gamma ray moderating borated Styrofoam intermediate block and gamma ray absorbing outer Cerrobend block;
d. methods of low secondary neutron and gamma ray absorption in with treatment room beamline shield consisting of an inner high neutron cross section metal incorporated silicon compound block, high energy gamma ray moderating borated Styrofoam intermediate block and gamma ray absorbing outer Cerrobend block;
e. methods of 90 degree bent laser wakefield Thompson scattering electron beam, collinear Compton scattering gamma ray and proton beam injection into five microbeam, nanobeam and minibeam generating tissue equivalent collimators and generating microbeams, nanobeams and minibeams for kGy microbeam, nanobeam and minibeam radiosurgery;
f. methods of neutron absorbing silicon containing metallic glass blocks making by silicon containing rice husk melted with chromium;
g. methods of high density tissue equivalent neutron absorbing metallic blocks glass making with melted chromium bound tannery waste;
h. methods of high density tissue equivalent neutron absorbing metallic blocks making by melting chromium cobalt hexamine trichloride and triiodide, cobalt hexamine perrhenate, chromium hexamine trichloride and chromium hexamine perrhenate;
i. methods of high density tissue equivalent neutron and gamma ray absorbing metallic gadolinium glass block making;
j. methods of secondary neutron and gamma radiation monitoring inside and outside the radiosurgery rooms with optical fiber dosimeters ;
k. methods of secondary neutron and gamma radiation absorption with patient specific high density tissue equivalent disposable bocks;
l. methods of patient specific high density tissue equivalent disposable bock making with Styrofoam cuts as in Cerrobend block making;
m. methods of patient specific high density tissue equivalent disposable block making that has safe operational level residual radioactivity within minutes after kGy electron beam radiosurgery;
n. methods of high density tissue equivalent patient specific field shaping block making that has a beam guiding inner section, a gamma ray moderating borated Styrofoam block bordering the inner section and a rectangular block for absorbing gamma ray;
o. methods of semi-permanent block making with rectangular Cerrobend or lead block attached with borated Styrofoam as high density patient specific field shaping inner block;
p. methods of high density patient specific field shaping block cut out of high density tissue equivalent glass composition by hotwire cutting;
q. methods of high density patient specific field shaping block cut out of high density tissue equivalent glass composition by hotwire cutting surrounded by rectangular borated Styrofoam block for high energy gamma ray moderation and rectangular Cerrobend block for gamma ray absorption;
r. methods of high density patient specific filed shaping block made by pouring molten high density tissue equivalent glass composition into a mold;
s. methods of microbeam, nanobeam and minibeam generating high density tissue equivalent primary collimator construction with microfocus beam guide, focusing anode, and focusing magnet;
t. methods of multiple simultaneous proton microbeam generation with a common isocentric point;
u. methods of isocentric interlacing parallel microbeam, nanobeam and minibeam generation from 0 degree and 90 degrees;
v. methods of generating isocentric parallel laser wakefield Thompson scattering electron beam and Compton scattering gamma ray from collinear electron beam and gamma ray;
w. methods of microbeam or nanobeam or minibeam generation out of spread out laser wakefield Thompson scattering electron beam and Compton scattering gamma ray from collinear electron beam and gamma ray in a cylindrical tissue equivalent primary collimator incorporated with a patient specific collimator;
x. methods of beam channeling through carbon nanotube pre-collimator;
y. methods of generating microbeam, nanobeam and minibeams from spread out beams by channeling them through carbon nanotube and patent specific tissue equivalent collimator;
z. methods of microbeam, nanobeam or minibeam generation in primary tissue equivalent collimator combined with multileaf collimator and surrounded by neutron moderating Styrofoam block with Cerrobend cover;
aa. methods of synchronized simultaneous switching and injecting the laser wakefield Thompson scattering electron beam, Compton scattering gamma ray with collinear electron beam and proton pencil beam from a storage ring into multiple microbeam, nanobeam and minibeam generating tissue equivalent collimators;
bb. methods of generating collinear electron beam and 2.45 and 14 MeV neutron beams from high energy electron beam in a drift tube chamber filled with puffs of deuterium-tritium gas mixture and their separation into very high energy electron beam for electron microbeam radiosurgery, and high flux neutron for generating radioisotopes;
cc. methods of removing circulating tumor cells, micro RNA, DNA and DNA fragments, exosomes, microsomes and nanosomes released by radiosurgery combined with chemotherapy by pulse flow aphaeresis;
dd. methods of pulse flow aphaeretic removal of radiosurgery and chemotherapy released circulating tumor cells;
ee. pulse flow aphaeretic removal of surgery, radiosurgery and chemotherapy released circulating tumor cells combined with microfiltration;
ff. methods of pulse flow apheresis combined with rapid flow cytometry for monitoring gamma H2AX containing circulating tumor cells, cancer cell DNA, DNA fragments, exosomes, nanosomes and telomere-telomerase after chemotherapy and radiosurgery;
gg. methods of pulse flow apheresis combined with immune affinity absorption for removal of surgery, radiosurgery and chemotherapy released circulating DNA/RNA/telomerase, exosomes, microsomes and nanosomes;
hh. methods of pulse flow apheresis combined with affinity chromatography with heparin coated cellulose activated charcoal for removal of surgery, radiosurgery and chemotherapy released circulating DNA/RNA/telomerase, exosomes, microsomes and nanosomes;
ii. methods of intermittent dual pulse flow apheresis as continuous flow apheresis of circulating tumor cells, RNA, DNA and DNA fragments, exosomes, microsomes;
jj. methods of continuous flow ultracentrifuge plasmapheresis for separation of plasma soluble nano particle cell debris, cell membranes, normal cell and tumor cell proteins, apoptotic bodies, DNA and RNAs, exosomes and nanosomes, telomere and telomerase, ATM and ATM kinase after pulsed flow apheresis;
kk. pulse flow apheresis of plasma chilled to 0° C. with cooling coils attached to pulsed flow apheresis plasma injector;
ll. several cycles of continuous flow ultracentrifugation plasmapheresis of pulse flow plasma from pulsed flow apheresis plasma injector within 12 hour continuous flow ultracentrifugation plasmapheresis with one continuous flow ultracentrifuge;
mm. methods of several cycles of plasmapheresis by continuous flow ultracentrifugation of pulse flow plasma from pulsed flow apheresis collected into a plasma injector;
nn. methods of complete removal of plasma soluble nano particle cell debris, cell membranes, normal cell and tumor cell proteins, apoptotic bodies, DNA and RNAs, exosomes and nanosomes, telomere and telomerase, ATM and ATM kinase in less than 12 hours continuous flow ultracentrifugation plasmapheresis with multiple continuous flow ultracentrifuges;
oo. methods of separation of plasma soluble nanoparticle cell debris, cell membranes, normal cell and tumor cell exosomes, proteins, apoptotic bodies, DNA and RNAs, microsomes and nanosomes, telomere and telomerase, ATM and ATM kinase by density gradient ultracentrifugation in a continuous flow ultracentrifuge rotor;
pp. methods of separating plasma soluble larger micro and nano particle cell debris, cell membranes, normal cell and tumor cell exosomes, proteins, apoptotic bodies, DNA and RNAs, microsomes, exosomes and nanosomes, telomere and telomerase, ATM and ATM kinase in center higher concentration gradient solution in an ultracentrifuge continuous flow rotor by ultracentrifugation;
qq. methods of separating plasma soluble larger micro and nano particle cell debris, cell membranes, normal cell and tumor cell exosomes, proteins, apoptotic bodies, DNA and RNAs, microsomes, exosomes and nanosomes, telomere and telomerase, ATM and ATM kinase in peripheral lower concentration gradient solution in an ultracentrifuge continuous flow rotor by ultracentrifugation;
rr. methods of directing plasma free of larger plasma soluble cellular components through top outlet of a continuous flow ultracentrifuge towards a series of affinity chromatography columns by continuous flow ultracentrifugation in a continuous flow ultracentrifuge rotor;
ss. methods of pulse flow apheresis combined with continuous flow ultracentrifugation aphaeresis and affinity immune chromatography for removal of blocking antibodies against tumor cell immune complexes;
tt. methods of pulse flow apheresis combined with continuous flow ultracentrifugation aphaeresis and affinity immune chromatography for removal of blocking antibodies against tumor cell exosome immune complexes;
uu. methods of pulse flow apheresis combined with continuous flow ultracentrifugation aphaeresis and affinity immune chromatography for removal of exosome immune complex causing paraneoplastic syndrome;
vv. methods of exosome apheresis by pulse flow apheresis combined with continuous flow ultracentrifugation apheresis and affinity immune chromatography for removal of exosome immune complex diseases;
ww. methods of exosome aphaeresis combined with continuous flow ultracentrifugation apheresis and affinity immune chromatography for removal of immune complex causing non-cancer diseases;
xx. methods of monitoring patient specific tumor microparticles, cell membranes, plasma soluble tumor associated proteins, apoptotic bodies, DNA and RNAs, microsomes, exosomes and nanosomes, telomere and telomerase, ATM and ATM kinase by atomic force microscopy combined with nanoparticle tracking analysis and disc centrifuge nanoparticle analysis and flow cytometry:
yy. methods of monitoring of antigen-antibody binding of a patent specific tumor microparticles, cell membranes, plasma soluble tumor associated proteins, apoptotic bodies, DNA and RNAs, microsomes, exosomes and nanosomes, telomere and telomerase, ATM and ATM kinase by atomic force microscopy combined with nanoparticle tracking analysis and disc centrifuge nanoparticle analysis and flow cytometry for purified plasma reinfusion back to patient;
zz. methods of therapeutic pulse flow apheresis combined with continuous flow ultracentrifugation plasmapheresis to inhibit epithelial mesenchymal transformation by removing microsomes, exosomes and nanosomes bound to white blood cells, platelets and soluble in plasma;
aaa. methods of therapeutic pulse flow apheresis combined with continuous flow ultracentrifugation plasmapheresis to overcome chemotherapy and radiosurgery resistance by removing chemotherapy and radiation sensitivity blocking microsomes, exosomes and nanosomes bound to white blood cells and platelets and soluble in plasma;
bbb. methods of therapeutic pulse flow apheresis combined with continuous flow ultracentrifugation plasmapheresis treatments for hematology-oncology diseases by removing microsomes, exosomes and nanosomes bound to white blood cells and platelets and soluble in plasma;
ccc. methods of therapeutic pulse flow apheresis combined with continuous flow ultracentrifugation plasmapheresis treatments for non-malignant diseases by removing microsomes, exosomes and nanosomes bound to white blood cells and platelets and soluble in plasma;
ddd. methods of making high density neutron and gamma ray absorbing metal glass composition for glass buildings by melting high density metal together with organic compounds;
eee. methods of construction of multi-beam kGy radiation therapy centers housed in cost-effective, attractive, above ground glass building with series of radiosurgical rooms for low-cost high quality cancer treatment;
fff. methods of construction of glass building cancer treatment center with housing a very high energy electron beam producing photocathode racetrack microtron with beamlines guiding the beam from basement to above ground glass building and the split beamlines attached to groups of five tissue equivalent collimators in each adjacent radiosurgical rooms for very high energy electron kGy microbeam, nanobeam and minibeam radiosurgery;
ggg. a photocathode racetrack microtron combined with a laser wakefield accelerator installed in basement section of a glass building with its split ten simultaneous beams magnetically guided to multiple above ground level glass radiosurgical rooms for daily high volume kGy radiosurgery;
hhh. methods of inhibition of circulating telomerase and increasing tumor cell apoptosis with epigallocatechin;
iii. methods of inhibition of circulating telomerase and increasing tumor cell apoptosis with epigallocatechin after broadbeam and microbeam radiosurgery and chemo-radiosurgery;
jjj. methods of converting ER negative breast cancer to ER positive breast cancer by delivery of heparin bound receptor complex to ER negative breast cancer;
kkk methods of converting estrogen receptor negative breast cancer to estrogen receptor positive breast cancer by exposing cell membrane by broadbeam and microbeam radiosurgery and delivery of heparin bound estrogen receptor complex to ER negative breast cancer cells;
lll. delivery of heparin bound receptor complex to ER negative breast cancer;
mmm. methods of removing Herceptin resistance by exosome removing pulse flow apheresis combined with continuous flow ultracentrifugation apheresis;
nnn. methods of removing Herceptin resistance by broadbeam and microbeam radiosurgery combined with HER exosome removing pulse flow apheresis and continuous flow ultracentrifuge apheresis;
ooo. methods of converting ER Negative Breast Cancer to ER Positive Breast cancer by delivery of heparin bound receptor complex to ER Negative Breast Cancer in combination with broadbeam and microbeam radiosurgery;
